(19) United States
(12) Patent Application Publication
(10) Pub. No.: US 2025/0340563 A1
(43) Pub. Date: Nov. 6, 2025

(54) FUSED PYRIDAZINE DERIVATIVES AS NLRP3 INHIBITORS

(71) Applicant: Takeda Pharmaceutical Company Limited, Osaka (JP)

(72) Inventors: Mark E. ADAMS, San Diego, CA (US); Jason W. BROWN, Burlington, MA (US); Edcon CHANG, San Diego, CA (US); Mingnam TANG, San Diego, CA (US); Holger MONENSCHEIN, San Diego, CA (US); Kristin SCHLEICHER, San Diego, CA (US); Huikai SUN, San Diego, CA (US); Feng ZHOU, San Diego, CA (US)

(21) Appl. No.: 18/854,298

(22) PCT Filed: Apr. 7, 2023
(86) PCT No.: PCT/IB2023/053561

§ 371 Date: Oct. 4, 2024

Related U.S. Application Data
(60) Provisional application No. 63/328,709, filed on Apr. 7, 2022.

(51) Int. Cl. C07D 491/052; A61K 31/5025

(52) U.S. Cl. CPC C07D 491/052; A61K 31/5025

(57) ABSTRACT

Disclosed are compounds of Formula (I), and pharmaceutically acceptable salts thereof, wherein α, β, m, R⁵, R⁶, R⁷, R⁸, R⁹, R¹⁰, R¹¹, R^{a},R^{b}, X¹, X², X³, X⁴ and X⁸ are defined in the specification. This disclosure also relates to materials and methods for preparing compounds of Formula (I), to pharmaceutical compositions which contain them, and to their use for treating diseases, disorders, and conditions associated with NLRP3.

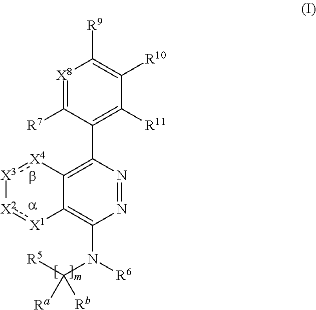

### FIELD OF THE INVENTION

This invention relates to fused pyridazine derivatives, including 1-amino-4-arylphthalazine, azaphthalazine and oxaphthalazine derivatives, which are inhibitors of the NLRP3 inflammasome, to pharmaceutical compositions which contain them, and to their use to treat diseases, disorders, and conditions associated with NLRP3, including neurodegenerative diseases, such as Parkinson's disease, and other diseases, disorders and conditions of the central nervous system (CNS).

### BACKGROUND OF THE INVENTION

More than 1% of the world's population suffers from neurodegenerative diseases, including Parkinson's disease (PD), Alzheimer's disease (AD), Huntington's disease (HD), amyotrophic lateral sclerosis (ALS) and prion disease, all of which lack effective therapies. The incidence of neurodegenerative diseases is expected to double in the coming decades, especially affecting countries with an aging population. See I. Fernindez-Cruz and E. Reynaud, “Proteasome Subunits Involved in Neurodegenerative Diseases,” Arch Med Res. 52(1):1-14 (2021).

One of the pathological hallmarks of neurodegenerative diseases is the aggregation of certain proteins into oligomers or fibrils. These conformational changes result in neurotoxicity, leading to inflammation and neurodegeneration. Although the clinical presentation of these diseases are heterogeneous, they often share common underlying mechanisms and pathophysiologies. See B. N. Dugger and D. W. Dickson, “Pathology of Neurodegenerative Diseases,” Cold Spring Harbor Perspect Biol 9(7):a028035 (2017). Indeed, systemic activation of the innate immune system, which is the first line of host defense against pathogens and tissue injury, and subsequent neuroinflammation play a key role in the onset and the progression of these diseases. See S. Amor, F. Puentes, D. Baker, et al., “Inflammation in neurodegenerative diseases,” Immunology 129(2):154-69 (2010). Neuroinflammation is a physiological response to exogenous and endogenous insults that target the central nervous system (CNS) and represents a protective response in the brain. However, excessive inflammatory responses are detrimental to the CNS. See L. I. Labzin, M. T. Heneka and E. Latz, “Innate Immunity and Neurodegeneration,” Annu Rev Med 69:437-449 (2018).

Microglia, which are myeloid cells of the CNS, play a major role during innate immune responses in the CNS. They express pattern recognition receptors (PRRs) which enable the host to recognize pathogen-associated molecular patterns (PAMPS) and host- or environment-derived danger-associated molecular patterns (DAMPS). See R. M. Ransohoff, M. A. Brown, “Innate immunity in the central nervous system,” J Clin Invest 122(4):1164-71 (2012). PRRs include Toll-like receptors, C-type lectin receptors, RIG-1 like receptors, and nucleotide-binding oligomerization domain-like receptors (NLRs). See P. Broz and V. M. Dixit, “Inflammasomes: mechanism of assembly, regulation and signaling,” Nat Rev Immunol 16(7):407-20 (2016). Engagement of PRRs activates a variety of inflammatory signaling pathways to eliminate infection and repair damaged tissue. The ongoing inflammation found in a variety of neurodegenerative diseases can be maintained by the key innate immune sensor for danger signals, the inflammasomes. There are several different inflammasomes, all defined by the PRRs they contain. Among the PRRs from the NLR family, the NLRs—NLRP1, NLRP3, NLRC4—and two other PRRs—Pyrin and AIM2—are known to form inflammasomes. See D. Zheng, T. Liwinski and E. Elinav, “Inflammasome activation and regulation: toward a better understanding of complex mechanisms,” Cell Discov 6:36 (2020).

The NLRP3 (nucleotide-binding domain (NOD)-, leucine-rich repeats-containing domain (LRR), and pyrin domain-containing 3) inflammasome has been the subject of intense interest in the past decade. See N. Kelley, D. Jeltema, Y. Duan, et al., “The NLRP3 Inflammasome: An Overview of Mechanisms of Activation and Regulation,” Int J Mol Sci 20(13):3328 (2019). The NLRP3 inflammasome consists of three main components: a pattern recognition receptor (PRR) protein, NLRP3; an apoptosis-associated speck-like protein (ASC) containing a caspase activation and recruitment domain (CARD), which functions as a central adaptor protein; and an inflammatory caspase, caspase-1. See Kelley et al. (2019). NLRP3 is comprised of three domains: an amino-terminal pyrin domain (PYD); a central NACHT domain, having ATPase activity that is vital for NLRP3 self-association and oligomerization; and a carboxy-terminal LLR domain. See Broz and Dixit (2016).

The activation of NLRP3 inflammasome involves a two-step process. A first “priming” signal is generated by the detection of PAMPs or DAMPs via TLRs. This priming signal results in NF-κB-dependent transcriptional upregulation of NLRP3 and pro-IL-1, but also controls post-translational modifications of NLRP3. See J. Yang, Z. Liu and T. S. Xiao, “Post-translational regulation of inflammasomes,” Cell Mol Immunol 14(1):65-79 (2017). The initial trigger is followed by a second “activation” signal (β-amyloid, α-synuclein and other proteinaceous insults, ATP, crystals, nucleic acids, toxins) that induces conformational change of the various inflammasome components to subsequently assemble and nucleate the oligomerization of monomeric NLRP3, leading to the formation and activation of the NLRP3 inflammasome. See A. Lu, V. G Magupalli, J. Ruan, et al., “Unified polymerization mechanism for the assembly of ASC-dependent inflammasomes,” Cell 156(6):1193-1206 (2014). This large multimeric protein acts via caspase-1 dependent proteolytic cleavage of several proteins, including pro-interleukin (pro-IL)-18 and pro-IL-10 to their mature inflammatory cytokines, IL-18 and IL-1β. See Kelley et al. (2019). Caspase-1 can also cleave gasdermin D (GSDMD), which facilitates GSDMD's insertion into cellular membranes to form pores, thus initiating a specific kind of cell death called pyroptosis that releases the soluble intracellular fraction which fuels the inflammatory response. See S. L. Fink and B. T Cookson, “Caspase-1-dependent pore formation during pyroptosis leads to osmotic lysis of infected host macrophages,” Cell Microbiol 8(11):1812-25 (2006).

Besides this “canonical” NLRP3 inflammasome activation pathway, a “noncanonical” NLRP3 activation pathway has been described in the literature. The noncanonical pathway involves the activation of caspase-4/5 (or its mouse ortholog caspase-11) by cytosolic LPS, the induction of pyroptosis through the cleavage of GSDMD, and the release of high mobility group box 1 protein (HMGB1), resulting in the production of IL-1β. See M. Lamkanfi and V. M. Dixit, “Mechanisms and functions of inflammasomes,” Cell 157(5):1013-22 (2014); F. Shi, Y. Yang, M. Kouadir M, et al., “Inhibition of phagocytosis and lysosomal acidification suppresses neurotoxic prion peptide-induced NALP3 inflammasome activation in BV2 microglia,” J Neuroimmunol 260(1-2):121-5 (2013). In both pathways, the activation of NLRP3 inflammasome results in the generation of the biologically active form of pro-inflammatory cytokines IL-1β and IL-18 that initiate inflammatory signaling cascades, contributing to neuroinflammation, neuronal injury and cell death. See S. M Allan, P. J. Tyrrell and N. J. Rothwell, “Interleukin-1 and neuronal injury,” Nat Rev Immunol, 5(8):629-40 (2005); A. Alboni, D. Cervia, S. Sugama, et al., “Interleukin 18 in the CNS,” J Neuroinflammation, 7:9 (2010).

Heterozygous gain of function mutations in the NLRP3 gene have been associated with the development of an autoinflammatory condition called cryopyrin-associated periodic syndromes (CAPS). See L. M. Booshehri and H. M. Hoffman, “CAPS and NLRP3,” J Clin Immunol 39(3):277-286 (2019). This is a rare inherited autoinflammatory disorder characterized by systemic, cutaneous, musculoskeletal and central nervous system inflammation, and is estimated to affect about 1 to 3 individuals per million people worldwide. See L. Cuisset, I. Jeru, B. Dumont, et al., “Mutations in the autoinflammatory cryopyrin-associated periodic syndrome gene: epidemiological study and lessons from eight years of genetic analysis in France,” Ann Rheum Dis 70(3):495-9 (2011); Erratum in: Ann Rheum Dis 71(7):1264 (2012). Clinicians classify CAPS disorders based on the severity of symptoms. The most severe form of CAPS is known as neonatal-onset multisystem inflammatory disease (NOMID/CINCA). An intermediate form of CAPS is called Muckle-Wells syndrome (MWS). The familial cold autoinflammatory syndrome (FCAS) is a milder form of CAPS, which is triggered by low temperatures. See Booshehri and Hoffman (2019). Current anti-IL-1 therapies (anakinra, rilonacept, canakinumab) have proven successful in treating CAPS, but clinical experience over the last decade has shown that some CAPS patients are less responsive over time and require higher or more frequent dosing or switching of therapies. See R. Caorsi, L. Lepore, F. Zulian, et al., “The schedule of administration of canakinumab in cryopyrin associated periodic syndrome is driven by the phenotype severity rather than the age,” Arthritis Res Ther 15(1):R33 (2013); S. Urien, C. Bardin, B. Bader-Meunier, et al., “Anakinra pharmacokinetics in children and adolescents with systemic-onset juvenile idiopathic arthritis and autoinflammatory syndromes,” BMC Pharmacol Toxicol 14:40 (2013).

Several small molecule inhibitors have recently been reported that block the NLRP3 inflammasome pathways. These include the prototype NLRP3 inhibitor MCC-950. See R. C. Coll, J. R. Hill, C. J. Day, et al., “MCC950 directly targets the NLRP3 ATP-hydrolysis motif for inflammasome inhibition,” Nat Chem Biol 15(6):556-559 (2019); R. C. Coll, A. A. Robertson, J. J. Chae, et al., “A small-molecule inhibitor of the NLRP3 inflammasome for the treatment of inflammatory diseases,” Nat Med 21(3):248-55 (2015). Other NLRP3 inhibitors include Bay 11-7082, CY-09, oridonin, tranilast, INF-39, glyburide and JC-124. See W. Jiang, M. Li, F. He, et al., “Inhibition of NLRP3 inflammasome attenuates spinal cord injury-induced lung injury in mice,” J Cell Physiol 234(5):6012-6022 (2019). MCC-950 has been used in many studies as a pharmacological tool to demonstrate NLRP3 inflammasome as a viable drug target to development therapeutics for human diseases. See S. E. Corcoran, R. Halai and M. A. Cooper, “Pharmacological Inhibition of the Nod-Like Receptor Family Pyrin Domain Containing 3 Inflammasome with MCC950,” Pharmacol Rev 73(3):968-1000 (2021).

Inhibitors of the NLRP3 inflammasome pathways are expected to be useful for treating neurodegenerative diseases, including Parkinson's disease, and for treating CAPS disorders associated with heterozygous gain of function mutations in the NLRP3 gene.

### SUMMARY OF THE INVENTION

This invention provides fused pyridazine derivatives, including 1-amino-4-arylphthalazine, azaphthalazine and oxaphthalazine derivatives, and pharmaceutically acceptable salts thereof. This invention also provides pharmaceutical compositions that contain the fused pyridazine derivatives and provides for their use to treat diseases, disorders and conditions associated with NLRP3, including Parkinson's disease and other neurodegenerative disorders of the central nervous system.

One aspect of the invention provides a compound of Formula 1:

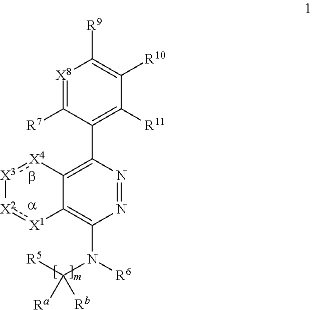

or a pharmaceutically acceptable salt thereof in which:
(A) α is a double bond and β is a double bond;
   X¹ is selected from N and CR¹;
   X² is selected from N and CR²;
   X³ is selected from N and CR³;
   X⁴ is selected from N and CR⁴; provided no more than one of X¹, X², X³ and X⁴ is N; and
   R¹, R², R³ and R⁴ are each independently selected from:
      (i) hydrogen, halo, hydroxy, cyano; and
      (ii) C₁₋₄ alkyl, C₁₋₄ alkoxy and C₃₋₆ cycloalkyl, each substituted with 0 to 3 substituents independently selected from halo; or
(B) α is a single bond and β is a single bond;
   X¹ is CH₂;
   X² is selected from NR² and CH₂;
   X³ is selected from NR³ and CH₂;
   X⁴ is CH₂; provided no more than one of X² and X³ is N; and
   R² and R³ are each independently selected from:
      (i) hydrogen; and
      (ii) C₁₋₄ alkyl, C₁₋₄ alkylcarbonyl, C₁₋₄ alkylsulfonyl and C₃₋₆ cycloalkyl, each substituted with 0 to 3 substituents independently selected from halo; or
(C) α is a double bond and β is a single bond;
   X¹ is CH;
   X² is N;
   X³ is absent;
   X⁴ is NR⁴; and
   R⁴ is selected from:
      (i) hydrogen; and
      (ii) C₁₋₄ alkyl, C₁₋₄ alkylcarbonyl, C₁₋₄ alkylsulfonyl and C₃₋₆ cycloalkyl, each substituted with 0 to 3 substituents independently selected from halo; or
(D) α is a single bond and β is a single bond; and
   X¹ is CH₂;
   X² is O and X³ is CH₂, or
   X² is CH₂ and X³ is 0; and
   X⁴ is CH₂;
m is selected from 0, 1 and 2;
each R^{a} and R^{b} is independently selected from hydrogen and C₁₋₄ alkyl, or R^{a} and R^{b}, together with a carbon atom to which both R^{a} and R^{b} are attached, form a C₃₋₆ cycloalkylidene, provided if m is 2, then no more than one R^{a} and R^{b}, together with the carbon atom to which R^{a} and R^{b} are attached, form a C₃₋₆ cycloalkylidene;
R⁵ is selected from:
   (a) C₃₋₈ cycloalkyl, which is substituted with 0 to 5 substituents independently selected from:
      (i) halo, hydroxy, cyano and oxo;
      (ii) amino, which is substituted with 0 to 2 substituents independently selected from C₁₋₄ alkyl; and
      (iii) C₁₋₄ alkyl, C₁₋₄ alkylcarbonyl and C₁₋₄ alkoxy, each substituted with 0 to 3 substituents independently selected from halo;
   provided:
      if m is 0, X¹, X², X³, X⁴ and X⁸ are each CH, a and R are both double bonds, R⁶, R⁷, R¹⁰ and R¹¹ are each hydrogen, and R⁹ is chloro, then R⁵ is not cyclopropyl;
      if m is 0, X¹, X², X³, X⁴ and X⁸ are each CH, a and R are both double bonds, R⁵ is cyclopropylmethyl, R⁶ is n-propyl and R¹⁰ is hydrogen, then R⁷ and R¹¹ are not both methyl or ethyl;
      if m is 0, X¹, X², X³, X⁴ and X⁸ are each CH, a and R are both double bonds, R⁵ is cyclopropylmethyl, R⁶ is n-propyl, R⁷ is methyl, and R¹⁰ and R¹¹ are each hydrogen, then R⁹ is not hydrogen or hydroxy;
      if m is 0, X¹, X², X³, X⁴ and X⁸ are each CH, a and R are both double bonds, R⁶ is n-propyl, R⁷ is methoxy, R⁹ and R¹¹ are each hydrogen, and R¹⁰ is chloro, then R⁵ is not cyclopropylmethyl;
      if m is 0, X¹, X², X³, X⁴ and X⁸ are each CH, a and R are both double bonds, R⁶ is n-propyl, R⁷, R¹⁰ and R¹¹ are each hydrogen, and R⁹ is chloro, then R⁵ is not cyclopropylmethyl;
      if m is 0, X¹, X², X³, X⁴ and X⁸ are each CH, a and R are both double bonds, R⁵ is cyclopropylmethyl, R⁷, R⁹ and R¹¹ are each methyl, and R¹⁰ is hydrogen, then R⁶ is not methyl, ethyl, n-propyl or methoxyethyl;
      if m is 0, X¹ and X⁴ are both CH, X² and X³ are both CH, α and β are both double bonds, R⁵ is cyclopentyl, and R⁶, R⁷, R⁹, R¹⁰ and R¹¹ are each hydrogen, then X⁸ is not N or CH;
      if m is 0, X¹, X², X³, X⁴ and X⁸ are each CH, a and R are both double bonds, and R⁶, R⁷, R⁹, R¹⁰ and R¹¹ are each hydrogen, then R⁵ is not cyclopentyl, cyclohexyl or cyclooctyl;
      if m is 1, X¹, X², X³, X⁴ and X⁸ are each CH, a and R are both double bonds, R⁵ is cyclohexyl, R^{b}, R⁶, R⁷, R⁹, R¹⁰ and R¹¹ are each hydrogen, then R^{a} is not methyl, ethyl, isopropyl, n-propyl;
      if m is 1, X¹, X², X³, X⁴ and X⁸ are each CH, a and R are both double bonds, R^{a} is methyl, R^{b}, R⁶, R⁷, R⁹ and R¹¹ are each hydrogen, and R¹⁰ is methoxy, then R⁵ is not cyclohexyl;
      if m is 1, X¹, X², X³, X⁴ and X⁸ are each CH, a and R are both double bonds, R^{a} is methyl, R^{b}, R⁶, R⁹, R¹⁰ and R¹¹ are each hydrogen, and R⁷ is methyl, then R⁵ is not cyclohexyl;
      if m is 0, X¹, X², X³, X⁴ and X⁸ are each CH, a and R are both double bonds, R⁵ is cyclohexyl, R⁶, R⁷ and R¹¹ are each hydrogen, and R¹⁰ is methyl, then R⁹ is not chloro or methyl;
      if m is 0, X¹, X², X³, X⁴ and X⁸ are each CH, a and R are both double bonds, R⁵ is cyclohexyl, R⁷ is methyl, R⁹ is hydroxy, and R¹⁰ and R¹¹ are each hydrogen, then R⁶ is not methyl or ethyl;
      if m is 0, X¹, X², X³, X⁴ and X⁸ are each CH, a and R are both double bonds, R⁶, R⁷, R⁹ and R¹¹ are each methyl, and R¹⁰ is hydrogen, then R⁵ is not cyclohexyl;
      if m is 0, X¹, X², X³, X⁴ and X⁸ are each CH, a and R are both double bonds, R⁶, R⁹ and R¹¹ are each hydrogen, and R⁷ and R¹⁰ are each methyl, then R⁵ is not cyclohexyl;
      if m is 0, X¹, X², X³, X⁴ and X⁸ are each CH, a and R are both double bonds, R⁶, R⁷, R¹⁰ and R¹¹ are each hydrogen, and R⁹ is methyl, then R⁵ is not cycloheptyl;
      if m is 0, X¹, X² and X⁴ are each CH₂, X³ is 0, X⁸ is CH, a and R are both single bonds, R⁶, R¹⁰ and R¹¹ are each hydrogen, R⁷ is hydroxy and R⁹ is CF₃ or OCF₃, then R⁵ is not 3-hydroxy-3-methylcyclobutyl;
      if m is 0, X¹, X³ and X⁴ are each CH₂, X² is 0, X⁸ is CH, a and R are both single bonds, R⁶, R¹⁰ and R¹¹ are each hydrogen, R⁷ is hydroxy and R⁹ is CF₃ or OCF₃, then R⁵ is not 3-hydroxy-3-methylcyclobutyl;
      if m is 0, X¹, X² and X⁴ are each CH₂, X³ is 0, X⁸ is CH, a and R are both single bonds, R⁶, R¹⁰ and R¹¹ are each hydrogen, R⁷ is hydroxy, and R⁹ is hydrogen, CF₃, OCF₃ or cyclobutyl, then R⁵ is not 2-hydroxycyclohexyl;
      if m is 0, X¹, X³ and X⁴ are each CH₂, X² is 0, X⁸ is CH, a and R are both single bonds, R⁶, R¹⁰ and R¹¹ are each hydrogen, R⁷ is hydroxy, and R⁹ is hydrogen, chloro, CF₃, CHF₂, OCF₃, OCHF₂, OCH₃ or cyclobutyl, then R⁵ is not 2-hydroxycyclohexyl;
      if m is 0, X¹, X² and X⁴ are each CH₂, X³ is 0, X⁸ is CH, a and R are both single bonds, R⁶ and R¹⁰ are each hydrogen, R⁷ is hydroxy, R⁹ is methyl, and R¹¹ is fluoro, then R⁵ is not 2-hydroxycyclohexyl;
      if m is 0, X¹, X³ and X⁴ are each CH₂, X² is 0, X⁸ is CH, a and R are both single bonds, R⁶ and R¹⁰ are each hydrogen, R⁷ is hydroxy, R⁹ is methyl, and R¹¹ is fluoro, then R⁵ is not 2-hydroxycyclohexyl;
      if m is 0, X¹, X³ and X⁴ are each CH₂, X² is 0, X⁸ is CH, a and R are both single bonds, R⁶ and R¹¹ are each hydrogen, R⁷ is hydroxy, and R⁹ is CF₃, and R¹⁰ is fluoro, then R⁵ is not 2-hydroxycyclohexyl;
      if m is 0, X¹, X² and X⁴ are each CH₂, X³ is 0, X⁸ is CH, a and R are both single bonds, R⁶, R¹⁰ and R¹¹ are each hydrogen, R⁷ is hydroxy, and R⁹ is CF₃, then R⁵ is not 2-cyanocyclohexyl or 2-aminocyclohexyl; and
      if m is 0, X¹, X³ and X⁴ are each CH₂, X² is 0, X⁸ is CH, a and R are both single bonds, R⁶, R¹⁰ and R¹¹ are each hydrogen, R⁷ is hydroxy, and R⁹ is CF₃, then R⁵ is not 2-cyanocyclohexyl;
   (b) C₃₋₈ heterocyclyl in which up to 3 carbon ring atoms are each independently substituted with 0 to 2 substituents independently selected from:
      (i) halo, hydroxy, cyano and oxo;
      (ii) amino, which is substituted with 0 to 2 substituents independently selected from C₁₋₄ alkyl; and
      (iii) C₁₋₄ alkyl, C₁₋₄ alkylcarbonyl and C₁₋₄ alkoxy, each substituted with 0 to 3 substituents independently selected from halo;
   and in which a nitrogen ring atom, if present, is unsubstituted or substituted with a substituent selected from:
      (i) C₁₋₄ alkyl, C₁₋₄ alkylcarbonyl and C₁₋₄ alkylsulfonyl, each substituted with 0 to 3 substituents independently selected from halo;
      (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
      (iii) phenyl-(CH₂)ₙ and pyridinyl-(CH₂)ₙ, which phenyl and pyridinyl moieties are substituted with 0 to 3 substituents independently selected from halo, hydroxy, cyano, C₁0.4 alkyl and C₁0.4 alkoxy;
   wherein the C₃₋₈ heterocyclyl has only one ring heteroatom, the ring heteroatom selected from nitrogen, oxygen, and sulfur; and
   n is selected from 0 and 1;
   provided:
      if m is 0, X¹, X³, X⁴ and X⁸ are each CH, X² is CR², a and R are both double bonds, R⁶, R⁷, R¹⁰ and R¹¹ are each hydrogen, and R² and R⁹ are each methoxy, then R⁵ is not: 1-(4-chloro-3-fluorobenzyl)piperidin-4-yl, 1-(4-methylbenzyl)piperidin-4-yl, 1-(3-fluoro-4-methylbenzyl)piperidin-4-yl, 1-(4-fluoro-3-methylbenzyl)piperidin-4-yl, 1-phenethylpiperidin-4-yl, 1-(2-methylbenzyl)piperidin-4-yl, 1-(3,5-dimethylbenzyl)piperidin-4-yl, 1-(4-methoxy-3-methylbenzyl)piperidin-4-yl, 1-(3,4-dichlorobenzyl)piperidin-4-yl, 1-(3-chloro-4-methylbenzyl)piperidin-4-yl, 1-(4-chlorobenzyl)piperidin-4-yl, 1-(3,4-difluorobenzyl)piperidin-4-yl, 1-(3,4-dimethoxybenzyl)piperidin-4-yl, 1-(4-bromo-3-methylbenzyl)piperidin-4-yl, 1-(2,4,6-trimethylbenzyl)piperidin-4-yl, 1-(4-chloro-3-methylbenzyl)piperidin-4-yl, 1-(3,4-dimethylbenzyl)piperidin-4-yl, 1-(3-methylbenzyl)piperidin-4-yl, 1-(4-ethylbenzyl)piperidin-4-yl, 1-(3-phenylpropyl)piperidin-4-yl, 1-(2,4-dichlorobenzyl)piperidin-4-yl, 1-(3-cyanobenzyl)piperidin-4-yl, 1-(3-chloro-4-fluorobenzyl)piperidin-4-yl, 1-(2,4-dimethylbenzyl)piperidin-4-yl, piperidin-4-yl, or 1-benzylpiperidin-4-yl;
      if m is 0, X¹, X², X³, X⁴ and X⁸ are each CH, a and R are both double bonds, and R⁶, R⁷, R⁹, R¹⁰ and R¹¹ are each hydrogen, then R⁵ is not piperidin-4-yl or 1-benzylpiperidin-4-yl;
      if m is 0, X¹, X², X³, X⁴ and X⁸ are each CH, a and R are both double bonds, and R⁶, R⁷, R¹⁰ and R¹¹ are each hydrogen, and R⁹ is methoxy, then R⁵ is not 1-benzylpiperidin-4-yl;
      if m is 0, X¹, X³ and X⁸ are each CH, X² is CR², X⁴ is CR⁴, a and R are both double bonds, R⁶, R⁷, R¹⁰ and R¹¹ are each hydrogen, and R², R⁴ and R⁹ are each methoxy, then R⁵ is not 1-benzylpiperidin-4-yl;
      if m is 0, X¹, X³, X⁴ and X⁸ are each CH, X² is CR², a and R are both double bonds, R⁶, R⁷, R⁹ and R¹⁰ are each hydrogen, and R² and R¹⁰ are each methoxy, then R⁵ is not 1-benzylpiperidin-4-yl;
      if m is 2, R^{a} and R^{b} at each occurrence is hydrogen, X¹, X², X³, X⁴ and X⁸ are each CH, a and R are both double bonds, and R⁶, R⁷, R⁹, R¹⁰ and R¹¹ are each hydrogen, then R⁵ is not 1-benzylpiperidin-4-yl;
      if m is 0, X¹, X², X³, X⁴ and X⁸ are each CH, a and R are both double bonds, R⁵ is pyrrolidin-3-yl, and R⁶, R⁷, R¹⁰ and R¹¹ are each hydrogen, then R⁹ is not hydrogen, fluoro or methyl;
      if m is 2, R^{a} and R^{b} at each occurrence is hydrogen, X¹, X², X³, X⁴ and X⁸ are each CH, a and R are both double bonds, R⁶, R⁷, R¹⁰ and R¹¹ are each hydrogen, and R⁹ is chloro, then R⁵ is not pyrrolidin-1-yl;
      if m is 1, X¹, X², X³, X⁴ and X⁸ are each CH, a and R are both double bonds, R⁵ is tetrahydrofuran-2-yl, and R^{a}, R^{b}, R⁶, R⁷, R¹⁰ and R¹¹ are each hydrogen, then R⁹ is not fluorine or methyl;
      if m is 0, X¹, X², X³, X⁴ and X⁸ are each CH, a and R are both double bonds, R⁶, R⁷, R¹⁰ and R¹¹ are each hydrogen, and R⁹ is methoxy, then R⁵ is not 6-oxaspiro[2.5]octan-1-yl;
      if m is 0, X¹, X² and X⁴ are each CH₂, X³ is 0, X⁸ is CH, a and R are both single bonds, R⁶, R¹⁰ and R¹¹ are each hydrogen, R⁷ is hydroxy and R⁹ is CF₃, then R⁵ is not piperidin-3-yl or 1-methylpiperidin-3-yl;
      if m is 0, X¹, X³ and X⁴ are each CH₂, X² is 0, X⁸ is CH, a and R are both single bonds, R⁶, R¹⁰ and R¹¹ are each hydrogen, R⁷ is hydroxy and R⁹ is CF₃, then R⁵ is not piperidin-3-yl or 1-methylpiperidin-3-yl;
      if m is 0, X¹, X² and X⁴ are each CH₂, X³ is 0, X⁸ is CH, a and R are both single bonds, R⁶ and R¹⁰ are each hydrogen, R⁷ is hydroxy, R⁹ is CF₃, methyl or chloro, and R¹¹ is fluoro, then R⁵ is not 1-methylpiperidin-3-yl; and
      if m is 0, X¹, X² and X⁴ are each CH₂, X³ is 0, X⁸ is CH, a and R are both single bonds, R⁶, R¹⁰ and R¹¹ are each hydrogen, R⁷ is hydroxy and R⁹ is methyl or chloro, then R⁵ is not 1-methylpiperidin-3-yl;
   (c) phenyl, which is substituted with 0 to 3 substituents independently selected from halo, hydroxy, cyano, C₁₋₄ alkyl and C₁₋₄ alkoxy, provided at least one of the substituents is hydroxy, and:
      if m is 0, X¹, X², X³, X⁴, and X⁸ are each CH, a and R are both double bonds, R⁵ is 3-hydroxyphenyl, and R⁶, R⁷, R¹⁰ and R¹¹ are each hydrogen, then R⁹ is not methyl, methoxy, ethoxy or chloro;
      if m is 0, X¹, X², X³, X⁴, and X⁸ are each CH, a and R are both double bonds, R⁵ is 2-hydroxyphenyl, and R⁶, R⁷, R¹⁰ and R¹¹ are each hydrogen, then R⁹ is not hydrogen, methyl, methoxy, chloro;
      if m is 0, X¹, X², X³, X⁴, and X⁸ are each CH, a and R are both double bonds, R⁵ is 2-hydroxyphenyl, and R⁶, R⁷ and R¹¹ are each hydrogen, then R⁹ and R¹⁰ are not both chloro;
      if m is 0, X¹, X², X³, X⁴, and X⁸ are each CH, a and R are both double bonds, R⁵ is 4-hydroxyphenyl, and R⁶, R⁷, R¹⁰ and R¹¹ are each hydrogen, then R⁹ is not hydrogen, hydroxy, chloro, methoxy, ethoxy, trifluoromethyl or tert-butyl;
      if m is 0, X¹, X², X³ and X⁴ are each CH, X⁸ is CH, a and R are both double bonds, R⁶, R⁷, R¹⁰ and R¹¹ are each hydrogen, and R⁹ is methyl, then R⁵ is not 3-hydroxyphenyl or 4-hydroxyphenyl;
      if m is 0, X¹, X², X³, X⁴, and X⁸ are each CH, a and R are both double bonds, R⁵ is unsubstituted phenyl, and R⁶, R⁷, R¹⁰ and R¹¹ are each hydrogen, then R⁹ is not hydrogen, chloro, hydroxy, methyl or methoxy;
      if m is 0, X¹, X², X³, X⁴, and X⁸ are each CH, a and R are both double bonds, R⁵ is unsubstituted phenyl, R⁶, R⁷ and R¹⁰ are each hydrogen, and R¹¹ is methyl, then R⁹ is not hydrogen or chloro;
      if m is 0, X¹, X², X³, X⁴, and X⁸ are each CH, a and R are both double bonds, R⁶, R⁷, R⁹ and R¹⁰ are each hydrogen, and R¹¹ is methyl, then R⁵ is not unsubstituted phenyl;
      if m is 0, X¹, X², X³ and X⁴ are each CH, X⁸ is N, a and R are both double bonds, and R⁶, R⁷, R⁹, R¹⁰ and R¹¹ are each hydrogen, then R⁵ is not unsubstituted phenyl;
      if m is 0, X¹, X⁴ and X⁸ are each CH, X² is CR², X³ is CR³, a and R are both double bonds, R⁵ is unsubstituted phenyl, and R⁶, R⁷, R⁹, R¹⁰ and R¹¹ are each hydrogen, then R² and R³ are not both methyl or not both methoxy; and
      if m is 1, X¹, X², X³, X⁴, and X⁸ are each CH, a and R are both double bonds, R^{a} and R^{b}, together with the carbon atom to which both R^{a} and R^{b} are attached, form a cyclopentylidene, and R⁶, R⁷, R⁹, R¹⁰ and R¹¹ are each hydrogen, then R⁵ is not unsubstituted phenyl;
R⁶ is selected from hydrogen and C₁₋₄ alkyl;
X⁸ is selected from N and CR¹;
R⁷, R⁸ and R¹¹ are each independently selected from:
   (i) hydrogen, halo, hydroxy and cyano;
   (ii) C₁₋₄ alkyl and C₁₋₄ alkoxy, each substituted with 0 to 3 substituents independently selected from halo; and
   (iii) C₃₋₈ cycloalkyl which is substituted with 0 to 3 substituents independently selected from halo, C₁₋₄ alkyl and C₁0.4 alkoxy; and
R⁹ and R¹⁰ are each independently selected from:
   (i) hydrogen, halo, hydroxy and cyano;
   (ii) C₁₋₄ alkyl and C₁₋₄ alkoxy, each substituted with 0 to 3 substituents independently selected from halo; and
   (iii) C₃₋₈ cycloalkyl which is substituted with 0 to 3 substituents independently selected from halo, C₁₋₄ alkyl and C₁₋₄ alkoxy; or
R⁹ and R¹⁰ form an ethan-1,2-dioxy moiety bridging the carbon atoms to which they are attached.

Another aspect of the invention provides a compound which is selected from the group of compounds described in the examples and their pharmaceutically acceptable salts.

A further aspect of the invention provides a compound or pharmaceutically acceptable salt as defined in the preceding paragraphs for use as a medicament.

An additional aspect of the invention provides a pharmaceutical composition which includes a compound of Formula 1 or a pharmaceutically acceptable salt thereof, or any one of the compounds or pharmaceutically acceptable salts defined in the preceding paragraphs; and a pharmaceutically acceptable excipient.

Another aspect of the invention provides a compound of Formula 1 or a pharmaceutically acceptable salt thereof, or any one of the compounds or pharmaceutically acceptable salts defined in the preceding paragraphs, for treatment of a disease, disorder or condition associated with NLRP3, including a disease, disorder or condition associated with a heterozygous gain of function mutation in the NLRP3 gene such as cryopyrin-associated periodic syndrome (CAPS).

A further aspect of the invention provides a use of a compound of Formula 1 or a pharmaceutically acceptable salt thereof, or any one of the compounds or pharmaceutically acceptable salts defined in the preceding paragraphs, for the manufacture of a medicament for the treatment of a disease, disorder or condition associated with NLRP3, including a disease, disorder or condition associated with a heterozygous gain of function mutation in the NLRP3 gene such as cryopyrin-associated periodic syndrome (CAPS).

An additional aspect of the invention provides a method for treating a disease, disorder or condition associated with NLRP3, including a disease, disorder or condition associated with a heterozygous gain of function mutation in the NLRP3 gene such as cryopyrin-associated periodic syndrome (CAPS), the method comprising administering to the subject an effective amount of a compound of Formula 1 or a pharmaceutically acceptable salt thereof, or any one of the compounds or pharmaceutically acceptable salts defined in the preceding paragraphs.

Another aspect of the invention provides a method for treating a cryopyrin-associated periodic syndrome (CAPS), including neonatal-onset multisystem inflammatory disease (NOMID/CINCA), Muckle-Wells syndrome (MWS), and familial cold autoinflammatory syndrome (FCAS), the method comprising administering to the subject an effective amount of a compound of Formula 1 or a pharmaceutically acceptable salt thereof, or any one of the compounds or pharmaceutically acceptable salts defined in the preceding paragraphs.

A further aspect of the invention provides a method for treating a disease, disorder or condition in a subject, the method comprising administering to the subject an effective amount of a compound of Formula 1 or a pharmaceutically acceptable salt thereof, or any one of the compounds or pharmaceutically acceptable salts defined in the preceding paragraphs, wherein the disease, disorder or condition is a neurodegenerative disease, disorder or condition.

An additional aspect of the invention provides a method for treating a disease, disorder or condition in a subject, the method comprising administering to the subject an effective amount of a compound of Formula 1 or a pharmaceutically acceptable salt thereof, or any one of the compounds or pharmaceutically acceptable salts defined in the preceding paragraphs, wherein the disease, disorder or condition is selected from Parkinson's disease, Alzheimer's disease, Huntington's disease, amyotrophic lateral sclerosis and prion disease.

Another aspect of the invention provides an effective amount of a compound of Formula 1 or a pharmaceutically acceptable salt thereof, or any one of the compounds or pharmaceutically acceptable salts defined in the preceding paragraphs; and at least one additional pharmacologically active agent.

### DETAILED DESCRIPTION OF THE INVENTION

Unless otherwise indicated, this disclosure uses definitions provided below.

“Substituted,” when used in connection with a chemical substituent or moiety (e.g., a substituted C₁₋₆ alkyl group or a substituted phenyl group), means that one or more hydrogen atoms of the substituent or moiety have been replaced with one or more non-hydrogen atoms or groups, provided valence requirements are met and a chemically stable compound results from the substitution. Unless otherwise indicated, a chemical substituent or moiety is not substituted (or further substituted). For example, referring to a phenyl group without indicating it is substituted, means the phenyl group does not include non-hydrogen substituents. Likewise, referring to a 2-fluorophenyl group without indicating it is substituted, means the 2-fluorophenyl group does not include additional non-hydrogen substituents beyond the 2-fluoro substituent.

“About” or “approximately,” when used in connection with a measurable numerical variable, refers to the indicated value of the variable and to all values of the variable that are within the experimental error of the indicated value or within ±10 percent of the indicated value, whichever is greater.

“Alkyl” refers to straight chain and branched saturated hydrocarbon groups, generally having a specified number of carbon atoms (e.g., C₁₋₄ alkyl refers to an alkyl group having 1 to 4 (i.e., 1, 2, 3 or 4) carbon atoms, C₁₋₆ alkyl refers to an alkyl group having 1 to 6 carbon atoms, and so on). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkanediyl” refers to divalent alkyl groups, where alkyl is defined above, and generally having a specified number of carbon atoms (e.g., C₁₋₄ alkanediyl refers to an alkanediyl group having 1 to 4 (i.e., 1, 2, 3 or 4) carbon atoms, C₁0.6 alkanediyl refers to an alkanediyl group having 1 to 6 carbon atoms, and so on). Examples of alkanediyl groups include methylene, ethane-1,1-diyl, ethane-1,2-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, isobutane-1,3-diyl, isobutane-1,1-diyl, isobutane-1,2-diyl, and the like.

“Alkenyl” refers to straight chain and branched hydrocarbon groups having one or more carbon-carbon double bonds, and generally having a specified number of carbon atoms. Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Alkoxy” refers to straight chain and branched saturated hydrocarbon groups attached through an oxygen atom, generally having a specified number of carbon atoms (e.g., C₁₋₄ alkoxy refers to an alkoxy group having 1 to 4 (i.e., 1, 2, 3 or 4) carbon atoms, C₁₋₆ alkoxy refers to an alkoxy group having 1 to 6 carbon atoms, and so on). Examples of alkoxy groups include methoxy, ethoxy, n-propoxy, i-propoxy, n-butoxy, s-butoxy, i-butoxy, t-butoxy, pent-1-yloxy, pent-2-yloxy, pent-3-yloxy, 3-methylbut-1-yloxy, 3-methylbut-2-yloxy, 2-methylbut-2-yloxy, 2,2,2-trimethyleth-1-yloxy, n-hexoxy, and the like.

“Alkylcarbonyl” and “alkylsulfonyl” refer to an alkyl group, as defined above, which is attached, respectively, through a carbonyl (C(O)) group or a sulfonyl (SO₂) group, and generally having a specified number of carbon atoms, including the carbon atom of the carbonyl group. For example, C₁₋₄ alkylcarbonyl refers to an alkylcarbonyl group having 1 to 4 (i.e., 1, 2, 3 or 4) carbon atoms, including the carbonyl moiety, C₁0.6 alkylsulfonyl refers to an alkylsulfonyl group having 1 to 6 carbon atoms, and so on. Examples of alkylcarbonyl groups include carbonyl (formyl), methylcarbonyl (acetyl), ethylcarbonyl, i-propylcarbonyl, n-propylcarbonyl, and the like. Examples of alkylsulfonyl groups include methylsulfonyl, ethylsulfonyl, i-propylsulfonyl, n-propylsulfonyl, and the like.

“Halo,” “halogen” and “halogeno” may be used interchangeably and refer to fluoro, chloro, bromo, and iodo.

“Haloalkyl,” “haloalkenyl,” and “haloalkynyl,” refer, respectively, to alkyl, alkenyl, and alkynyl groups substituted with one or more halogen atoms, where alkyl, alkenyl, and alkynyl are defined above, and generally having a specified number of carbon atoms. Examples of haloalkyl groups include fluoromethyl, difluoromethyl, trifluoromethyl, chloromethyl, dichloromethyl, trichloromethyl, 1-fluoroethyl, 1,1-difluoroethyl, 1-chloroethyl, 1,1-dichloroethyl, 1-fluoro-1-methylethyl, 1-chloro-1-methylethyl, and the like.

“Cycloalkyl” refers to saturated monocyclic and bicyclic hydrocarbon groups, generally having a specified number of carbon atoms that comprise the ring or rings (e.g., C₃₋₈ cycloalkyl refers to a cycloalkyl group having 3 to 8 carbon atoms as ring members). Bicyclic hydrocarbon groups may include isolated rings (two rings sharing no carbon atoms), spiro rings (two rings sharing one carbon atom), fused rings (two rings sharing two carbon atoms and the bond between the two common carbon atoms), and bridged rings (two rings sharing two carbon atoms, but not a common bond). The cycloalkyl group may be attached through any ring atom unless such attachment would violate valence requirements, and where indicated, may optionally include one or more non-hydrogen substituents unless such substitution would violate valence requirements.

Examples of monocyclic cycloalkyl groups include cyclopropyl, cyclobutyl, cyclopentyl, cyclohexyl, and the like. Examples of fused bicyclic cycloalkyl groups include bicyclo[2.1.0]pentanyl (i.e., bicyclo[2.1.0]pentan-1-yl, bicyclo[2.1.0]pentan-2-yl, and bicyclo[2.1.0]pentan-5-yl), bicyclo[3.1.0]hexanyl, bicyclo[3.2.0]heptanyl, bicyclo[4.1.0]heptanyl, bicyclo[3.3.0]octanyl, bicyclo[4.2.0]octanyl, bicyclo[4.3.0]nonanyl, bicyclo[4.4.0]decanyl, and the like. Examples of bridged cycloalkyl groups include bicyclo[2.1.1]hexanyl, bicyclo[2.2.1]heptanyl, bicyclo[3.1.1]heptanyl, bicyclo[2.2.2]octanyl, bicyclo[3.2.1]octanyl, bicyclo[4.1.1]octanyl, bicyclo[3.3.1]nonanyl, bicyclo[4.2.1]nonanyl, bicyclo[3.3.2]decanyl, bicyclo[4.2.2]decanyl, bicyclo[4.3.1]decanyl, bicyclo[3.3.3]undecanyl, bicyclo[4.3.2]undecanyl, bicyclo[4.3.3]dodecanyl, and the like. Examples of spiro cycloalkyl groups include spiro[3.3]heptanyl, spiro[2.4]heptanyl, spiro[3.4]octanyl, spiro[2.5]octanyl, spiro[3.5]nonanyl, and the like. Examples of isolated bicyclic cycloalkyl groups include those derived from bi(cyclobutane), cyclobutanecyclopentane, bi(cyclopentane), cyclobutanecyclohexane, cyclopentanecyclohexane, bi(cyclohexane), etc.

“Cycloalkanediyl” refers to divalent cycloalkyl groups, where cycloalkyl is defined above, and generally having a specified number of carbon atoms (e.g., C₃₋₅ cycloalkanediyl refers to a cycloalkanediyl group having 3 to 5 (i.e., 3, 4 or 5) carbon atoms, C₃0.6 cycloalkanediyl refers to a cycloalkanediyl group having 3 to 6 carbon atoms, and so on). Examples of cycloalkanediyl groups include cyclopropane-1,1-diyl, cyclopropane-1,2-diyl, cyclobutane-1,1-diyl, cyclobutane-1,2-diyl, and the like.

“Cycloalkylidene” refers to a divalent monocyclic cycloalkyl group, where cycloalkyl is defined above, which is attached through a single carbon atom of the group, and generally having a specified number of carbon atoms that comprise the ring (e.g., C₃0.6 cycloalkylidene refers to a cycloalkylidene group having 3 to 6 carbon atoms as ring members). Examples include cyclopropylidene, cyclobutylidene, cyclopentylidene, and cyclohexylidene.

“Cycloalkenyl” refers to partially unsaturated monocyclic and bicyclic hydrocarbon groups, generally having a specified number of carbon atoms that comprise the ring or rings. As with cycloalkyl groups, the bicyclic cycloalkenyl groups may include isolated, spiro, fused, or bridged rings. Similarly, the cycloalkenyl group may be attached through any ring atom, and where indicated, may optionally include one or more non-hydrogen substituents unless such attachment or substitution would violate valence requirements. Examples of cycloalkenyl groups include the partially unsaturated analogs of the cycloalkyl groups described above, such as cyclobutenyl (i.e., cyclobuten-1-yl and cyclobuten-3-yl), cyclopentenyl, cyclohexenyl, bicyclo[2.2.1]hept-2-enyl, and the like.

“Aryl” refers to fully unsaturated monocyclic aromatic hydrocarbons and to polycyclic hydrocarbons having at least one aromatic ring, both monocyclic and polycyclic aryl groups generally having a specified number of carbon atoms that comprise their ring members (e.g., C₆₋₁₄ aryl refers to an aryl group having 6 to 14 carbon atoms as ring members). The group may be attached through any ring atom, and where indicated, may optionally include one or more non-hydrogen substituents unless such attachment or substitution would violate valence requirements. Examples of aryl groups include phenyl, biphenyl, cyclobutabenzenyl, indenyl, naphthalenyl, benzocycloheptanyl, biphenylenyl, fluorenyl, groups derived from cycloheptatriene cation, and the like.

“Arylene” refers to divalent aryl groups, where aryl is defined above. Examples of arylene groups include o-phenylene (i.e., benzene-1,2-diyl).

“Heterocycle” and “heterocyclyl” may be used interchangeably and refer to saturated or partially unsaturated monocyclic or bicyclic groups having ring atoms composed of carbon atoms and one or more heteroatoms independently selected from nitrogen, oxygen, and sulfur. Both the monocyclic and bicyclic groups generally have a specified number of carbon atoms in their ring or rings (e.g., C₂₋₆ heterocyclyl refers to a heterocyclyl group having 2 to 6 carbon atoms and, e.g., 1 to 4 heteroatoms, as ring members). As with bicyclic cycloalkyl groups, bicyclic heterocyclyl groups may include isolated rings, spiro rings, fused rings, and bridged rings. The heterocyclyl group may be attached through any ring atom, and where indicated, may optionally include one or more non-hydrogen substituents unless such attachment or substitution would violate valence requirements or result in a chemically unstable compound. Examples of heterocyclyl groups include oxiranyl, thiiranyl, aziridinyl (e.g., aziridin-1-yl and aziridin-2-yl), oxetanyl, thietanyl, azetidinyl, tetrahydrofuranyl, tetrahydrothienyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thiazepanyl, 1,4-diazepanyl, 3,4-dihydro-2H-pyranyl, 3,6-dihydro-2H-pyranyl, 2H-pyranyl, 1,2-dihydropyridinyl, 1,2,3,4-tetrahydropyridinyl, 1,2,5,6-tetrahydropyridinyl, 1,6-dihydropyrimidinyl, 1,2,3,4-tetrahydropyrimidinyl, and 1,2-dihydropyrazolo[1,5-d][1,2,4]triazinyl.

“Heterocycle-diyl” refers to heterocyclyl groups which are attached through two ring atoms of the group, where heterocyclyl is defined above. They generally have a specified number of carbon atoms in their ring or rings (e.g., C₂₋₆ heterocycle-diyl refers to a heterocycle-diyl group having 2 to 6 carbon atoms and, e.g., 1 to 4 heteroatoms, as ring members). Examples of heterocycle-diyl groups include the multivalent analogs of the heterocycle groups described above, such as morpholine-3,4-diyl, pyrrolidine-1,2-diyl, 1-pyrrolidinyl-2-ylidene, 1-pyridinyl-2-ylidene, 1-(4H)-pyrazolyl-5-ylidene, 1-(3H)-imidazolyl-2-ylidene, 3-oxazolyl-2-ylidene, 1-piperidinyl-2-ylidene, 1-piperazinyl-6-ylidene, and the like.

“Heteroaromatic” and “heteroaryl” may be used interchangeably and refer to unsaturated monocyclic aromatic groups and to polycyclic groups having at least one aromatic ring, each of the groups having ring atoms composed of carbon atoms and one or more heteroatoms independently selected from nitrogen, oxygen, and sulfur. Both the monocyclic and polycyclic groups generally have a specified number of carbon atoms as ring members (e.g., C₁0.9 heteroaryl refers to a heteroaryl group having 1 to 9 carbon atoms and, e.g., 1 to 4 heteroatoms, as ring members) and may include any bicyclic group in which any of the above-listed monocyclic heterocycles are fused to a benzene ring. The heteroaryl group may be attached through any ring atom (or ring atoms for fused rings), and where indicated, may optionally include one or more non-hydrogen substituents unless such attachment or substitution would violate valence requirements or result in a chemically unstable compound.

Examples of heteroaryl groups include monocyclic groups such as pyrrolyl (e.g., pyrrol-1-yl, pyrrol-2-yl, and pyrrol-3-yl), furanyl, thienyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, and pyrazinyl.

Examples of heteroaryl groups also include bicyclic groups such as benzofuranyl, isobenzofuranyl, benzothienyl, benzo[c]thienyl, 1H-indolyl, 3H-indolyl, isoindolyl, 1H-isoindolyl, indolinyl, isoindolinyl, benzimidazolyl, 1H-indazolyl, 2H-indazolyl, benzotriazolyl, 1H-pyrrolo[2,3-b]pyridinyl, 1H-pyrrolo[2,3-c]pyridinyl, 1H-pyrrolo[3,2-c]pyridinyl, 1H-pyrrolo[3,2-b]pyridinyl, 3H-imidazo[4,5-b]pyridinyl, 3H-imidazo[4,5-c]pyridinyl, 1H-pyrazolo[4,3-b]pyridinyl, 1H-pyrazolo[4,3-c]pyridinyl, 1H-pyrazolo[3,4-c]pyridinyl, 1H-pyrazolo[3,4-b]pyridinyl, 7H-purinyl, indolizinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, 1,2,3,4-tetrahydropyrido[2,3-b]pyrazinyl, 2,3-dihydrobenzo[b][1,4]dioxinyl, 3,4-dihydro-2H-pyrido[3,2-b][1,4]oxazinyl, 2,3-dihydro-1H-benzo[d]imidazolyl, benzo[d]thiazolyl, 2,3-dihydro-1H-pyrrolo[2,3-b]pyridinyl, [1,2,4]triazolo[1,5-a]pyridinyl, 2,3-dihydro-1H-imidazo[4,5-b]pyridinyl, tetrazolo[1,5-a]pyridinyl, 7H-pyrrolo[2,3-d]pyrimidinyl, pyrazolo[1,5-a]pyrimidinyl, imidazo[1,2-a]pyrimidinyl, 4,5-dihydro-1H-pyrazolo[3,4-d]pyrimidinyl, 2,3,6,7-tetrahydro-1H-purinyl, 5H-pyrrolo[2,3-b]pyrazinyl, imidazo[1,2-a]pyrazinyl, imidazo[1,2-b]pyridazinyl, and 4,5,6,7-tetrahydropyrazolo[1,5-a]pyrazinyl.

“Heteroarylene” refers to heteroaryl groups which are attached through two ring atoms of the group, where heteroaryl is defined above. They generally have a specified number of carbon atoms in their ring or rings (e.g., C₃₋₅ heteroarylene refers to a heteroarylene group having 3 to 5 carbon atoms and, e.g., 1 to 4 heteroatoms, as ring members). Examples of heteroarylene groups include the multivalent analogs of the heteroaryl groups described above, such as pyridine-2,3-diyl, pyridine-3,4-diyl, pyrazole-4,5-diyl, pyrazole-3,4-diyl, and the like.

“Oxo” refers to a double bonded oxygen (═O).

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate), and arylsulfonates (e.g., tosylate, brosylate, closylate, and nosylate). Others include carbonates, halide ions, carboxylate anions, phenolate ions, and alkoxides. Some stronger bases, such as NH₂⁻ and OH can be made better leaving groups by treatment with an acid. Common electrofugal leaving groups include the proton, CO₂, and metals.

“Opposite enantiomer” refers to a molecule that is a non-superimposable mirror image of a reference molecule, which may be obtained by inverting all the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomer” and “stereoisomers” of a compound with given stereochemical configuration refer to the opposite enantiomer of the compound and to any diastereoisomers, including geometrical isomers (Z/E) of the compound. For example, if a compound has S,R,Z stereochemical configuration, its stereoisomers would include its opposite enantiomer having R,S,Z configuration, and its diastereomers having S,S,Z configuration, R,R,Z configuration, S,R,E configuration, R,S,E configuration, S,S,E configuration, and R,R,E configuration. If the stereochemical configuration of a compound is not specified, then “stereoisomer” refers to any one of the possible stereochemical configurations of the compound.

“Substantially pure stereoisomer” and variants thereof refer to a sample containing a compound having a specific stereochemical configuration and which comprises at least about 95% of the sample.

“Pure stereoisomer” and variants thereof refer to a sample containing a compound having a specific stereochemical configuration and which comprises at least about 99.5% of the sample.

“Subject” refers to a mammal, including a human.

“Pharmaceutically acceptable” substances refer to those substances which are suitable for administration to subjects.

“Treating” refers to reversing, alleviating, inhibiting the progress of, or preventing a disease, disorder or condition to which such term applies, or to reversing, alleviating, inhibiting the progress of, or preventing one or more symptoms of such disease, disorder or condition.

“Treatment” refers to the act of “treating,” as defined immediately above.

“Drug,” “drug substance,” “active pharmaceutical ingredient,” and the like, refer to a compound (e.g., compounds of Formula 1, including subgeneric compounds and compounds specifically named in the specification) that may be used for treating a subject in need of treatment.

“Effective amount” of a drug, “therapeutically effective amount” of a drug, and the like, refer to the quantity of the drug that may be used for treating a subject and may depend on the weight and age of the subject and the route of administration, among other things.

“Excipient” refers to any diluent or vehicle for a drug.

“Pharmaceutical composition” refers to the combination of one or more drug substances and one or more excipients.

“Drug product,” “pharmaceutical dosage form,” “dosage form,” “final dosage form” and the like, refer to a pharmaceutical composition suitable for treating a subject in need of treatment and generally may be in the form of tablets, capsules, sachets containing powder or granules, liquid solutions or suspensions, patches, films, and the like.

“Condition associated with NLRP3” and similar phrases relate to a disease, disorder or condition in a subject for which inhibition of the NLRP3 inflammasome pathway may provide a therapeutic or prophylactic benefit.

The following abbreviations may be used in the specification: Ac (acetyl); Ac₂0 (acetic anhydride); ACN (acetonitrile); AIBN (azo-bis-isobutyronitrile); AmPhos (bis(di-tert-butyl(4-dimethylaminophenyl)phosphine)dichloropalladium(II)); API (active pharmaceutical ingredient); aq (aqueous); BINAP (2,2′-bis(diphenylphosphino)-1,1′-binaphthyl); Boc (tert-butoxycarbonyl); BrettPhos (2-(dicyclohexylphosphino)3,6-dimethoxy-2′,4′,6′-triisopropyl-1,1′-biphenyl); BrettPhos-Pd-G3 ([(2-di-cyclohexylphosphino-3,6-dimethoxy-2′,4′,6′-triisopropyl-1,1′-biphenyl)-2-(2′-amino-1,1′-biphenyl)]palladium(II)methanesulfonate); BTMPO (NN-bis(2,4,6-trimethoxyphenyl)oxalamide); Cbz (carbobenzyloxy); dba (dibenzylideneacetone); DBU (1,8-diazabicyclo[5.4.0]undec-7-ene); DCC (1,3-dicyclohexylcarbodiimide); DCE (1,1-dichloroethane); DCM (dichloromethane); DEA (diethylamine); DIAD (diisopropyl azodicarboxylate); DIPEA (N,N-diisopropylethylamine, Hunig's Base); DMA (N,N-dimethylacetamide); DMAP (4-dimethylaminopyridine); DME (1,2-dimethoxyethane); DMF (N,N-dimethylformamide); DMP (Dess-Martin periodinane); DMSO (dimethylsulfoxide); dppf (1,1′-bis(diphenylphosphino)ferrocene); DTT (dithiothreitol); EC₅₀ (effective concentration at half maximal response); EDA (ethoxylated dodecyl alcohol, Brj®35); EDC (N-(3-dimethylaminopropyl)-N-ethylcarbodiimide); EDTA (ethylenediaminetetraacetic acid); ee (enantiomeric excess); ELS (evaporative light scattering); eq (equivalents); Et (ethyl); Et₃N (triethylamine); EtOAc (ethyl acetate); EtOH (ethanol); FA (formic acid); HATU (2-(3H-[1,2,3]triazolo[4,5-b]pyridin-3-yl)-1,1,3,3-tetramethyluronium hexafluorophosphate(V)); HEPES (4-(2-hydroxyethyl)piperazine-1-ethanesulfonic acid); HOAc (acetic acid); HOBt (1H-benzo[d][1,2,3]triazol-1-ol); IC₅₀ (concentration at 50% inhibition); IPA (isopropanol); IPAc (isopropyl acetate); IPE (isopropylether); LDA (lithium diisopropylamide); LiHMDS (lithium bis(trimethylsilyl)amide); mCPBA (m-chloroperoxybenzoic acid); Me (methyl); MeOH (methanol); MTBE (methyl tert-butyl ether); mp (melting point); NaOt-Bu (sodium tertiary butoxide); NMM (N-methylmorpholine); NMP 1-methylpyrrolidin-2-one); OTf (triflate); PE (petroleum ether); Ph (phenyl); pEC₅₀ (−log₁₀(EC₅₀), where EC₅₀ is given in molar (M) units); pIC₅₀ (−log₁₀(IC₅₀), where IC₅₀ is given in molar (M) units); Pr (propyl); c-Pr (cyclopropyl), i-Pr (isopropyl); PTFE (polytetrafluoroethylene); PyBOP ((benzotriazol-1-yloxy)tripyrrolidinophosphonium hexafluorophosphate); PyBroP® (bromotripyrrolidinophosphonium hexafluorophosphate); PCy₃ (tricyclohexylphosphine); R-BINAP ((R)-(+)-2,2′-bis(diphenylphosphino)-1,1′-binaphthyl); RT (room temperature, approximately 20° C. to 25° C.); SFC (supercritical fluid chromatography); T3P (2,4,6-tripropyl-1,3,5,2,4,6-trioxatriphosphinane 2,4,6-trioxide); TBSOTf (tert-butyldimethylsilyl trifluoromethanesulfonate); TCEP (tris(2-carboxyethyl)phosphine); TFA (trifluoroacetic acid); TFAA (2,2,2-trifluoroacetic anhydride); THE (tetrahydrofuran); TMS (trimethylsilyl); Tris buffer (2-amino-2-hydroxymethyl-propane-1,3-diol buffer); XPhos (2-dicyclohexylphosphino-2′,4′,6′-triisopropylbiphenyl); and XPhos-Pd-G2 (chloro(2-dicyclohexylphosphino-2′,4′,6′-triisopropyl-1,1′-biphenyl)[2-(2′-amino-1,1′-biphenyl)]palladium(II)).

As described, below, this disclosure concerns compounds of Formula 1 and their pharmaceutically acceptable salts. This disclosure also concerns materials and methods for preparing compounds of Formula 1, pharmaceutical compositions which contain them, and the use of compounds of Formula 1 and their pharmaceutically acceptable salts (optionally in combination with other pharmacologically active agents) for treating diseases, disorders or conditions of the CNS, including neurodegenerative diseases such as Parkinson's disease, Alzheimer's disease, and other diseases, disorders or conditions associated with NLRP3.

The compounds of Formula 1, and pharmaceutically acceptable salts thereof, include those in which:
(1) (A) α is a double bond and R is a double bond;
   X¹ is selected from N and CR¹;
   X² is selected from N and CR²;
   X³ is selected from N and CR³;
   X⁴ is selected from N and CR⁴; provided no more than one of X¹, X², X³ and
      X⁴ is N; and
   R¹, R², R³ and R⁴ are each independently selected from:
      (i) hydrogen, halo, hydroxy, cyano; and
      (ii) C₁₋₄ alkyl, C₁₋₄ alkoxy and C₃₋₆ cycloalkyl, each substituted with 0 to 3 substituents independently selected from halo; or
   (B) α is a single bond and R is a single bond;
      X¹ is CH₂;
      X² is selected from NR² and CH₂;
      X³ is selected from NR³ and CH₂;
      X⁴ is CH₂; provided no more than one of X² and X³ is N; and
      R² and R³ are each independently selected from:
         (i) hydrogen; and
         (ii) C₁₋₄ alkyl, C₁₋₄ alkylcarbonyl, C₁₋₄ alkylsulfonyl and C₃₋₆ cycloalkyl, each substituted with 0 to 3 substituents independently selected from halo; or
   (C) a is a double bond and p is a single bond;
      X¹ is CH;
      X² is N;
      X³ is absent;
      X⁴ is NR⁴; and
      R⁴ is selected from:
         (i) hydrogen; and
         (ii) C₁₋₄ alkyl, C₁₋₄ alkylcarbonyl, C₁₋₄ alkylsulfonyl and C₃₋₆ cycloalkyl, each substituted with 0 to 3 substituents independently selected from halo;
   (D) α is a single bond and β is a single bond; and
      X¹ is CH₂;
      X² is O and X³ is CH₂, or
      X² is CH₂ and X³ is O; and
      X⁴ is CH₂;
m is selected from 0, 1 and 2;
   each R^{a} and R^{b} is independently selected from hydrogen and C₁₋₄ alkyl, or R^{a} and R^{b}, together with a carbon atom to which both R^{a} and R^{b} are attached, form a C₃₋₆ cycloalkylidene, provided if m is 2, then no more than one R^{a} and R^{b}, together with the carbon atom to which R^{a} and R^{b} are attached, form a C₃₋₆ cycloalkylidene;
   R⁵ is selected from:
      (a) C₃₋₈ cycloalkyl, which is substituted with 0 to 5 substituents independently selected from:
         (i) halo, hydroxy, cyano and oxo;
         (ii) amino, which is substituted with 0 to 2 substituents independently selected from C₁₋₄ alkyl; and
         (iii) C₁₋₄ alkyl, C₁₋₄ alkylcarbonyl and C₁₋₄ alkoxy, each substituted with 0 to 3 substituents independently selected from halo;
      provided:
         if m is 0, X¹, X², X³, X⁴ and X⁸ are each CH, a and R are both double bonds, R⁶, R⁷, R¹⁰ and R¹¹ are each hydrogen, and R⁹ is chloro, then R⁵ is not cyclopropyl;
         if m is 0, X¹, X², X³, X⁴ and X⁸ are each CH, a and R are both double bonds, R⁵ is cyclopropylmethyl, R⁶ is n-propyl and R¹⁰ is hydrogen, then R⁷ and R¹¹ are not both methyl or ethyl;
         if m is 0, X¹, X², X³, X⁴ and X⁸ are each CH, a and R are both double bonds, R⁵ is cyclopropylmethyl, R⁶ is n-propyl, R⁷ is methyl, and R¹⁰ and R¹¹ are each hydrogen, then R⁹ is not hydrogen or hydroxy;
         if m is 0, X¹, X², X³, X⁴ and X⁸ are each CH, a and R are both double bonds, R⁶ is n-propyl, R⁷ is methoxy, R⁹ and R¹¹ are each hydrogen, and R¹⁰ is chloro, then R⁵ is not cyclopropylmethyl;
         if m is 0, X¹, X², X³, X⁴ and X⁸ are each CH, a and R are both double bonds, R⁶ is n-propyl, R⁷, R¹⁰ and R¹¹ are each hydrogen, and R⁹ is chloro, then R⁵ is not cyclopropylmethyl;
         if m is 0, X¹, X², X³, X⁴ and X⁸ are each CH, a and R are both double bonds, R⁵ is cyclopropylmethyl, R⁷, R⁹ and R¹¹ are each methyl, and R¹⁰ is hydrogen, then R⁶ is not methyl, ethyl, n-propyl or methoxyethyl;
         if m is 0, X¹ and X⁴ are both CH, X² and X³ are both CCl, α and β are both double bonds, R⁵ is cyclopentyl, and R⁶, R⁷, R⁹, R¹⁰ and R¹¹ are each hydrogen, then X⁸ is not N or CH;
         if m is 0, X¹, X², X³, X⁴ and X⁸ are each CH, a and R are both double bonds, and R⁶, R⁷, R⁹, R¹⁰ and R¹¹ are each hydrogen, then R⁵ is not cyclopentyl, cyclohexyl or cyclooctyl;
         if m is 1, X¹, X², X³, X⁴ and X⁸ are each CH, a and R are both double bonds, R⁵ is cyclohexyl, R^{b}, R⁶, R⁷, R⁹, R¹⁰ and R¹¹ are each hydrogen, then R^{a} is not methyl, ethyl, isopropyl, n-propyl;
         if m is 1, X¹, X², X³, X⁴ and X⁸ are each CH, a and R are both double bonds, R^{a} is methyl, R^{b}, R⁶, R⁷, R⁹ and R¹¹ are each hydrogen, and R¹⁰ is methoxy, then R⁵ is not cyclohexyl;
         if m is 1, X¹, X², X³, X⁴ and X⁸ are each CH, a and R are both double bonds, R^{a} is methyl, R^{b}, R⁶, R⁹, R¹⁰ and R¹¹ are each hydrogen, and R⁷ is methyl, then R⁵ is not cyclohexyl;
         if m is 0, X¹, X², X³, X⁴ and X⁸ are each CH, a and R are both double bonds, R⁵ is cyclohexyl, R⁶, R⁷ and R¹¹ are each hydrogen, and R¹⁰ is methyl, then R⁹ is not chloro or methyl;
         if m is 0, X¹, X², X³, X⁴ and X⁸ are each CH, a and R are both double bonds, R⁵ is cyclohexyl, R⁷ is methyl, R⁹ is hydroxy, and R¹⁰ and R¹¹ are each hydrogen, then R⁶ is not methyl or ethyl;
         if m is 0, X¹, X², X³, X⁴ and X⁸ are each CH, a and R are both double bonds, R⁶, R⁷, R⁹ and R¹¹ are each methyl, and R¹⁰ is hydrogen, then R⁵ is not cyclohexyl;
         if m is 0, X¹, X², X³, X⁴ and X⁸ are each CH, a and R are both double bonds, R⁶, R⁹ and R¹¹ are each hydrogen, and R⁷ and R¹⁰ are each methyl, then R⁵ is not cyclohexyl;
         if m is 0, X¹, X², X³, X⁴ and X⁸ are each CH, a and R are both double bonds, R⁶, R⁷, R¹⁰ and R¹¹ are each hydrogen, and R⁹ is methyl, then R⁵ is not cycloheptyl;
         if m is 0, X¹, X² and X⁴ are each CH₂, X³ is 0, X⁸ is CH, a and R are both single bonds, R⁶, R¹⁰ and R¹¹ are each hydrogen, R⁷ is hydroxy and R⁹ is CF₃ or OCF₃, then R⁵ is not 3-hydroxy-3-methylcyclobutyl;
         if m is 0, X¹, X³ and X⁴ are each CH₂, X² is 0, X⁸ is CH, a and R are both single bonds, R⁶, R¹⁰ and R¹¹ are each hydrogen, R⁷ is hydroxy and R⁹ is CF₃ or OCF₃, then R⁵ is not 3-hydroxy-3-methylcyclobutyl;
         if m is 0, X¹, X² and X⁴ are each CH₂, X³ is 0, X⁸ is CH, a and R are both single bonds, R⁶, R¹⁰ and R¹¹ are each hydrogen, R⁷ is hydroxy, and R⁹ is hydrogen, CF₃, OCF₃ or cyclobutyl, then R⁵ is not 2-hydroxycyclohexyl;
         if m is 0, X¹, X³ and X⁴ are each CH₂, X² is 0, X⁸ is CH, a and R are both single bonds, R⁶, R¹⁰ and R¹¹ are each hydrogen, R⁷ is hydroxy, and R⁹ is hydrogen, chloro, CF₃, CHF₂, OCF₃, OCHF₂, OCH₃ or cyclobutyl, then R⁵ is not 2-hydroxycyclohexyl;
         if m is 0, X¹, X² and X⁴ are each CH₂, X³ is 0, X⁸ is CH, a and R are both single bonds, R⁶ and R¹⁰ are each hydrogen, R⁷ is hydroxy, R⁹ is methyl, and R¹¹ is fluoro, then R⁵ is not 2-hydroxycyclohexyl;
         if m is 0, X¹, X³ and X⁴ are each CH₂, X² is 0, X⁸ is CH, a and R are both single bonds, R⁶ and R¹⁰ are each hydrogen, R⁷ is hydroxy, R⁹ is methyl, and R¹¹ is fluoro, then R⁵ is not 2-hydroxycyclohexyl;
         if m is 0, X¹, X³ and X⁴ are each CH₂, X² is 0, X⁸ is CH, a and R are both single bonds, R⁶ and R¹¹ are each hydrogen, R⁷ is hydroxy, and R⁹ is CF₃, and R¹⁰ is fluoro, then R⁵ is not 2-hydroxycyclohexyl;
         if m is 0, X¹, X² and X⁴ are each CH₂, X³ is 0, X⁸ is CH, a and R are both single bonds, R⁶, R¹⁰ and R¹¹ are each hydrogen, R⁷ is hydroxy, and R⁹ is CF₃, then R⁵ is not 2-cyanocyclohexyl or 2-aminocyclohexyl; and
      if m is 0, X¹, X³ and X⁴ are each CH₂, X² is 0, X⁸ is CH, a and R are both single bonds, R⁶, R¹⁰ and R¹¹ are each hydrogen, R⁷ is hydroxy, and R⁹ is CF₃, then R⁵ is not 2-cyanocyclohexyl; (b) C₃₋₈ heterocyclyl in which up to 3 carbon ring atoms are each independently substituted with 0 to 2 substituents independently selected from:
         (i) halo, hydroxy, cyano and oxo;
         (ii) amino, which is substituted with 0 to 2 substituents independently selected from C₁₋₄ alkyl; and
         (iii) C₁₋₄ alkyl, C₁₋₄ alkylcarbonyl and C₁₋₄ alkoxy, each substituted with 0 to 3 substituents independently selected from halo;
      and in which a nitrogen ring atom, if present, is unsubstituted or substituted with a substituent selected from:
         (i) C₁₋₄ alkyl, C₁₋₄ alkylcarbonyl and C₁₋₄ alkylsulfonyl, each substituted with 0 to 3 substituents independently selected from halo;
         (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
         (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
      wherein the C₃₋₈ heterocyclyl has only one ring heteroatom, the ring heteroatom selected from nitrogen, oxygen, and sulfur; and n is selected from 0 and 1;
      provided:
         if m is 0, X¹, X³, X⁴ and X⁸ are each CH, X² is CR², a and R are both double bonds, R, R⁷, R¹⁰ and R¹¹ are each hydrogen, and R² and R⁹ are each methoxy, then R⁵ is not: 1-(4-chloro-3-fluorobenzyl)piperidin-4-yl, 1-(4-methylbenzyl)piperidin-4-yl, 1-(3-fluoro-4-methylbenzyl)piperidin-4-yl, 1-(4-fluoro-3-methylbenzyl)piperidin-4-yl, 1-phenethylpiperidin-4-yl, 1-(2-methylbenzyl)piperidin-4-yl, 1-(3,5-dimethylbenzyl)piperidin-4-yl, 1-(4-methoxy-3-methylbenzyl)piperidin-4-yl, 1-(3,4-dichlorobenzyl)piperidin-4-yl, 1-(3-chloro-4-methylbenzyl)piperidin-4-yl, 1-(4-chlorobenzyl)piperidin-4-yl, 1-(3,4-difluorobenzyl)piperidin-4-yl, 1-(3,4-dimethoxybenzyl)piperidin-4-yl, 1-(4-bromo-3-methylbenzyl)piperidin-4-yl, 1-(2,4,6-trimethylbenzyl)piperidin-4-yl, 1-(4-chloro-3-methylbenzyl)piperidin-4-yl, 1-(3,4-dimethylbenzyl)piperidin-4-yl, 1-(3-methylbenzyl)piperidin-4-yl, 1-(4-ethylbenzyl)piperidin-4-yl, 1-(3-phenylpropyl)piperidin-4-yl, 1-(2,4-dichlorobenzyl)piperidin-4-yl, 1-(3-cyanobenzyl)piperidin-4-yl, 1-(3-chloro-4-fluorobenzyl)piperidin-4-yl, 1-(2,4-dimethylbenzyl)piperidin-4-yl, piperidin-4-yl, or 1-benzylpiperidin-4-yl;
         if m is 0, X¹, X², X³, X⁴ and X⁸ are each CH, a and R are both double bonds, and R⁶, R⁷, R⁹, R¹⁰ and R¹¹ are each hydrogen, then R⁵ is not piperidin-4-yl or 1-benzylpiperidin-4-yl;
         if m is 0, X¹, X², X³, X⁴ and X⁸ are each CH, a and R are both double bonds, and R⁶, R⁷, R¹⁰ and R¹¹ are each hydrogen, and R⁹ is methoxy, then R⁵ is not 1-benzylpiperidin-4-yl;
         if m is 0, X¹, X³ and X⁸ are each CH, X² is CR², X⁴ is CR⁴, a and R are both double bonds, R⁶, R⁷, R¹⁰ and R¹¹ are each hydrogen, and R², R⁴ and R⁹ are each methoxy, then R⁵ is not 1-benzylpiperidin-4-yl;
         if m is 0, X¹, X³, X⁴ and X⁸ are each CH, X² is CR², a and R are both double bonds, R⁶, R⁷, R⁹ and R¹⁰ are each hydrogen, and R² and R¹⁰ are each methoxy, then R⁵ is not 1-benzylpiperidin-4-yl;
         if m is 2, R^{a} and R^{b} at each occurrence is hydrogen, X¹, X², X³, X⁴ and X⁸ are each CH, a and R are both double bonds, and R⁶, R⁷, R⁹, R¹⁰ and R¹¹ are each hydrogen, then R⁵ is not 1-benzylpiperidin-4-yl;
         if m is 0, X¹, X², X³, X⁴ and X⁸ are each CH, a and R are both double bonds, R⁵ is pyrrolidin-3-yl, and R⁶, R⁷, R¹⁰ and R¹¹ are each hydrogen, then R⁹ is not hydrogen, fluoro or methyl;
         if m is 2, R^{a} and R^{b} at each occurrence is hydrogen, X¹, X², X³, X⁴ and X⁸ are each CH, a and R are both double bonds, R⁶, R⁷, R¹⁰ and R¹¹ are each hydrogen, and R⁹ is chloro, then R⁵ is not pyrrolidin-1-yl;
         if m is 1, X¹, X², X³, X⁴ and X⁸ are each CH, a and R are both double bonds, R⁵ is tetrahydrofuran-2-yl, and R^{a}, R^{b}, R⁶, R⁷, R¹⁰ and R¹¹ are each hydrogen, then R⁹ is not fluorine or methyl;
         if m is 0, X¹, X², X³, X⁴ and X⁸ are each CH, a and R are both double bonds, R⁶, R⁷, R¹⁰ and R¹¹ are each hydrogen, and R⁹ is methoxy, then R⁵ is not 6-oxaspiro[2.5]octan-1-yl;
         if m is 0, X¹, X² and X⁴ are each CH₂, X³ is 0, X⁸ is CH, a and R are both single bonds, R⁶, R¹⁰ and R¹¹ are each hydrogen, R⁷ is hydroxy and R⁹ is CF₃, then R⁵ is not piperidin-3-yl or 1-methylpiperidin-3-yl;
         if m is 0, X¹, X³ and X⁴ are each CH₂, X² is 0, X⁸ is CH, a and R are both single bonds, R⁶, R¹⁰ and R¹¹ are each hydrogen, R⁷ is hydroxy and R⁹ is CF₃, then R⁵ is not piperidin-3-yl or 1-methylpiperidin-3-yl;
         if m is 0, X¹, X² and X⁴ are each CH₂, X³ is 0, X⁸ is CH, a and R are both single bonds, R⁶ and R¹⁰ are each hydrogen, R⁷ is hydroxy, R⁹ is CF₃, methyl or chloro, and R¹¹ is fluoro, then R⁵ is not 1-methylpiperidin-3-yl; and
         if m is 0, X¹, X² and X⁴ are each CH₂, X³ is 0, X⁸ is CH, a and R are both single bonds, R⁶, R¹⁰ and R¹¹ are each hydrogen, R⁷ is hydroxy and R⁹ is methyl or chloro, then R⁵ is not 1-methylpiperidin-3-yl;
      (c) phenyl, which is substituted with 0 to 3 substituents independently selected from halo, hydroxy, cyano, C₁₋₄ alkyl and C₁₋₄ alkoxy, provided at least one of the substituents is hydroxy, and:
         if m is 0, X¹, X², X³, X⁴, and X⁸ are each CH, a and R are both double bonds, R⁵ is 3-hydroxyphenyl, and R⁶, R⁷, R¹⁰ and R¹¹ are each hydrogen, then R⁹ is not methyl, methoxy, ethoxy or chloro;
         if m is 0, X¹, X², X³, X⁴, and X⁸ are each CH, a and R are both double bonds, R⁵ is 2-hydroxyphenyl, and R⁶, R⁷, R¹⁰ and R¹¹ are each hydrogen, then R⁹ is not hydrogen, methyl, methoxy, chloro;
         if m is 0, X¹, X², X³, X⁴, and X⁸ are each CH, a and R are both double bonds, R⁵ is 2-hydroxyphenyl, and R⁶, R⁷ and R¹¹ are each hydrogen, then R⁹ and R¹⁰ are not both chloro;
         if m is 0, X¹, X², X³, X⁴, and X⁸ are each CH, a and R are both double bonds, R⁵ is 4-hydroxyphenyl, and R⁶, R⁷, R¹⁰ and R¹¹ are each hydrogen, then R⁹ is not hydrogen, hydroxy, chloro, methoxy, ethoxy, trifluoromethyl or tert-butyl;
         if m is 0, X¹, X², X³ and X⁴ are each CBr, X⁸ is CH, a and R are both double bonds, R⁶, R⁷, R¹⁰ and R¹¹ are each hydrogen, and R⁹ is methyl, then R⁵ is not 3-hydroxyphenyl or 4-hydroxyphenyl;
         if m is 0, X¹, X², X³, X⁴, and X⁸ are each CH, a and R are both double bonds, R⁵ is unsubstituted phenyl, and R⁶, R⁷, R¹⁰ and R¹¹ are each hydrogen, then R⁹ is not hydrogen, chloro, hydroxy, methyl or methoxy;
         if m is 0, X¹, X², X³, X⁴, and X⁸ are each CH, a and R are both double bonds, R⁵ is unsubstituted phenyl, R⁶, R⁷ and R¹⁰ are each hydrogen, and R¹¹ is methyl, then R⁹ is not hydrogen or chloro;
         if m is 0, X¹, X², X³, X⁴, and X⁸ are each CH, a and R are both double bonds, R⁶, R⁷, R⁹ and R¹⁰ are each hydrogen, and R¹¹ is methyl, then R⁵ is not unsubstituted phenyl;
         if m is 0, X¹, X², X³ and X⁴ are each CH, X⁸ is N, a and R are both double bonds, and R⁶, R⁷, R⁹, R¹⁰ and R¹¹ are each hydrogen, then R⁵ is not unsubstituted phenyl;
         if m is 0, X¹, X⁴ and X⁸ are each CH, X² is CR², X³ is CR³, a and R are both double bonds, R⁵ is unsubstituted phenyl, and R⁶, R⁷, R⁹, R¹⁰ and R¹¹ are each hydrogen, then R² and R³ are not both methyl or not both methoxy; and
         if m is 1, X¹, X², X³, X⁴, and X⁸ are each CH, a and R are both double bonds, R^{a} and R^{b}, together with the carbon atom to which both R^{a} and R^{b} are attached, form a cyclopentylidene, and R⁶, R⁷, R⁹, R¹⁰ and R¹¹ are each hydrogen, then R⁵ is not unsubstituted phenyl;
   R⁶ is selected from hydrogen and C₁₋₄ alkyl;
   X⁸ is selected from N and CR¹;
   R⁷, R⁸ and R¹¹ are each independently selected from:
      (i) hydrogen, halo, hydroxy and cyano;
      (ii) C₁₋₄ alkyl and C₁₋₄ alkoxy, each substituted with 0 to 3 substituents independently selected from halo; and
      (iii) C₃₋₈ cycloalkyl which is substituted with 0 to 3 substituents independently selected from halo, C₁₋₄ alkyl and C₁₋₄ alkoxy; and
   R⁹ and R¹⁰ are each independently selected from:
      (i) hydrogen, halo, hydroxy and cyano;
      (ii) C₁₋₄ alkyl and C₁₋₄ alkoxy, each substituted with 0 to 3 substituents independently selected from halo; and
      (iii) C₃₋₈ cycloalkyl which is substituted with 0 to 3 substituents independently selected from halo, C₁₋₄ alkyl and C₁₋₄ alkoxy; or R⁹ and R¹⁰ form an ethan-1,2-dioxy moiety bridging the carbon atoms to which they are attached.
   In addition to embodiment (1) in the preceding paragraph, the compounds of Formula 1 include those in which:
(2) α is a double bond and p is a double bond;
   X¹ is selected from N and CR¹;
   X² is selected from N and CR²;
   X³ is selected from N and CR³;
   X⁴ is selected from N and CR⁴; provided no more than one of X¹, X², X³ and X⁴ are N; and
   R¹, R², R³ and R⁴ are each independently selected from:
      (i) hydrogen, halo, hydroxy, cyano; and
      (ii) C₁₋₄ alkyl, C₁₋₄ alkoxy and C₃₋₆ cycloalkyl, each substituted with 0 to 3 substituents independently selected from halo.

In addition to embodiment (2) in the preceding paragraph, the compounds of Formula 1 include those in which:
(3) X¹ is CR¹, X² is CR², X³ is CR³ and X⁴ is CR⁴.

In addition to embodiment (3) in the preceding paragraph, the compounds of Formula 1 include those in which R¹, R², R³ and R⁴ are each independently selected from:
(4) (i) hydrogen; and
   (ii) C₁₋₄ alkyl and C₁₋₄ alkoxy, each substituted with 0 to 3 substituents independently selected from halo;
(5) (i) hydrogen; and
   (ii) C₁₋₃ alkyl and C₁₋₃ alkoxy, each substituted with 0 to 3 substituents independently selected from halo;
(6) (i) hydrogen; and
   (ii) C₁₋₃ alkyl and C₁₋₃ alkoxy, each unsubstituted;
(7) hydrogen, methyl and methoxy;
(8) hydrogen and methyl; or
(9) hydrogen.

In addition to embodiment (2) above, the compounds of Formula 1 include those in which:
(10) X¹ is N, X² is CR², X³ is CR³ and X⁴ is CR⁴.

In addition to embodiment (10) in the preceding paragraph, the compounds of Formula 1 include those in which R², R³ and R⁴ are each independently selected from:
(11) (i) hydrogen; and
   (ii) C₁₋₄ alkyl and C₁₋₄ alkoxy, each substituted with 0 to 3 substituents independently selected from halo;
(12) (i) hydrogen; and
   (ii) C₁₋₃ alkyl and C₁₋₃ alkoxy, each substituted with 0 to 3 substituents independently selected from halo;
(13) (i) hydrogen; and
   (ii) C₁₋₃ alkyl and C₁₋₃ alkoxy, each unsubstituted;
(14) hydrogen, methyl and methoxy;
(15) hydrogen and methyl; or
(16) hydrogen.

In addition to embodiment (2) above, the compounds of Formula 1 include those in which:
(17) X¹ is CR¹, X² is N, X³ is CR³ and X⁴ is CR⁴.

In addition to embodiment (17) in the preceding paragraph, the compounds of Formula 1 include those in which R¹, R³ and R⁴ are each independently selected from:
(18) (i) hydrogen; and
   (ii) C₁₋₄ alkyl and C₁₋₄ alkoxy, each substituted with 0 to 3 substituents independently selected from halo;
(19) (i) hydrogen; and
   (ii) C₁₋₃ alkyl and C₁₋₃ alkoxy, each substituted with 0 to 3 substituents independently selected from halo;
(20) (i) hydrogen; and
   (ii) C₁₋₃ alkyl and C₁₋₃ alkoxy, each unsubstituted;
(21) hydrogen, methyl and methoxy;
(22) hydrogen and methyl; or
(23) hydrogen.

In addition to embodiment (2) above, the compounds of Formula 1 include those in which:
(24) X¹ is CR¹, X² is CR², X³ is N and X⁴ is CR⁴.

In addition to embodiment (24) in the preceding paragraph, the compounds of Formula 1 include those in which R¹, R² and R⁴ are each independently selected from:
(25) (i) hydrogen; and
   (ii) C₁₋₄ alkyl and C₁₋₄ alkoxy, each substituted with 0 to 3 substituents independently selected from halo;
(26) (i) hydrogen; and
   (ii) C₁₋₃ alkyl and C₁₋₃ alkoxy, each substituted with 0 to 3 substituents independently selected from halo;
(27) (i) hydrogen; and
   (ii) C₁₋₃ alkyl and C₁₋₃ alkoxy, each unsubstituted;
(28) hydrogen, methyl and methoxy;
(29) hydrogen and methyl; or
(30) hydrogen.

In addition to embodiment (2) above, the compounds of Formula 1 include those in which:
(31) X¹ is CR¹, X² is CR², X³ is CR³ and X⁴ is N.

In addition to embodiment (31) in the preceding paragraph, the compounds of Formula 1 include those in which R¹, R² and R³ are each independently selected from:
(32) (i) hydrogen; and
   (ii) C₁₋₄ alkyl and C₁₋₄ alkoxy, each substituted with 0 to 3 substituents independently selected from halo;
(33) (i) hydrogen; and
   (ii) C₁₋₃ alkyl and C₁₋₃ alkoxy, each substituted with 0 to 3 substituents independently selected from halo;
(34) (i) hydrogen; and
   (ii) C₁₋₃ alkyl and C₁₋₃ alkoxy, each unsubstituted;
(35) hydrogen, methyl and methoxy;
(36) hydrogen and methyl; or
(37) hydrogen.

In addition to embodiment (1) above, the compounds of Formula 1 include those in which:
(38) α is a single bond and p is a single bond;
   X¹ is CH₂;
   X² is selected from NR² and CH₂;
   X³ is selected from NR³ and CH₂;
   X⁴ is CH₂; provided no more than one of X² and X³ is N; and
   R² and R³ are each independently selected from:
      (i) hydrogen; and
      (ii) C₁₋₄ alkyl, C₁₋₄ alkylcarbonyl, C₁₋₄ alkylsulfonyl and C₃₋₆ cycloalkyl, each substituted with 0 to 3 substituents independently selected from halo.

In addition to embodiment (38) in the preceding paragraph, the compounds of Formula 1 include those in which:
(39) X² is CH₂ and X³ is CH₂; or
(40) X² is NR² and X³ is CH₂.

In addition to embodiment (40) in the preceding paragraph, the compounds of Formula 1 include those in which R² is selected from:
(41) C₁₋₄ alkyl, C₁₋₄ alkylcarbonyl, C₁₋₄ alkylsulfonyl and C₃₋₆ cycloalkyl, each substituted with 0 to 3 substituents independently selected from halo;
(42) C₁₋₃ alkyl, C₁₋₃ alkylcarbonyl, C₁₋₃ alkylsulfonyl and C₃₋₆ cycloalkyl, each substituted with 0 to 3 substituents independently selected from halo;
(43) C₁₋₃ alkyl, C₁₋₃ alkylcarbonyl, C₁₋₃ alkylsulfonyl and C₃₋₆ cycloalkyl, each unsubstituted;
(44) methyl, ethyl, methylcarbonyl, ethylcarbonyl, methylsulfonyl, ethylsulfonyl, cyclopropyl and cyclobutyl;
(45) methyl, ethyl, methylcarbonyl, methylsulfonyl, and cyclopropyl; or
(46) methyl and methylcarbonyl.

In addition to embodiment (38) above, the compounds of Formula 1 include those in which:
(47) X² is CH₂ and X³ is NR³.

In addition to embodiment (47) in the preceding paragraph, the compounds of Formula 1 include those in which R³ is selected from:
(48) C₁₋₄ alkyl, C₁₋₄ alkylcarbonyl, C₁₋₄ alkylsulfonyl and C₃₋₆ cycloalkyl, each substituted with 0 to 3 substituents independently selected from halo;
(49) C₁₋₃ alkyl, C₁₋₃ alkylcarbonyl, C₁₋₃ alkylsulfonyl and C₃₋₆ cycloalkyl, each substituted with 0 to 3 substituents independently selected from halo;
(50) C₁₋₃ alkyl, C₁₋₃ alkylcarbonyl, C₁₋₃ alkylsulfonyl and C₃₋₆ cycloalkyl, each unsubstituted;
(51) methyl, ethyl, methylcarbonyl, ethylcarbonyl, methylsulfonyl, ethylsulfonyl, cyclopropyl and cyclobutyl;
(52) methyl, ethyl, methylcarbonyl, methylsulfonyl, and cyclopropyl; or
(53) methyl and methylcarbonyl.

In addition to embodiment (1) above, the compounds of Formula 1 include those in which:
(54) α is a double bond and β is a single bond;
   X¹ is CH;
   X² is N;
   X³ is absent;
   X⁴ is NR⁴; and
   R⁴ is selected from:
      (i) hydrogen; and
      (ii) C₁₋₄ alkyl, C₁₋₄ alkylcarbonyl, C₁₋₄ alkylsulfonyl and C₃₋₆ cycloalkyl, each substituted with 0 to 3 substituents independently selected from halo.

In addition to embodiment (54) in the preceding paragraph, the compounds of Formula 1 include those in which R⁴ is selected from:
(55) C₁₋₄ alkyl, C₁₋₄ alkylcarbonyl, C₁₋₄ alkylsulfonyl and C₃₋₆ cycloalkyl, each substituted with 0 to 3 substituents independently selected from halo;
(56) C₁₋₃ alkyl, C₁₋₃ alkylcarbonyl, C₁₋₃ alkylsulfonyl and C₃₋₆ cycloalkyl, each substituted with 0 to 3 substituents independently selected from halo;
(57) C₁₋₃ alkyl, C₁₋₃ alkylcarbonyl, C₁₋₃ alkylsulfonyl and C₃₋₆ cycloalkyl, each unsubstituted;
(58) methyl, ethyl, methylcarbonyl, ethylcarbonyl, methylsulfonyl, ethylsulfonyl, cyclopropyl and cyclobutyl;
(59) methyl, ethyl, methylcarbonyl, methylsulfonyl, and cyclopropyl; or
(60) methyl and methylcarbonyl.

In addition to embodiment (1) above, the compounds of Formula 1 include those in which:
(61) α is a single bond and β is a single bond; and
   X¹ is CH₂;
   X² is O and X³ is CH₂, or
   X² is CH₂ and X³ is O; and
   X⁴ is CH₂.

In addition to embodiment (61) in the preceding paragraph, the compounds of Formula 1 include those in which:
(62) X² is O and X³ is CH₂; or
(63) X² is CH₂ and X³ is O.

In addition to any one of embodiments (1) to (63) above, the compounds of Formula 1 include those in which m is:
(64) 0; or
(65) 1 or 2.

In addition to embodiment (65) in the preceding paragraph, the compounds of Formula 1 include those in which:
(66) each R^{a} and R^{b} is independently selected from hydrogen and C₁₋₄ alkyl;
(67) each R^{a} and R^{b} is independently selected from hydrogen and C₁₋₃ alkyl;
(68) each R^{a} and R^{b} is independently selected from hydrogen and methyl;
(69) each R^{a} is methyl and each R^{b} is hydrogen;
(70) each R^{a} is methyl and each R^{b} is methyl;
(71) each R^{a} is hydrogen and each R^{b} is hydrogen;
(72) each R^{a} and R^{b} is independently selected from hydrogen and C₁₋₄ alkyl, or R^{a} and R^{b}, together with a carbon atom to which both R^{a} and R^{b} are attached, form a cyclopropylidene or cyclobutylidene, provided if m is 2, then no more than one R^{a} and R^{b}, together with the carbon atom to which R^{a} and R^{b} are attached, form a cyclopropylidene or cyclobutylidene;
(73) each R^{a} and R^{b} is independently selected from hydrogen and C₁₋₄ alkyl, or R^{a} and R^{b}, together with a carbon atom to which both R^{a} and R^{b} are attached, form a cyclopropylidene, provided if m is 2, then no more than one R^{a} and R^{b}, together with the carbon atom to which R^{a} and R^{b} are attached, form a cyclopropylidene;
(74) each R^{a} and R^{b} is independently selected from hydrogen and C₁₋₃ alkyl, or R^{a} and R^{b}, together with a carbon atom to which both R^{a} and R^{b} are attached, form a cyclopropylidene, provided if m is 2, then no more than one R^{a} and R^{b}, together with the carbon atom to which R^{a} and R^{b} are attached, form a cyclopropylidene.
(75) each R^{a} and R^{b} is independently selected from hydrogen and methyl, or R^{a} and R^{b}, together with a carbon atom to which both R^{a} and R^{b} are attached, form a cyclopropylidene, provided if m is 2, then no more than one R^{a} and R^{b}, together with the carbon atom to which R^{a} and R^{b} are attached, form a cyclopropylidene; or
(76) each R^{a} and R^{b} is hydrogen, or R^{a} and R^{b}, together with a carbon atom to which both R^{a} and R^{b} are attached, form a cyclopropylidene, provided if m is 2, then no more than one R^{a} and R^{b}, together with the carbon atom to which R^{a} and R^{b} are attached, form a cyclopropylidene.

In addition to any one of embodiments (1) to (76) above, the compounds of Formula 1 include those in which R⁵ is:
(77) C₃₋₈ cycloalkyl, which is substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(78) C₄₋₇ cycloalkyl, which is substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(79) a cycloalkyl selected from cyclopropyl, cyclobutyl, cyclopentyl, cyclohexyl, bicyclo[2.2.1]heptan-1-yl and spiro[3.3]heptan-2-yl, each substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(80) a cycloalkyl selected from cyclobutyl, cyclohexyl, bicyclo[2.2.1]heptan-1-yl and spiro[3.3]heptan-2-yl, each substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(81) a cycloalkyl selected from cyclobutyl, cyclohexyl and bicyclo[2.2.1]heptan-1-yl, each substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(82) a cycloalkyl which is cyclobutyl substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(83) a cycloalkyl which is cyclohexyl substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo; or
(84) a cycloalkyl which is bicyclo[2.2.1]heptan-1-yl substituted with 0 to 5 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo.

In addition to any one of embodiments (77) to (84) in the preceding paragraph, the compounds of Formula 1 include those in which the R⁵ cycloalkyl is substituted with 0 to 5 substituents independently selected from:
(85) (i) halo, hydroxy and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(86) (i) halo and hydroxy;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(87) (i) hydroxy and fluoro;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(88) (i) hydroxy;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(89) (i) halo, hydroxy, cyano and oxo;
   (ii) amino, which is substituted with 0 to 2 substituents independently selected from C₁₋₃ alkyl; and
   (iii) C₁₋₄ alkyl, C₁₋₄ alkylcarbonyl and C₁₋₄ alkoxy, each substituted with 0 to 3 substituents independently selected from halo;
(90) (i) halo, hydroxy, cyano and oxo;
   (ii) amino, which is substituted with 0 to 2 substituents independently selected from methyl; and
   (iii) C₁₋₄ alkyl, C₁₋₄ alkylcarbonyl and C₁₋₄ alkoxy, each substituted with 0 to 3 substituents independently selected from halo;
(91) (i) halo, hydroxy, cyano and oxo;
   (ii) amino, which is unsubstituted; and
   (iii) C₁₋₄ alkyl, C₁₋₄ alkylcarbonyl and C₁₋₄ alkoxy, each substituted with 0 to 3 substituents independently selected from halo;
(92) (i) halo, hydroxy, cyano and oxo;
   (ii) amino, which is substituted with 0 to 2 substituents independently selected from C₁₋₄ alkyl; and
   (iii) C₁₋₃ alkyl, C₁₋₃ alkylcarbonyl and C₁₋₃ alkoxy, each substituted with 0 to 3 substituents independently selected from halo;
(93) (i) halo, hydroxy, cyano and oxo;
   (ii) amino, which is substituted with 0 to 2 substituents independently selected from C₁₋₄ alkyl; and
   (iii) methyl, methylcarbonyl, ethylcarbonyl, methoxy and ethoxy, each substituted with 0 to 3 substituents independently selected from halo;
(94) (i) halo, hydroxy, cyano and oxo;
   (ii) amino, which is substituted with 0 to 2 substituents independently selected from C₁₋₄ alkyl; and
   (iii) methyl and methoxy, each substituted with 0 to 3 substituents independently selected from halo;
(95) (i) hydroxy;
   (ii) amino, which is substituted with 0 to 2 substituents independently selected from methyl; and
   (iii) methyl and methoxy, each substituted with 0 to 3 substituents independently selected from halo;
(96) (i) hydroxy;
   (ii) amino, which is substituted with 0 to 2 substituents independently selected from methyl; and
   (iii) methyl and methoxy, each substituted with 0 to 3 substituents independently selected from fluoro; or
(97) hydroxy, dimethylamino, methyl, trifluoromethyl and methoxy.

In addition to any one of embodiments (77) to (97) in the preceding paragraphs, the compounds of Formula 1 include those in which the R⁵ cycloalkyl is:
(98) substituted with 0 to 4 substituents;
(99) substituted with 0 to 3 substituents;
(100) substituted with 0 to 2 substituents;
(101) substituted with 0 to 1 substituents; or
(102) is unsubstituted.

In addition to any one of embodiments (1) to (76) above, the compounds of Formula 1 include those in which R⁵ is:
(103) C₃₋₈ heterocyclyl in which up to 3 carbon ring atoms are each independently substituted with 0 to 2 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
and in which a nitrogen ring atom, if present, is unsubstituted or substituted with a substituent selected from:
   (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(104) C₃₋₈ heterocyclyl in which the ring heteroatom is selected from nitrogen and oxygen and up to 3 carbon ring atoms are each independently substituted with 0 to 2 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
and in which a nitrogen ring atom, if present, is unsubstituted or substituted with a substituent selected from:
   (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(105) C₃₋₈ heterocyclyl in which the ring heteroatom is nitrogen and up to 3 carbon ring atoms are each independently substituted with 0 to 2 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
and in which the nitrogen ring atom is unsubstituted or substituted with a substituent selected from:
   (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(106) a heterocyclyl selected from azetidinyl, piperidinyl, 1-azabicyclo[2.2.1]heptanyl, quinuclidinyl, pyrrolidinyl, 3-azabicyclo[3.1.0]hexan-1-yl and 2-oxabicyclo[2.2.1]heptan-4-yl in which up to 3 carbon ring atoms of the R⁵ heterocyclyl are each independently substituted with 0 to 2 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
and in which a nitrogen ring atom of the R⁵ heterocyclyl, if present, is unsubstituted or substituted with a substituent selected from:
   (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy; or
(107) a heterocyclyl selected from azetidin-1-yl, piperidin-2-yl, piperidin-3-yl, 1-azabicyclo[2.2.1]heptan-3-yl, quinuclidine-3-yl, pyrrolidin-1-yl, pyrrolidin-2-yl, pyrrolidin-3-yl, 3-azabicyclo[3.1.0]hexan-1-yl and 2-oxabicyclo[2.2.1]heptan-4-yl in which up to 3 carbon ring atoms of the R⁵ heterocyclyl are each independently substituted with 0 to 2 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
and in which a nitrogen ring atom of the R⁵ heterocyclyl, if present, is unsubstituted or substituted with a substituent selected from:
   (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy.
(108) a heterocyclyl selected from piperidinyl in which up to 3 carbon ring atoms of the R⁵ heterocyclyl are each independently substituted with 0 to 2 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
and in which the nitrogen ring atom of the R⁵ heterocyclyl, if available, is unsubstituted or substituted with a substituent selected from:
   (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy.
(109) a heterocyclyl selected from piperidin-3-yl in which up to 3 carbon ring atoms of the R⁵ heterocyclyl are each independently substituted with 0 to 2 substituents independently selected from:
   (i) halo, hydroxy, cyano and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
and in which the nitrogen ring atom of the R⁵ heterocyclyl is unsubstituted or substituted with a substituent selected from:
   (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy.

In addition to any one of embodiments (103) to (109) in the preceding paragraph, the compounds of Formula 1 include those in which up to 3 carbon ring atoms of the R⁵ heterocyclyl are each independently substituted with 0 to 2 substituents independently selected from:
(110) (i) halo, hydroxy and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(111) (i) halo and oxo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(112) (i) halo;
   (ii) amino, which is substituted with 0 to 2 substituents independently selected from C₁₋₄ alkyl; and
   (iii) C₁₋₄ alkyl, C₁₋₄ alkylcarbonyl and C₁₋₄ alkoxy, each substituted with 0 to 3 substituents independently selected from halo;
(113) (i) halo, hydroxy, cyano and oxo;
   (ii) amino, which is substituted with 0 to 2 substituents independently selected from C₁₋₃ alkyl; and
   (iii) C₁₋₄ alkyl, C₁₋₄ alkylcarbonyl and C₁₋₄ alkoxy, each substituted with 0 to 3 substituents independently selected from halo;
(114) (i) halo, hydroxy, cyano and oxo;
   (ii) amino, which is substituted with 0 to 2 substituents independently selected from methyl; and
   (iii) C₁₋₄ alkyl, C₁₋₄ alkylcarbonyl and C₁₋₄ alkoxy, each substituted with 0 to 3 substituents independently selected from halo;
(115) (i) halo, hydroxy, cyano and oxo;
   (ii) amino, which is unsubstituted; and
   (iii) C₁₋₄ alkyl, C₁₋₄ alkylcarbonyl and C₁₋₄ alkoxy, each substituted with 0 to 3 substituents independently selected from halo;
(116) (i) halo, hydroxy, cyano and oxo;
   (ii) amino, which is substituted with 0 to 2 substituents independently selected from C₁₋₄ alkyl; and
   (iii) C₁₋₃ alkyl, C₁₋₃ alkylcarbonyl and C₁₋₃ alkoxy, each substituted with 0 to 3 substituents independently selected from halo;
(117) (i) halo, hydroxy, cyano and oxo;
   (ii) amino, which is substituted with 0 to 2 substituents independently selected from C₁₋₄ alkyl; and
   (iii) methyl, methylcarbonyl, ethylcarbonyl, methoxy and ethoxy, each substituted with 0 to 3 substituents independently selected from halo;
(118) (i) halo, hydroxy, cyano and oxo;
   (ii) amino, which is substituted with 0 to 2 substituents independently selected from C₁₋₄ alkyl; and
   (iii) methyl and methoxy, each substituted with 0 to 3 substituents independently selected from halo;
(119) (i) halo and oxo;
   (ii) amino, which is substituted with 0 to 2 substituents independently selected from methyl; and
   (iii) methyl and methoxy, each substituted with 0 to 3 substituents independently selected from halo;
(120) (i) halo and oxo;
   (ii) amino, which is substituted with 0 to 2 substituents independently selected from methyl; and
   (iii) methyl and methoxy, each substituted with 0 to 3 substituents independently selected from fluoro;
(121) halo, oxo, methyl, ethyl, propyl, isopropyl, methoxy, ethoxy, propoxy and isopropoxy;
(122) halo, oxo, methyl, ethyl, propyl and isopropyl;
(123) fluoro, oxo, methyl, ethyl, propyl and isopropyl;
(124) halo; or
(125) fluoro.

In addition to any one of embodiments (103) to (125) in the preceding paragraphs, the compounds of Formula 1 include those in which:
(126) up to 2 carbon ring atoms of the R⁵ heterocyclyl are each substituted;
(127) up to 1 of the carbon ring atoms of the R⁵ heterocyclyl is substituted; or
(128) at least one of the carbon ring atoms of the R⁵ heterocyclyl is substituted;
(129) none of the carbon ring atoms of the R⁵ heterocyclyl is substituted.

In addition to any one of embodiments (103) to (129) in the preceding paragraphs, the compounds of Formula 1 include those in which R⁵ is C₃₋₈ heterocyclyl in which a nitrogen ring atom, if present, is unsubstituted or substituted with a substituent selected from:
(130) (i) C₁₋₃ alkyl, C₁₋₃ alkylcarbonyl and C₁₋₃ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(131) (i) C₁₋₃ alkyl, methylcarbonyl, ethylcarbonyl, methylsulfonyl and ethylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(132) (i) methyl, ethyl, isopropyl, methylcarbonyl and methylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(133) (i) methyl, ethyl, isopropyl, methylcarbonyl, methylsulfonyl, each substituted with 0 to 3 substituents selected from fluoro;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(134) (i) methyl, ethyl, isopropyl, methylcarbonyl, methylsulfonyl, each unsubstituted;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(135) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(136) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅ cycloalkyl moiety is substituted with 0 to 3 substituents independently selected from halo, C₁₋₃ alkyl, C₁₋₃ alkylcarbonyl, C₁₋₃ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(137) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅ cycloalkyl moiety is substituted with 0 to 3 substituents independently selected from halo, C₁₋₃ alkyl and C₁₋₃ alkoxy; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(138) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅ cycloalkyl moiety is substituted with 0 to 3 substituents independently selected from C₁₋₃ alkyl and C₁₋₃ alkoxy; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(139) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅cycloalkyl moiety is substituted with 0 to 3 substituents independently selected from halo, methyl and methoxy; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(140) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅ cycloalkyl moiety is substituted with 0 to 3 substituents independently selected from fluoro, methyl and methoxy; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(141) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅ cycloalkyl moiety is substituted with 0 to 3 substituents independently selected from methyl and methoxy; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(142) (i) C₁₋₄ alkyl, C₁₋₄ alkylcarbonyl and C₁₋₄ alkylsulfonyl, each substituted with 0 to 3 substituents independently selected from halo;
   (ii) C₃₋₅ cycloalkyl-(CH₂)ₙ, which C₃₋₅ cycloalkyl moiety is unsubstituted; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy;
(143) (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₃ alkyl and C₁₋₃ alkoxy;
(144) (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, cyano, methyl and methoxy;
(145) (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from halo, hydroxy, methyl and methoxy;
(146) (i) C₁₋₄ alkyl, C₁₋₄ alkylcarbonyl and C₁₋₄ alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from fluoro, chloro, hydroxy, methyl and methoxy;
(147) (i) C₁₋₄ alkyl, C₁₋₄ alkylcarbonyl and C₁0.4 alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are substituted with 0 to 3 substituents independently selected from fluoro, hydroxy, methyl and methoxy;
(148) (i) C₁₋₄ alkyl, C₁₋₄ alkylcarbonyl and C₁0.4 alkylsulfonyl, each substituted with 0 to 3 substituents independently selected from halo;
   (ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
   (iii) phenyl-(CH₂)ₙ and pyridinyl-(CH₂)ₙ, which phenyl and pyridinyl moieties are unsubstituted;
(149) (i) methyl, ethyl, isopropyl, methylcarbonyl, methylsulfonyl, each substituted with 0 to 3 substituents selected from fluoro;
   (ii) C₃₋₅ cycloalkyl-(CH₂)ₙ, which C₃₋₅ cycloalkyl moiety is substituted with 0 to 3 substituents independently selected from fluoro, methyl and methoxy; and
   (iii) phenyl-(CH₂)ₙ and pyridinyl-(CH₂)ₙ, which phenyl and pyridinyl moieties are substituted with 0 to 3 substituents independently selected from fluoro, hydroxy, methyl and methoxy;
(150) (i) methyl, ethyl, isopropyl, methylcarbonyl, methylsulfonyl, each substituted with 0 to 3 substituents selected from fluoro;
   (ii) C₃₋₅ cycloalkyl-(CH₂)ₙ, which C₃₋₅ cycloalkyl moiety is substituted with 0 to 3 substituents independently selected from methyl and methoxy; and
   (iii) phenyl-(CH₂)ₙ and pyridinyl-(CH₂)ₙ, which phenyl and pyridinyl moieties are substituted with 0 to 3 substituents independently selected from fluoro, hydroxy, methyl and methoxy;
(151) (i) methyl, ethyl, isopropyl, methylcarbonyl, methylsulfonyl, each substituted with 0 to 3 substituents selected from fluoro;
   (ii) C₃₋₅ cycloalkyl-(CH₂)ₙ, which C₃₋₅ cycloalkyl moiety is substituted with 0 to 3 substituents independently selected from methyl and methoxy; and
   (iii) phenyl-(CH₂)ₙ and pyridinyl-(CH₂)ₙ, which phenyl and pyridinyl moieties are unsubstituted.
(152) (i) methyl, ethyl, isopropyl, each substituted with 0 to 3 substituents selected from fluoro; and
   (ii) C₃₋₅ cycloalkyl-(CH₂)ₙ, which C₃₋₅ cycloalkyl moiety is substituted with 0 to 3 substituents independently selected from methyl and methoxy;
(153) (i) methyl, ethyl and isopropyl; and
   (ii) C₃₋₅ cycloalkyl-(CH₂)ₙ, which C₃₋₅ cycloalkyl moiety is substituted with 0 to 3 substituents independently selected from methyl and methoxy;
(154) methyl, ethyl, isopropyl and C₃₋₅ cycloalkyl-(CH₂)ₙ; or
(155) methyl, ethyl, isopropyl and cyclopropyl.

In addition to any one of embodiments (103) to (155) in the preceding paragraphs, the compounds of Formula 1 include those in which:
(156) R⁵ is C₃₋₈ heterocyclyl and n is 0; or
(157) R⁵ is C₃₋₈ heterocyclyl and n is 1.

In addition to any one of embodiments (1) to (76) above, the compounds of Formula 1 include those in which R⁵ is phenyl, which is:
(158) substituted with 0 to 3 substituents independently selected from halo, hydroxy, cyano, C₁₋₄ alkyl and C₁₋₄ alkoxy, provided at least one of the substituents is hydroxy;
(159) substituted with 0 to 3 substituents independently selected from halo, hydroxy, cyano, C₁₋₃ alkyl and C₁₋₃ alkoxy, provided at least one of the substituents is hydroxy;
(160) substituted with 0 to 3 substituents independently selected from halo, hydroxy, cyano, methyl and methoxy, provided at least one of the substituents is hydroxy;
(161) substituted with 0 to 2 substituents independently selected from halo, hydroxy, cyano, methyl and methoxy, provided at least one of the substituents is hydroxy;
(162) unsubstituted or substituted with hydroxy; or
(163) unsubstituted.

In addition to any one of embodiments (1) to (163) in the preceding paragraphs, the compounds of Formula 1 include those in which R⁶ is selected from:
(164) hydrogen and C₁₋₃ alkyl;
(165) hydrogen and methyl;
(166) methyl; or
(167) hydrogen.

In addition to any one of embodiments (1) to (167) in the preceding paragraphs, the compounds of Formula 1 include those in which:
(168) X⁸ is CR⁸.

In addition to any one of embodiments (1) to (168) in the preceding paragraphs, the compounds of Formula 1 include those in which R⁷, R⁸ and R¹¹ are each independently selected from:
(169) (i) hydrogen, halo and hydroxy;
   (ii) C₁₋₄ alkyl and C₁₋₄ alkoxy, each substituted with 0 to 3 substituents independently selected from halo; and
   (iii) C₃₋₈ cycloalkyl which is substituted with 0 to 3 substituents independently selected from halo, C₁₋₄ alkyl and C₁₋₄ alkoxy;
(170) (i) hydrogen, halo and hydroxy; and
   (ii) C₁₋₄ alkyl and C₁₋₄ alkoxy, each substituted with 0 to 3 substituents independently selected from halo;
(171) (i) hydrogen, halo and hydroxy; and
   (ii) C₁₋₃ alkyl and C₁₋₃ alkoxy, each substituted with 0 to 3 substituents independently selected from halo;
(172) (i) hydrogen, halo and hydroxy; and
   (ii) methyl and methoxy, each substituted with 0 to 3 substituents independently selected from halo; or
(173) (i) hydrogen, halo and hydroxy; and
   (ii) methyl and methoxy, each substituted with 0 to 3 fluoro.

In addition to any one of embodiments (1) to (168) above, the compounds of Formula 1 include those in which R⁷ and R⁸ are both hydrogen, and R¹¹ is selected from:
(174) (i) hydrogen, halo and hydroxy; and
   (ii) C₁₋₃ alkyl and C₁₋₃ alkoxy, each substituted with 0 to 3 substituents independently selected from halo;
(175) (i) hydrogen, halo and hydroxy; and
   (ii) methyl and methoxy, each substituted with 0 to 3 substituents independently selected from halo; or
(176) (i) hydrogen, halo and hydroxy; and
   (ii) methyl and methoxy, each substituted with 0 to 3 fluoro.

In addition to any one of embodiments (1) to (176) in the preceding paragraphs, the compounds of Formula 1 include those in which R⁹ and R¹⁰ are each independently selected from:
(177) (i) hydrogen, halo, hydroxy and cyano;
   (ii) C₁₋₄ alkyl and C₁₋₄ alkoxy, each substituted with 0 to 3 substituents independently selected from halo; and
   (iii) C₃₋₈ cycloalkyl which is substituted with 0 to 3 substituents independently selected from halo, C₁₋₄ alkyl and C₁₋₄ alkoxy;
(178) (i) hydrogen, halo, hydroxy and cyano;
   (ii) C₁₋₄ alkyl and C₁₋₃ alkoxy, each substituted with 0 to 3 substituents independently selected from halo; and
   (iii) C₃₋₈ cycloalkyl which is substituted with 0 to 3 substituents independently selected from halo, C₁₋₄ alkyl and C₁₋₄ alkoxy;
(179) (i) hydrogen, halo, hydroxy and cyano;
   (ii) C₁₋₄ alkyl and methoxy, each substituted with 0 to 3 substituents independently selected from halo; and
   (iii) C₃₋₈ cycloalkyl which is substituted with 0 to 3 substituents independently selected from halo, C₁₋₄ alkyl and C₁₋₄ alkoxy;
(180) (i) hydrogen, halo, hydroxy and cyano;
   (ii) C₁₋₄ alkyl and methoxy, each substituted with 0 to 3 fluoro; and
   (iii) C₃₋₈ cycloalkyl which is substituted with 0 to 3 substituents independently selected from halo, C₁₋₄ alkyl and C₁₋₄ alkoxy;
(181) (i) hydrogen, halo, hydroxy and cyano;
   (ii) C₁₋₄ alkyl and C₁₋₄ alkoxy, each substituted with 0 to 3 substituents independently selected from halo; and
   (iii) C₃₋₅ cycloalkyl which is substituted with 0 to 3 substituents independently selected from halo, C₁₋₄ alkyl and C₁₋₄ alkoxy;
(182) (i) hydrogen, halo, hydroxy and cyano;
   (ii) C₁₋₄ alkyl and C₁₋₄ alkoxy, each substituted with 0 to 3 substituents independently selected from halo; and
   (iii) C₃₋₅ cycloalkyl which is substituted with 0 to 3 substituents independently selected from C₁₋₄ alkyl and C₁₋₄ alkoxy;
(183) (i) hydrogen, halo, hydroxy and cyano;
   (ii) C₁₋₄ alkyl and C₁₋₄ alkoxy, each substituted with 0 to 3 substituents independently selected from halo; and
   (iii) C₃₋₅ cycloalkyl which is substituted with 0 to 3 substituents independently selected from C₁₋₃ alkyl and C₁₋₃ alkoxy;
(184) (i) hydrogen, halo, hydroxy and cyano;
   (ii) C₁₋₄ alkyl and C₁₋₄ alkoxy, each substituted with 0 to 3 substituents independently selected from halo; and
   (iii) C₃₋₅ cycloalkyl which is substituted with 0 to 3 substituents independently selected from methyl and methoxy;
(185) (i) hydrogen, halo, hydroxy and cyano;
   (ii) C₁₋₄ alkyl and C₁₋₄ alkoxy, each substituted with 0 to 3 substituents independently selected from halo; and
   (iii) cyclopropyl and cyclobutyl, each substituted with 0 to 3 substituents independently selected from methyl and methoxy;
(186) (i) hydrogen, halo, hydroxy and cyano;
   (ii) C₁₋₄ alkyl and C₁₋₃ alkoxy, each substituted with 0 to 3 substituents independently selected from halo; and
   (iii) C₃₋₅ cycloalkyl which is substituted with 0 to 3 substituents independently selected from C₁₋₃ alkyl and C₁₋₃ alkoxy;
(187) (i) hydrogen, halo, hydroxy and cyano;
   (ii) C₁₋₄ alkyl and C₁₋₃ alkoxy, each substituted with 0 to 3 fluoro; and
   (iii) C₃₋₅ cycloalkyl which is substituted with 0 to 3 substituents independently selected from C₁₋₃ alkyl and C₁₋₃ alkoxy; or
(188) (i) hydrogen, halo, hydroxy and cyano;
   (ii) C₁₋₄ alkyl and C₁₋₃ alkoxy, each substituted with 0 to 3 fluoro; and
   (iii) C₃₋₅ cycloalkyl which is substituted with 0 to 3 substituents independently selected from methyl and methoxy.

Compounds of Formula 1 include embodiments (1) through (188) described in the preceding paragraphs and compounds specifically named in the examples, may exist as salts, complexes, solvates, hydrates, and liquid crystals. Likewise, compounds of Formula 1 that are salts may exist as complexes, solvates, hydrates, and liquid crystals.

Compounds of Formula 1 may form pharmaceutically acceptable complexes, salts, solvates and hydrates. These salts include acid addition salts (including di-acids) and base salts. Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium, potassium, magnesium, calcium, zinc, and aluminum. Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2002).

Pharmaceutically acceptable salts may be prepared using various methods. For example, a compound of Formula 1 may be reacted with an appropriate acid or base to give the desired salt. Alternatively, a precursor of the compound of Formula 1 may be reacted with an acid or base to remove an acid- or base-labile protecting group or to open a lactone or lactam group of the precursor. Additionally, a salt of the compound of Formula 1 may be converted to another salt (or free form) through treatment with an appropriate acid or base or through contact with an ion exchange resin. Following reaction, the salt may be isolated by filtration if it precipitates from solution, or by evaporation to recover the salt. The degree of ionization of the salt may vary from completely ionized to almost non-ionized.

Compounds of Formula 1 may exist in a continuum of solid states ranging from fully amorphous to fully crystalline. The term “amorphous” refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”).

Compounds of Formula 1 may also exist in unsolvated and solvated forms. The term “solvate” describes a molecular complex comprising the compound and one or more pharmaceutically acceptable solvent molecules (e.g., ethanol). The term “hydrate” is a solvate in which the solvent is water. Pharmaceutically acceptable solvates include those in which the solvent may be isotopically substituted (e.g., D₂O, acetone-d₆, DMSO-d₆).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

Compounds of Formula 1 may also exist as multi-component complexes (other than salts and solvates) in which the compound (drug) and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun. (2004) 17:1889-1896. For a general review of multi-component complexes, see J. K. Haleblian, J. Pharm. Sci. (1975) 64(8):1269-88.

When subjected to suitable conditions, compounds of Formula 1 may exist in a mesomorphic state (mesophase or liquid crystal). The mesomorphic state lies between the true crystalline state and the true liquid state (either melt or solution). Mesomorphism arising as the result of a change in temperature is described as “thermotropic” and mesomorphism resulting from the addition of a second component, such as water or another solvent, is described as “lyotropic.” Compounds that have the potential to form lyotropic mesophases are described as “amphiphilic” and include molecules which possess a polar ionic moiety (e.g., —COO⁻Na⁺, —COO⁻K⁺, —SO₃⁻Na⁺) or polar non-ionic moiety (such as —N⁻N⁺(CH₃)₃). See, e.g., N. H. Hartshorne and A. Stuart, Crystals and the Polarizing Microscope (4th ed, 1970).

Each compound of Formula 1 may exist as polymorphs, stereoisomers, tautomers, or some combination thereof, may be isotopically-labeled, may result from the administration of a prodrug, or form a metabolite following administration.

“Prodrugs” refer to compounds having little or no pharmacological activity that can, when metabolized in vivo, undergo conversion to compounds having desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of compounds of Formula 1 having carboxylic acid, hydroxy, or amino functional groups, respectively. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds of Formula 1 having methyl, alkoxy, tertiary amino, secondary amino, phenyl, and amide groups, respectively.

Compounds of Formula 1 may exist as stereoisomers that result from the presence of one or more stereogenic centers, one or more double bonds, or both. The stereoisomers may be pure, substantially pure, or mixtures. Such stereoisomers may also result from acid addition or base salts in which the counter-ion is optically active, for example, when the counter-ion is D-lactate or L-lysine.

Compounds of Formula 1 may exist as tautomers, which are isomers resulting from tautomerization. Tautomeric isomerism includes, for example, imine-enamine, keto-enol, oxime-nitroso, and amide-imidic acid tautomerism.

Compounds of Formula 1 may exhibit more than one type of isomerism.

Geometrical (cis/trans) isomers may be separated by conventional techniques such as chromatography and fractional crystallization.

Conventional techniques for preparing or isolating a compound having a specific stereochemical configuration include chiral synthesis from a suitable optically pure precursor or resolution of the racemate (or the racemate of a salt or derivative) using, for example, chiral high-pressure liquid chromatography (HPLC). Alternatively, the racemate (or a racemic precursor) may be reacted with a suitable optically active compound, for example, an alcohol, or, in the case where the compound of Formula 1 contains an acidic or basic moiety, an acid or base such as tartaric acid or 1-phenylethylamine. The resulting diastereomeric mixture may be separated by chromatography, fractional crystallization, etc., and the appropriate diastereoisomer converted to the compound having the requisite stereochemical configuration. For a further discussion of techniques for separating stereoisomers, see E. L. Eliel and S. H. Wilen, Stereochemistry of Organic Compounds (1994).

Compounds of Formula 1 may possess isotopic variations, in which at least one atom is replaced by an atom having the same atomic number, but an atomic mass different from the atomic mass usually found in nature. Isotopes suitable for inclusion in compounds of Formula 1 include, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen, such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labeled compounds may be prepared by processes analogous to those described elsewhere in the disclosure using an appropriate isotopically-labeled reagent in place of a non-labeled reagent.

The compounds of Formula 1 may be prepared using the techniques described below. Some of the methods and examples may omit details of common reactions, including oxidations, reductions, and so on, separation techniques (extraction, evaporation, precipitation, chromatography, filtration, trituration, crystallization, and the like), and analytical procedures, which are known to persons of ordinary skill in the art of organic chemistry. The details of such reactions and techniques can be found in several treatises, including Richard Larock, Comprehensive Organic Transformations (1999), and the multi-volume series edited by Michael B. Smith and others, Compendium of Organic Synthetic Methods (1974 etseq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a di-acid, etc.). In addition, in some instances, reaction intermediates may be used in subsequent steps without isolation or purification (i.e., in situ).

In the methods and examples below, certain compounds may be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and so on, see T. W. Greene and P. G. Wuts, Protecting Groups in Organic Chemistry (1999) and P. Kocienski, Protective Groups (2000).

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about room temperature (RT) and ambient pressure, but depending on reaction kinetics, yields, and so on, some reactions may be run at elevated pressures or employ higher temperatures (e.g., reflux conditions) or lower temperatures (e.g., −78° C. to 0° C.). Any reference in the disclosure and claims to a stoichiometric range, a temperature range, a pH range, etc., whether expressly using the word “range,” also includes the indicated endpoints.

Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane, cyclohexane, methylcyclohexane); aromatic hydrocarbons (e.g., benzene, toluene, xylenes); halogenated hydrocarbons (e.g., methylene chloride, chloroform, carbon tetrachloride); aliphatic alcohols (e.g., methanol, ethanol, propan-1-ol, propan-2-ol, butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane, 1,2-diethoxy-ethane, 1-methoxy-2-(2-methoxy-ethoxy)-ethane, 1-ethoxy-2-(2-ethoxy-ethoxy)-ethane, tetrahydrofuran, 1,4-dioxane); ketones (e.g., acetone, methyl ethyl ketone); esters (methyl acetate, ethyl acetate); nitrogen-containing solvents (e.g., formamide, N,N-dimethylformamide, acetonitrile, N-methyl-pyrrolidone, pyridine, quinoline, nitrobenzene); sulfur-containing solvents (e.g., carbon disulfide, dimethyl sulfoxide, tetrahydro-thiophene-1,1-dioxide); and phosphorus-containing solvents (e.g., hexamethylphosphoric triamide).

In the schemes, below, substituent identifiers (e.g., α, β, m, R⁵, R⁶, R⁷, R⁹, R¹⁰, R¹¹, R^{a}, R^{b}, X¹, X², X³, X⁴ and X⁸) are as defined above for Formula 1. As mentioned earlier, some of the starting materials and intermediates may include protecting groups, which are removed prior to the final product. In such cases, the substituent identifier refers to moieties defined in Formula 1 and to those moieties with appropriate protecting groups. For example, a starting material or intermediate in the synthetic methods may include a potentially reactive (secondary) amine. In such cases, the amine would include the moiety with or without, say, a Boc or Cbz group attached to the amine.

Schemes A and B show general methods for preparing compounds of Formula 1. In accordance with Scheme A, a 1,4-dihalophthalazine derivative or analog (A1 in which, e.g., X is Cl) is reacted with an amine (A2) in the presence of a base (e.g., DIPEA, K₂CO₃, etc.) and solvent (e.g., ACN, DMSO, NMP, etc.) at elevated temperature (e.g., 80° C. to 150° C.) to give a halophthalazine amine (A3). The amine (A3) is subsequently reacted with a diboronic acid or ester (A4 in which, e.g., each R¹² is H or C₁₋₄ alkyl) in the presence of a palladium catalyst (e.g., Pd(PPH₃)₄, Pd(dppf)Cl₂, Pd(dppf)Cl₂·CH₂Cl₂, AmPhos PdCl₂, XPhos PdCl₂, etc.), base (e.g., Na₂CO₃, K₂CO₃, Cs₂CO₃, KF, etc.) and one or more solvents (e.g., 1,4-dioxane, DMF, ACN, EtOH, H₂O, etc.) at elevated temperature (e.g., 50-110° C.) to give the compound of Formula 1, directly or indirectly, e.g., after removal of protecting groups, further elaboration of functional groups, separation of stereoisomers or regioisomers, etc.

Alternatively, as shown in Scheme B, the 1,4-dihalophthalazine derivative or analog (A1) may be first reacted with the diboronic acid or ester (A4) in the presence of a palladium catalyst, base and solvent as noted for Scheme A. The resulting aromatic-substituted halophthalazine (B1) is then reacted with the amine (A2) in the presence of a base and solvent at elevated temperature, as described for Scheme A, to give the compound of Formula 1, either directly or after removal of protecting groups, further elaboration of functional groups, separation of stereoisomers or regioisomers, etc.

The methods depicted in the schemes may be varied as desired. For example, protecting groups may be added or removed and products may be further elaborated via, for example, alkylation, acylation, hydrolysis, oxidation, reduction, amidation, sulfonation, alkynation, and the like to give the desired final product. Furthermore, any intermediate or final product which comprises mixture of stereoisomers may be optionally purified by chiral column chromatography (e.g., supercritical fluid chromatography) or by derivatization with optically-pure reagents as described above to give a desired stereoisomer.

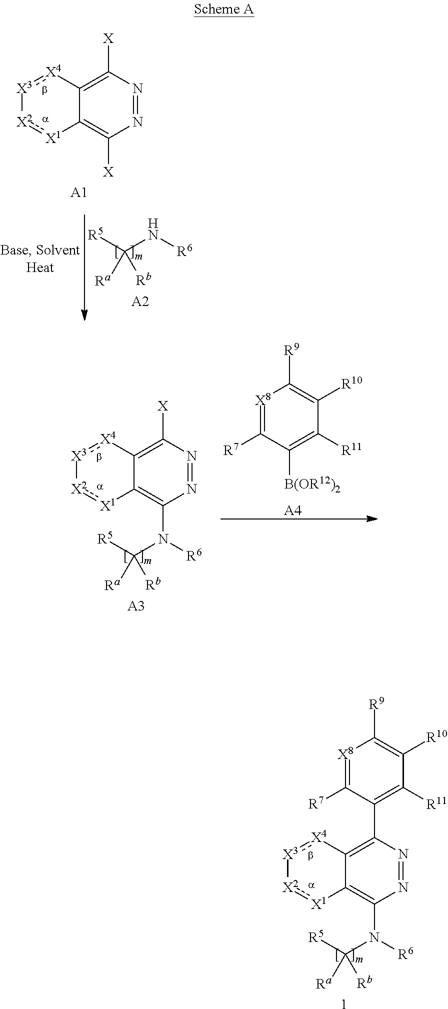

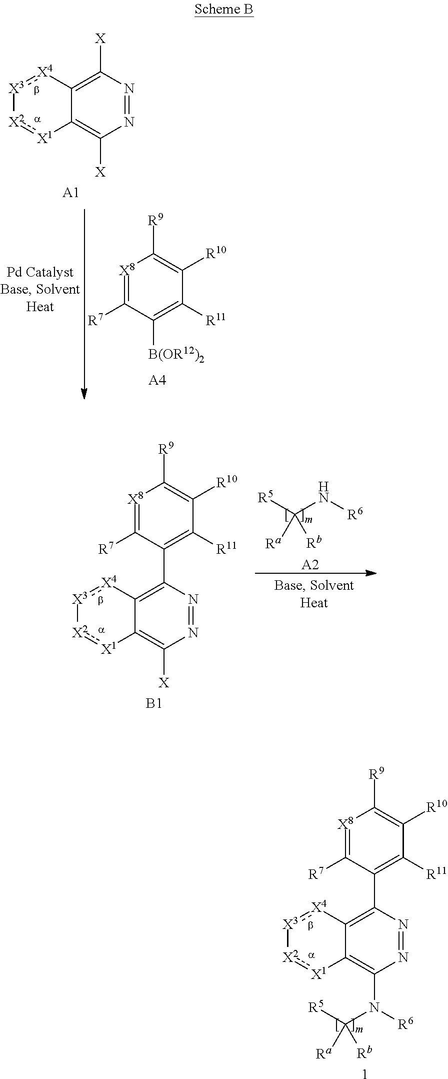

Compounds of Formula 1, which include compounds named above, and their pharmaceutically acceptable complexes, salts, solvates and hydrates, should be assessed for their biopharmaceutical properties, such as solubility and solution stability across pH, permeability, and the like, to select an appropriate dosage form and route of administration. Compounds that are intended for pharmaceutical use may be administered as crystalline or amorphous products, and may be obtained, for example, as solid plugs, powders, or films by methods such as precipitation, crystallization, freeze drying, spray drying, evaporative drying, microwave drying, or radio frequency drying.

Compounds of Formula 1 may be administered alone or in combination with one another or with one or more pharmacologically active compounds which are different than the compounds of Formula 1. Generally, one or more of these compounds are administered as a pharmaceutical composition (a formulation) in association with one or more pharmaceutically acceptable excipients. The choice of excipients depends on the mode of administration, the effect of the excipient on solubility and stability, and the nature of the dosage form, among other things. Useful pharmaceutical compositions and methods for their preparation may be found, for example, in A. R. Gennaro (ed.), Remington: The Science and Practice of Pharmacy (20th ed., 2000).

Compounds of Formula 1 may be administered orally. Oral administration may involve swallowing in which case the compound enters the bloodstream via the gastrointestinal tract. Alternatively, or additionally, oral administration may involve mucosal administration (e.g., buccal, sublingual, supralingual administration) such that the compound enters the bloodstream through the oral mucosa.

Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches. Liquid formulations include suspensions, solutions, syrups and elixirs. Such formulations may be employed as fillers in soft or hard capsules (made, e.g., from gelatin or hydroxypropylmethylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

Compounds of Formula 1 may also be used in fast-dissolving, fast-disintegrating dosage forms such as those described in Liang and Chen, Expert Opinion in Therapeutic Patents (2001) 11(6):981-986.

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁₋₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate.

Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet.

Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet.

Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant. For a discussion of blending, granulation, milling, screening, tableting, coating, as well as a description of alternative techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997).

Consumable oral films for human or veterinary use are pliable water-soluble or water-swellable thin film dosage forms which may be rapidly dissolving or mucoadhesive. In addition to the API, a typical film includes one or more film-forming polymers, binders, solvents, humectants, plasticizers, stabilizers or emulsifiers, viscosity-modifying agents, and solvents. Other film ingredients may include anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation may perform more than one function.

In addition to dosing requirements, the amount of API in the film may depend on its solubility. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80 wt % of the film.

Film dosage forms are typically prepared by evaporative drying of thin aqueous films coated onto a peelable backing support or paper, which may be carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14.

Compounds of Formula 1 may also be administered directly into the blood stream, muscle, or an internal organ of the subject. Suitable techniques for parenteral administration include intravenous, intraarterial, intraperitoneal, intrathecal, intraventricular, intraurethral, intrasternal, intracranial, intramuscular, intrasynovial, and subcutaneous administration. Suitable devices for parenteral administration include needle injectors, including microneedle injectors, needle-free injectors, and infusion devices.

Parenteral formulations are typically aqueous solutions which may contain excipients such as salts, carbohydrates and buffering agents (e.g., pH of from about 3 to about 9). For some applications, however, compounds of Formula 1 may be more suitably formulated as a sterile non-aqueous solution or as a dried form to be used in conjunction with a suitable vehicle such as sterile, pyrogen-free water. The preparation of parenteral formulations under sterile conditions (e.g., by lyophilization) may be readily accomplished using standard pharmaceutical techniques.

The solubility of compounds which are used in the preparation of parenteral solutions may be increased through appropriate formulation techniques, such as the incorporation of solubility-enhancing agents. Formulations for parenteral administration may be formulated to be immediate or modified release. Modified release formulations include delayed, sustained, pulsed, controlled, targeted, and programmed release. Thus, compounds of Formula 1 may be formulated as a suspension, a solid, a semi-solid, or a thixotropic liquid for administration as an implanted depot providing modified release of the active compound. Examples of such formulations include drug-coated stents and semi-solids and suspensions comprising drug-loaded poly(DL-lactic-coglycolic)acid (PGLA) microspheres.

Compounds of Formula 1 may also be administered topically, intradermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers may include alcohol, water, mineral oil, liquid petrolatum, white petrolatum, glycerin, polyethylene glycol and propylene glycol. Topical formulations may also include penetration enhancers. See, e.g., Finnin and Morgan, J. Pharm. Sci. 88(10):955-958 (1999).

Other means of topical administration include delivery by electroporation, iontophoresis, phonophoresis, sonophoresis and microneedle or needle-free (e.g. Powderject™ and Bioject™) injection. Formulations for topical administration may be formulated to be immediate or modified release as described above.

Compounds of Formula 1 may also be administered intranasally or by inhalation, typically in the form of a dry powder, an aerosol spray, or nasal drops. An inhaler may be used to administer the dry powder, which comprises the API alone, a powder blend of the API and a diluent, such as lactose, or a mixed component particle that includes the API and a phospholipid, such as phosphatidylcholine. For intranasal use, the powder may include a bioadhesive agent, e.g., chitosan or cyclodextrin. A pressurized container, pump, sprayer, atomizer, or nebulizer, may be used to generate the aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) which serve as a propellant, and an optional surfactant, such as sorbitan trioleate, oleic acid, or an oligolactic acid. An atomizer using electrohydrodynamics may be used to produce a fine mist.

Prior to use in a dry powder or suspension formulation, the drug product is usually comminuted to a particle size suitable for delivery by inhalation (typically 90% of the particles, based on volume, having a largest dimension less than 5 microns). This may be achieved by any appropriate size reduction method, such as spiral jet milling, fluid bed jet milling, supercritical fluid processing, high pressure homogenization, or spray drying.

Capsules, blisters and cartridges (made, for example, from gelatin or hydroxypropylmethyl cellulose) for use in an inhaler or insufflator may be formulated to contain a powder mixture of the active compound, a suitable powder base such as lactose or starch, and a performance modifier such as L-leucine, mannitol, or magnesium stearate. The lactose may be anhydrous or monohydrated. Other suitable excipients include dextran, glucose, maltose, sorbitol, xylitol, fructose, sucrose, and trehalose.

A suitable solution formulation for use in an atomizer using electrohydrodynamics to produce a fine mist may contain from about 1 g to about 20 mg of the API per actuation and the actuation volume may vary from about 1 μL to about 100 μL. A typical formulation may comprise one or more compounds of Formula 1, propylene glycol, sterile water, EtOH, and NaCl. Alternative solvents, which may be used instead of propylene glycol, include glycerol and polyethylene glycol.

Formulations for inhaled administration, intranasal administration, or both, may be formulated to be immediate or modified release using, for example, PGLA. Suitable flavors, such as menthol and levomenthol, or sweeteners, such as saccharin or sodium saccharin, may be added to formulations intended for inhaled/intranasal administration.

In the case of dry powder inhalers and aerosols, the dosage unit is determined by means of a valve that delivers a metered amount. Units are typically arranged to administer a metered dose or “puff” containing from about 10 g to about 1000 g of the API. The overall daily dose will typically range from about 100 g to about 10 mg which may be administered in a single dose or, more usually, as divided doses throughout the day.

The active compounds may be administered rectally or vaginally, e.g., in the form of a suppository, pessary, or enema. Cocoa butter is a traditional suppository base, but various alternatives may be used as appropriate. Formulations for rectal or vaginal administration may be formulated to be immediate or modified release as described above.

Compounds of Formula 1 may also be administered directly to the eye or ear, typically in the form of drops of a micronized suspension or solution in isotonic, pH-adjusted, sterile saline. Other formulations suitable for ocular and aural administration include ointments, gels, biodegradable implants (e.g. absorbable gel sponges, collagen), non-biodegradable implants (e.g. silicone), wafers, lenses, and particulate or vesicular systems, such as niosomes or liposomes. The formulation may include one or more polymers and a preservative, such as benzalkonium chloride. Typical polymers include crossed-linked polyacrylic acid, polyvinylalcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations may also be delivered by iontophoresis. Formulations for ocular or aural administration may be formulated to be immediate or modified release as described above.

To improve their solubility, dissolution rate, taste-masking, bioavailability, or stability, compounds of Formula 1 may be combined with soluble macromolecular entities, including cyclodextrin and its derivatives and polyethylene glycol-containing polymers. For example, API-cyclodextrin complexes are generally useful for most dosage forms and routes of administration. Both inclusion and non-inclusion complexes may be used. As an alternative to direct complexation with the API, the cyclodextrin may be used as an auxiliary additive, i.e. as a carrier, diluent, or solubilizer. Alpha-, beta- and gamma-cyclodextrins are commonly used for these purposes. See, e.g., WO 91/11172, WO 94/02518, and WO 98/55148.

As noted above, one or more compounds of Formula 1, including compounds specifically named above, and their pharmaceutically active complexes, salts, solvates and hydrates, may be combined with each other or with one or more other active pharmaceutically active compounds to treat various diseases, conditions and disorders. In such cases, the active compounds may be combined in a single dosage form as described above or may be provided in the form of a kit which is suitable for coadministration of the compositions. The kit comprises (1) two or more different pharmaceutical compositions, at least one of which contains a compound of Formula 1; and (2) a device for separately retaining the two pharmaceutical compositions, such as a divided bottle or a divided foil packet. An example of such a kit is the familiar blister pack used for the packaging of tablets or capsules. The kit is suitable for administering different types of dosage forms (e.g., oral and parenteral) or for administering different pharmaceutical compositions at separate dosing intervals, or for titrating the different pharmaceutical compositions against one another. To assist with patient compliance, the kit typically comprises directions for administration and may be provided with a memory aid.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient (e.g., an infant) whose mass falls outside of this weight range.

As noted above, the compounds of Formula 1 may be used to treat diseases, disorders, and conditions for which inhibition of the NLRP3 inflammasome pathway is indicated, including diseases, disorders or conditions associated with a heterozygous gain of function mutation in the NLRP3 gene, such as a cryopyrin-associated periodic syndrome (CAPS). These may include neonatal-onset multisystem inflammatory disease (NOMID/CINCA), Muckle-Wells syndrome (MWS), and familial cold autoinflammatory syndrome (FCAS).

The compounds of Formula 1 may be used to treat neurodegenerative diseases, disorders, and conditions associated with NLRP3. These may include Parkinson's disease, Huntington's disease, amyotrophic lateral sclerosis, prion disease, Alzheimer's disease, and other forms of dementia (i.e., major or mild neurocognitive disorders) associated with one or more medical conditions, including frontotemporal lobar degeneration, Lewy body disease, vascular disease, traumatic brain injury, substance or medication use, HIV infection, prion disease, Parkinson's disease, and Huntington's disease. The compounds of Formula 1 may also be used to treat major or mild neurocognitive disorders associated with depression, schizophrenia, bipolar disorder, and autism.

The claimed and disclosed compounds may be combined with one or more other pharmacologically active compounds or therapies to treat one or more disorders, diseases or conditions for which inhibition of the NLRP3 inflammasome pathway is indicated. Such combinations may offer significant therapeutic advantages, including fewer side effects, improved ability to treat underserved patient populations, or synergistic activity. For example, compounds of Formula 1, which include compounds specifically named above, and their pharmaceutically acceptable complexes, salts, solvates and hydrates, may be administered simultaneously, sequentially or separately in combination with one or more compounds or therapies for treating Alzheimer's disease, including beta-secretase inhibitors, gamma-secretase inhibitors, HMG-CoA reductase inhibitors, nonsteroidal anti-inflammatory drugs (NSAIDs, such as apazone, aspirin, celecoxib, diclofenac (with and without misoprostol), diflunisal, etodolac, fenoprofen, flurbiprofen, ibuprofen, indomethacin, ketoprofen, meclofenamate sodium, mefenamic acid, meloxicam, nabumetone, naproxen, oxaprozin, phenylbutazone, piroxicam, choline and magnesium salicylates, salsalate, and sulindac), vitamin E, and anti-amyloid antibodies. Specific examples of compounds used to treat Alzheimer's disease include donepezil, rivastigmine, memantine, and galantamine.

In addition to drugs used to improve cognition, the compounds of Formula 1 may be combined with sedatives, hypnotics, anxiolytics, antipsychotics, tranquilizers, and other medications that are used in the treatment of Alzheimer's disease. For example, the compounds of Formula 1 may be combined with one or more agents for treating depression (antidepressants) and/or schizophrenia (atypical or typical antipsychotics) including amitriptyline, amoxapine, aripiprazole, asenapine, bupropion, chlordiazepoxide, citalopram, chlorpromazine, clozapine, desipramine, desvenlafaxine, doxepin, duloxetine, escitalopram, fluoxetine, fluoxetine, fluphenazine, haloperidol, iloperidone, imipramine, isocarboxazid, lamotrigine, levomilnacipran, lurasidone, mirtazapine, nefazodone, nortriptyline, olanzapine, paliperidone, paroxetine, perphenazine, phenelzine, protriptyline, quetiapine, risperidone, selegiline, sertraline, tranylcypromine, trazodone, trimipramine, venlafaxine, vilazodone, and vortioxetine, and ziprasidone.

Likewise, the compounds of Formula 1 may be combined with one or more agents for treating anxiety (anxiolytics) including benzodiazepines (alprazolam, chlordiazepoxide, clobazepam, clonazepam, clorazepate, diazepam, estazolam, flurazepam, lorazepam, midazolam, oxazepam, prazepam, quazepam, temazepam, and triazolam), antihistamines (hydroxyzine), non-benzodiazepines (eszopiclone, zaleplon, zolpidem, and zopiclone) and buspirone.

The compounds of Formula 1 may also be combined with one or more agents for treating epilepsy (antiepileptics or anticonvulsants) including acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, and zonisamide.

### Biological Activity

The biological activity of the compound of Formula 1 with respect to NLRP3 may be determined using the following in vitro methods.

### IL-1β Assay (Reported as IC₅₀)

Monocytic THP-1 cells (ATCC: TIB-202) are maintained in accordance with the provider's instructions in RPMI media (Life Technologies, Cat #A10491-01); RPMI is supplemented with 10% heat inactivated fetal bovine serum (Hyclone Cat #SH30396.03). The cells are differentiated into macrophages by the addition of 25 ng/mL IFN-7 (PeproTech, Cat #300-02-100UG) for 24 hours at 37° C./5% CO₂. Media is exchanged with fresh media with no FBS, and the cells are treated with 50 ng/mL LPS (priming step) for 24 hours at 37° C./5% CO₂ (LPS-EK: Invivogen, Cat #tlrl-peklps). Media is exchanged with fresh media with no FBS. The cells are plated at 40,000 cells per well in 384-well flat-bottom cell culture plates (Costar 3764) containing compounds (added in 1:1000) in a 1:3.16 serial dilution series in DMSO and are incubated for 30 minutes at 37° C./5% CO₂. The NLRP3 inflammasome is activated with the addition of 2.5 mM ATP (Sigma Cat #A3377) and the cells are incubated for 2 hours at 37° C./5% CO₂. At the end of the incubation period, 40 μL supernatant is removed, and IL-10 levels are monitored using an ELISA (Human IL-10 ELISA, R&D systems, Cat #DY201) in accordance with the manufacturer's instructions.

### Description of the TNF-α Assay (Reported as IC₅₀)

Monocytic THP-1 cells (ATCC: TIB-202) are maintained in accordance with the provider's instructions in RPMI media (Life Technologies, Cat #A10491-01); RPMI is supplemented with 10% heat inactivated fetal bovine serum (Hyclone Cat #SH30396.03). The cells are differentiated into macrophages by the addition of 25 ng/mL IFN-γ for 24 hours at 37° C./5% CO₂. Media is exchanged with fresh media with no FBS. The cells are plated at 40,000 cells per well in 384-well flat-bottom cell culture plates (Costar 3764) containing compounds (added in 1:1000) in a 1:3.16 serial dilution series in DMSO and are incubated for 30 minutes at 37° C./5% CO₂. The NF-κB pathway is activated with the addition of 50 ng/mL LPS and the cells are incubated for 3 hours at 37° C./5% CO₂. At the end of the incubation period, supernatant (40 μL) is removed, and IL-10 levels are monitored using an ELISA (Human TNF-α ELISA, R&D systems, Cat #DY210) according to the manufacturer's instructions.

### Data Interpretation

The IC₅₀ values are calculated from a plot of percentage of inhibition versus the inhibitor concentration by a logistics curve fit according to: Y=[Bottom+(Top−Bottom)]/(1+10\hat{ }[(Log IC₅₀−X)·Hill Slope], where Y is the % inhibition at the inhibitor concentration, X, “Bottom” is the lowest inhibition value, i.e. 0%, “Top” is the maximum inhibition value, i.e. 100%, and the “Hill Slope” describes the slope of the sigmoidal curve between the “Bottom” and “Top” values. The curve fitting was conducted with internally developed software.

The following in vitro assay may be used to assess the ability of a compound of Formula 1 to enter the CNS through the blood-brain barrier.

### MDCK-MDR1 Assay (Reported as Apparent Permeability and Efflux Ratio)

Madine-Darby Canine Kidney (MDCK) cells transfected with Multidrug resistance protein 1 (MDR1) are maintained in accordance with the provider's instructions in Dulbecco's Modified Eagle media (DMEM, Fisher Scientific Cat #10569044). DMEM is supplemented with 10% heat inactivated fetal bovine serum (Gibco Cat #16000-044), Penicillin-Streptomycin (100 units/mL) (Gibco Cat #15140122) and an inducer of P-gp, colchicine (200 nM) (Sigma Cat #C9754). The cells are seeded onto the apical side of HTS-Transwell-96 Plates (0.4 μm pore size, Corning Cat #3381) at a density of 6.25×10³ cells per well with 75 μL and 250 μL of DMEM media in apical and basolateral wells, respectively, and are incubated at 37° C./5% CO₂. Fresh DMEM media is exchanged in the apical and basolateral compartments after 72 hours and cells are allowed to grow into a monolayer for 144 hours before beginning the experimental incubation. Incubations are performed in Hanks' Balanced Salt Solution (HBSS, Fisher Scientific Cat #14025134) at pH 7.4 with 1% Bovine Serum Albumin (Sigma, Cat #A9418) and 10 mM HEPES (Fisher Scientific, Cat #15630080). DMEM media is removed and cells are rinsed with warm (37° C.) HBSS. HBSS with test compound at 1p M substrate concentration (0.1% v/v DMSO) is added to either the apical or basolateral compartment (75 μL or 250 μL, respectively) and blank HBSS buffer is added to the compartment which lacks test compound in singlicate. The cells are incubated for 60 minutes at 37° C./5% CO₂. At the end of the incubation period, 50 μL of sample is removed from each receiver compartment and diluted into 150 μL of acetonitrile (Fisher Scientific, Cat #A996SK4)+0.1% formic acid (Sigma, Cat #F0507). The samples are centrifuged at 2000 rcf for 10 minutes at 4° C., after which 100 μL of supernatant is transferred to a new microplate and diluted with 100 μL of HPLC grade water (Fisher Scientific, Cat #W64). Samples are analyzed using a triple quadrupole mass spectrometer API-5500QTrap (ABSciex, Serial No. AU23291006) along with associated autosampler and high performance liquid chromatography pump instrumentation optimized for the detection of test articles through a Kinetix 2.1×50 mm C18 100 Å column (Phenomenex, Cat #00B-4605-AN).

Apparent permeability (Pₐₚₚ) values and efflux ratio are calculated using the following equations:

$${P}_{app A - B} ( nm \times {sec}^{- 1} ) = \frac{{Conc}_{BL} \times 0.25 \times 10000}{A \times t \times 10} ;$$

$${P}_{app B - A} ( nm \times {sec}^{- 1} ) = \frac{{Conc}_{AP} \times 0.075 \times 10000}{A \times t \times 10} ;$$

$$ER = \frac{{P}_{app B - A}}{{P}_{app A - B}} ;$$

where P_{app A-B} is the apparent permeability from the apical well to basolateral well; P_{app B-A} is the apparent permeability from the basolateral well to the apical well; Conc_{BL} is the basolateral well concentration; Conc_{AP} is the apical well concentration; A is the well surface area (cm²), which for the above assay is 0.143 cm²; t is the incubation time (seconds), which for the above assay is 3600 seconds; and ER is the P-gp mediated efflux ratio.

### EXAMPLES

The following examples are intended to be illustrative and non-limiting and represent specific embodiments of the present invention.

¹H Nuclear magnetic resonance (NMR) spectra were obtained for many of the compounds in the following examples. Characteristic chemical shifts (δ) are given in parts-per-million downfield from tetramethylsilane using conventional abbreviations for designation of major peaks, including s (singlet), d (doublet), t (triplet), q (quartet), m (multiplet), and br (broad). The following abbreviations are used for common solvents: CDCl₃ (deuterochloroform), DMSO-d₆ (deuterodimethylsulfoxide), CD₃OD (deuteromethanol), CD₃CN (deuteroacetonitrile), and THF-d₈ (deuterotetrahydrofuran). The mass spectra (m/z for [M+H]⁺) were recorded using electrospray ionization (ESI-MS) or atmospheric pressure chemical ionization (APCI-MS) mass spectrometry.

Where indicated, intermediate preparations and example compounds are purified by TIPLC. Tables 1 to 3 list the column, mobile phases and gradients used for some of the HIPLC separations.

TABLE 1
HPLC Method A

| | |
|---|---|
| Column | Phenomenex Gemini ® C18 |
| Mobile Phase | ACN (0.035% TFA) and water (0.005% TFA) |
| Gradient | 10% to 100% ACN (unless indicated otherwise) |

TABLE 2
HPLC Method B

| | |
|---|---|
| Column | Xtimate C18; Boston Green ODS; Welch Xtimate C18; |
| | Waters Xbridge BEH C18; YMC-Triart Prep C18; |
| | Phenomenex Gemini-NX |
| Mobile Phase | Water (0.225% FA) and ACN |
| Gradient | 15% to 45% ACN gradient over 10 minutes (unless |
| | indicated otherwise) |

TABLE 3
HPLC Method C

| | |
|---|---|
| Column | Phenomenex Gemini ® C18; Waters 2525 or 2545; |
| | Boston Prime C18; Phenomenex Gemini-NX C18 |
| Mobile Phase | 10 mM NH₄HCO₃ in water and ACN (pH = 9.5-10) or |
| | water (0.05% NH₃H₂O + 10 mM NH₄HCO₃) and ACN |
| Gradient | 55% to 85% ACN (unless indicated otherwise) |

The preparations and examples may employ supercritical fluid chromatography (SFC) to separate enantiomers. Table 4 lists equipment, materials, and conditions for some of the SFC separations.

TABLE 4
SFC Method

| | |
|---|---|
| Column | OD-H (21 mm × 250 mm) 5 micron or AS-H column |
| | with 30% isopropyl alcohol |
| Mobile Phase | CO₂ and EtOH + 0.05% DEA (diethyl amine) or CO₂ |
| | and MeOH + 0.05% DEA |

Besides 1JPLC, some of the preparations and examples may employ flash chromatography or preparative thin layer chromatography (TLC). Preparative TLC is typically carried out on silica gel 60 F₂₅₄ plates.

After isolation by chromatography, the solvent may be removed and the product cried in a centrifugal evaporator (e.g., GeneVac™), rotary evaporator, evacuated flask, etc. Reactions in an inert (e.g., nitrogen) or reactive (e.g., H₂) atmosphere are typically carried out at a pressure of about 1 atmosphere (14.7 psi).

### Preparation 1: 5,5-difluoro-1-methylpiperidin-3-amine

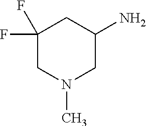

### Step 1: Tert-Butyl (5,5-difluoro-1-methylpiperidin-3-yl)carbamate

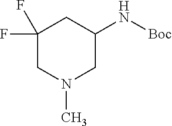 [SMILES: CN1CC(NC(=O)OC(C)(C)C)CC(F)(F)C1]

A mixture of tert-butyl (5,5-difluoropiperidin-3-yl)carbamate (500 mg, 2.12 mmol) and aqueous formaldehyde (343.49 mg, 4.23 mmol, 315.12 μL, 37% purity) in THF (1 mL) was stirred at 25° C. for 1 hour. Formic acid (203.34 mg, 4.23 mmol) was added drop-wise and the mixture was stirred at 70° C. for 1 hour. The reaction mixture was quenched with NH₃·H₂O (5 mL) and concentrated under reduced pressure. The residue was purified by flash chromatography (ISCO® SepaFlash® 12 g silica gel column) using a gradient of 0 to 20% DCM in MeOH (35 mL/min) to give the title compound (310 mg, crude). ESI-MS m/z [M+H]⁺ 251.1.

### Step 2: 5,5-difluoro-1-methylpiperidin-3-amine

A mixture of tert-butyl (5,5-difluoro-1-methylpiperidin-3-yl)carbamate (310 mg, 1.24 mmol) and HCl in dioxane (4 M, 5 mL) was stirred at 20° C. for 15 hours and then concentrated under reduced pressure. The resulting residue was triturated with EtOAc (15 mL) for 30 minutes and filtered. The filter cake was dried under vacuum to give an HCl salt of the title compound as a colorless oil (240 mg, crude). ESI-MS m/z [M+H]⁺ 151.1

### Preparation 2: 1-cyclopropylpiperidin-3-amine

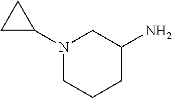

### Step 1: Tert-Butyl (1-cyclopropylpiperidin-3-yl)carbamate

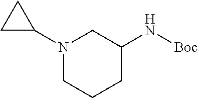 [SMILES: CC(C)(C)OC(=O)NC1CCCN(C2CC2)C1]

To a mixture of tert-butyl piperidin-3-ylcarbamate (3 g, 14.98 mmol) in THF (72 mL) and MeOH (8.1 mL) were added molecular sieve 4 Å (3 g) and (1-ethoxycyclopropoxy)trimethylsilane (7.83 g, 44.94 mmol, 9.03 mL), followed by acetic acid (10.79 g, 179.75 mmol, 10.28 mL) and NaBH₃CN (2.82 g, 44.94 mmol). The mixture was stirred at 65° C. for 16 hours. The resulting suspension was filtered and concentrated. The crude product was diluted with aq NaHCO₃ (50 mL) and extracted with DCM (100 mL×3). The organic layer was dried over anhydrous Na₂SO₄, filtered and concentrated under reduced pressure and the resulting residue was purified by column chromatography (DCM/MeOH=1:0 to 10:1). The title compound was obtained as a colorless oil (6.5 g, 90%). ¹H NMR (400 MHz, CD₃Cl) δ ppm 4.92-5.27 (m, 1H), 3.73 (br s, 1H), 2.49-2.81 (m, 4H), 1.67 (br s, 4H), 1.55 (br s, 1H), 1.45 (s, 9H), 0.47 (br d, J=6.27 Hz, 4H).

### Step 2: 1-cyclopropylpiperidin-3-amine

To a mixture of tert-butyl (1-cyclopropylpiperidin-3-yl)carbamate (4.9 g, 20.39 mmol) was added HCl in dioxane (4 M, 50.97 mL) in one portion at 25° C. under N₂. The mixture was stirred at 25° C. for 1 hour. Excess HCl/dioxane was removed under reduced pressure to give a yellow solid, which was triturated with EtOAc (80 mL). The solids were collected by filtration and dried under vacuum to give the dihydrochloride salt of the title compound as a white solid (2.8 g, 64%). ¹H NMR (400 MHz, DMSO-d₆) δ ppm 0.65-0.96 (m, 2H), 1.02-1.24 (m, 2H), 1.45-1.69 (m, 1H), 1.90 (br d, J=3.26 Hz, 2H), 2.03-2.20 (m, 1H), 2.84-3.10 (m, 1H), 2.89-3.00 (m, 1H), 3.40-3.52 (m, 1H), 3.45 (br d, J=11.80 Hz, 1H), 3.61 (br d, J=9.03 Hz, 2H), 8.70 (br s, 3H), 11.49 (br s, 1H).

### Preparation 3: (R)-1-cyclopropylpiperidin-3-amine

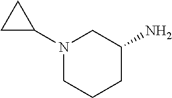

### Step 1: Tert-Butyl (R)-(1-cyclopropylpiperidin-3-yl)carbamate

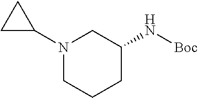 [SMILES: CC(C)(C)OC(=O)N[C@@H]1CCCN(C2CC2)C1]

To tert-butyl (R)-piperidin-3-ylcarbamate (3 g, 14.98 mmol) in THE (72 mL) and MeOH (8.1 mL) were added molecular sieve 4 Å (3 g, 14.98 mmol), (1-ethoxycyclopropoxy)trimethylsilane (7.83 g, 44.94 mmol, 9.03 mL), acetic acid (10.79 g, 179.75 mmol, 10.28 mL) and NaBH₃CN (2.82 g, 44.94 mmol). The mixture was stirred at 65° C. for 16 hours. LC-MS showed the desired product was formed as the major component and TLC (DCM/MeOH=10:1) showed the reaction was complete (R_{f} for desired product=0.43). The suspension was filtered and concentrated. The crude was diluted with aq NaHCO₃ (50 mL) and extracted with DCM (100 mL×3). The organic layers were combined, dried over anhydrous Na₂SO₄, filtered and concentrated under reduced pressure. The resulting residue was purified by column chromatography (SiO₂, DCM/MeOH=1:0 to 10:1) to give an HCl salt of the title compound as a white solid (3.5 g, 97%). ¹H NMR (400 MHz, DMSO-d₆) δ ppm 0.27 (br s, 2H), 0.39 (br d, J=3.51 Hz, 2H), 1.04-1.20 (m, 1H), 1.37 (s, 9H), 1.52-1.72 (m, 3H), 1.87-2.12 (m, 2H), 2.68-2.95 (m, 2H), 3.21-3.32 (m, 1H), 6.51-6.79 (m, 1H); ESI-MS m/z [M+H]⁺ 241.2.

### Step 2: (R)-1-cyclopropylpiperidin-3-amine

To tert-butyl (R)-(1-cyclopropylpiperidin-3-yl)carbamate (0.5 g, 2.08 mmol) was added HCl in dioxane (4 M, 10 mL). The mixture was stirred at 25° C. for 2 hours. LC-MS showed the starting material was consumed. The reaction mixture was concentrated under reduced pressure to give an HCl salt of the title compound (350 mg, 95.2%) which was used without further purification. ¹H NMR (400 MHz, DMSO-d₆) δ ppm 0.81 (br d, J=6.48 Hz, 2H), 1.04-1.24 (m, 2H), 1.46-1.67 (m, 1H), 1.89 (br s, 2H), 2.00-2.14 (m, 1H), 2.79-3.20 (m, 4H), 3.54-3.71 (m, 1H), 8.59 (br s, 2H); ESI-MS m/z [M+H]⁺ 141.1.

### Preparation 4: (R)-1-(1-methylcyclopropyl)piperidin-3-amine

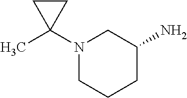

### Step 1: Tert-Butyl (R)-(1-acetylpiperidin-3-yl)carbamate

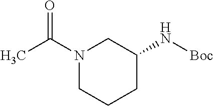 [SMILES: CC(=O)N1CCC[C@@H](NC(=O)OC(C)(C)C)C1]

To a mixture of tert-butyl (R)-piperidin-3-ylcarbamate (1 g, 4.99 mmol) and DIPEA (1.94 g, 14.98 mmol, 2.61 mL) in DCM (15 mL) was added Ac₂O (560.71 mg, 5.49 mmol, 514.42 μL). The mixture was stirred at 20° C. for 12 hours. TLC (DCM/MeOH=10:1) indicated the starting material was totally consumed (R_{f}=0.25) and one main new spot was formed (R_{f}=0.53). The mixture was washed with H₂O (20 mL) and concentrated in vacuo, and the resulting residue was purified by flash chromatography (ISCO® SepaFlash® 24 g silica gel column) using a gradient of 0 to 3% MeOH in DCM (35 m/min). The title compound was obtained as a white solid (1 g, 83%). ¹H NMR (400 MHz, DMSO-d₆) δ ppm 1.38 (m, 11H), 1.59-1.83 (m, 2H), 1.90-2.02 (m, 3H), 2.42 (dd, J=12.1, 10.2 Hz, 1H), 2.89-3.09 (m, 1H), 3.18-3.30 (m, 1H), 3.59 (br t, J=13.3 Hz, 1H), 3.71-4.17 (m, 1H), 6.75-7.06 (m, 1H).

### Step 2: Tert-Butyl (R)-(1-(1-methylcyclopropyl)piperidin-3-yl)carbamate

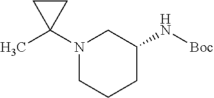 [SMILES: CC(C)(C)OC(=O)N[C@@H]1CCCN(C2(C)CC2)C1]

To a solution of tert-butyl (R)-(1-acetylpiperidin-3-yl)carbamate (900 mg, 3.71 mmol) and Ti(i-PrO)₄ (2.11 g, 7.43 mmol, 2.19 mL) in THE (15 mL) was added dropwise EtMgBr (3 M, 6.19 mL) at 0° C. The mixture was stirred at 20° C. for 12 hours. TLC (EtOAc/MeOH=20:1) indicated about 40% of starting material remained (R_{f}=0.68) and one major new spot was detected (R_{f}=0.66). The reaction mixture was quenched with H₂O (2 mL) and filtered. The filtrate was concentrated in vacuo and the resulting residue was purified by flash chromatography (ISCO® SepaFlash® 20 g silica gel column) using a gradient of 0 to 5% MeOH in DCM (35 mL/min). The title compound was obtained as a colorless gum (160 mg, 16.9%). ¹H NMR (400 MHz, DMSO-d₆) δ ppm 0.29 (br s, 2H), 0.41 (br s, 2H), 0.97 (s, 3H), 1.03-1.23 (m, 2H), 1.37 (s, 9H), 1.48-1.74 (m, 2H), 2.02-2.14 (m, 1H), 2.18-2.29 (m, 1H), 2.59 (br d, J=11.3 Hz, 1H), 2.75 (br d, J=6.9 Hz, 1H), 3.25 (br s, 1H), 6.60 (br d, J=7.1 Hz, 1H).

### Step 3: (R)-1-(1-methylcyclopropyl)piperidin-3-amine

A mixture of tert-butyl (R)-(1-(1-methylcyclopropyl)piperidin-3-yl)carbamate (160 mg, 629.01 μmol) in HCl/dioxane (4 M, 5 mL) was stirred at 20° C. for 12 hours. TLC (EtOAc/MeOH=20:1) indicated the starting material was consumed completely (R_{f}=0.66). The mixture was concentrated in vacuo to give an HCl salt of the title compound as a yellow solid (120 mg, crude) which was used without further purification. ¹H NMR (400 MHz, DMSO-d₆) δ ppm 0.72-0.82 (m, 2H), 1.31 (s, 5H), 1.51-1.66 (m, 1H), 1.74-2.14 (m, 3H), 3.06-3.33 (m, 3H), 3.40-3.50 (m, 1H), 3.62-3.94 (m, 1H), 8.53 (br s, 2H), 11.27 (br s, 1H).

### Preparation 5: (3R,5R)-1-cyclopropyl-5-fluoropiperidin-3-amine

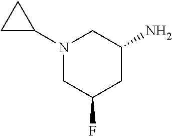 [SMILES: N[C@@H]1C[C@@H](F)CN(C2CC2)C1]

### Step 1: Tert-Butyl ((3R,5R)-1-cyclopropyl-5-fluoropiperidin-3-yl)carbamate

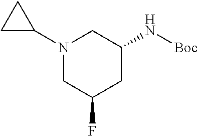 [SMILES: CC(C)(C)OC(=O)N[C@@H]1C[C@@H](F)CN(C2CC2)C1]

A solution of tert-butyl ((3R,5R)-5-fluoropiperidin-3-yl)carbamate (0.327 g, 1.50 mmol) in THF (5 mL), MeOH (5 mL) and acetic acid (1.0 mL) was treated with (1-ethoxycyclopropoxy)trimethylsilane (0.523 g, 3.00 mmol), followed by sodium cyanoborohydride (0.283 g, 4.50 mmol). The reaction mixture was stirred at 60° C. overnight and then partitioned between EtOAc and saturated aqueous NaHCO₃. The aqueous phase was extracted with EtOAc. The organic layers were combined, dried over Na₂SO₄, filtered and concentrated in vacuo. The resulting residue was purified by flash chromatography with ELS detection (ISCO® RediSep Rf Gold® 40 g silica gel column) using a gradient of 0 to 100% EtOAc in heptane. The product-containing fractions were evaporated to give the title compound as a white solid (0.333 g, 86%). ESI-MS m/z [M+H]⁺ 259.0.

### Step 2: (3R,5R)-1-cyclopropyl-5-fluoropiperidin-3-amine

A solution of tert-butyl ((3R,5R)-1-cyclopropyl-5-fluoropiperidin-3-yl)carbamate (0.333 g, 1.29 mmol) in dioxane (6.45 mL) was treated with HCl (4 M in dioxane, 1.61 mL, 6.45 mmol). The reaction mixture was stirred at room temperature over the weekend. A small amount of MeOH was added to ensure the mixture was homogeneous. The reaction mixture was concentrated to dryness in vacuo to give the dihydrochloride salt of the title compound (351.5 mg crude, 298 mg theoretical, assumed quantitative and 84% purity) which was used without additional purification. ESI-MS m/z [M+H]⁺ 159.2.

### Preparation 6: 3-((4-chlorophthalazin-1-yl)amino)phenol

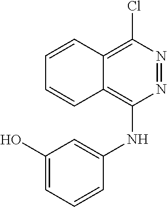 [SMILES: Oc1cccc(Nc2nnc(Cl)c3ccccc23)c1]

To a solution of 3-aminophenol (548.27 mg, 5.02 mmol) in EtOH (10 mL) was added 1,4-dichlorophthalazine (1 g, 5.02 mmol). The mixture was stirred at 80° C. for 15 hours. LC-MS showed 1,4-dichlorophthalazine was completely consumed. The reaction mixture was concentrated under reduced pressure and the resulting residue was purified by flash chromatography (ISCO® SepaFlash® 24 g silica gel column) using a gradient of 0 to 50% EtOAc in petroleum ether (30 mL/min). The title compound was obtained as a white solid (778.7 mg, crude). ESI-MS m/z [M+H]⁺ 271.9.

### Preparation 7: 2-((4-chlorophthalazin-1-yl)amino)phenol

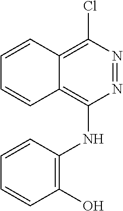 [SMILES: Oc1ccccc1Nc1nnc(Cl)c2ccccc12]

The title compound was made like Preparation 6, using 2-aminophenol and 1,4-dichlorophthalazine, and was obtained as a yellow solid. ESI-MS m/z [M+H]⁺ 272.

### Preparation: 8: 3-((4-chlorophthalazin-1-yl)amino)phenol

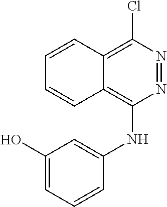 [SMILES: Oc1cccc(Nc2nnc(Cl)c3ccccc23)c1]

A solution of 3-aminophenol (200 mg, 1.83 mmol) and 1,4-dichlorophthalazine (364.78 mg, 1.83 mmol) in EtOH (5 mL) was stirred at reflux (80° C.) for 5 hours. The reaction mixture was then concentrated and purified by flash chromatography (ISCO® SepaFlash® 24 g silica gel column) using a gradient of 0 to 60% EtOAc in petroleum ether (35 mL/min). The title compound was obtained as a yellow solid (300 mg, 60.3%). ESI-MS m/z [M+H]⁺ 272.0.

### Preparation 9: 3-((1-chloropyrido[3,4-d]pyridazin-4-yl)amino)phenol and 3-((4-chloropyrido[3,4-d]pyridazin-1-yl)amino)phenol

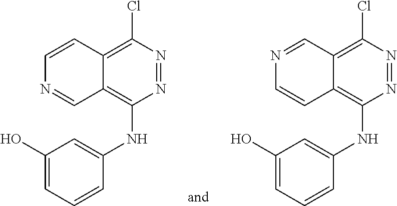 [SMILES: Oc1cccc(Nc2nnc(Cl)c3ccncc23)c1 ; Oc1cccc(Nc2nnc(Cl)c3cnccc23)c1]

A mixture of 1,4-dichloropyrido[3,4-d]pyridazine (100 mg, 499.94 μmol), 3-aminophenol (54.56 mg, 499.94 μmol) and TFA (114.01 mg, 999.88 μmol, 74.03 μL) in tert-BuOH (8 mL) was stirred at 25° C. for 16 hours. LC-MS showed one main peak with desired m/z. The reaction mixture was filtered to give a residue, which was purified by preparative HPLC (Method B) to give a mixture of the title compounds as a yellow solid (63.3 mg, 46.4%). ESI-MS m/z [M+H]⁺ 273.0.

### Preparation 10: 4-chloro-N-(1-methylpiperidin-3-yl)phthalazin-1-amine

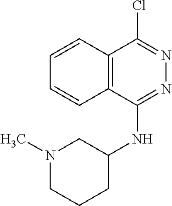 [SMILES: CN1CCCC(Nc2nnc(Cl)c3ccccc23)C1]

A mixture of 1-methylpiperidin-3-amine (586 mg, 4.88 mmol), 1,4-dichlorophthalazine (1022 mg, 4.88 mmol) and sodium carbonate (1044 mg, 9.76 mmol) in DMF (10 mL) was stirred in a microwave reactor on normal absorbance for 30 minutes at 130° C. The reaction mixture was then diluted with H₂O (15 mL) and the product was extracted with EtOAc (40 mL×2). The organic extracts were combined, dried over Na₂SO₄, filtered, rinsed with EtOAc, and concentrated by rotary evaporation to provide the crude product (6.09 g) as a yellow-orange oil. The crude material was dissolved in toluene (2 mL) and purified by medium pressure chromatography (RediSep Rf Gold® 80 g silica gel column) using a gradient of 0 to 15% MeOH in DCM. The pure fractions were combined, concentrated by rotary evaporation, and dried in vacuo to provide the title compound as a yellow foam (544 mg, 40.3%). ¹H NMR (400 MHz, CDCl₃) δ ppm 1.63-3.02 (m, 11H), 4.53-4.80 (m, 1H), 5.81-6.16 (m, 1H), 7.75-8.01 (m, 3H), 8.11-8.23 (m, 1H); ESI-MS m/z [M+H]⁺ 277.0.

### Preparation 11: (R)-4-chloro-N-(1-methylpiperidin-3-yl)phthalazin-1-amine

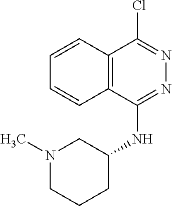 [SMILES: CN1CCC[C@@H](Nc2nnc(Cl)c3ccccc23)C1]

A mixture of 1,4-dichlorophthalazine (500 mg, 2.51 mmol), (R)-1-methylpiperidin-3-amine (344.22 mg, 3.01 mmol) and DIPEA (649.34 mg, 5.02 mmol, 875.13 μL) in DMSO (4 mL) was stirred at 100° C. for 12 hours. LC-MS showed one main peak with desired m/z. The reaction mixture was filtered and the filtrate was purified by preparative HPLC (Method B) to give a formic acid salt of the title compound as a yellow solid (360 mg, crude). ESI-MS m/z [M+H]⁺ 277.

### Preparation 12: 4-chloro-N-methyl-N-(1-methylpiperidin-3-yl)phthalazin-1-amine

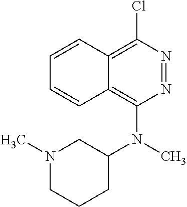

To a 5 mL vial equipped with stir bar were added N,1-dimethylpiperidin-3-amine (107 mg, 0.835 mmol), sodium carbonate (177 mg, 1.669 mmol) and 1,4-dichlorophthalazine (166 mg, 0.835 mmol) in DMF (2 mL). The mixture was stirred at 130° C. in a microwave reactor (Biotage® Initiator) for 30 minutes. The reaction mixture was diluted in water (30 mL) and extracted with EtOAc (30 mL×2). The organic layers were combined, washed with brine (10 mL), dried over sodium sulfate, filtered, and concentrated under reduced pressure. The residue was purified by flash chromatography (ISCO® RediSep Rf Gold® 24 g silica gel column) using a gradient of 0 to 20% MeOH in DCM. The product-containing fractions were concentrated under reduced pressure to give the title compound (14.7 mg, 6.1%). ESI-MS m/z [M+H]⁺ 291.2.

### Preparation 13: (R)-4-chloro-8-methyl-N-(1-methylpiperidin-3-yl)phthalazin-1-amine and (R)-4-chloro-5-methyl-N-(1-methylpiperidin-3-yl)phthalazin-1-amine

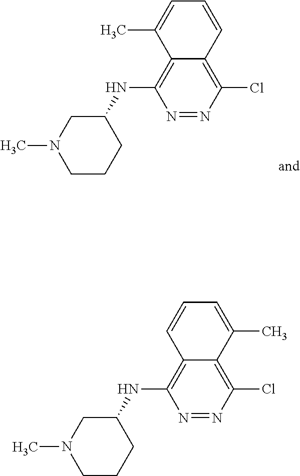

### Step 1: 5-methylphthalazine-1,4-diol

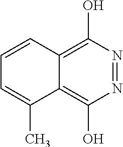

To a solution of 4-methylisobenzofuran-1,3-dione (3 g, 18.50 mmol) in EtOH (30 mL) was added hydrazine hydrate (2.78 g, 55.51 mmol, 2.70 mL). The mixture was stirred at 100° C. for 12 hours. LC-MS showed product of the desired mass was obtained. The reaction mixture was concentrated under reduced pressure to remove solvent. The residue was triturated with EtOH (50 mL) for 30 minutes and filtered. The filter cake was dried in vacuo to give the title compound as a white solid (2.6 g, 87%). ¹H NMR (400 MHz, DMSO-d₆) δ ppm 2.80 (s, 3H), 7.56 (d, J=7.4 Hz, 1H), 7.68 (t, J=7.7 Hz, 1H), 7.89 (d, J=7.9 Hz, 1H); ESI-MS m/z [M+H]⁺ 177.1.

### Step 2: 1,4-dichloro-5-methylphthalazine

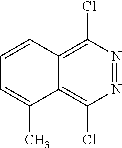

A mixture of 5-methylphthalazine-1,4-diol (500 mg, 2.84 mmol), POCl₃ (12.38 g, 80.71 mmol, 7.50 mL) and DIPEA (1.83 g, 14.19 mmol, 2.47 mL) was stirred at 100° C. for 2 hours. LC-MS showed the desired product was formed as the major component. The mixture was concentrated in vacuo and the resulting residue was purified by flash chromatography (ISCO® SepaFlash® 24 g silica gel column) using a gradient of 0 to 30% EtOAc in petroleum ether (35 m/min). The title compound was obtained as a white solid (170 mg, 28.1%). ¹H NMR (400 MHz, DMSO-d₆) δ ppm 3.00 (s, 3H), 8.01-8.13 (m, 2H), 8.24 (dd, J=7.8, 1.1 Hz, 1H).

### Step 3: (R)-4-chloro-8-methyl-N-(1-methylpiperidin-3-yl)phthalazin-1-amine and (R)-4-chloro-5-methyl-N-(1-methylpiperidin-3-yl)phthalazin-1-amine

A mixture of 1,4-dichloro-5-methylphthalazine (170 mg, 797.89 μmol), (R)-1-methylpiperidin-3-amine (136.66 mg, 1.20 mmol) and DIPEA (206.24 mg, 1.60 mmol, 277.95 μL) in DMSO (2 mL) was stirred at 100° C. for 12 hours. LC-MS showed one main peak with desired m/z. The mixture was filtered. The filtrate was concentrated and purified by preparative HPLC (YMC-Actus Triart C18-5 μm, 30 mm×150 mm column) using a gradient of 50 to 70% ACN in water (with 0.05% NH₃H₂O) to give a mixture of the title compounds as a yellow solid (50 mg, crude). ESI-MS m/z [M+H]⁺ 291.1.

### Preparation 14: 1-chloro-N-(1-methylpiperidin-3-yl)pyrido[3,4-d]pyridazin-4-amine and 4-chloro-N-(1-methylpiperidin-3-yl)pyrido[3,4-d]pyridazin-1-amine

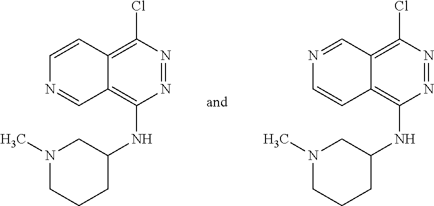 [SMILES: CN1CCCC(Nc2nnc(Cl)c3ccncc23)C1 ; CN1CCCC(Nc2nnc(Cl)c3cnccc23)C1]

To a solution of 1,4-dichloropyrido[3,4-d]pyridazine (500 mg, 2.50 mmol) in DMF (10 mL) were added Cs₂CO₃ (1.63 g, 5.00 mmol) and 1-methylpiperidin-3-amine (1.08 g, 5.75 mmol, 2 HCl). The mixture was stirred at 100° C. for 10 hours and then concentrated under vacuum. The resulting residue was purified by preparative HPLC (Method B) to give a mixture of the title compounds as a white solid (150 mg, 21.6%). ESI-MS m/z [M+H]⁺ 278.2.

### Preparation 15: (R)-4-chloro-N-(1-methylpiperidin-3-yl)pyrido[3,4-d]pyridazin-1-amine and (R)-1-chloro-N-(1-methylpiperidin-3-yl)pyrido[3,4-d]pyridazin-4-amine

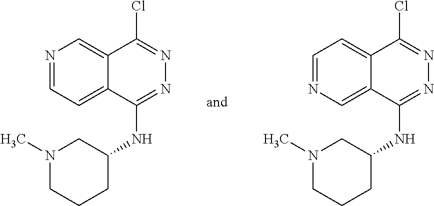 [SMILES: CN1CCC[C@@H](Nc2nnc(Cl)c3cnccc23)C1 ; CN1CCC[C@@H](Nc2nnc(Cl)c3ccncc23)C1]

A mixture of the title compounds was made like Preparation 14, using (R)-1-methylpiperidin-3-amine (100 mg, 875.75 μmol) and 1,4-dichloropyrido[3,4-d]pyridazine (175.17 mg, 875.75 μmol), and was obtained as a yellow solid (120 mg, 48.8%). ESI-MS m/z [M+H]⁺ 278.0.

### Preparation 16: (R)-1-chloro-N-(1-methylpiperidin-3-yl)pyrido[3,4-d]pyridazin-4-amine

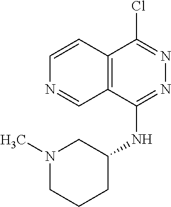 [SMILES: CN1CCC[C@@H](Nc2nnc(Cl)c3ccncc23)C1]

To a yellow suspension of (R)-1-methylpiperidin-3-amine dihydrochloride (1871 mg, 10.0 mmol) and 1,4-dichloropyrido[3,4-d]pyridazine (2000 mg, 10.0 mmol) in DMSO (20.499 mL) was added DIPEA (8.7 mL, 50.0 mmol) at room temperature. The reaction mixture was stirred at room temperature for 5 minutes and then stirred in an oil bath at 100° C. for 21 hours. The reaction mixture was cooled to room temperature, diluted with H₂O (80 mL) and extracted with EtOAc (160 mL×2). The organic extracts were combined, dried over Na₂SO₄, filtered, rinsed with EtOAc, and concentrated by rotary evaporation to provide a crude mixture of products as a red oil (4.0 g). The crude mixture was dissolved in toluene (6 mL) and purified by medium pressure chromatography (Shoko Scientific Purif-Pack® NH-60 μm, 46×220 mm, 200 g spherical silica gel column) using a gradient of 0 to 100% EtOAc in heptane. The early fractions were combined, concentrated by rotary evaporation and dried in vacuo to provide a first crop of the title compound as a yellow-orange foam (0.797 g). The later fractions were combined, concentrated by rotary evaporation and dried in vacuo to provide an impure mixture of products (1.093 g). The impure mixture was dissolved in toluene (5 mL) and purified by medium pressure chromatography (Shoko Scientific Purif-Pack® NH-60 μm, 46 mm×110 mm, 120 g spherical silica gel column) using a gradient of 0 to 100% EtOAc in heptane. The early fractions were combined, concentrated by rotary evaporation and dried in vacuo to provide a second crop (0.394 g) of the title compound as an orange foam (total 1191 mg, 42.9%). ¹H NMR (400 MHz, DMSO-d₆) δ ppm 1.42 (qd, J=11.80, 4.02 Hz, 1H), 1.51-1.65 (m, 1H), 1.70-1.79 (m, 1H), 1.85-2.01 (m, 3H), 2.20 (s, 3H), 2.66-2.75 (m, 1H), 3.04 (br dd, J=10.29, 3.51 Hz, 1H), 4.28-4.39 (m, 1H), 7.74 (d, J=7.53 Hz, 1H), 7.85 (dd, J=5.65, 0.88 Hz, 1H), 9.05 (d, J=5.52 Hz, 1H), 9.80 (d, J=0.75 Hz, 1H); ESI-MS m/z [M+H]⁺ 278.1.

### Preparation 17: cis-3-((1-chloropyrido[3,4-d]pyridazin-4-yl)amino)-1-methylcyclobutan-1-ol and cis-3-((4-chloropyrido[3,4-d]pyridazin-1-yl)amino)-1-methylcyclobutan-1-ol

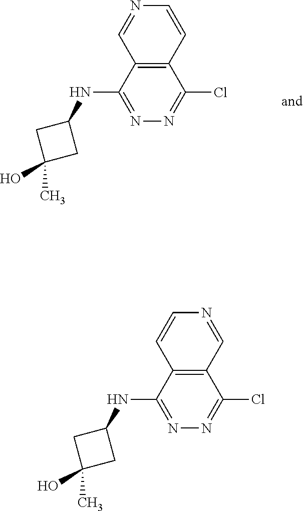 [SMILES: C[C@]1(O)C[C@@H](Nc2nnc(Cl)c3ccncc23)C1 ; C[C@]1(O)C[C@@H](Nc2nnc(Cl)c3cnccc23)C1]

To a mixture of cis-3-amino-1-methyl-cyclobutanol hydrochloride (69 mg, 0.500 mmol) and 1,4-dichloropyrido[3,4-d]pyridazine (100 mg, 0.500 mmol) in DMSO (1.0249 mL) was added DIPEA (0.44 mL, 2.50 mmol) at room temperature. The reaction mixture was stirred at room temperature for 5 minutes and then stirred in an oil bath at 100° C. for 3 hours. The reaction mixture was cooled to room temperature, diluted with water (4 mL) and the crude products were extracted with EtOAc (8 mL×3). The organic extracts were combined, dried over Na₂SO₄, filtered, rinsed with EtOAc, and concentrated via rotary evaporation to provide a crude mixture of products as a yellow-orange oil (0.27 g). The crude mixture was dissolved in toluene (2 mL) and purified via medium pressure chromatography (HP RediSep Rf Gold® 12 g silica gel column) using a gradient of 0 to 100% EtOAc in heptane. The product-containing fractions were combined, concentrated via rotary evaporation, and dried in vacuo to give a mixture of the title compounds as a yellow-orange solid (97.4 mg). ¹H NMR (400 MHz, DMSO-d₆) δ ppm 1.32 (s, 3H), 2.07-2.24 (m, 2H), 2.42-2.48 (m, 2H), 4.04-4.27 (m, 1H), 4.89-5.13 (m, 1H), 7.85 (dd, J=5.65, 0.88 Hz, 1H), 8.22 (br d, J=5.77 Hz, 1H), 9.05 (d, J=5.52 Hz, 1H), 9.80 (d, J=0.75 Hz, 1H); ESI-MS m/z [M+H]⁺ 265.0.

### Preparation 18: 4-((1-chloropyrido[3,4-d]pyridazin-4-yl)amino)bicyclo[2.2.1]heptan-1-ol

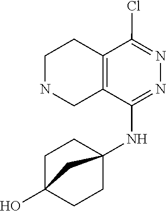 [SMILES: O[C@]12CC[C@](Nc3nnc(Cl)c4c3CNCC4)(CC1)C2]

The title compound was made like Preparation 17, using 4-aminonorbornan-1-ol hydrochloride (27 mg, 0.165 mmol) and 1,4-dichloropyrido[3,4-d]pyridazine (33 mg, 0.165 mmol), and was obtained as a yellow solid (21 mg, 43%). ¹H NMR (400 MHz, DMSO-d₆) δ ppm 1.56-1.68 (m, 2H), 1.69-1.83 (m, 2H), 2.00 (s, 2H), 2.05-2.19 (m, 4H), 4.87-5.05 (m, 1H), 7.84 (dd, J=5.52, 1.00 Hz, 1H), 7.90 (s, 1H), 9.04 (d, J=5.77 Hz, 1H), 9.82 (d, J=0.75 Hz, 1H). ESI-MS m/z [M+H]⁺ 291.0.

### Preparation 19: 1-chloro-5-methyl-N-(1-methylpiperidin-3-yl)pyrido[3,4-d]pyridazin-4-amine and 4-chloro-5-methyl-N-(1-methylpiperidin-3-yl)pyrido[3,4-d]pyridazin-1-amine

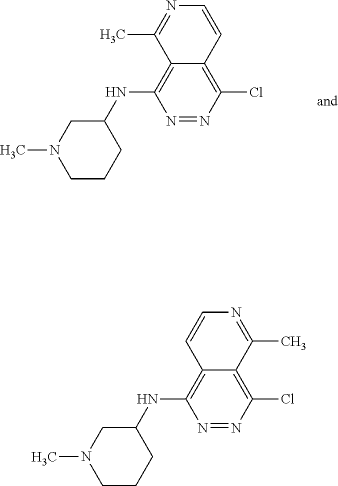

### Step 1: Dimethyl 2-methylpyridine-3,4-dicarboxylate

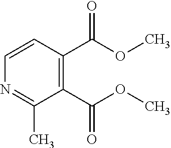

To a mixture of dimethyl 2-chloropyridine-3,4-dicarboxylate (1 g, 4.36 mmol) and Pd(dppf)Cl₂·CH₂Cl₂ (177.83 mg, 217.75 μmol) in dioxane (25 mL) was added dimethylzinc (1 M, 13.07 mL, 3 eq). The reaction mixture was heated at 80° C. for 12 hours. LC-MS showed the starting material was consumed and desire MS was observed. The reaction mixture was quenched with water (40 mL) and filtered through a pad of Celite®, which was rinsed with water (10 mL) and EtOAc (30 mL). The filtrate was extracted with EtOAc (50 mL×2). The organic phase was dried over Na₂SO₄ and concentrated under vacuum. The resulting residue was purified by flash chromatography (ISCO® SepaFlash® 12 g silica gel column) using a gradient of 20 to 25% EtOAc in petroleum ether (40 m/min). The title compound was obtained as a yellow solid (800 mg, 71.6% yield, 81.6% purity). ESI-MS m/z [M+H]⁺ 210.2.

### Step 2: 5-methylpyrido[3,4-d]pyridazine-1,4-diol

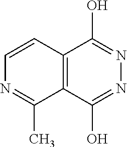

A solution of dimethyl 2-methylpyridine-3,4-dicarboxylate (800 mg, 3.82 mmol) in N₂H₄.H₂O (9.68 g, 189.58 mmol, 81.53 μL, 98% purity) was stirred at 70° C. for 12 hours. LC-MS showed the starting material was consumed. The reaction mixture was concentrated under vacuum to give a solid, which was triturated with ACN (30 mL) to give the title compound as a yellow solid (780 mg, crude).

### Step 3: 1,4-dichloro-5-methylpyrido[3,4-d]pyridazine

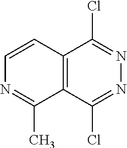

A mixture of 5-methylpyrido[3,4-d]pyridazine-1,4-diol (150 mg, 846.69 μmol) and DIPEA (547.15 mg, 4.23 mmol, 737.39 μL) in POCl₃ (5 mL) was stirred at 100° C. for 1 hour. The mixture was concentrated in vacuo. The residue was diluted with DCM/DIPEA (1:1, 50 mL) and concentrated in vacuo. The residue was purified by flash chromatography (ISCO® SepaFlash® 12 g silica gel column) using a gradient of 0 to 1% MeOH in DCM (25 m/min). The title compound was obtained as a yellow solid (100 mg, 55.2%). ESI-MS m/z [M+H]⁺ 214.

### Step 4: 1-chloro-5-methyl-N-(1-methylpiperidin-3-yl)pyrido[3,4-d]pyridazin-4-amine and 4-chloro-5-methyl-N-(1-methylpiperidin-3-yl)pyrido[3,4-d]pyridazin-1-amine

A mixture of 1,4-dichloro-5-methylpyrido[3,4-d]pyridazine (100 mg, 467.18 μmol), 1-methylpiperidin-3-amine (174.83 mg, 934.36 μmol, 2 HCl) and DIPEA (301.90 mg, 2.34 mmol, 406.87 μL) in DMSO (1 mL) was stirred at 90° C. for 12 hours. The reaction mixture was purified by preparative HPLC (Method B) to give a mixture of the title compounds as a yellow gum (90 mg). ESI-MS m/z [M+H]⁺ 292.

### Preparation 20: 1-chloro-7-methyl-N-(1-methylpiperidin-3-yl)pyrido[3,4-d]pyridazin-4-amine and 4-chloro-7-methyl-N-(1-methylpiperidin-3-yl)pyrido[3,4-d]pyridazin-1-amine

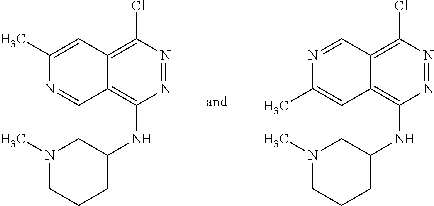 [SMILES: Cc1cc2c(Cl)nnc(NC3CCCN(C)C3)c2cn1 ; Cc1cc2c(NC3CCCN(C)C3)nnc(Cl)c2cn1]

### Step 1: Dimethyl 6-methylpyridine-3,4-dicarboxylate

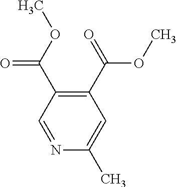

To a mixture of dimethyl 6-chloropyridine-3,4-dicarboxylate (740 mg, 3.22 mmol) and Pd(dppf)Cl₂·CH₂Cl₂ (131.59 mg, 161.14 μmol) in dioxane (20 mL) was added dimethylzinc (1 M, 9.67 mL, 3 eq). The mixture was heated at 80° C. for 12 hours and then quenched with water (40 mL). The reaction mixture was filtered through a pad of Celite®, which was rinsed with water (10 mL) and EtOAc (30 mL). The filtrate was extracted with EtOAc (50 mL×2). The organic phase was dried over anhydrous Na₂SO₄ and concentrated under vacuum to give a crude product. The residue was purified by flash chromatography (ISCO® SepaFlash® 12 g silica gel column) using a gradient of 19 to 21% EtOAc in petroleum ether (40 m/min). The title compound was obtained as a yellow solid (540 mg, 71.0%). ESI-MS m/z [M+H]⁺ 210.2.

### Step 2: 7-methylpyrido[3,4-d]pyridazine-1,4-diol

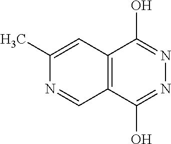

A solution of dimethyl 6-methylpyridine-3,4-dicarboxylate (540 mg, 2.58 mmol) in N₂H₄.H₂O (7.40 g, 144.95 mmol, 7.19 mL, 98% purity) was stirred at 70° C. for 12 hours. The reaction mixture was concentrated under vacuum to give a solid, which was triturated with ACN (30 mL) to give the title compound as a yellow solid (580 mg, crude). ESI-MS m/z [M+H]⁺ 178.1.

### Step 3: 1,4-dichloro-7-methylpyrido[3,4-d]pyridazine

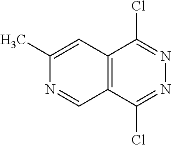

A mixture of 7-methylpyrido[3,4-d]pyridazine-1,4-diol (200 mg, 1.13 mmol) and DIPEA (729.51 mg, 5.64 mmol, 983.17 μL) in POCl₃ (5 mL) was stirred at 100° C. for 1 hour. The reaction mixture was concentrated in vacuo and the resulting residue was purified by flash chromatography (ISCO® SepaFlash® 12 g silica gel column) using a gradient of 0 to 1% MeOH in DCM (25 mL/min). The title compound was obtained as a yellow solid (200 mg, 82.8%). ESI-MS m/z [M+H]⁺ 214.

### Step 4: 1-chloro-7-methyl-N-(1-methylpiperidin-3-yl)pyrido[3,4-d]pyridazin-4-amine and 4-chloro-7-methyl-N-(1-methylpiperidin-3-yl)pyrido[3,4-d]pyridazin-1-amine

A mixture of 1,4-dichloro-7-methylpyrido[3,4-d]pyridazine (200 mg, 934.36 μmol), 1-methylpiperidin-3-amine (349.66 mg, 1.87 mmol, 2 HCl) and DIPEA (603.78 mg, 4.67 mmol, 813.72 μL) in DMSO (2 mL) was stirred at 100° C. for 1 hour. The reaction mixture was purified by preparative HPLC (Method C) to give a mixture of the title compounds as a yellow solid (100 mg). ESI-MS m/z [M+H]⁺ 292.

### Preparation 21: 1-chloro-7-methoxy-N-(1-methylpiperidin-3-yl)pyrido[3,4-d]pyridazin-4-amine and 4-chloro-7-methoxy-N-(1-methylpiperidin-3-yl)pyrido[3,4-d]pyridazin-1-amine

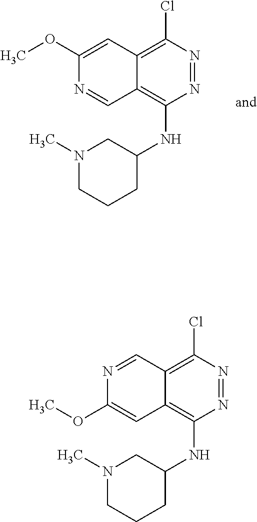 [SMILES: COc1cc2c(Cl)nnc(NC3CCCN(C)C3)c2cn1 ; COc1cc2c(NC3CCCN(C)C3)nnc(Cl)c2cn1]

### Step 1: Dimethyl Pyridine-3,4-dicarboxylate

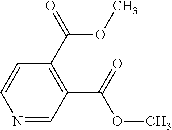

To a mixture of pyridine-3,4-dicarboxylic acid (10 g, 59.84 mmol) in MeOH (200 mL) was added H₂SO₄ (8 mL). The mixture was stirred at 80° C. for 16 hours and then concentrated under reduced pressure. The concentrate was diluted with water (20 mL) and neutralized with solid NaHCO₃ until no CO₂ evolved. The aqueous phase was extracted with EtOAc (200 mL×3) and the organic layers were combined, washed with H₂O, dried over Na₂SO₄, filtered and concentrated under reduced pressure. The resulting residue was purified by flash chromatography (ISCO® SepaFlash® 80 g silica gel column) using a gradient of 0 to 50% EtOAc in petroleum ether (45 mL/min). The title compound was obtained as a colorless oil (8.0 g, 53%). ESI-MS m/z [M+H]⁺ 196.1.

### Step 2: 3,4-bis(methoxycarbonyl)pyridine 1-oxide

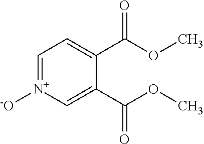

A mixture of dimethyl pyridine-3,4-dicarboxylate (8.0 g, 40.99 mmol) and mCPBA (12.48 g, 61.48 mmol, 85% purity) in DCM (150 mL) was stirred at 20° C. for 12 hours and was then filtered and concentrated under reduced pressure. The resulting residue was purified by flash chromatography (ISCO® SepaFlash® 80 g silica gel column) using a gradient of 0 to 99% EtOAc in petroleum ether (45 mL/min). The title compound was obtained as a white solid (5.3 g, 61%). ESI-MS m/z [M+H]⁺ 212.1.

### Step 3: Dimethyl 6-chloropyridine-3,4-dicarboxylate and dimethyl 2-chloropyridine-3,4-dicarboxylate

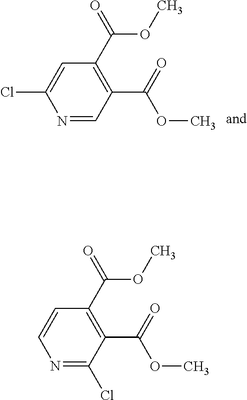

A mixture of dimethyl 3,4-bis(methoxycarbonyl)pyridine 1-oxide (2.3 g, 10.89 mmol) and POCl₃ (16.70 g, 108.92 mmol, 10.12 mL) in CH₃Cl (13 mL) was stirred at 85° C. for 18 hours. The reaction mixture was then filtered and concentrated under reduced pressure to give a residue, which was neutralized with solid NaHCO₃ until no CO₂ evolved. The aqueous phase was extracted with EtOAc (30 mL×3). The organic layers were combined, washed with H₂O (20 mL×3), dried over Na₂SO₄, filtered and concentrated under reduced pressure. The resulting residue was purified by flash chromatography (ISCO® SepaFlash® 40 g silica gel column) using a gradient of 0 to 20% EtOAc in petroleum ether (60 mL/min) to give dimethyl 6-chloropyridine-3, 4-dicarboxylate as a white solid (850 mg, 33.3%) and dimethyl 2-chloropyridine-3,4-dicarboxylate as a white solid (780 mg, 27.4%). ESI-MS m/z [M+H]⁺ 230.

### Step 4: Dimethyl 6-methoxypyridine-3,4-dicarboxylate

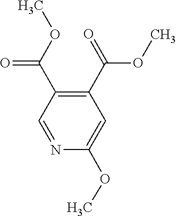 [SMILES: COC(=O)c1cnc(OC)cc1C(=O)OC]

A solution of dimethyl 6-chloropyridine-3,4-dicarboxylate (2.60 g, 11.32 mmol) and NaOMe (3.06 g, 56.62 mmol) in MeOH (20 mL) was stirred at 70° C. for 16 hours. The reaction mixture was acidified with aq HCl to pH 4. The aqueous phase was extracted with EtOAc (30 mL×3) and the organic layers were combined, washed with water (10 mL×3), dried over Na₂SO₄, filtered and concentrated under reduced pressure. The title compound was obtained as a white solid (2 g, 75% yield, 95% purity). ESI-MS m/z [M+H]⁺ 226.

### Step 5: 7-methoxypyrido[3,4-d]pyridazine-1,4-diol

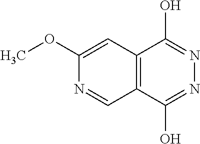

A solution of dimethyl 6-methoxypyridine-3,4-dicarboxylate (1 g, 4.44 mmol) and NH₂NH₂·H₂O (1.56 g, 31.16 mmol, 1.51 mL) in EtOH (10 mL) was stirred at 60° C. for 1 hour under N₂ atmosphere. The reaction mixture was filtered and concentrated under reduced pressure to give a residue, which was purified by recrystallization from EtOAc (10 mL) to give the title compound as a yellow solid (500 mg, 56.5%). ESI-MS m/z [M+H]⁺ 194.

### Step 6: 1,4-dichloro-7-methoxypyrido[3,4-d]pyridazine

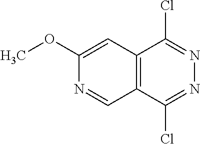

A mixture of 7-methoxypyrido[3,4-d]pyridazine-1,4-diol (500 mg, 2.59 mmol) in POCl₃ (5 mL) was degassed and purged with N₂ (3×) and then stirred at 100° C. for 1 hour under N₂ atmosphere, filtered and concentrated under reduced pressure. The concentrate was dissolved in DCM (15 mL), neutralized to pH 7 by dropwise addition of DIPEA, and concentrated. The residue was purified by flash chromatography (ISCO® SepaFlash® 4 g silica gel column) using a gradient of 0 to 30% EtOAc in petroleum ether (60 m/min). The title compound was obtained as a red solid (110 mg, 17.7%). ESI-MS m/z [M+H]⁺ 230.

### Step 7: 1-chloro-7-methoxy-N-(1-methylpiperidin-3-yl)pyrido[3,4-d]pyridazin-4-amine and 4-chloro-7-methoxy-N-(1-methylpiperidin-3-yl)pyrido[3,4-d]pyridazin-1-amine

A mixture of 1,4-dichloro-7-methoxypyrido[3,4-d]pyridazine (110 mg, 444.68 μmol), 1-methylpiperidin-3-amine dihydrochloride (166.41 mg, 889.37 μmol) and Cs₂CO₃ (579.55 mg, 1.78 mmol) in DMF (1.5 mL) was degassed and purged with N₂ (3×). The mixture was stirred at 100° C. for 16 hours under N₂ atmosphere and then filtered, concentrated under reduced pressure, and purified by preparative HPLC (Method A) to give 1-chloro-7-methoxy-N-(1-methylpiperidin-3-yl)pyrido[3,4-d]pyridazin-4-amine as a yellow solid (50 mg, 37%) and 4-chloro-7-methoxy-N-(1-methylpiperidin-3-yl)pyrido[3,4-d]pyridazin-1-amine as a yellow solid (30 mg, 22%). ESI-MS m/z [M+H]⁺ 308.1.

### Preparation 22: (R)-4-chloro-N-(1-(2-fluoroethyl)piperidin-3-yl)phthalazin-1-amine

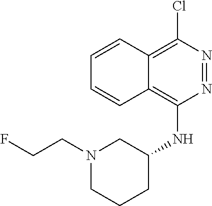 [SMILES: FCCN1CCC[C@@H](Nc2nnc(Cl)c3ccccc23)C1]

### Step 1: Tert-Butyl (R)-(1-(2-fluoroethyl)piperidin-3-yl)carbamate

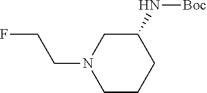 [SMILES: CC(C)(C)OC(=O)N[C@@H]1CCCN(CCF)C1]

To a mixture of tert-butyl (R)-piperidin-3-ylcarbamate (15 g, 74.90 mmol) and 1-bromo-2-fluoroethane (19.02 g, 149.79 mmol) in ACN (80 mL) were added NaI (5.61 g, 37.45 mmol) and K₂CO₃ (51.76 g, 374.48 mmol). The mixture was stirred at 15° C. for 12 hours and then diluted with H₂O (150 mL) and extracted with EtOAc (200 mL×2). The organic layers were combined, washed with brine (200 mL), dried over Na₂SO₄, filtered and concentrated under reduced pressure. The residue was purified by flash chromatography (ISCO® SepaFlash® 220 g silica gel column) using a gradient of 0 to 100% EtOAc in petroleum ether (85 m/min). The title compound was obtained as a white solid (14.3 g, 77.5%). ESI-MS m/z [M+H]⁺ 247.1.

### Step 2: (R)-1-(2-fluoroethyl)piperidin-3-amine

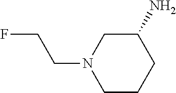

A mixture of tert-butyl (R)-(1-(2-fluoroethyl)piperidin-3-yl)carbamate (7 g, 28.42 mmol) in HCl/dioxane (4 M, 20 mL) was stirred at 18° C. for 12 hours and then concentrated under reduced pressure to give a dihydrochloride salt of the title compound as a white solid (6.1 g, 98% yield, 100% purity). ELSD-MS m/z [M+H]⁺ 147.1.

### Step 3: (R)-4-chloro-N-(1-(2-fluoroethyl)piperidin-3-yl)phthalazin-1-amine

To a mixture of (R)-1-(2-fluoroethyl)piperidin-3-amine (1.5 g, 10.26 mmol) and 1,4-dichlorophthalazine (2.45 g, 12.31 mmol) in DMSO (10 mL) was added DIPEA (5.30 g, 41.04 mmol, 7.15 mL). The mixture was stirred at 100° C. for 12 hours and then diluted with H₂O (50 mL) and extracted with EtOAc (50 mL×3). The organic layers were combined, washed with brine (150 mL), dried over Na₂SO₄, filtered and concentrated under reduced pressure. The residue was purified by flash chromatography (ISCO® SepaFlash® 40 g silica gel column) using a gradient of 0 to 100% EtOAc in petroleum ether (35 mL/min). The title compound was obtained as a yellow solid (1.1 g, crude). ESI-MS m/z [M+H]⁺ 309.

### Preparation 23: (R)-1-chloro-N-(1-(2-fluoroethyl)piperidin-3-yl)pyrido[3,4-d]pyridazin-4-amine

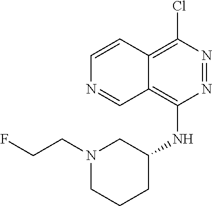 [SMILES: FCCN1CCC[C@@H](Nc2nnc(Cl)c3ccncc23)C1]

A mixture of (R)-1-(2-fluoroethyl)piperidin-3-amine (1.5 g, 10.26 mmol) and 1,4-dichloropyrido[3,4-d]pyridazine (2.46 g, 12.31 mmol) in DMSO (10 mL) was added DIPEA (5.30 g, 41.04 mmol, 7.15 mL). The mixture was stirred at 100° C. for 12 hours and then diluted with H₂O (50 mL) and extracted with EtOAc (50 mL×3). The organic layers were combined, washed with brine (150 mL), dried over Na₂SO₄, filtered and concentrated under reduced pressure. The residue was purified by flash chromatography (ISCO® SepaFlash® 40 g silica gel column) using a gradient of 0 to 100% EtOAc in petroleum ether (40 mL/min). The title compound was obtained as a brown oil (2 g, crude). ESI-MS m/z [M+H]⁺ 310.

### Preparation 24: 1-chloro-N-(1-cyclopropylpiperidin-3-yl)pyrido[3,4-d]pyridazin-4-amine and 4-chloro-N-(1-cyclopropylpiperidin-3-yl)pyrido[3,4-d]pyridazin-1-amine

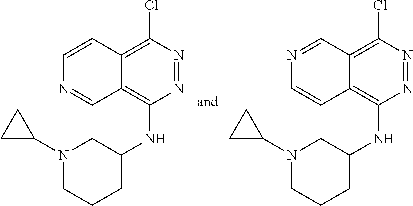 [SMILES: Clc1nnc(NC2CCCN(C3CC3)C2)c2cnccc12 ; Clc1nnc(NC2CCCN(C3CC3)C2)c2ccncc12]

To a mixture of 1,4-dichloropyrido[3,4-d]pyridazine (300 mg, 1.50 mmol) and 1-cyclopropylpiperidin-3-amine (479.58 mg, 2.25 mmol, 2 HCl) in DMSO (3 mL) was added DIPEA (1.94 g, 15.00 mmol, 2.61 mL) in one portion at 25° C. under N₂. The mixture was stirred at 100° C. for 12 hours and then purified by preparative HPLC (Method C) to give 1-chloro-N-(1-cyclopropylpiperidin-3-yl)pyrido[3,4-d]pyridazin-4-amine as a yellow solid (160 mg, 26.6%); ¹H NMR (400 MHz, DMSO-d₆) δ ppm 0.30-0.37 (m, 2H), 0.38-0.47 (m, 2H), 1.39-1.56 (m, 2H), 1.63-1.69 (m, 1H), 1.69-1.77 (m, 1H), 1.99 (br d, J=9.54 Hz, 1H), 2.18 (br t, J=10.29 Hz, 2H), 2.90 (br d, J=11.04 Hz, 1H), 3.24 (br d, J=7.53 Hz, 1H), 4.25 (br d, J=6.78 Hz, 1H), 7.74 (br d, J=7.53 Hz, 1H), 7.84 (d, J=5.52 Hz, 1H), 9.01-9.10 (m, 1H), 9.78 (s, 1H); ESI-MS m/z [M+H]⁺ 304.1; and 4-chloro-N-(1-cyclopropylpiperidin-3-yl)pyrido[3,4-d]pyridazin-1-amine as a yellow solid (96 mg, 16%); ¹H NMR (400 MHz, DMSO-d₆) δ ppm 0.29-0.46 (m, 4H), 1.39-1.56 (m, 2H), 1.64 (tt, J=6.56, 3.48 Hz, 1H), 1.69-1.78 (m, 1H), 1.99 (br d, J=9.29 Hz, 1H), 2.16 (br t, J=10.29 Hz, 2H), 2.89 (br d, J=11.04 Hz, 1H), 3.23 (br dd, J=10.42, 3.64 Hz, 1H), 4.20-4.30 (m, 1H), 7.73 (d, J=7.53 Hz, 1H), 7.82-7.89 (m, 1H), 9.05 (d, J=5.52 Hz, 1H), 9.78 (s, 1H); ESI-MS m/z [M+H]⁺ 304.1.

### Preparation 25: 4-chloro-N-((3R,5R)-1-cyclopropyl-5-fluoropiperidin-3-yl)phthalazin-1-amine

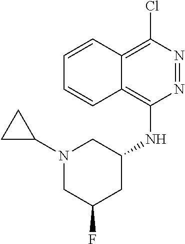 [SMILES: F[C@@H]1C[C@@H](Nc2nnc(Cl)c3ccccc23)CN(C2CC2)C1]

To a mixture of (3R,5R)-1-cyclopropyl-5-fluoropiperidin-3-amine dihydrochloride (214 mg, 0.778 mmol) and DIPEA (543 μL, 3.11 mmol) in NMP (3.89 mL) was added 1,4-dichlorophthalazine (155 mg, 0.778 mmol). The mixture was stirred at 125° C. overnight and then diluted with water and aq NaHCO₃. The organic phase was extracted with EtOAc (3×), washed with aq NaHCO₃, dried over Na₂SO₄ and concentrated in vacuo. The residue was purified by flash chromatography (ISCO® RediSep Rf Gold® 24 g silica gel column) using a gradient of 10 to 100% EtOAc in heptanes. The product-containing fractions were concentrated in vacuo to give the title compound as an orange oil (81.5 mg, 33%). ESI-MS m/z [M+H]⁺ 321.3.

### Preparation 26: 1-chloro-4-(4-chlorophenoxy)phthalazine

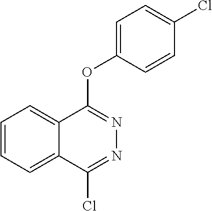 [SMILES: Clc1ccc(Oc2nnc(Cl)c3ccccc23)cc1]

To a solution of 1,4-dichlorophthalazine (1 g, 5.02 mmol), 4-chlorophenol (645.89 mg, 5.02 mmol, 493.05 μL) in DMF (10 mL) was added K₂CO₃ (1.39 g, 10.05 mmol). The mixture was stirred at 25° C. for 15 hours. LC-MS showed one main peak with desired m/z. The reaction mixture was concentrated under reduced pressure to remove solvent, dissolved in H₂O (30 mL) and extracted with EtOAc (30 mL). The organic phases were combined, dried over Na₂SO₄, filtered, concentrated under reduced pressure to give the title compound (1.09 g, crude). ESI-MS m/z [M+H]⁺ 290.9.

### Preparation 27: 1-chloro-4-(4-chloro-2-fluorophenyl)phthalazine

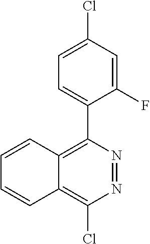 [SMILES: Fc1cc(Cl)ccc1-c1nnc(Cl)c2ccccc12]

A mixture of 1,4-dichlorophthalazine (1 g, 5.02 mmol), (4-chloro-2-fluorophenyl)boronic acid (963.65 mg, 5.53 mmol), Pd(dppf)Cl₂·CH₂Cl₂ (410.29 mg, 502.42 μmol) and Na₂CO₃ (1.60 g, 15.07 mmol) in dioxane (2 mL) was degassed and purged with N₂ (3×). The mixture was stirred at 100° C. for 1 hour under N₂ atmosphere and then concentrated under vacuum. The crude product was purified by flash chromatography (ISCO® SepaFlash® 12 g silica gel column) using a gradient of 0 to 15% EtOAc in petroleum ether (45 m/min). The title compound was obtained as a white solid (500 mg, 33.9%). ESI-MS m/z [M+H]⁺ 292.9.

### Preparation 28: 1-chloro-4-(4-chlorophenyl)phthalazine

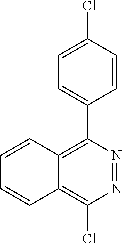 [SMILES: Clc1ccc(-c2nnc(Cl)c3ccccc23)cc1]

The title compound (2.6 g, 38%) was made like Preparation 27, using 1,4-dichlorophthalazine (5 g, 25.12 mmol) and (4-chlorophenyl)boronic acid (3.93 g, 25.12 mmol). ESI-MS m/z [M+H]⁺ 274.8.

### Preparation 29: 4-chloro-1-(4-methoxyphenyl)pyrido[3,4-d]pyridazine

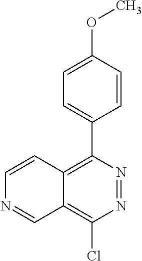 [SMILES: COc1ccc(-c2nnc(Cl)c3cnccc23)cc1]

### Step 1: 4-(4-methoxybenzoyl)nicotinic Acid

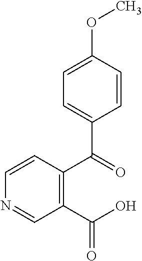 [SMILES: COc1ccc(C(=O)c2ccncc2C(=O)O)cc1]

To a solution of furo[3,4-c]pyridine-1,3-dione (5 g, 33.53 mmol) in THF (50 mL) was added bromo-(4-methoxyphenyl)magnesium (0.5 M, 53.65 mL) dropwise at −78° C. over a 30-minute period. The reaction mixture was stirred at −78° C. for 1 hour under N₂ atmosphere. LC-MS showed one main peak with the desired mass. The reaction was quenched by slow addition of H₂O (20 mL). The mixture was stirred for 30 minutes and then filtered. The filter cake was rinsed with THE (15 mL×3) and dried under vacuum to give the title compound as a white solid (6 g, 70%). ¹H NMR (400 MHz, DMSO-d₆) δ 3.87 (s, 3H), 6.93-7.10 (m, 2H), 7.18-7.36 (m, 1H), 7.49-7.74 (m, 2H), 8.60-8.83 (m, 1H), 9.10-9.30 (m, 1H); ESI-MS m/z [M+H]⁺ 258.2.

### Step 2: 1-(4-methoxyphenyl)pyrido[3,4-d]pyridazin-4-ol

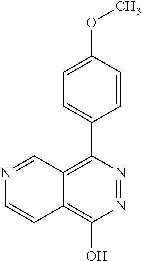 [SMILES: COc1ccc(-c2nnc(O)c3ccncc23)cc1]

A mixture of 4-(4-methoxybenzoyl)nicotinic acid (2 g, 7.77 mmol, 1 eq), hydrazine hydrochloride (798.93 mg, 11.66 mmol) and Et₃N (1.57 g, 15.55 mmol, 2.16 mL) in EtOH (10 mL) was stirred at 90° C. for 12 hours. LC-MS showed one peak with the desired mass. The reaction mixture was concentrated, triturated with ACN (25 mL) and filtered. The filter cake was collected and dried to give the title compound as a white solid (4.3 g, crude). ¹H NMR (400 MHz, DMSO-d₆) δ ppm 3.85 (s, 3H), 7.09-7.16 (m, 2H), 7.53-7.64 (m, 3H), 8.96-9.04 (m, 1H), 9.46-9.57 (m, 1H); ESI-MS m/z [M+H]⁺ 254.1.

### Step 3: 4-chloro-1-(4-methoxyphenyl)pyrido[3,4-d]pyridazine

To a mixture of 1-(4-methoxyphenyl)pyrido[3,4-d]pyridazin-4-ol (1 g, 3.95 mmol) in POCl₃ (10 mL) was added DIPEA (1.53 g, 11.85 mmol, 2.06 mL). The mixture was stirred at 100° C. for 1 hour. LC-MS showed one peak with the desired mass. The reaction mixture was concentrated under reduced pressure, dissolved in DCM (5 mL) and adjusted to pH 7-8 by addition of DIPEA. The mixture was concentrated and then purified by flash chromatography (ISCO® SepaFlash® 20 g silica gel column) using a gradient of 0 to 35% EtOAc in petroleum ether (40 mL/min). The title compound was obtained as a yellow solid (320 mg, 1.18 mmol, 29.8% yield, 90% purity). ¹H NMR (400 MHz, DMSO-d₆) δ ppm 3.84-3.93 (m, 3H), 7.15-7.28 (m, 2H), 7.82 (br d, J=8.76 Hz, 2H), 7.91-8.01 (m, 1H), 9.14-9.26 (m, 1H), 9.73-9.80 (m, 1H); ESI-MS m/z [M+H]⁺ 271.9.

### Preparation 30: 8-chloro-5-(4-methoxyphenyl)pyrido[2,3-d]pyridazine

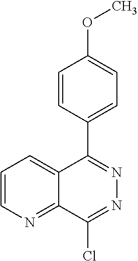 [SMILES: COc1ccc(-c2nnc(Cl)c3ncccc23)cc1]

To a solution of 5,8-dichloropyrido[2,3-d]pyridazine (200 mg, 999.88 μmol), (4-methoxyphenyl)boronic acid (151.94 mg, 999.88 μmol) and Na₂CO₃ (211.95 mg, 2.00 mmol) in toluene (5 mL) and H₂O (1.25 mL) was added Pd(PPh₃)₄ (115.54 mg, 99.99 μmol) in one portion. The mixture was degassed and purged with N₂ (3×) and then stirred at 90° C. for 12 hours under N₂. The reaction mixture was filtered and the filtrate was concentrated and purified by preparative HPLC (Method B). The title compound was obtained as a yellow solid (43 mg, 16%). ¹H NMR (400 MHz, DMSO-d₆) δ ppm 3.89 (s, 3H), 7.21 (d, J=8.40 Hz, 2H), 7.74 (d, J=8.40 Hz, 2H), 8.09-8.12 (m, 1H), 8.50 (d, J=8.40 Hz, 1H), 9.42 (d, J=2.80 Hz, 1H); ESI-MS m/z [M+H]⁺ 272.0.

### Preparation 31: 1-chloro-4-(4-methoxyphenyl)-5,6,7,8-tetrahydrophthalazine

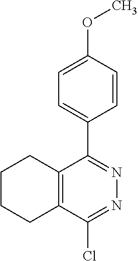 [SMILES: COc1ccc(-c2nnc(Cl)c3c2CCCC3)cc1]

A mixture of 1,4-dichloro-5,6,7,8-tetrahydrophthalazine (160 mg, 787.91 μmol), (4-methoxyphenyl)boronic acid (179.59 mg, 1.18 mmol), Pd(PPh₃)₄ (91.05 mg, 78.79 μmol), KF (91.55 mg, 1.58 mmol, 36.92 μL, 2 eq) and K₃PO₄ (334.49 mg, 1.58 mmol) in toluene (5 mL) was degassed and purged with N₂ (3×). The mixture was stirred at 130° C. for 1 hour under N₂ atmosphere and then concentrated under vacuum. The crude product was purified by preparative HPLC (Method B). The title compound was obtained as a white solid (110 mg, 50.8%). ESI-MS m/z [M+H]⁺ 275.0.

### Preparation 32: 4-chloro-7-(4-methoxyphenyl)-1-methyl-1H-pyrazolo[3,4-d]pyridazine

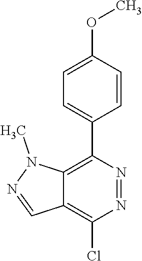 [SMILES: COc1ccc(-c2nnc(Cl)c3cnn(C)c23)cc1]

### Step 1: methyl 5-(chlorocarbonyl)-1-methyl-1H-pyrazole-4-carboxylate

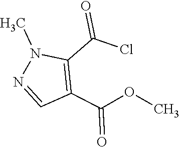

To a 100 mL round-bottomed flask charged with 4-(methoxycarbonyl)-1-methyl-1H-pyrazole-5-carboxylic acid (0.5 g, 2.72 mmol) in THF (10 mL) was added dropwise sulfurous dichloride (0.394 mL, 5.43 mmol). The mixture was heated under reflux overnight and then concentrated to dryness to give the title compound, which was used without further purification.

### Step 2: methyl 5-(4-methoxybenzoyl)-1-methyl-1H-pyrazole-4-carboxylate

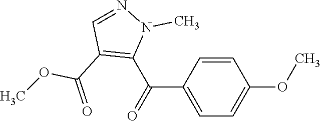

To a 100 mL round-bottomed flask charged with methyl 5-(chlorocarbonyl)-1-methyl-1H-pyrazole-4-carboxylate (0.551 g, 2.72 mmol) in toluene (6 mL) was added dropwise a solution of 1-methylpyrrolidin-2-one (0.261 mL, 2.72 mmol) in toluene (6 mL) at 0° C. The mixture was stirred at 0° C. for 15 minutes and then cooled to −10° C. Next, (4-methoxyphenyl)magnesium bromide (4.53 mL, 2.267 mmol) in toluene was added dropwise. The mixture was stirred at 0° C. for 1 hour and then quenched with saturated NH₄Cl and extracted with EtOAc. The organic phases were combined, washed with brine, dried over MgSO₄ and concentrated to give the title compound as a brown syrup, which was used without further purification. ESI-MS m/z [M+H]⁺ 275.1.

### Step 3: 7-(4-methoxyphenyl)-1-methyl-1H-pyrazolo[3,4-d]pyridazin-4-ol

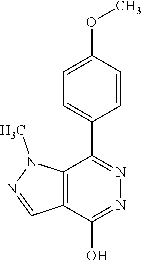 [SMILES: COc1ccc(-c2nnc(O)c3cnn(C)c23)cc1]

To a 150 mL pressure vial were added methyl 5-(4-methoxybenzoyl)-1-methyl-1H-pyrazole-4-carboxylate (0.620 g, 2.26 mmol) and hydrazine HCl (0.929 g, 13.56 mmol) in acetic acid (8 mL) to give a brown suspension. The reaction mixture was heated at reflux for 5 days and then concentrated under reduced pressure. The residue was neutralized with saturated aq NaHCO₃ and the aqueous phase extracted with EtOAc. The organic phases were combined, washed with brine, dried over MgSO₄ and concentrated to give the title compound as a brown solid (0.162 g, 28%). ESI-MS m/z [M+H]⁺ 257.1.

### Step 4: 4-chloro-7-(4-methoxyphenyl)-1-methyl-1H-pyrazolo[3,4-d]pyridazine

To a 100 mL round-bottomed flask were added 7-(4-methoxyphenyl)-1-methyl-1H-pyrazolo[3,4-d]pyridazin-4-ol (0.162 g, 0.632 mmol), POCl₃ (0.236 mL, 2.53 mmol), and N,N-dimethylaniline (0.321 mL, 2.53 mmol) in dioxane (8 mL) to give a tan solution. The mixture was heated at 90° C. for 30 minutes and then concentrated under reduced pressure. The residue was treated with saturated aq NaHCO₃ and extracted with EtOAc. The organic phases were combined, washed with brine, dried over MgSO₄, concentrated, and dried under high vacuum to give the title compound (crude) as a tan solid (180 mg, 100% yield). ESI-MS m/z [M+H]⁺ 275.3.

### Preparation 33: Tert-Butyl (3R,5S)-3-((4-chlorophthalazin-1-yl)amino)-5-fluoropiperidine-1-carboxylate

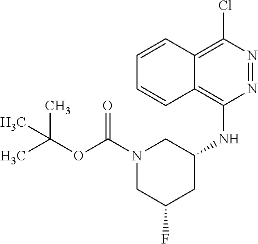 [SMILES: CC(C)(C)OC(=O)N1C[C@@H](F)C[C@@H](Nc2nnc(Cl)c3ccccc23)C1]

A mixture of tert-butyl (3R,5S)-3-amino-5-fluoro-piperidine-1-carboxylate (370 mg, 1.70 mmol), 1,4-dichlorophthalazine (404.88 mg, 2.03 mmol) and DIPEA (1.10 g, 8.48 mmol, 1.48 mL) in DMSO (5 mL) was stirred at 100° C. for 12 hours. The mixture was diluted with H₂O (30 mL) and extracted with EtOAc (30 mL×3). The organic layers were combined, dried over anhydrous Na₂SO₄, filtered and concentrated under vacuum. The resulting residue was purified by flash chromatography (ISCO® SepaFlash® 20 g silica gel column) using a gradient of 0 to 50% EtOAc in petroleum ether (40 m/min). The title compound was obtained as a yellow oil (310 mg, crude). ESI-MS m/z [M+H]⁺ 381.1.

### Preparation 34: Tert-Butyl (3R,5R)-3-((4-chlorophthalazin-1-yl)amino)-5-fluoropiperidine-1-carboxylate

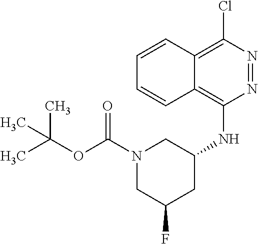 [SMILES: CC(C)(C)OC(=O)N1C[C@H](F)C[C@@H](Nc2nnc(Cl)c3ccccc23)C1]

The title compound was made like Preparation 33, using tert-butyl (3R,5R)-3-amino-5-fluoropiperidine-1-carboxylate (0.873 g, 4.00 mmol) and 1,4-dichlorophthalazine (0.796 g, 4.00 mmol), and was obtained as a light-yellow foam (0.630 g, 41%). ESI-MS m/z [M+H]⁺ 381.3.

### PREPARATION 35: Tert-Butyl (3R,5R)-3-((4-(4-chlorophenyl)phthalazin-1-yl)amino)-5-fluoropiperidine-1-carboxylate

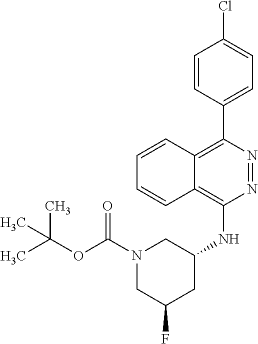 [SMILES: CC(C)(C)OC(=O)N1C[C@H](F)C[C@@H](Nc2nnc(-c3ccc(Cl)cc3)c3ccccc23)C1]

The title compound was made like Preparation 33, using tert-butyl (3R,5R)-3-((4-chlorophthalazin-1-yl)amino)-5-fluoropiperidine-1-carboxylate (0.240 g, 0.630 mmol), 2-(4-chlorophenyl)-4,4,5,5-tetramethyl-1,3,2-dioxaborolane (0.188 g, 0.788 mmol) and aq K₂CO₃ (2 M, 0.945 mL, 1.89 mmol). The title compound was obtained as an off-white solid (75.6 mg, 26%). ESI-MS m/z [M+H]⁺ 457.3.

### Preparation 36: Tert-Butyl (3R,5R)-3-fluoro-5-((4-(4-methoxyphenyl)phthalazin-1-yl)amino)piperidine-1-carboxylate

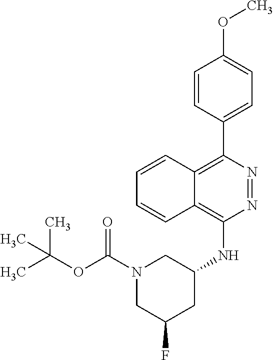 [SMILES: COc1ccc(-c2nnc(N[C@@H]3C[C@@H](F)CN(C(=O)OC(C)(C)C)C3)c3ccccc23)cc1]

The title compound was made like Preparation 33, using tert-butyl (3R,5R)-3-((4-chlorophthalazin-1-yl)amino)-5-fluoropiperidine-1-carboxylate (0.152 g, 0.400 mmol), 2-(4-methoxyphenyl)-4,4,5,5-tetramethyl-1,3,2-dioxaborolane (0.117 g, 0.500 mmol) and aq K₂CO₃ (2 M, 0.600 mL, 1.20 mmol). The title compound was obtained as a light-yellow semisolid (100.8 mg, 56%). ESI-MS m/z [M+H]⁺ 453.4.

### Preparation 37: Tert-Butyl (3R,5R)-3-fluoro-5-((4-(2-fluoro-4-methoxyphenyl)phthalazin-1-yl)amino)piperidine-1-carboxylate

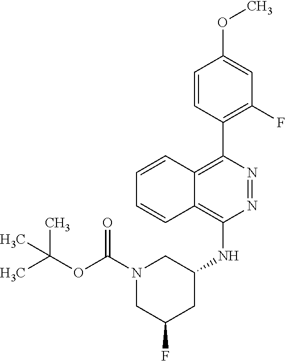 [SMILES: COc1ccc(-c2nnc(N[C@@H]3C[C@@H](F)CN(C(=O)OC(C)(C)C)C3)c3ccccc23)c(F)c1]

The title compound was made like Preparation 33, using (2-fluoro-4-methoxyphenyl)boronic acid. ESI-MS m/z [M+H]⁺ 471.4.

Preparation 38: Tert-Butyl (3R,5R)-3-((1-chloropyrido[3,4-d]pyridazin-4-yl)amino)-5-fluoropiperidine-1-carboxylate and tert-butyl (3R,5R)-3-((4-chloropyrido[3,4-d]pyridazin-1-yl)amino)-5-fluoropiperidine-1-carboxylate

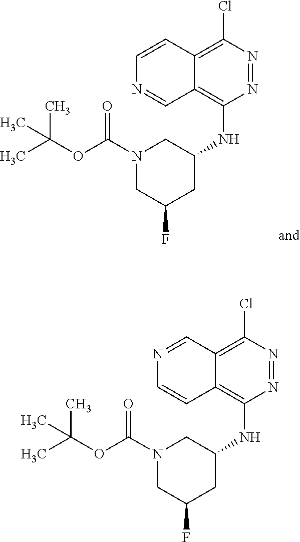 [SMILES: CC(C)(C)OC(=O)N1C[C@H](F)C[C@@H](Nc2nnc(Cl)c3ccncc23)C1 ; CC(C)(C)OC(=O)N1C[C@H](F)C[C@@H](Nc2nnc(Cl)c3cnccc23)C1]

A mixture of 1,4-dichloropyrido[3,4-d]pyridazine (100 mg, 499.94 μmol), tert-butyl (3R,5R)-3-amino-5-fluoropiperidine-1-carboxylate (163.68 mg, 749.91 μmol), DIPEA (323.07 mg, 2.50 mmol, 435.40 μL) and NaI (74.94 mg, 499.94 μmol) in DMSO (2 mL) was degassed and purged with N₂ (3×). The mixture was stirred at 90° C. for 1 hour under N₂ atmosphere and then diluted with H₂O (30 mL) and extracted with EtOAc (40 mL×2). The organic layers were combined, dried over anhydrous Na₂SO₄ and filtered. The filtrate was evaporated under vacuum to give a mixture of the title compounds as a red solid (500 mg, crude). ESI-MS m/z [M+H]⁺ 382.

### Preparation 39: Tert-Butyl (3R,5S)-3-((4-(4-chlorophenyl)phthalazin-1-yl)amino)-5-fluoropiperidine-1-carboxylate

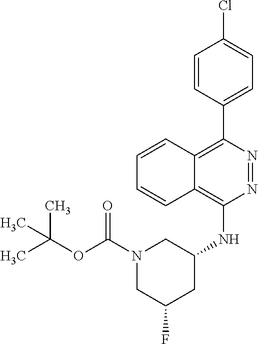 [SMILES: CC(C)(C)OC(=O)N1C[C@@H](F)C[C@@H](Nc2nnc(-c3ccc(Cl)cc3)c3ccccc23)C1]

To a solution of tert-butyl (3R,5S)-3-((4-chlorophthalazin-1-yl)amino)-5-fluoropiperidine-1-carboxylate (270 mg, 708.95 μmol), (4-chlorophenyl)boronic acid (144.12 mg, 921.64 μmol) and Cs₂CO₃ (461.98 mg, 1.42 mmol) in dioxane (5 mL) and H₂O (1 mL) was added Pd(dppf)Cl₂ (103.75 mg, 141.79 μmol). The mixture was stirred at 100° C. for 12 hours under N₂ and then filtered. The filtrate was concentrated under vacuum and purified by column chromatography (SiO₂, DCM/MeOH=1:0 to 20:1). The title compound was obtained as a yellow solid (320 mg, crude). ESI-MS m/z [M+H]⁺ 457.2.

### Preparation 40: Tert-Butyl (3R,5R)-3-((4-(4-chloro-2-fluorophenyl)phthalazin-1-yl)amino)-5-fluoropiperidine-1-carboxylate

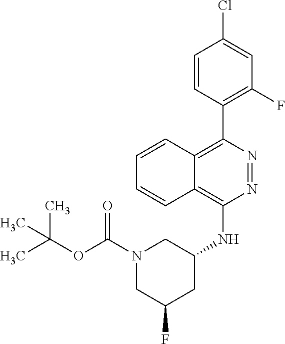 [SMILES: CC(C)(C)OC(=O)N1C[C@H](F)C[C@@H](Nc2nnc(-c3ccc(Cl)cc3F)c3ccccc23)C1]

To a solution of 1-chloro-4-(4-chloro-2-fluorophenyl)phthalazine (100 mg, 341.15 μmol) in dioxane (1 mL) were added BINAP (10.62 mg, 17.06 μmol), tert-butyl (3R,5R)-3-amino-5-fluoropiperidine-1-carboxylate (89.36 mg, 409.38 μmol), Pd(OAc)₂ (7.66 mg, 34.12 μmol) and Cs₂CO₃ (333.46 mg, 1.02 mmol). The mixture was stirred at 100° C. for 1 hour. The reaction mixture was concentrated under vacuum and the resulting residue was purified by flash chromatography (ISCO® SepaFlash® 12 g silica gel column) using a gradient of 0 to 45% EtOAc in petroleum ether (85 m/min). The title compound was obtained as an orange solid (55 mg, 34%). ESI-MS m/z [M+H]⁺ 475.2.

### Preparation 41: N-((3R,5R)-5-fluoropiperidin-3-yl)-4-(4-methoxyphenyl)phthalazin-1-amine

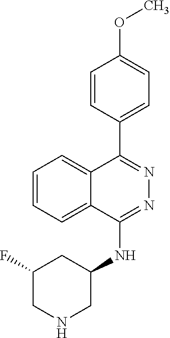 [SMILES: COc1ccc(-c2nnc(N[C@H]3CNC[C@H](F)C3)c3ccccc23)cc1]

A solution of tert-butyl (3R,5R)-3-fluoro-5-((4-(4-methoxyphenyl)phthalazin-1-yl)amino)piperidine-1-carboxylate (0.358 g, 0.791 mmol) in dioxane (1.98 mL) was treated with HCl (4 M in dioxane, 1.98 mL, 7.91 mmol). The mixture was stirred at room temperature for 45 minutes. A few drops of MeOH were added and the mixture was stirred for another 15 minutes. The reaction mixture was concentrated in vacuo. The residue was taken up in MeOH and filtered through a hydrophilic PTFE 0.45 m syringe filter (VWR®). The filtrate was purified by preparative HPLC (Method C) over two injections. The product-containing fractions were evaporated to give the title compound as a yellow-orange oil (0.191 g, 68%). ESI-MS m/z [M+H]⁺ 353.3.

Preparation 42: Tert-Butyl (3R,5R)-3-fluoro-5-((1-(4-methoxyphenyl)pyrido[3,4-d]pyridazin-4-yl)amino)piperidine-1-carboxylate and tert-butyl (3R,5R)-3-fluoro-5-((4-(4-methoxyphenyl)pyrido[3,4-d]pyridazin-1-yl)amino)piperidine-1-carboxylate

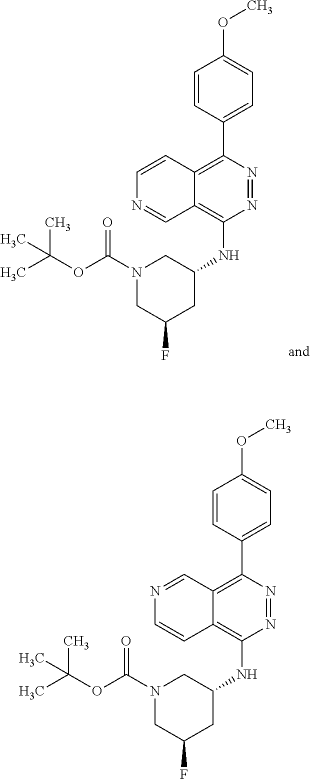 [SMILES: COc1ccc(-c2nnc(N[C@@H]3C[C@@H](F)CN(C(=O)OC(C)(C)C)C3)c3cnccc23)cc1 ; COc1ccc(-c2nnc(N[C@@H]3C[C@@H](F)CN(C(=O)OC(C)(C)C)C3)c3ccncc23)cc1]

A mixture of tert-butyl (3R,5R)-3-((1-chloropyrido[3,4-d]pyridazin-4-yl)amino)-5-fluoropiperidine-1-carboxylate and tert-butyl (3R,5R)-3-((4-chloropyrido[3,4-d]pyridazin-1-yl)amino)-5-fluoropiperidine-1-carboxylate (190 mg, 497.60 μmol), (4-methoxyphenyl)boronic acid (113.42 mg, 746.40 μmol), Pd(dppf)Cl₂ (40.64 mg, 49.76 μmol) and Cs₂CO₃ (324.26 mg, 995.20 μmol) in dioxane (1 mL) was degassed and purged with N₂ (3×). The mixture was stirred at 90° C. for 1 hour under N₂ and then dispersed in H₂O (30 mL) and extracted with EtOAc (40 mL×2). The organic layers were combined, dried over anhydrous Na₂SO₄ and filtered. The filtrate was concentrated in vacuo and purified by preparative HPLC (Method B) to give tert-butyl (3R,5R)-3-fluoro-5-((1-(4-methoxyphenyl)pyrido[3,4-d]pyridazin-4-yl)amino)piperidine-1-carboxylate (60 mg, 24.8% yield, 93.3% purity) and tert-butyl (3R,5R)-3-fluoro-5-((4-(4-methoxyphenyl)pyrido[3,4-d]pyridazin-1-yl)amino)piperidine-1-carboxylate (20 mg, 8.6% yield, 97.1% purity) as white solids. ESI-MS m/z [M+H]⁺ 454.3.

Preparation 43: Tert-Butyl (3R,5R)-3-((1-(4-chlorophenyl)pyrido[3,4-d]pyridazin-4-yl)amino)-5-fluoropiperidine-1-carboxylate and tert-butyl (3R,5R)-3-((4-(4-chlorophenyl)pyrido[3,4-d]pyridazin-1-yl)amino)-5-fluoropiperidine-1-carboxylate

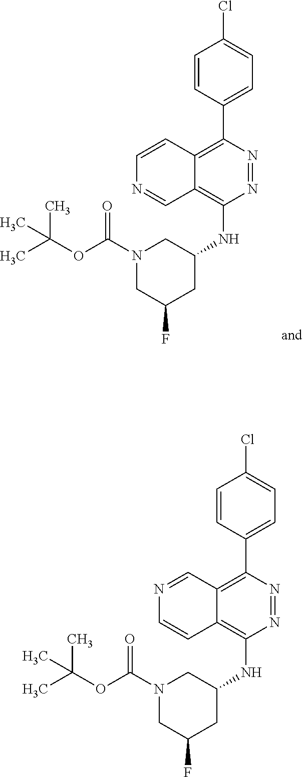 [SMILES: CC(C)(C)OC(=O)N1C[C@H](F)C[C@@H](Nc2nnc(-c3ccc(Cl)cc3)c3ccncc23)C1 ; CC(C)(C)OC(=O)N1C[C@H](F)C[C@@H](Nc2nnc(-c3ccc(Cl)cc3)c3cnccc23)C1]

The title compounds were made like Preparation 42, using a mixture of tert-butyl (3R,5R)-3-((1-chloropyrido[3,4-d]pyridazin-4-yl)amino)-5-fluoropiperidine-1-carboxylate (200 mg, 523.79 μmol) and tert-butyl (3R,5R)-3-((4-chloropyrido[3,4-d]pyridazin-1-yl)amino)-5-fluoropiperidine-1-carboxylate, and (4-chlorophenyl)boronic acid (122.86 mg, 785.69 μmol). Each of (3R,5R)-tert-butyl 3-((4-(4-chlorophenyl)pyrido[3,4-d]pyridazin-1-yl)amino)-5-fluoropiperidine-1-carboxylate (20 mg, 8.2%) and (3R,5R)-tert-butyl 3-((1-(4-chlorophenyl)pyrido[3,4-d]pyridazin-4-yl)amino)-5-fluoropiperidine-1-carboxylate (45 mg, crude) were obtained as white solids. ESI-MS m/z [M+H]⁺ 458.2.

Preparation 44: Tert-Butyl (3R,5R)-3-fluoro-5-((1-(4-fluoro-2-methoxyphenyl)pyrido[3,4-d]pyridazin-4-yl)amino)piperidine-1-carboxylate and tert-butyl (3R,5R)-3-fluoro-5-((4-(4-fluoro-2-methoxyphenyl)pyrido[3,4-d]pyridazin-1-yl)amino)piperidine-1-carboxylate

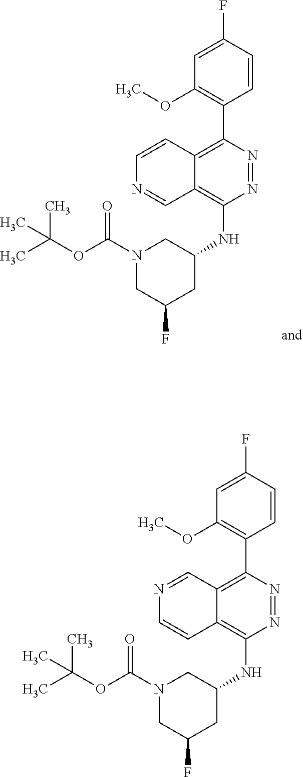 [SMILES: COc1cc(F)ccc1-c1nnc(N[C@@H]2C[C@@H](F)CN(C(=O)OC(C)(C)C)C2)c2cnccc12 ; COc1cc(F)ccc1-c1nnc(N[C@@H]2C[C@@H](F)CN(C(=O)OC(C)(C)C)C2)c2ccncc12]

The title compounds were made like Preparation 42, using a mixture of tert-butyl (3R,5R)-3-((1-chloropyrido[3,4-d]pyridazin-4-yl)amino)-5-fluoropiperidine-1-carboxylate and tert-butyl (3R,5R)-3-((4-chloropyrido[3,4-d]pyridazin-1-yl)amino)-5-fluoropiperidine-1-carboxylate (300 mg, 785.69 μmol), and (4-fluoro-2-methoxyphenyl)boronic acid (267.05 mg, 1.57 mmol). The title compounds tert-butyl (3R,5R)-3-fluoro-5-((1-(4-fluoro-2-methoxyphenyl)pyrido[3,4-d]pyridazin-4-yl)amino)piperidine-1-carboxylate (110 mg, 28.8% yield, 97% purity) and tert-butyl (3R,5R)-3-fluoro-5-((4-(4-fluoro-2-methoxyphenyl)pyrido[3,4-d]pyridazin-1-yl)amino)piperidine-1-carboxylate (60 mg, μmol, 8.1% yield, 50% purity) were obtained as white solids. ESI-MS m/z [M+H]⁺ 472.1.

### Preparation 45: Tert-Butyl (3R,5R)-3-fluoro-5-((1-(2-fluoro-4-methoxyphenyl)pyrido[3,4-d]pyridazin-4-yl)amino)piperidine-1-carboxylate

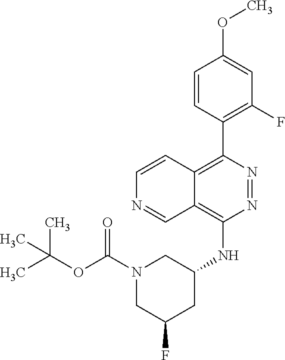 [SMILES: COc1ccc(-c2nnc(N[C@@H]3C[C@@H](F)CN(C(=O)OC(C)(C)C)C3)c3cnccc23)c(F)c1]

The title compound was made like Preparation 42, using a mixture of tert-butyl (3R,5R)-3-((1-chloropyrido[3,4-d]pyridazin-4-yl)amino)-5-fluoropiperidine-1-carboxylate and tert-butyl (3R,5R)-3-((4-chloropyrido[3,4-d]pyridazin-1-yl)amino)-5-fluoropiperidine-1-carboxylate (650 mg, 1.70 mmol), and (2-fluoro-4-methoxyphenyl)boronic acid (578.60 mg, 3.40 mmol). The title compound was obtained as a white solid (170 mg, 20.8% yield, 98% purity). ESI-MS m/z [M+H]⁺ 472.1.

Preparation 46: Tert-Butyl (3R,5R)-3-((1-(4-chloro-2-fluorophenyl)pyrido[3,4-d]pyridazin-4-yl)amino)-5-fluoropiperidine-1-carboxylate and tert-butyl (3R,5R)-3-((4-(4-chloro-2-fluorophenyl)pyrido[3,4-d]pyridazin-1-yl)amino)-5-fluoropiperidine-1-carboxylate

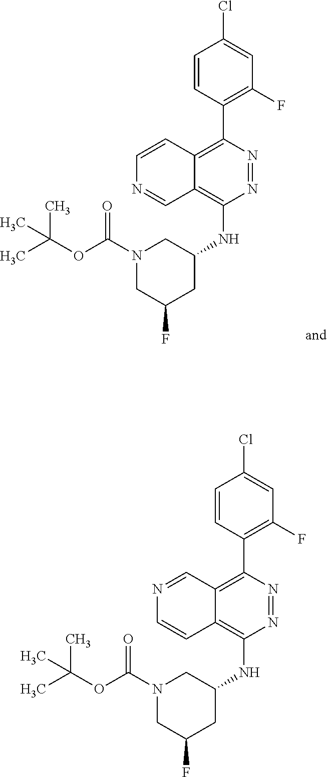 [SMILES: CC(C)(C)OC(=O)N1C[C@H](F)C[C@@H](Nc2nnc(-c3ccc(Cl)cc3F)c3ccncc23)C1 ; CC(C)(C)OC(=O)N1C[C@H](F)C[C@@H](Nc2nnc(-c3ccc(Cl)cc3F)c3cnccc23)C1]

A mixture of the title compounds was made like Preparation 42, using a mixture of tert-butyl (3R,5R)-3-((1-chloropyrido[3,4-d]pyridazin-4-yl)amino)-5-fluoropiperidine-1-carboxylate and tert-butyl (3R,5R)-3-((4-chloropyrido[3,4-d]pyridazin-1-yl)amino)-5-fluoropiperidine-1-carboxylate (200 mg, 523.79 μmol), and (4-chloro-2-fluorophenyl)boronic acid (182.66 mg, 1.05 mmol). The mixture of the title compounds was obtained as a yellow solid (110 mg, 39.7%). ESI-MS m/z [M+H]⁺ 476.3.

### Preparation 47: Tert-Butyl 5-((4-(4-chlorophenyl)phthalazin-1-yl)amino)-3,3-difluoropiperidine-1-carboxylate

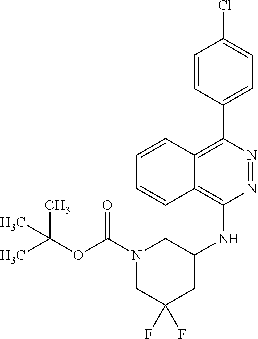 [SMILES: CC(C)(C)OC(=O)N1CC(Nc2nnc(-c3ccc(Cl)cc3)c3ccccc23)CC(F)(F)C1]

A mixture of tert-butyl 5-amino-3,3-difluoropiperidine-1-carboxylate (118 mg, 0.500 mmol) and 1-chloro-4-(4-chlorophenyl)phthalazine (275 mg, 0.500 mmol) in NMP (2.5 mL) was treated with K₂CO₃ (207 mg, 1.50 mmol). The reaction mixture was stirred at 150° C. for 2 days and then diluted with water and extracted with EtOAc (3×). The organic phases were combined, washed with saturated aq NaHCO₃, dried over Na₂SO₄, filtered, and concentrated under reduced pressure to give the (crude) title compound as a brown oil (237 mg). ESI-MS m/z [M+H]⁺ 475.3.

### Preparation 48: 1-(4-chlorophenyl)-N-(piperidin-3-yl)pyrido[3,4-d]pyridazin-4-amine

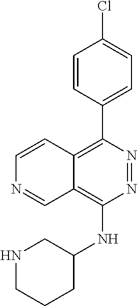 [SMILES: Clc1ccc(-c2nnc(NC3CCCNC3)c3cnccc23)cc1]

### Step 1: Tert-Butyl 3-((1-chloropyrido[3,4-d]pyridazin-4-yl)amino)piperidine-1-carboxylate

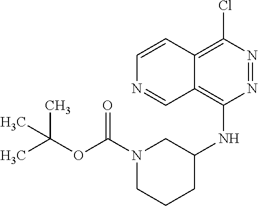 [SMILES: CC(C)(C)OC(=O)N1CCCC(Nc2nnc(Cl)c3ccncc23)C1]

A mixture of 1,4-dichloropyrido[3,4-d]pyridazine (0.200 g, 1 mmol), tert-butyl 3-aminopiperidine-1-carboxylate (0.200 g, 1.000 mmol) and K₂CO₃ (0.415 g, 3.00 mmol) in DMSO (3.33 mL) was stirred at 100° C. overnight. The reaction mixture was filtered and the filtrate was purified by preparative HPLC. The title compound was obtained as an off-white solid. ESI-MS m/z [M+H]⁺ 364.4.

### Step 2: Tert-Butyl 3-((1-(4-chlorophenyl)pyrido[3,4-d]pyridazin-4-yl)amino)piperidine-1-carboxylate

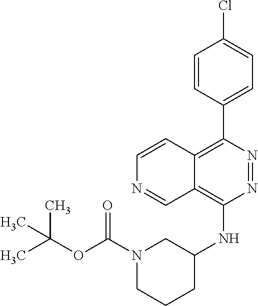 [SMILES: CC(C)(C)OC(=O)N1CCCC(Nc2nnc(-c3ccc(Cl)cc3)c3ccncc23)C1]

A mixture of tert-butyl 3-((1-chloropyrido[3,4-d]pyridazin-4-yl)amino)piperidine-1-carboxylate (0.364 g, 1.00 mmol), (4-chlorophenyl)boronic acid (0.156 g, 1.00 mmol), Pd(PPh₃)₄ (0.116 g, 0.100 mmol) and K₂CO₃ (0.415 g, 3.00 mmol) in toluene (3.47 mL) and water (0.694 mL) was degassed and purged with N₂. The mixture was stirred at 90° C. for 1 hour under N₂ atmosphere and then concentrated under reduced pressure. DMF (3 mL) was added and the mixture was filtered. The filtrate was purified by preparative HPLC. The title compound was obtained as an off-white solid (0.440 g). ESI-MS m/z [M+H]⁺ 440.3.

### Step 3: 1-(4-chlorophenyl)-N-(piperidin-3-yl)pyrido[3,4-d]pyridazin-4-amine

A mixture of tert-butyl 3-((1-(4-chlorophenyl)pyrido[3,4-d]pyridazin-4-yl)amino)piperidine-1-carboxylate (0.440 g, 1 mmol) and TFA (1 mL, 12.98 mmol) in DCM (10 mL) was stirred at room temperature overnight. The reaction was concentrated in vacuo to give the title compound as an off-white solid. ESI-MS m/z [M+H]⁺ 340.2.

### Preparation 49: 1,4-dichloro-7,8-dihydro-5H-pyrano[3,4-d]pyridazine

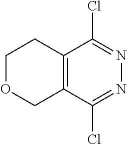 [SMILES: Clc1nnc(Cl)c2c1CCOC2]

### Step 1: Methyl 4-(((trifluoromethyl)sulfonyl)oxy)-5,6-dihydro-2H-pyran-3-carboxylate

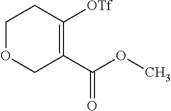 [SMILES: COC(=O)C1=C(OS(=O)(=O)C(F)(F)F)CCOC1]

To a solution of methyl 4-oxotetrahydropyran-3-carboxylate (8 g, 50.58 mmol) in THE (100 mL) was added NaH (2.43 g, 60.70 mmol, 60% purity) at −78° C. After stirring for 1 hour, a solution of N-(5-chloro-2-pyridyl)-1,1,1-trifluoro-N-(trifluoromethylsulfonyl)methanesulfonamide (23.84 g, 60.70 mmol) in THE (50 mL) was added dropwise, and the mixture was stirred at 20° C. for 12 hours. TLC (petroleum ether/EtOAc=5:1) indicated the starting material was completely consumed. The mixture was quenched with saturated aq NH₄Cl (300 mL) and extracted with EtOAc (100 mL×2). The combined organic layers were concentrated in vacuo. The resulting crude product was purified by flash chromatography (ISCO® SepaFlash® 80 g silica gel column) using a gradient of 0 to 15% EtOAc in petroleum ether (60 mL/min) to give the title compound as a colorless oil (11 g, 75%). ¹H NMR (400 MHz, CDCl₃) δ ppm 2.51-2.55 (m, 2H), 3.81 (s, 3H), 3.87-3.90 (t, J=5.6 Hz, 2H), 4.43-4.45 (t, J=2.8 Hz, 2H).

### Step 2: dimethyl 5,6-dihydro-2H-pyran-3,4-dicarboxylate

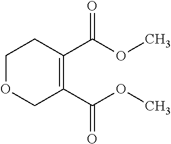

A mixture of methyl 4-(((trifluoromethyl)sulfonyl)oxy)-5,6-dihydro-2H-pyran-3-carboxylate (8 g, 27.57 mmol), Pd(dppf)Cl₂·CH₂Cl₂ (2.25 g, 2.76 mmol) and DIPEA (10.69 g, 82.70 mmol, 14.40 mL) in MeOH (120 mL) was stirred at 60° C. for 12 hours under CO (20 psi). LC-MS showed starting material was consumed completely and one main peak with desired m/z. The reaction mixture was filtered and the filtrate concentrated in vacuo. The resulting crude product was purified by flash chromatography (ISCO® SepaFlash® 80 g silica gel column) using a gradient of 0 to 15% EtOAc in petroleum ether (60 mL/min) to give the title compound (5 g, 91%). ¹H NMR (400 MHz, CDCl₃) δ ppm 2.44-2.47 (m, 2H), 3.78 (s, 3H), 3.79-3.82 (m, 5H), 4.34-4.35 (t, J=2.8 Hz, 2H); ESI-MS m/z [M+H]⁺ 201.1.

### Step 3: 5,6-dihydro-2H-pyran-3,4-dicarboxylic Acid

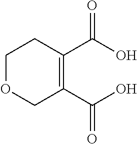 [SMILES: O=C(O)C1=C(C(=O)O)COCC1]

To a mixture of dimethyl 5,6-dihydro-2H-pyran-3,4-dicarboxylate (6 g, 29.97 mmol), NaOH (7.19 g, 179.83 mmol) in H₂O (60 mL) and MeOH (60 mL) was stirred at 50° C. for 1 hour. TLC indicated the starting material was consumed completely. The mixture was concentrated in vacuo to remove MeOH. The aqueous layer was extracted with DCM (30 mL×2) and the organic layer was discarded. To the aqueous layer was added HCl (3 M, 60 mL) and the mixture was evaporated in vacuo to dryness. The residue was triturated with DCM/MeOH (10:1, 500 mL×2) for 30 minutes and filtered. The filtrate was dried over Na₂SO₄, filtered and concentrated in vacuo. The residue was diluted with DCM/MeOH (100 mL), dried over Na₂SO₄, filtered and concentrated in vacuo to give the title compound as a yellow oil (4.7 g, crude). ¹H NMR (400 MHz, CDCl₃) δ ppm 2.51 (s, 2H), 3.79-3.85 (m, 2H), 4.40 (s, 2H), 8.76 (s, 2H).

### Step 4: 6,7-dihydro-3H-furo[3,4-c]pyran-1,3(4H)-dione

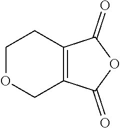 [SMILES: O=C1OC(=O)C2=C1CCOC2]

A solution of 5,6-dihydro-2H-pyran-3,4-dicarboxylic acid (4.7 g, 27.30 mmol) in Ac₂0 (109.00 g, 1.07 mol, 100 mL) was stirred at 100° C. for 4 hours. TLC showed one major new spot with lower polarity. The mixture was concentrated in vacuo to give the title compound as a yellow oil (4.8 g, crude).

### Step 5: 7,8-dihydro-5H-pyrano[3,4-d]pyridazine-1,4-diol

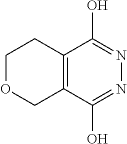 [SMILES: Oc1nnc(O)c2c1CCOC2]

A mixture of 6,7-dihydro-3H-furo[3,4-c]pyran-1,3(4H)-dione (4.8 g, 31.14 mmol) and NH₂NH₂.H₂O (6.54 g, 128.00 mmol, 6.35 mL, 98% purity) in THE (100 mL) was stirred at 70° C. for 3 hours. A white suspension was observed. The mixture was concentrated in vacuo and the resulting residue was triturated with EtOH (100 mL) for 30 minutes and filtered. The filter cake was dried in vacuo to give the title compound as a white solid (2.8 g, 53%). ¹H NMR (400 MHz, DMSO-d₆) δ ppm 2.37-2.39 (t, J=5.6 Hz, 2H), 3.77-3.79 (t, J=5.6 Hz, 2H), 4.32 (s, 2H).

### Step 6: 1,4-dichloro-7,8-dihydro-5H-pyrano[3,4-d]pyridazine

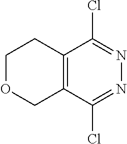 [SMILES: Clc1nnc(Cl)c2c1CCOC2]

A mixture of 7,8-dihydro-5H-pyrano[3,4-d]pyridazine-1,4-diol (2.7 g, 16.06 mmol), POCl₃ (89.10 g, 581.10 mmol, 54.00 mL) and DIPEA (10.38 g, 80.29 mmol, 13.98 mL) was stirred at 100° C. for 2 hours. TLC showed one major new spot with lower polarity. The mixture was concentrated in vacuo and the resulting residue was diluted with DCM (50 mL). The mixture was adjusted to pH 8 with DIPEA at 0° C., added to saturated aq NaHCO₃ (50 mL) drop-wise at 0° C. and extracted with DCM/MeOH (10:1, 80 mL×2). The combined organic layers were dried over Na₂SO₄, filtered and concentrated in vacuo. The residue was purified by flash chromatography (ISCO® SepaFlash® 80 g silica gel column) using a gradient of 0 to 50% EtOAc in petroleum ether (60 mL/min). The title compound was obtained as a white solid (1.7 g, 52%). ¹H NMR (400 MHz, DMSO-d₆) δ ppm 2.76-7.78 (t, J=5.6 Hz, 2H), 3.93-3.96 (t, J=5.6 Hz, 2H), 4.66 (s, 2H).

### Preparation 50: (R)-4-chloro-N-(1-methylpiperidin-3-yl)-7,8-dihydro-5H-pyrano[3,4-d]pyridazin-1-amine and (R)-1-chloro-N-(1-methylpiperidin-3-yl)-7,8-dihydro-5H-pyrano[3,4-d]pyridazin-4-amine

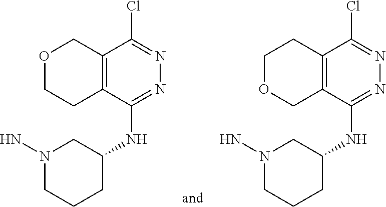 [SMILES: NN1CCC[C@@H](Nc2nnc(Cl)c3c2CCOC3)C1 ; NN1CCC[C@@H](Nc2nnc(Cl)c3c2COCC3)C1]

A vial was charged with 1,4-dichloro-7,8-dihydro-5H-pyrano[3,4-d]pyridazine (500 mg, 2.44 mmol), (R)-1-methylpiperidin-3-amine (556.90 mg, 4.88 mmol) and DIPEA (1.58 g, 12.19 mmol, 2.12 mL) in NMP (5 mL). The vial was sealed and the reaction mixture was heated at 170° C. in a microwave reactor for 1 hour. LC-MS showed a peak with desired m/z. The reaction mixture was purified by preparative HPLC (Xtimate C18-10 μm, 40 mm×150 mm column) using a gradient of 15 to 45% ACN in water (with 0.05% NH₃H₂O) to give a mixture of the title compounds as a yellow gum (400 mg). ESI-MS m/z [M+H]⁺ 283.1.

### Preparation 51: 4-chloro-1-(4-methoxyphenyl)-7,8-dihydro-5H-pyrano[3,4-d]pyridazine and 1-chloro-4-(4-methoxyphenyl)-7,8-dihydro-5H-pyrano[3,4-d]pyridazine

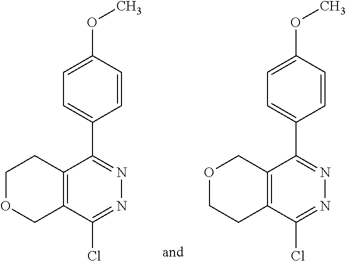 [SMILES: COc1ccc(-c2nnc(Cl)c3c2CCOC3)cc1 ; COc1ccc(-c2nnc(Cl)c3c2COCC3)cc1]

A mixture of 1,4-dichloro-7,8-dihydro-5H-pyrano[3,4-d]pyridazine (100 mg, 487.71 μmol), (4-methoxyphenyl)boronic acid (88.93 mg, 585.25 μmol), Pd(dppf)Cl₂·CH₂Cl₂ (79.66 mg, 97.54 μmol) and Na₂CO₃ (155.07 mg, 1.46 mmol) in dioxane (2 mL) and H₂O (0.5 mL) was degassed and purged with N₂ (3×) and then stirred at 100° C. for 2 hours under N₂ atmosphere. LC-MS showed the desired product was formed. The reaction mixture was concentrated in vacuo to give the crude product, which was purified by flash chromatography (ISCO® SepaFlash® 4 g silica gel column) using a gradient of 0 to 25% EtOAc in petroleum ether (18 m/min). A mixture of the title compounds was obtained as a white solid (60 mg). ¹H NMR (400 MHz, DMSO-d₆) δ 2.75-2.90 (m, 4H), 3.83 (s, 8H), 4.57-4.76 (m, 4H), 6.90-7.22 (m, 4H), 7.37-7.84 (m, 4H).

### Preparation 52: (R)-4-chloro-N-(1-methylpiperidin-3-yl)-7,8-dihydro-5H-pyrano[3,4-d]pyridazin-1-amine

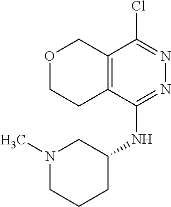 [SMILES: CN1CCC[C@@H](Nc2nnc(Cl)c3c2CCOC3)C1]

### Preparation 53: (R)-1-chloro-N-(1-methylpiperidin-3-yl)-7,8-dihydro-5H-pyrano[3,4-d]pyridazin-4-amine

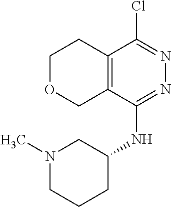 [SMILES: CN1CCC[C@@H](Nc2nnc(Cl)c3c2COCC3)C1]

To a solution of 1,4-dichloro-7,8-dihydro-5H-pyrano[3,4-d]pyridazine (200 mg, 975.41 μmol) in NMP (3 mL) were added DIPEA (630.33 mg, 4.88 mmol, 849.50 μL) and (R)-1-methylpiperidin-3-amine (365.02 mg, 1.95 mmol, 2HCl). The mixture was stirred at 170° C. for 1 hour in a microwave reactor and then purified by preparative HPLC (Phenomenex C18-3 μm, 30 mm×75 mm column) using a gradient of 13 to 43% ACN in water (10 mM NH₄HCO₃) to give a mixture of the title compounds as a white solid (70 mg, crude). The title compounds were separated by chiral SFC (DAICEL CHIRALPAK® AD-10 μm, 30 mm×250 mm column) using a mobile phase of CO₂ and 35% EtOH (with 0.1% NH₃H₂O). The title compound of Preparation 52 was obtained as a white solid (32 mg, 12%). ¹H NMR (400 MHz, DMSO-d₆) δ ppm 1.23 (s, 1H), 1.42 (br dd, J 6.19, 2.56 Hz, 1H), 1.60 (br d, J=11.51 Hz, 1H), 1.71-1.93 (m, 1H), 2.28-2.41 (m, 2H) 2.55-2.73 (m, 1H), 2.86 (br d, J=2.88 Hz, 1H), 3.10 (br s, 1H), 3.87 (t, J=5.63 Hz, 1H), 4.14-4.31 (m, 1H), 4.43 (br s, 1H), 5.98 (br d, J=1.75 Hz, 1H); ESI-MS m/z [M+H]⁺ 283.1. The title compound of Preparation 53 was obtained as a white solid (28 mg, 10%). ¹H NMR (400 MHz, DMSO-d₆) δ ppm 1.41 (br d, J=9.63 Hz, 1H), 1.48-1.61 (m, 1H), 1.65-1.77 (m, 1H), 1.78-1.90 (m, 1H), 2.06 (br d, J=7.13 Hz, 2H) 2.20-2.37 (m, 4H), 2.62-2.76 (m, 1H), 2.97 (br d, J=8.13 Hz, 1H), 3.90 (t, J=5.50 Hz, 2H), 4.13-4.25 (m, 1H), 4.50 (s, 2H), 5.00 (s, 1H), 6.06 (br d, J=7.25 Hz, 1H), 7.35 (s, 1H); ESI-MS m/z [M+H]⁺ 283.1.

### Preparation 54: 4-chloro-N-((3R,5R)-5-fluoro-1-methylpiperidin-3-yl)-7,8-dihydro-5H-pyrano[3,4-d]pyridazin-1-amine

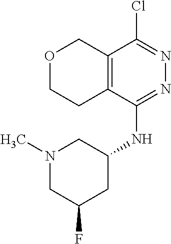 [SMILES: CN1C[C@H](F)C[C@@H](Nc2nnc(Cl)c3c2CCOC3)C1]

### Preparation 55: 1-chloro-N-((3R,5R)-5-fluoro-1-methylpiperidin-3-yl)-7,8-dihydro-5H-pyrano[3,4-d]pyridazin-4-amine

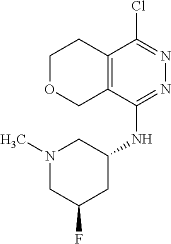 [SMILES: CN1C[C@H](F)C[C@@H](Nc2nnc(Cl)c3c2COCC3)C1]

Step 1: Tert-Butyl (3R,5R)-3-((4-chloro-7,8-dihydro-5H-pyrano[3,4-d]pyridazin-1-yl)amino)-5-fluoropiperidine-1-carboxylate and tert-butyl (3R,5R)-3-((1-chloro-7,8-dihydro-5H-pyrano[3,4-d]pyridazin-4-yl)amino)-5-fluoropiperidine-1-carboxylate

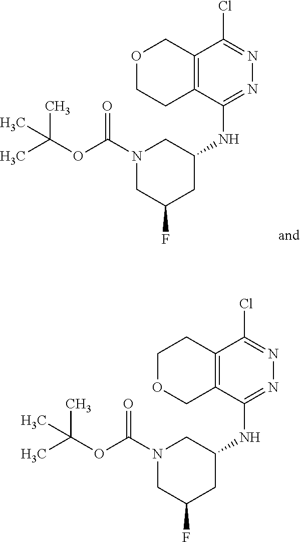 [SMILES: CC(C)(C)OC(=O)N1C[C@H](F)C[C@@H](Nc2nnc(Cl)c3c2CCOC3)C1 ; CC(C)(C)OC(=O)N1C[C@H](F)C[C@@H](Nc2nnc(Cl)c3c2COCC3)C1]

A mixture 1,4-dichloro-7,8-dihydro-5H-pyrano[3,4-d]pyridazine (3 g, 14.63 mmol), tert-butyl (3R,5R)-3-amino-5-fluoropiperidine-1-carboxylate (3.83 g, 17.56 mmol), BINAP (1.82 g, 2.93 mmol), Cs₂CO₃ (9.53 g, 29.26 mmol) and Pd₂(dba)₃ (1.34 g, 1.46 mmol) in toluene (20 mL) was stirred at 100° C. for 12 hours under N₂. LC-MS showed one main peak with desired m/z. The mixture was filtered and the filter cake was washed with DCM/MeOH (10:1, 60 mL). The filtrate was concentrated in vacuo and the residue was purified by flash chromatography (ISCO® SepaFlash® 40 g silica gel column) using a gradient of 0 to 10% MeOH in DCM (24 mL/min) to give a mixture of the title compounds as a yellow solid (2.9 g, crude). ESI-MS m/z [M+H]⁺ 387.0.

Step 2: 4-chloro-N-((3R,5R)-5-fluoro-1-methylpiperidin-3-yl)-7,8-dihydro-5H-pyrano[3,4-d]pyridazin-1-amine and 1-chloro-N-((3R,5R)-5-fluoro-1-methylpiperidin-3-yl)-7,8-dihydro-5H-pyrano[3,4-d]pyridazin-4-amine

A mixture of tert-butyl (3R,5R)-3-((4-chloro-7,8-dihydro-5H-pyrano[3,4-d]pyridazin-1-yl)amino)-5-fluoropiperidine-1-carboxylate and tert-butyl (3R,5R)-3-((1-chloro-7,8-dihydro-5H-pyrano[3,4-d]pyridazin-4-yl)amino)-5-fluoropiperidine-1-carboxylate (2.8 g) and formaldehyde (434.71 mg, 14.48 mmol, 398.82 μL) in formic acid (50 mL) was stirred at 80° C. for 1.5 hours. LC-MS showed one main peak with desired m/z. The mixture was concentrated in vacuo and the resulting residue was diluted with H₂O (30 mL) and extracted with DCM (10 mL×2). The organic layer was discarded. The aqueous layer was adjusted to pH 8-9 with NaHCO₃ and extracted with DCM/MeOH (10:1, 50 mL×3). The combined organic layers were concentrated in vacuo to give a crude product. The title compounds were separated by chiral SFC (DAICEL CHIRALPAK® AD-10 μm, 30 mm×250 mm column) using a mobile phase of CO₂ and 50% EtOH (with 0.1% NH₃H₂O). The title compound of Preparation 54 was obtained as a yellow solid (380 mg, 44.4%). ¹H NMR (400 MHz, DMSO-d₆) δ ppm 1.62-1.82 (m, 1H), 1.91 (br t, J=10.0 Hz, 1H), 2.16 (br s, 2H), 2.20 (s, 3H), 2.43 (br t, J=5.4 Hz, 2H), 2.80-3.00 (m, 2H), 3.90 (t, J=5.6 Hz, 2H), 4.37-4.57 (m, 3H), 4.84-5.05 (m, 1H), 6.08 (br d, J=7.9 Hz, 1H); ESI-MS m/z [M+H]⁺ 301.0. The title compound of Preparation 55 was obtained as a yellow solid (310 mg, 36.3%). ¹H NMR (400 MHz, DMSO-d₆) δ ppm 1.55-1.77 (m, 1H), 1.87 (t, J=10.1 Hz, 1H), 2.00-2.18 (m, 2H), 2.20 (s, 3H), 2.60 (br t, J=5.4 Hz, 2H), 2.83-3.01 (m, 2H), 3.88 (t, J=5.6 Hz, 2H), 4.37-4.52 (m, 3H), 4.76-5.09 (m, 1H), 5.90 (d, J=7.8 Hz, 1H); ESI-MS m/z [M+H]⁺ 301.0.

### Preparation 56: 4-chloro-N-((3R,5R)-5-fluoro-1-methylpiperidin-3-yl)-7,8-dihydro-5H-pyrano[3,4-d]pyridazin-1-amine (Alternative Synthesis)

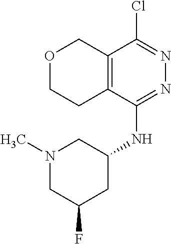 [SMILES: CN1C[C@H](F)C[C@@H](Nc2nnc(Cl)c3c2CCOC3)C1]

### Preparation 57: 1-chloro-N-((3R,5R)-5-fluoro-1-methylpiperidin-3-yl)-7,8-dihydro-5H-pyrano[3,4-d]pyridazin-4-amine (Alternative Synthesis)

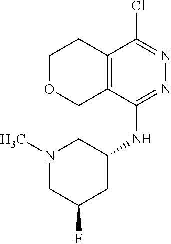 [SMILES: CN1C[C@H](F)C[C@@H](Nc2nnc(Cl)c3c2COCC3)C1]

To a solution of 1,4-dichloro-7,8-dihydro-5H-pyrano[3,4-d]pyridazine (9 g, 43.89 mmol), (3R,5R)-5-fluoro-1-methylpiperidin-3-amine (10.80 g, 52.67 mmol, 2HCl) in toluene (270 mL) were added BINAP (8.20 g, 13.17 mmol), Pd(OAc)₂ (1.97 g, 8.78 mmol) and Cs₂CO₃ (71.51 g, 219.47 mmol). The mixture was stirred at 120° C. for 12 hours under N₂. LC-MS showed starting material was consumed and desired mass was obtained. The reaction mixture was evaporated to dryness and the resulting residue was purified by flash chromatography (ISCO® SepaFlash® 220 g silica gel column) using a gradient of 0 to 6% DCM in MeOH (100 mL/min) to give a mixture of the desired compounds as a yellow solid (7 g, purity 93%). The products were separated by SFC (DAICEL CHIRALPAK® AD-10 μm, 50 mm×250 mm column) using a mobile phase of CO₂ and 30% IPA (with 0.1% NH₃H₂O). The title compound of Preparation 56 was obtained as a yellow solid (2.8 g, 21% yield, 99% purity). ¹H NMR (400 MHz, DMSO-d₆) δ ppm 1.62-1.82 (m, 1H), 1.91 (br t, J=10.01 Hz, 1H), 2.03-2.24 (m, 5H), 2.40-2.47 (m, 2H), 2.79-2.98 (m, 2H), 3.90 (t, J=5.57 Hz, 2H), 4.36-4.55 (m, 3H), 4.78-5.07 (m, 1H), 6.00-6.14 (m, 1H); ESI-MS m/z [M+H]⁺ 301.1. The title compound of Preparation 57 was obtained as a yellow solid (3.2 g, 24% yield, 97% purity). ¹H NMR (400 MHz, DMSO-d₆) δ ppm 1.57-1.75 (m, 1H), 1.81-1.92 (m, 1H), 2.04-2.21 (m, 5H), 2.59 (br t, J=5.44 Hz, 2H), 2.81-2.97 (m, 2H), 3.82-3.91 (m, 2H), 4.37-4.50 (m, 3H), 4.83-5.02 (m, 1H), 5.89 (d, J=7.88 Hz, 1H); ESI-MS m/z [M+H]⁺ 301.1.

### Example 1: 3-((4-(4-methoxyphenyl)phthalazin-1-yl)amino)phenol

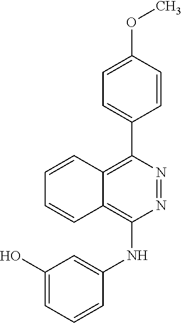 [SMILES: COc1ccc(-c2nnc(Nc3cccc(O)c3)c3ccccc23)cc1]

To a solution of 3-((4-chlorophthalazin-1-yl)amino)phenol (100 mg, 368.05 μmol) and (4-methoxyphenyl)boronic acid (67.11 mg, 441.66 μmol) in toluene (0.5 mL), EtOH (0.1 mL) and H₂O (0.1 mL) were added Pd(PPh₃)₄ (127.59 mg, 110.42 μmol) and Na₂CO₃ (117.03 mg, 1.10 mmol) at 25° C. The reaction mixture was stirred under N₂ atmosphere at 100° C. for 1 hour and then concentrated under reduced pressure and purified by preparative HPLC (Method B). The title compound was obtained as a yellow solid (28.3 mg, 21.5% yield, 96% purity). ¹H NMR (400 MHz, CD₃Cl) δ ppm 3.88 (s, 3H), 6.43 (br d, J=8.1 Hz, 1H), 6.76 (d, J=8.6 Hz, 1H), 7.03 (d, J=8.6 Hz, 2H), 7.09 (t, J=8.1 Hz, 1H), 7.57 (d, J=8.7 Hz, 3H), 7.74-7.86 (m, 2H), 7.92 (d, J=7.5 Hz, 1H), 8.22 (d, J=7.7 Hz, 1H); ESI-MS m/z [M+H]⁺ 344.1.

### Example 2: 4-(6-(difluoromethyl)pyridin-3-yl)-N-(1-methylpiperidin-3-yl)phthalazin-1-amine

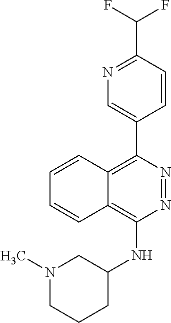 [SMILES: CN1CCCC(Nc2nnc(-c3ccc(C(F)F)nc3)c3ccccc23)C1]

A mixture of 4-chloro-N-(1-methylpiperidin-3-yl)phthalazin-1-amine (20 mg, 0.072 mmol), 2-(difluoromethyl)-5-(4,4,5,5-tetramethyl-1,3,2-dioxaborolan-2-yl)pyridine (28 mg, 0.11 mmol), aq K₂CO₃ (3M, 20 mg, 0.14 mmol) and AmPhos PdCl₂ (5 mg, 7 μmol) in ACN (0.8 mL) was heated at 80° C. for 18 hours and then filtered and purified by preparative HPLC (Method C). The title compound was obtained as a solid (15 mg, 52%). ¹H NMR (400 MHz, CD₃OD) δ ppm 1.64-1.85 (m, 2H), 1.87-1.96 (m, 1H), 2.04-2.13 (m, 1H), 2.29-2.44 (m, 5H), 2.69-2.80 (m, 1H), 3.11-3.22 (m, 1H), 4.58-4.68 (m, 1H), 6.71-7.02 (m, 1H), 7.83-7.89 (m, 1H), 7.89-8.01 (m, 3H), 8.26-8.32 (m, 1H), 8.37-8.44 (m, 1H), 8.89-8.97 (m, 1H). ESI-MS m/z [M+H]⁺ 370.2.

### Example 3: 3-((4-(4-(1-methylcyclopropyl)phenyl)phthalazin-1-yl)amino)phenol

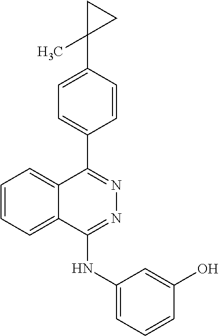 [SMILES: CC1(c2ccc(-c3nnc(Nc4cccc(O)c4)c4ccccc34)cc2)CC1]

The title compound was prepared like Example 2, using 3-((4-chlorophthalazin-1-yl)amino)phenol and 4,4,5,5-tetramethyl-2-(4-(1-methylcyclopropyl) phenyl)-1,3,2-dioxaborolane, and was obtained as an orange solid (2.9 mg, 13.4%). ESI-MS m/z [M+H]⁺ 368.2.

### Example 4: 3-((4-(4-(1-(trifluoromethyl)cyclopropyl)phenyl)phthalazin-1-yl)amino)phenol

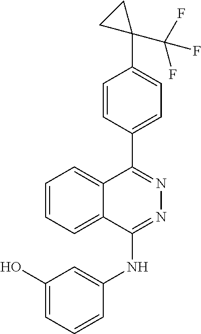 [SMILES: Oc1cccc(Nc2nnc(-c3ccc(C4(C(F)(F)F)CC4)cc3)c3ccccc23)c1]

The title compound was prepared like Example 2, using 3-((4-chlorophthalazin-1-yl)amino)phenol and 4,4,5,5-tetramethyl-2-(4-(1-(trifluoromethyl) cyclopropyl)phenyl)-1,3,2-dioxaborolane, and was obtained as a yellow solid (6 mg, 24.2%). ¹H NMR (400 MHz, CD₃OD) δ ppm 1.16-1.28 (m, 2H), 1.43-1.54 (m, 2H), 6.93-7.02 (m, 1H), 7.06-7.11 (m, 2H), 7.44 (t, J=7.67 Hz, 1H), 7.70-7.79 (m, 4H), 8.12-8.26 (m, 3H), 8.79 (d, J=7.28 Hz, 1H). ESI-MS m/z [M+H]⁺ 422.2.

### Example 5: 3-((4-(4-(tert-butyl)phenyl)phthalazin-1-yl)amino)phenol

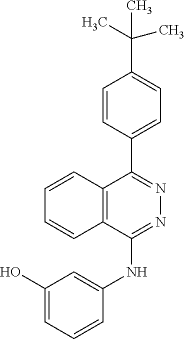 [SMILES: CC(C)(C)c1ccc(-c2nnc(Nc3cccc(O)c3)c3ccccc23)cc1]

The title compound was prepared like Example 2, using 3-((4-chlorophthalazin-1-yl)amino)phenol and 2-(4-tert-butylphenyl)-4,4,5,5-tetramethyl-1,3,2-dioxaborolane, and was obtained as an orange solid (1.7 mg, 7.8%). ¹H NMR (400 MHz, CD₃OD) δ ppm 1.44 (s, 9H), 6.93 (d, J=7.57 Hz, 1H), 7.08-7.13 (m, 2H), 7.42 (t, J=7.49 Hz, 1H), 7.64-7.74 (m, 4H), 8.17-8.28 (m, 3H), 8.78 (d, J=7.61 Hz, 1H). ESI-MS m/z [M+H]⁺ 370.2.

### Example 6: 3-((4-(3-cyclobutylphenyl)phthalazin-1-yl)amino)phenol

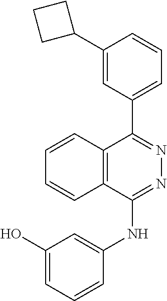 [SMILES: Oc1cccc(Nc2nnc(-c3cccc(C4CCC4)c3)c3ccccc23)c1]

The title compound was prepared like Example 2, using 3-((4-chlorophthalazin-1-yl)amino)phenol and 2-(3-cyclobutylphenyl)-4,4,5,5-tetramethyl-1,3,2-dioxaborolane, and was obtained as a light-yellow solid (2.5 mg, 11.5%). ESI-MS m/z [M+H]⁺ 368.2.

### Example 7: 3-((4-(4-isopropylphenyl)phthalazin-1-yl)amino)phenol

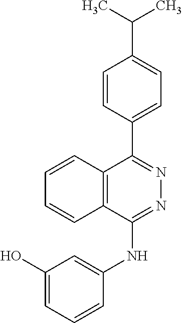 [SMILES: CC(C)c1ccc(-c2nnc(Nc3cccc(O)c3)c3ccccc23)cc1]

The title compound was prepared like Example 2, using 3-((4-chlorophthalazin-1-yl)amino)phenol and 2-(4-isopropylphenyl)-4,4,5,5-tetramethyl-1,3,2-dioxaborolane, and was obtained as a light-yellow solid (2.1 mg, 10.0%). ESI-MS m/z [M+H]⁺ 356.2.

### Example 8: 3-((4-(4-cyclopropyl-2-fluorophenyl)phthalazin-1-yl)amino)phenol

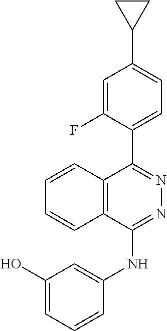 [SMILES: Oc1cccc(Nc2nnc(-c3ccc(C4CC4)cc3F)c3ccccc23)c1]

The title compound was prepared like Example 2, using 3-((4-chlorophthalazin-1-yl)amino)phenol and 2-(4-cyclopropyl-2-fluoro-phenyl)-4,4,5,5-tetramethyl-1,3,2-dioxaborolane, and was obtained as an orange solid (1.0 mg, 4.6%). ESI-MS m/z [M+H]⁺ 372.15.

### Example 9: 3-((4-(6-(tert-butyl)pyridin-3-yl)phthalazin-1-yl)amino)phenol

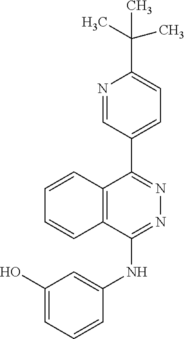 [SMILES: CC(C)(C)c1ccc(-c2nnc(Nc3cccc(O)c3)c3ccccc23)cn1]

The title compound was prepared like Example 2, using 3-((4-chlorophthalazin-1-yl)amino)phenol and 2-tert-butyl-5-(4,4,5,5-tetramethyl-1,3,2-dioxaborolan-2-yl)pyridine, and was obtained as a yellow solid (3.0 mg, 13.8%). ESI-MS m/z [M+H]⁺ 371.2.

### Example 10: 3-((4-(3-cyclopropylphenyl)phthalazin-1-yl)amino)phenol

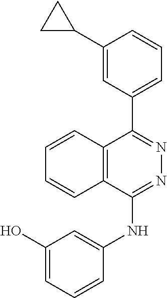 [SMILES: Oc1cccc(Nc2nnc(-c3cccc(C4CC4)c3)c3ccccc23)c1]

The title compound was prepared like Example 2, using 3-((4-chlorophthalazin-1-yl)amino)phenol and 2-(3-cyclopropylphenyl)-4,4,5,5-tetramethyl-1,3,2-dioxaborolane, and was obtained as an orange solid (2.1 mg, 10.1%). ESI-MS m/z [M+H]⁺ 354.2.

### Example 11: 4-(4-fluorophenyl)-N-(1-methylpiperidin-3-yl)phthalazin-1-amine

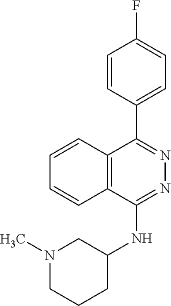 [SMILES: CN1CCCC(Nc2nnc(-c3ccc(F)cc3)c3ccccc23)C1]

The title compound was prepared like Example 2, using 4-chloro-N-(1-methylpiperidin-3-yl)phthalazin-1-amine and 2-(4-fluorophenyl)-4,4,5,5-tetramethyl-1,3,2-dioxaborolane, and was obtained as a yellow solid (25.7 mg, 98%). ESI-MS m/z [M+H]⁺ 337.2.

### Example 12: N-(1-methylpiperidin-3-yl)-4-(o-tolyl)phthalazin-1-amine

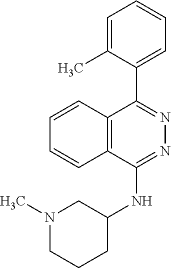 [SMILES: Cc1ccccc1-c1nnc(NC2CCCN(C)C2)c2ccccc12]

The title compound was prepared like Example 2, using 4-chloro-N-(1-methylpiperidin-3-yl)phthalazin-1-amine and o-tolylboronic acid, and was obtained as an orange solid (8.7 mg, 38.9%). ESI-MS m/z [M+H]⁺ 333.2.

### Example 13: N-(1-methylpiperidin-3-yl)-4-(4-(trifluoromethyl)phenyl)phthalazin-1-amine

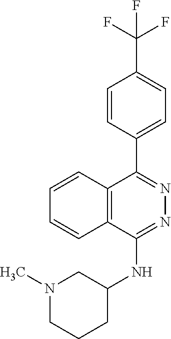 [SMILES: CN1CCCC(Nc2nnc(-c3ccc(C(F)(F)F)cc3)c3ccccc23)C1]

The title compound was prepared like Example 2, using 4-chloro-N-(1-methylpiperidin-3-yl)phthalazin-1-amine and (4-(trifluoromethyl)phenyl)boronic acid, and was obtained as a yellow solid (25.6 mg, 98.4%). ¹H NMR (400 MHz, CD₃OD) δ ppm 1.84-2.13 (m, 2H), 2.16-2.43 (m, 2H), 2.92-3.05 (m, 3H), 3.05-3.21 (m, 2H), 3.64 (br d, J=10.54 Hz, 1H), 3.86-4.02 (m, 1H), 4.57-4.75 (m, 1H), 7.95-8.00 (m, 2H), 8.00-8.05 (m, 2H), 8.08-8.13 (m, 1H), 8.19 (td, J=7.72, 1.13 Hz, 1H), 8.23-8.30 (m, 1H), 8.71 (br d, J=7.53 Hz, 1H); ESI-MS m/z [M+H]⁺ 387.2.

### Example 14: 4-(2-chlorophenyl)-N-(1-methylpiperidin-3-yl)phthalazin-1-amine

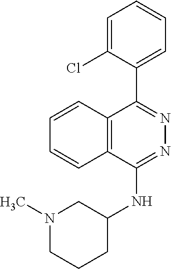 [SMILES: CN1CCCC(Nc2nnc(-c3ccccc3Cl)c3ccccc23)C1]

The title compound was prepared like Example 2, using 4-chloro-N-(1-methylpiperidin-3-yl)phthalazin-1-amine and (2-chlorophenyl)boronic acid, and was obtained as a light-yellow solid (4.5 mg, 19%). ESI-MS m/z [M+H]⁺ 353.15.

### Example 15: N-(1-methylpiperidin-3-yl)-4-(p-tolyl)phthalazin-1-amine

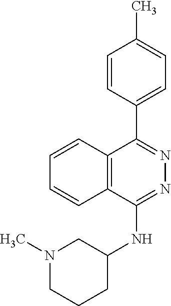 [SMILES: Cc1ccc(-c2nnc(NC3CCCN(C)C3)c3ccccc23)cc1]

The title compound was prepared like Example 2, using 4-chloro-N-(1-methylpiperidin-3-yl)phthalazin-1-amine and p-tolylboronic acid, and was obtained as a yellow solid (19 mg, 85%). ¹H NMR (400 MHz, CD₃OD) δ ppm 1.82-2.10 (m, 2H), 2.15-2.42 (m, 2H), 2.55 (s, 3H), 2.91-3.13 (m, 2H), 3.00 (s, 3H), 3.56-3.68 (m, 1H), 3.90-4.05 (m, 1H), 4.58-4.72 (m, 1H), 7.56-7.60 (m, 2H), 7.67 (d, J=8.16 Hz, 2H), 8.16-8.24 (m, 2H), 8.31 (br t, J=7.22 Hz, 1H), 8.65 (br d, J=8.03 Hz, 1H); ESI-MS m/z [M+H]f 333.2.

### Example 16: 4-(4-cyclopropylphenyl)-N-(1-methylpiperidin-3-yl)phthalazin-1-amine

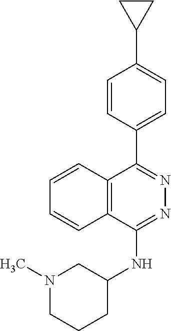 [SMILES: CN1CCCC(Nc2nnc(-c3ccc(C4CC4)cc3)c3ccccc23)C1]

The title compound was prepared like Example 2, using 4-chloro-N-(1-methylpiperidin-3-yl)phthalazin-1-amine and (4-cyclopropylphenyl)boronic acid, and was obtained as an orange solid (24.5 mg, 100%). ¹H NMR (400 MHz, CD₃OD) δ ppm 0.85-0.92 (m, 2H), 1.10-1.20 (m, 2H), 1.88 (td, J=12.30, 2.38 Hz, 1H), 1.95-2.08 (m, 1H), 2.08-2.16 (m, 1H), 2.17-2.26 (m, 1H), 2.32-2.40 (m, 1H), 2.87-3.14 (m, 2H), 3.00 (s, 3H), 3.62 (br d, J=11.17 Hz, 1H), 3.91-4.04 (m, 1H), 4.58-4.75 (m, 1H), 7.44 (m, J=8.28 Hz, 2H), 7.66 (m, J=8.16 Hz, 2H), 8.16-8.26 (m, 2H), 8.26-8.37 (m, 1H), 8.65 (br d, J=8.16 Hz, 1H); ESI-MS m/z [M+H]⁺ 359.2.

### Example 17: 4-(3-chlorophenyl)-N-(1-methylpiperidin-3-yl)phthalazin-1-amine

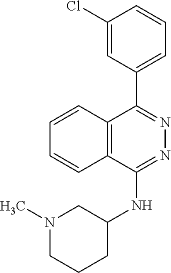 [SMILES: CN1CCCC(Nc2nnc(-c3cccc(Cl)c3)c3ccccc23)C1]

The title compound was prepared like Example 2, using 4-chloro-N-(1-methylpiperidin-3-yl)phthalazin-1-amine and (3-chlorophenyl)boronic acid, and was obtained as a light-yellow solid (1.0 mg, 4.2%). ESI-MS m/z [M+H]⁺ 353.2.

### Example 18: N-(1-methylpiperidin-3-yl)-4-(m-tolyl)phthalazin-1-amine

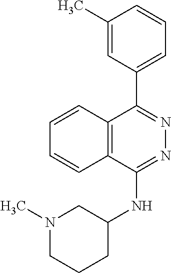 [SMILES: Cc1cccc(-c2nnc(NC3CCCN(C)C3)c3ccccc23)c1]

The title compound was prepared like Example 2, using 4-chloro-N-(1-methylpiperidin-3-yl)phthalazin-1-amine and m-tolylboronic acid, and was obtained as a white solid (8.0 mg, 36%). ESI-MS m/z [M+H]⁺ 333.2.

### Example 19: 3-((4-(4-cyclopropylphenyl)phthalazin-1-yl)amino)phenol

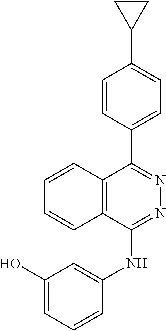 [SMILES: Oc1cccc(Nc2nnc(-c3ccc(C4CC4)cc3)c3ccccc23)c1]

The title compound was prepared like Example 2, using 3-((4-chlorophthalazin-1-yl)amino)phenol and (4-cyclopropylphenyl)boronic acid, and was obtained as an orange solid (3.4 mg, 9.7%). ¹H NMR (400 MHz, CD₃OD) δ ppm 0.78-0.86 (m, 2H), 1.07-1.13 (m, 2H), 2.02-2.09 (m, 1H), 6.90 (d, J=7.53 Hz, 1H), 7.09 (d, J=7.86 Hz, 1H), 7.08 (s, 1H), 7.31-7.42 (m, 3H), 7.58 (d, J=8.28 Hz, 2H), 8.13-8.25 (m, 3H), 8.76 (d, J=8.12 Hz, 1H); ESI-MS m/z [M+H]⁺ 354.2.

### Example 20: 4-(6-(tert-butyl)pyridin-3-yl)-N-(1-methylpiperidin-3-yl)phthalazin-1-amine

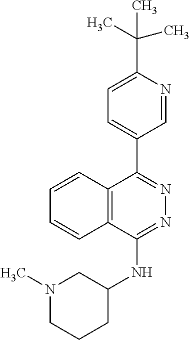 [SMILES: CN1CCCC(Nc2nnc(-c3ccc(C(C)(C)C)nc3)c3ccccc23)C1]

The title compound was prepared like Example 2, using 4-chloro-N-(1-methylpiperidin-3-yl)phthalazin-1-amine and 2-tert-butyl-5-(4,4,5,5-tetramethyl-1,3,2-dioxaborolan-2-yl)pyridine, and was obtained as a solid (28 mg, 100%). ¹H NMR (400 MHz, CD₃OD) δ ppm 1.36-1.40 (m, 9H), 1.55-1.75 (m, 2H), 1.76-1.85 (m, 1H), 1.93-2.03 (m, 1H), 2.14-2.28 (m, 2H), 2.15-2.29 (m, 5H), 2.57-2.67 (m, 1H), 2.97-3.09 (m, 1H), 4.48-4.57 (m, 1H), 7.58-7.62 (m, 1H), 7.74-7.82 (m, 2H), 7.81-7.89 (m, 1H), 7.92-7.97 (m, 1H), 8.26-8.31 (m, 1H), 8.63-8.68 (m, 1H).

### Example 21: 4-(4-(difluoromethyl)phenyl)-N-(1-methylpiperidin-3-yl)phthalazin-1-amine

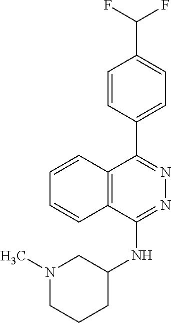 [SMILES: CN1CCCC(Nc2nnc(-c3ccc(C(F)F)cc3)c3ccccc23)C1]

The title compound was prepared like Example 2, using 4-chloro-N-(1-methylpiperidin-3-yl)phthalazin-1-amine and 2-(4-(difluoromethyl)phenyl)-4,4,5,5-tetramethyl-1,3,2-dioxaborolane. ESI-MS m/z [M+H]⁺ 369.2.

### Example 22: N-(1-methylpiperidin-3-yl)-4-(6-(trifluoromethyl)pyridin-3-yl)phthalazin-1-amine

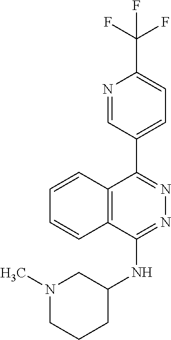 [SMILES: CN1CCCC(Nc2nnc(-c3ccc(C(F)(F)F)nc3)c3ccccc23)C1]

The title compound was prepared like Example 2, using 4-chloro-N-(1-methylpiperidin-3-yl)phthalazin-1-amine and 2-tert-butyl-5-(4,4,5,5-tetramethyl-1,3,2-dioxaborolan-2-yl)pyridine. ESI-MS m/z [M+H]⁺ 388.1.

### Example 23: (R)-4-(4-((1-methylpiperidin-3-yl)amino)pyrido[3,4-d]pyridazin-1-yl)phenol

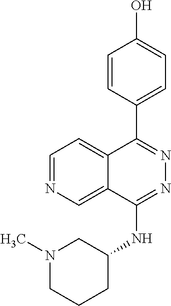 [SMILES: CN1CCC[C@@H](Nc2nnc(-c3ccc(O)cc3)c3ccncc23)C1]

The title compound was prepared like Example 2, using (R)-1-chloro-N-(1-methylpiperidin-3-yl)pyrido[3,4-d]pyridazin-4-amine (25 mg, 0.090 mmol) and (4-hydroxyphenyl)boronic acid (19 mg, 0.14 mmol), and was obtained as a solid (3.6 mg, 11%). ¹H NMR (400 MHz, CD₃OD) δ ppm 1.62-1.81 (m, 2H), 1.82-1.92 (m, 1H), 2.03-2.12 (m, 1H), 2.25-2.36 (m, 5H), 2.64-2.76 (m, 1H), 3.06-3.17 (m, 1H), 4.56-4.65 (m, 1H), 6.95-7.00 (m, 2H), 7.45-7.51 (m, 2H), 7.77-7.80 (m, 1H), 8.85-8.89 (m, 1H), 9.66-9.71 (m, 1H); ESI-MS m/z [M+H]⁺ 336.1.

### Example 24: (R)-5-(4-((1-methylpiperidin-3-yl)amino)pyrido[3,4-d]pyridazin-1-yl)-2-(trifluoromethoxy)benzonitrile

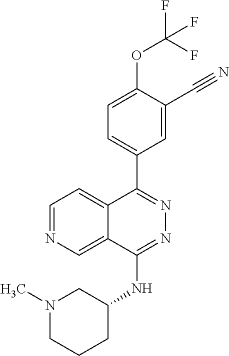 [SMILES: CN1CCC[C@@H](Nc2nnc(-c3ccc(OC(F)(F)F)c(C#N)c3)c3ccncc23)C1]

The title compound was prepared like Example 2, by using (R)-1-chloro-N-(1-methylpiperidin-3-yl)pyrido[3,4-d]pyridazin-4-amine (25 mg, 0.090 mmol) and 5-(4,4,5,5-tetramethyl-1,3,2-dioxaborolan-2-yl)-2-(trifluoromethoxy)benzonitrile (42 mg, 0.14 mmol), and was obtained as a solid (10 mg, 25%). ¹H NMR (400 MHz, CD₃OD), δ ppm 1.61-1.81 (m, 2H), 1.83-1.93 (m, 1H), 2.02-2.12 (m, 1H), 2.26-2.39 (m, 5H), 2.63-2.76 (m, 1H), 3.04-3.17 (m, 1H), 4.59-4.68 (m, 1H), 7.71-7.74 (m, 1H), 7.75-7.80 (m, 1H), 8.07-8.14 (m, 1H), 8.18-8.24 (m, 1H), 8.18-8.23 (m, 1H), 8.18-8.24 (m, 1H), 8.89-8.95 (m, 1H), 9.70-9.78 (m, 1H); ESI-MS m/z [M+H]⁺ 429.2.

### Example 25: (R)-1-(3-fluoro-4-methoxyphenyl)-N-(1-methylpiperidin-3-yl)pyrido[3,4-d]pyridazin-4-amine

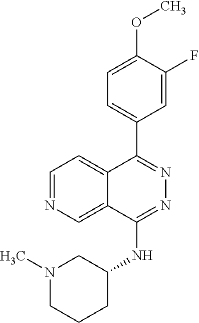 [SMILES: COc1ccc(-c2nnc(N[C@@H]3CCCN(C)C3)c3cnccc23)cc1F]

The title compound was prepared like Example 2, using (R)-1-chloro-N-(1-methylpiperidin-3-yl)pyrido[3,4-d]pyridazin-4-amine and (3-fluoro-4-methoxy-phenyl)boronic acid. ESI-MS m/z [M+H]⁺ 368.1.

### Example 26: (R)-1-(2,3-dihydrobenzo[b][1,4]dioxin-6-yl)-N-(1-methylpiperidin-3-yl)pyrido[3,4-d]pyridazin-4-amine

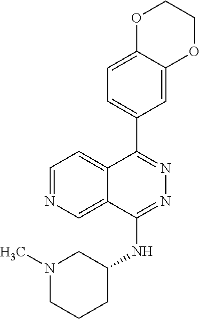 [SMILES: CN1CCC[C@@H](Nc2nnc(-c3ccc4c(c3)OCCO4)c3ccncc23)C1]

The title compound was prepared like Example 2, using (R)-1-chloro-N-(1-methylpiperidin-3-yl)pyrido[3,4-d]pyridazin-4-amine and 2,3-dihydro-1,4-benzodioxin-6-ylboronic acid. ESI-MS m/z [M+H]⁺ 378.1.

### Example 27: (R)-1-(4-(difluoromethoxy)phenyl)-N-(1-methylpiperidin-3-yl)pyrido[3,4-d]pyridazin-4-amine

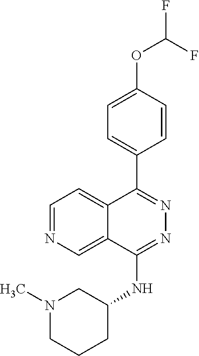 [SMILES: CN1CCC[C@@H](Nc2nnc(-c3ccc(OC(F)F)cc3)c3ccncc23)C1]

The title compound was prepared like Example 2, using (R)-1-chloro-N-(1-methylpiperidin-3-yl)pyrido[3,4-d]pyridazin-4-amine and (4-(difluoromethoxy)phenyl)boronic acid. ESI-MS m/z [M+H]⁺ 386.1.

### Example 28: (R)-2-(4-((1-methylpiperidin-3-yl)amino)pyrido[3,4-d]pyridazin-1-yl)phenol

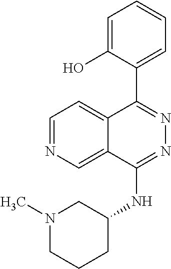 [SMILES: CN1CCC[C@@H](Nc2nnc(-c3ccccc3O)c3ccncc23)C1]

The title compound was prepared like Example 2, using (R)-1-chloro-N-(1-methylpiperidin-3-yl)pyrido[3,4-d]pyridazin-4-amine and (2-hydroxyphenyl)boronic acid as the reaction substrates. ESI-MS m/z [M+H]⁺ 336.1.

### Example 29: (R)-2-fluoro-4-(4-((1-methylpiperidin-3-yl)amino)pyrido[3,4-d]pyridazin-1-yl)phenol

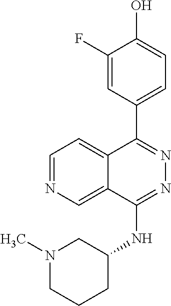 [SMILES: CN1CCC[C@@H](Nc2nnc(-c3ccc(O)c(F)c3)c3ccncc23)C1]

The title compound was prepared like Example 2, using (R)-1-chloro-N-(1-methylpiperidin-3-yl)pyrido[3,4-d]pyridazin-4-amine and (3-fluoro-4-hydroxy-phenyl)boronic acid. ESI-MS m/z [M+H]⁺ 354.2.

### Example 30: (R)—N-(1-methylpiperidin-3-yl)-1-(p-tolyl)pyrido[3,4-d]pyridazin-4-amine

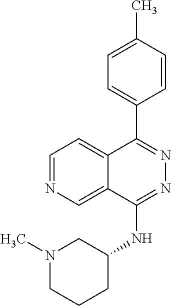 [SMILES: Cc1ccc(-c2nnc(N[C@@H]3CCCN(C)C3)c3cnccc23)cc1]

The title compound was prepared like Example 2, using (R)-1-chloro-N-(1-methylpiperidin-3-yl)pyrido[3,4-d]pyridazin-4-amine and p-tolylboronic acid. ESI-MS m/z [M+H]+ 334.2.

### Example 31: (R)-1-(4-chloro-2-methylphenyl)-N-(1-methylpiperidin-3-yl)pyrido[3,4-d]pyridazin-4-amine

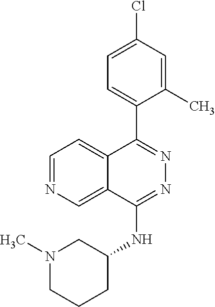

The title compound was prepared like Example 2, using (R)-1-chloro-N-(1-methylpiperidin-3-yl)pyrido[3,4-d]pyridazin-4-amine and (4-chloro-2-methyl-phenyl)boronic acid. ¹H NMR (400 MHz, CD₃OD) δ ppm 1.77-2.07 (m, 2H), 2.11-2.15 (m, 3H), 2.16-2.25 (m, 1H), 2.30-2.41 (m, 1H), 2.85-3.11 (m, 5H), 3.53-3.66 (m, 1H), 3.93-4.11 (m, 1H), 4.63-4.74 (m, 1H), 7.33-7.38 (m, 1H), 7.39-7.46 (m, 2H), 7.48-7.54 (m, 1H), 8.94-9.03 (m, 1H), 9.75-9.99 (m, 1H); ESI-MS m/z [M+H]+ 368.1.

### Example 32: (R)—N-(1-methylpiperidin-3-yl)-1-(4-(trifluoromethoxy)phenyl)pyrido[3,4-d]pyridazin-4-amine

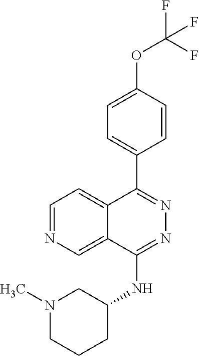 [SMILES: CN1CCC[C@@H](Nc2nnc(-c3ccc(OC(F)(F)F)cc3)c3ccncc23)C1]

The title compound was prepared like Example 2, using (R)-1-chloro-N-(1-methylpiperidin-3-yl)pyrido[3,4-d]pyridazin-4-amine and (4-(trifluoromethoxy)phenyl)boronic acid. ¹H NMR (400 MHz, CD₃OD) δ ppm 1.77-2.09 (m, 2H), 2.14-2.39 (m, 2H), 2.86-3.09 (m, 5H), 3.52-3.65 (m, 1H), 3.93-4.08 (m, 1H), 4.63-4.76 (m, 1H), 7.49-7.58 (m, 2H), 7.75-7.86 (m, 3H), 8.98-9.05 (m, 1H), 9.74-9.91 (m, 1H); ESI-MS m/z [M+H]⁺ 404.1.

### Example 33: (R)-1-(2-fluoro-4-(trifluoromethyl)phenyl)-N-(1-methylpiperidin-3-yl)pyrido[3,4-d]pyridazin-4-amine

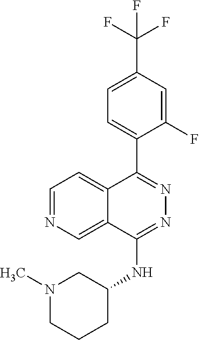 [SMILES: CN1CCC[C@@H](Nc2nnc(-c3ccc(C(F)(F)F)cc3F)c3ccncc23)C1]

The title compound was prepared like Example 2, using (R)-1-chloro-N-(1-methylpiperidin-3-yl)pyrido[3,4-d]pyridazin-4-amine and (2-fluoro-4-(trifluoromethyl)phenyl)boronic acid. ¹H NMR (400 MHz, CD₃OD) δ ppm 1.79-2.08 (m, 2H), 2.16-2.38 (m, 2H), 2.86-3.08 (m, 5H), 3.55-3.64 (m, 1H), 3.98-4.10 (m, 1H), 4.69-4.79 (m, 1H), 7.47-7.57 (m, 1H), 7.73-7.80 (m, 2H), 7.80-7.86 (m, 1H), 8.91-8.99 (m, 1H), 9.71-9.85 (m, 1H); ESI-MS m/z [M+H]⁺ 406.1.

### Example 34: (R)—N-(1-methylpiperidin-3-yl)-1-(4-(trifluoromethyl)phenyl)pyrido[3,4-d]pyridazin-4-amine

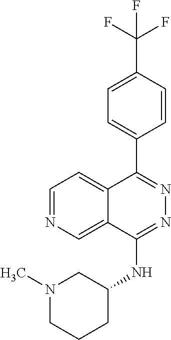 [SMILES: CN1CCC[C@@H](Nc2nnc(-c3ccc(C(F)(F)F)cc3)c3ccncc23)C1]

The title compound was prepared like Example 2, using (R)-1-chloro-N-(1-methylpiperidin-3-yl)pyrido[3,4-d]pyridazin-4-amine and (4-(trifluoromethyl)phenyl)boronic acid. ESI-MS m/z [M+H]⁺ 388.1.

### Example 35: (R)-1-(4-methoxyphenyl)-N-(1-methylpiperidin-3-yl)pyrido[3,4-d]pyridazin-4-amine

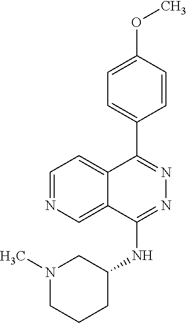

The title compound was prepared like Example 2, using (R)-1-chloro-N-(1-methylpiperidin-3-yl)pyrido[3,4-d]pyridazin-4-amine and (4-methoxyphenyl)boronic acid. ESI-MS m/z [M+H]+ 350.2.

### Example 36: (R)-5-methoxy-2-(4-((1-methylpiperidin-3-yl)amino)pyrido[3,4-d]pyridazin-1-yl)phenol

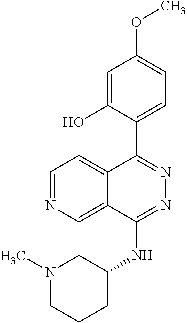

The title compound was prepared like Example 2, using (R)-1-chloro-N-(1-methylpiperidin-3-yl)pyrido[3,4-d]pyridazin-4-amine and (2-hydroxy-4-methoxyphenyl)boronic acid. ESI-MS m/z [M+H]⁺ 366.1.

### Example 37: (R)-1-(4-(difluoromethyl)phenyl)-N-(1-methylpiperidin-3-yl)pyrido[3,4-d]pyridazin-4-amine

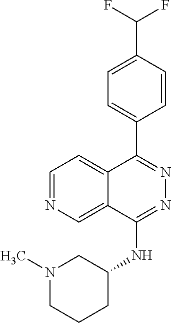 [SMILES: CN1CCC[C@@H](Nc2nnc(-c3ccc(C(F)F)cc3)c3ccncc23)C1]

The title compound was prepared like Example 2, using (R)-1-chloro-N-(1-methylpiperidin-3-yl)pyrido[3,4-d]pyridazin-4-amine and (4-(difluoromethyl)phenyl)boronic acid. ESI-MS m/z [M+H]⁺ 370.1.

### Example 38: 3-((4-(p-tolyl)phthalazin-1-yl)amino)phenol

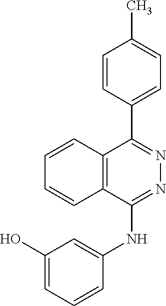 [SMILES: Cc1ccc(-c2nnc(Nc3cccc(O)c3)c3ccccc23)cc1]

To a solution of 3-((4-chlorophthalazin-1-yl)amino)phenol (400 mg, 1.47 mmol) and p-tolylboronic acid (300.23 mg, 2.21 mmol) in toluene (0.5 mL), EtOH (0.1 mL) and H₂O (0.1 mL) were added Pd(PPh₃)₄ (510.37 mg, 441.66 μmol) and Na₂CO₃ (468.11 mg, 4.42 mmol) at 25° C. The reaction mixture was stirred at 90° C. for 1 hour under N₂ atmosphere and then concentrated under reduced pressure. The resulting residue was diluted with saturated aq NaHCO₃ (30 mL), extracted with DCM (40 mL) and filtered through a pad of Celite®. The filtrate was concentrated under reduced pressure and purified by preparative HPLC (Method B) to give a formic acid salt of the title compound as a yellow solid (151.1 mg, 42.82% yield, 96.98% purity). ¹H NMR (400 MHz, DMSO-d₆) δ ppm 2.43 (s, 3H), 6.46 (br d, J=8.03 Hz, 1H), 7.13 (t, J=8.16 Hz, 1H), 7.26-7.42 (m, 3H), 7.51-7.66 (m, 3H), 7.85-8.04 (m, 3H), 8.14 (s, 1H), 8.65 (d, J=8.28 Hz, 1H), 8.94-9.64 (m, 2H); ESI-MS m/z [M+H]⁺ 328.1.

### Example 39: 2-((4-(4-methoxyphenyl)phthalazin-1-yl)amino)phenol

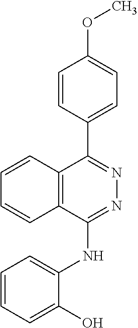 [SMILES: COc1ccc(-c2nnc(Nc3ccccc3O)c3ccccc23)cc1]

The title compound was prepared like Example 38, using 2-((4-chlorophthalazin-1-yl)amino)phenol (300 mg, 883.32 μmol, 80% purity) and (4-methoxyphenyl)boronic acid (201.34 mg, 1.32 mmol), and was obtained as a yellow solid (17.5 mg, 14.6%). ¹H NMR (400 MHz, CD₃Cl) 6 ppm 3.91 (s, 3H), 6.87-6.96 (m, 1H), 7.07 (d, J=8.6 Hz, 2H), 7.15 (d, J=3.7 Hz, 2H), 7.19 (d, J=7.7 Hz, 1H), 7.61 (d, J=8.6 Hz, 2H), 7.82-7.88 (m, 1H), 7.89-7.95 (m, 1H), 8.02 (br d, J=7.5 Hz, 1H), 8.17 (br s, 1H). ESI-MS m/z [M+H]⁺ 344.2.

### Example 40: 3-((1-(4-methoxyphenyl)pyrido[3,4-d]pyridazin-4-yl)amino)phenol

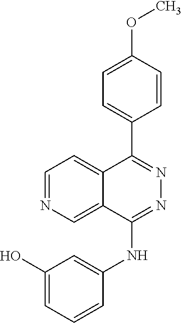 [SMILES: COc1ccc(-c2nnc(Nc3cccc(O)c3)c3cnccc23)cc1]

### Example 41: 3-((4-(4-methoxyphenyl)pyrido[3,4-d]pyridazin-1-yl)amino)phenol

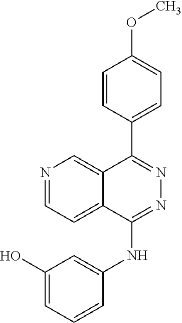 [SMILES: COc1ccc(-c2nnc(Nc3cccc(O)c3)c3ccncc23)cc1]

The title compounds were prepared like Example 38, using a mixture of 3-((1-chloropyrido[3,4-d]pyridazin-4-yl)amino)phenol and 3-((4-chloropyrido[3,4-d]pyridazin-1-yl)amino)phenol (53.3 mg, 195.46 μmol), and (4-methoxyphenyl)boronic acid (29.70 mg, 195.46 μmol). The title compound of Example 40 was obtained as a yellow solid (18 mg, 27%). ¹H NMR (400 MHz, DMSO-d₆) δ ppm 3.80 (s, 3H), 6.40-6.47 (m, 1H), 7.05-7.13 (m, 3H), 7.23-7.29 (m, 1H), 7.53 (t, J=2.13 Hz, 1H), 7.58-7.62 (m, 2H), 7.69 (dd, J=5.69, 0.69 Hz, 1H), 8.93 (d, J=5.63 Hz, 1H), 9.36 (s, 1H), 9.42-9.48 (m, 1H), 9.91-9.96 (m, 1H); ESI-MS m/z [M+H]⁺ 345.1. The title compound of Example 41 was obtained as a yellow solid (18 mg, 27%). ¹H NMR (400 MHz, DMSO-d₆) δ ppm 3.88 (s, 3H), 6.46-6.52 (m, 1H), 7.12-7.20 (m, 4H), 7.32-7.36 (m, 1H), 7.61-7.65 (m, 2H), 7.69-7.75 (m, 2H), 8.52-8.57 (m, 1H), 9.06-9.12 (m, 1H), 9.28 (s, 1H), 9.32 (s, 1H), 9.40-9.45 (m, 1H); ESI-MS m/z [M+H]⁺ 345.1.

### Example 42: N-(1-methylpiperidin-3-yl)-4-phenylphthalazin-1-amine

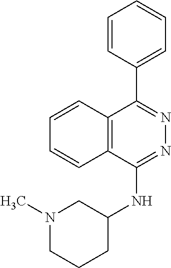 [SMILES: CN1CCCC(Nc2nnc(-c3ccccc3)c3ccccc23)C1]

A mixture of 4-chloro-N-(1-methylpiperidin-3-yl)phthalazin-1-amine (25 mg, 0.0903 mmol), 4,4,5,5-tetramethyl-2-phenyl-1,3,2-dioxaborolane (18 mg, 0.0903 mmol) and Pd(dppf)Cl₂ (6.4 mg, 0.00903 mmol) in 1,4-dioxane (0.4 mL) and 3M K₂CO₃ (0.15 mL, 0.452 mmol) was stirred at 60° C. for 22 hours using a heating block to furnish a yellow-orange solution. The reaction mixture was concentrated via rotary evaporation, dissolved in DMF (1 mL), filtered through a 0.45 μm nylon membrane filter (VWR), rinsed with DMF (0.5 mL) and purified by preparative HPLC (Method B). The pure fractions were combined and concentrated via rotary evaporation at 45° C. The resulting mixture was dried in vacuo to give the title compound as a white solid (16 mg, 56%). ¹H NMR (400 MHz, DMSO-d₆) δ ppm 1.47 (qd, J=11.71, 3.76 Hz, 1H), 1.54-1.69 (m, 1H), 1.86-2.05 (m, 3H), 2.23 (s, 3H), 2.68-2.79 (m, 1H), 3.10 (br d, J=8.28 Hz, 1H), 4.38-4.50 (m, 1H), 7.10 (br d, J=7.53 Hz, 1H), 7.47-7.58 (m, 3H), 7.58-7.63 (m, 2H), 7.75-7.80 (m, 1H), 7.84 (td, J=7.53, 1.25 Hz, 1H), 7.87-7.94 (m, 1H), 8.43 (d, J=8.03 Hz, 1H); ESI-MS m/z [M+H]⁺ 319.1.

### Example 43: 4-(4-chloro-2-fluorophenyl)-N-(1-methylpiperidin-3-yl)phthalazin-1-amine

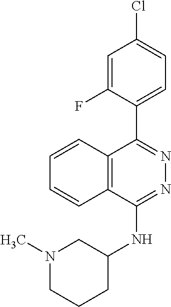 [SMILES: CN1CCCC(Nc2nnc(-c3ccc(Cl)cc3F)c3ccccc23)C1]

A mixture of 4-chloro-N-(1-methylpiperidin-3-yl)phthalazin-1-amine (100 mg, 309.80 μmol, formic acid salt), (4-chloro-2-fluorophenyl)boronic acid (81.03 mg, 464.70 μmol), Pd(dppf)Cl₂·CH₂Cl₂ (50.60 mg, 61.96 μmol) and Cs₂CO₃ (302.82 mg, 929.40 μmol) in dioxane (5 mL) and H₂O (1 mL) was stirred at 100° C. for 15 hours under N₂. The reaction mixture was filtered and the filtrate was purified by preparative HPLC (Method B). The title compound was obtained as a brown solid (49.1 mg, 37.3%). ¹H NMR (400 MHz, DMSO-d₆) δ ppm 1.43-1.70 (m, 2H), 1.74-1.84 (m, 1H), 1.94-2.12 (m, 3H), 2.29 (s, 3H), 2.79 (br d, J=11.1 Hz, 1H), 4.46 (br s, 1H), 7.31 (br d, J=6.4 Hz, 1H), 7.44-7.52 (m, 2H), 7.55-7.68 (m, 2H), 7.79-7.95 (m, 2H), 8.20 (s, 1H), 8.45 (d, J=8.1 Hz, 1H). ESI-MS m/z [M+H]+ 371.

### Example 44: 4-(4-chloro-3-fluorophenyl)-N-(1-methylpiperidin-3-yl)phthalazin-1-amine

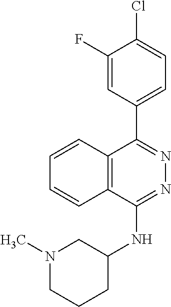 [SMILES: CN1CCCC(Nc2nnc(-c3ccc(Cl)c(F)c3)c3ccccc23)C1]

The title compound was prepared like Example 43, using 4-chloro-N-(1-methylpiperidin-3-yl)phthalazin-1-amine (100 mg, 309.80 μmol, formic acid salt) and (4-chloro-3-fluorophenyl)boronic acid (64.82 mg, 371.76 μmol), and was obtained as a yellow solid (10.6 mg, 9.13% yield, 99% purity). ¹H NMR (400 MHz, DMSO-d₆) δ ppm 1.37-1.50 (m, 1H), 1.52-1.66 (m, 1H), 1.69-1.78 (m, 1H), 1.88-2.01 (m, 3H), 2.21 (s, 3H), 2.72 (br d, J=11.5 Hz, 1H), 3.07 (br d, J=8.6 Hz, 1H), 4.41 (br s, 1H), 7.21 (br d, J=7.3 Hz, 1H), 7.46 (d, J=8.4 Hz, 1H), 7.65 (dd, J=10.1, 1.8 Hz, 1H), 7.70-7.80 (m, 2H), 7.83 (t, J=7.5 Hz, 1H), 7.86-7.94 (m, 1H), 8.17 (s, 1H), 8.42 (d, J=8.2 Hz, 1H); ESI-MS m/z [M+H]+ 371.3.

### Example 45: 4-(4-methoxyphenyl)-N-methyl-N-(1-methylpiperidin-3-yl)phthalazin-1-amine

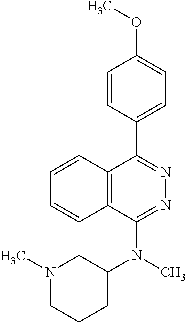

The title compound was prepared like Example 43, using 4-chloro-N-methyl-N-(1-methylpiperidin-3-yl)phthalazin-1-amine (14.7 mg, 0.051 mmol) and (4-methoxyphenyl)boronic acid (11.5 mg, 0.076 mmol), and was obtained as a light-yellow film (1.4 mg, 6.9% yield, ˜90% purity). ¹H NMR (400 MHz, CD₃OD) δ ppm 1.57-1.71 (m, 1H), 1.76-1.94 (m, 2H), 1.98-2.09 (m, 2H), 2.35 (s, 3H), 2.45 (t, J=10.9 Hz, 1H), 2.85 (br d, J=10.8 Hz, 1H), 3.11-3.23 (m, 4H), 3.92 (s, 3H), 3.97-4.09 (m, 1H), 7.11-7.21 (m, 2H), 7.58-7.68 (m, 2H), 7.85-7.93 (m, 1H), 7.94-8.05 (m, 2H), 8.21 (d, J=8.2 Hz, 1H); ESI-MS m/z [M+H]+ 363.20.

### Example 46: 4-(4-chloro-2-methylphenyl)-N-(1-methylpiperidin-3-yl)phthalazin-1-amine

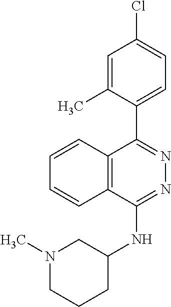 [SMILES: Cc1cc(Cl)ccc1-c1nnc(NC2CCCN(C)C2)c2ccccc12]

A mixture of 4-chloro-N-(1-methylpiperidin-3-yl)phthalazin-1-amine (0.083 g, 0.3 mmol), (4-chloro-2-methylphenyl)boronic acid (0.051 g, 0.300 mmol)), Pd(PPh₃)₄ (0.035 g, 0.030 mmol) and K₂CO₃ (0.124 g, 0.900 mmol) in toluene (1.042 mL) and water (0.208 mL) was degassed and purged with N₂ (3×). The mixture was stirred at 90° C. for 1 hour under N₂ atmosphere and then concentrated under reduced pressure. DMF (2 mL) was added. The mixture was filtered and the filtrate was purified by preparative HPLC. The title compound was obtained as an off-white solid (3.8 mg, 3.5%). ¹H NMR (400 MHz, CD₃OD) δ ppm 0.64-0.71 (m, 1H), 0.91-0.99 (m, 1H), 1.15-1.21 (m, 1H), 1.53-1.58 (m, 1H), 1.70-1.79 (m, 1H), 1.83-1.89 (m, 1H), 2.04-2.06 (m, 3H), 2.32-2.36 (m, 3H), 2.60-2.76 (m, 1H), 3.05-3.20 (m, 1H), 4.53-4.61 (m, 1H), 7.13-7.21 (m, 1H), 7.25-7.29 (m, 1H), 7.33-7.35 (m, 1H), 7.41-7.44 (m, 1H), 7.78-7.81 (m, 1H), 7.88 (s, 1H), 8.32 (d, J=8.28 Hz, 1H); ESI-MS m/z [M+H]⁺ 367.4.

### Example 47: 1-(4-chloro-2-methoxyphenyl)-N-(1-methylpiperidin-3-yl)pyrido[3,4-d]pyridazin-4-amine

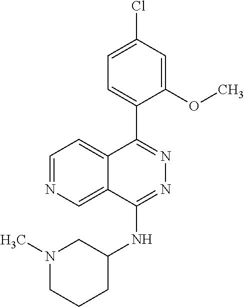

and

### Example 48: 4-(4-chloro-2-methoxyphenyl)-N-(1-methylpiperidin-3-yl)pyrido[3,4-d]pyridazin-1-amine

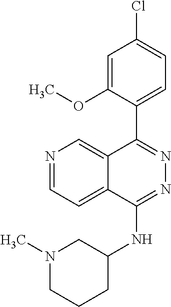 [SMILES: COc1cc(Cl)ccc1-c1nnc(NC2CCCN(C)C2)c2ccncc12]

The title compounds were prepared like Example 46, using a mixture of 1-chloro-N-(1-methylpiperidin-3-yl)pyrido[3,4-d]pyridazin-4-amine and 4-chloro-N-(1-methylpiperidin-3-yl)pyrido[3,4-d]pyridazin-1-amine (0.056 g, 0.2 mmol), and (4-chloro-2-methoxyphenyl)boronic acid (0.037 g, 0.200 mmol). The title compound of Example 47 was obtained as an off-white solid (11.8 mg, 15.4%). ¹H NMR (400 MHz, CD₃OD) δ ppm 1.74-1.90 (m, 1H), 1.91-2.01 (m, 1H), 2.09-2.21 (m, 1H), 2.25-2.36 (m, 1H), 2.79-2.97 (m, 4H), 2.98-3.06 (m, 1H), 3.50-3.61 (m, 1H), 3.73 (s, 3H), 3.84-4.03 (m, 1H), 4.54-4.66 (m, 1H), 7.20 (dd, J=8.09, 1.82 Hz, 1H), 7.30 (d, J=1.76 Hz, 1H), 7.42 (d, J=8.16 Hz, 1H), 7.56 (d, J=5.52 Hz, 1H), 9.02 (d, J=5.52 Hz, 1H), 9.84 (br s, 1H); ESI-MS m/z [M+H]+ 384.2. The title compound of Example 48 was obtained as an off-white solid (3.9 mg, 5.1%). ¹H NMR (400 MHz, CD₃OD) δ ppm 1.76-2.05 (m, 2H), 2.12-2.22 (m, 1H), 2.26-2.35 (m, 1H), 2.82-2.91 (m, 1H), 2.91-2.96 (m, 3H), 2.97-3.06 (m, 1H), 3.53-3.62 (m, 1H), 3.75-3.81 (m, 3H), 3.88-4.03 (m, 1H), 4.57-4.69 (m, 1H), 7.23-7.29 (m, 1H), 7.34-7.38 (m, 1H), 7.46-7.54 (m, 1H), 8.29-8.54 (m, 1H), 9.02-9.08 (m, 1H), 9.09-9.15 (m, 1H); ESI-MS m/z [M+H]+ 384.2.

### Example 49: 1-(4-chloro-2-methylphenyl)-N-(1-methylpiperidin-3-yl)pyrido[3,4-d]pyridazin-4-amine

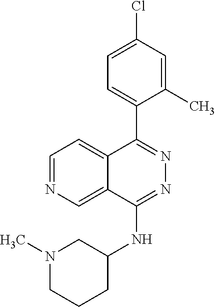

and

### Example 50: 4-(4-chloro-2-methylphenyl)-N-(1-methylpiperidin-3-yl)pyrido[3,4-d]pyridazin-1-amine

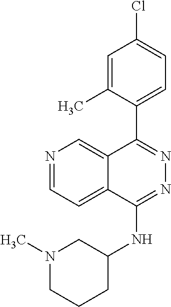 [SMILES: Cc1cc(Cl)ccc1-c1nnc(NC2CCCN(C)C2)c2ccncc12]

The title compounds were prepared like Example 46, using a mixture of 1-chloro-N-(1-methylpiperidin-3-yl)pyrido[3,4-d]pyridazin-4-amine and 4-chloro-N-(1-methylpiperidin-3-yl)pyrido[3,4-d]pyridazin-1-amine (0.056 g, 0.2 mmol), and (4-chloro-2-methylphenyl)boronic acid (0.034 g, 0.200 mmol). The title compound of Example 49 was obtained as an off-white solid (11.5 mg, 15.6%). ¹H NMR (400 MHz, CD₃OD) δ ppm 1.76-1.93 (m, 1H), 1.93-2.03 (m, 1H), 2.10-2.13 (m, 3H), 2.13-2.23 (m, 1H), 2.26-2.39 (m, 1H), 2.80-3.08 (m, 5H), 3.50-3.63 (m, 1H), 3.86-4.05 (m, 1H), 4.58-4.72 (m, 1H), 7.31-7.37 (m, 1H), 7.40-7.47 (m, 2H), 7.48-7.52 (m, 1H), 9.02 (d, J=5.52 Hz, 1H), 9.85 (br s, 1H); ESI-MS m/z [M+H]⁺ 368.2. The title compound of Example 50 was obtained as an off-white solid (2.7 mg, 3.7%). ¹H NMR (400 MHz, CD₃OD) δ ppm 1.78-2.05 (m, 2H), 2.13-2.16 (m, 3H), 2.16-2.23 (m, 1H), 2.27-2.36 (m, 1H), 2.83-2.91 (m, 1H), 2.92-2.97 (m, 3H), 2.97-3.07 (m, 1H), 3.54-3.62 (m, 1H), 3.93-4.04 (m, 1H), 4.59-4.71 (m, 1H), 7.39-7.43 (m, 1H), 7.44-7.49 (m, 1H), 7.52-7.57 (m, 1H), 8.28-8.54 (m, 1H), 8.88-8.95 (m, 1H), 9.08-9.14 (m, 1H); ESI-MS m/z [M+H]⁺ 368.2.

### Example 51: 1-(2,4-dichlorophenyl)-N-(1-methylpiperidin-3-yl)pyrido[3,4-d]pyridazin-4-amine

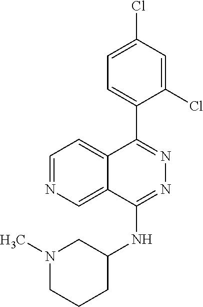 [SMILES: CN1CCCC(Nc2nnc(-c3ccc(Cl)cc3Cl)c3ccncc23)C1]

and

### Example 52: 4-(2,4-dichlorophenyl)-N-(1-methylpiperidin-3-yl)pyrido[3,4-d]pyridazin-1-amine

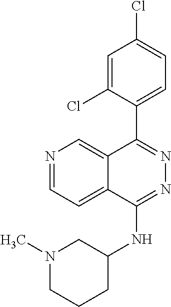 [SMILES: CN1CCCC(Nc2nnc(-c3ccc(Cl)cc3Cl)c3cnccc23)C1]

The title compounds were prepared like Example 46, using a mixture of 1-chloro-N-(1-methylpiperidin-3-yl)pyrido[3,4-d]pyridazin-4-amine and 4-chloro-N-(1-methylpiperidin-3-yl)pyrido[3,4-d]pyridazin-1-amine (0.056 g, 0.2 mmol), and (2,4-dichlorophenyl)boronic acid (0.038 g, 0.200 mmol). The title compound of Example 51 was obtained as an off-white solid (1.7 mg, 2.2%). ¹H NMR (400 MHz, CD₃OD) δ ppm 1.79-2.02 (m, 2H), 2.14-2.23 (m, 1H), 2.28-2.36 (m, 1H), 2.86-2.92 (m, 1H), 2.92-2.98 (m, 3H), 2.98-3.07 (m, 1H), 3.53-3.63 (m, 1H), 3.96-4.05 (m, 1H), 4.67-4.76 (m, 1H), 7.35-7.41 (m, 1H), 7.50-7.55 (m, 1H), 7.56-7.62 (m, 1H), 7.73-7.77 (m, 1H), 8.90-8.94 (m, 1H), 9.69-9.85 (m, 1H); ESI-MS m/z [M+H]⁺ 388.2. The title compound of Example 52 was obtained as an off-white solid (1 mg, 1%). ¹H NMR (400 MHz, CD₃OD) δ ppm 1.84-2.03 (m, 2H), 2.15-2.22 (m, 1H), 2.27-2.34 (m, 1H), 2.83-2.89 (m, 1H), 2.91-2.98 (m, 3H), 2.98-3.06 (m, 1H), 3.56-3.60 (m, 1H), 3.99-4.05 (m, 1H), 4.64-4.72 (m, 1H), 7.55-7.64 (m, 2H), 7.75-7.79 (m, 1H), 8.19-8.25 (m, 1H), 8.83-8.88 (m, 1H), 8.98-9.04 (m, 1H); ESI-MS m/z [M+H]⁺ 388.2.

### Example 53: (R)-4-(2-fluoro-4-methoxyphenyl)-N-(1-methylpiperidin-3-yl)phthalazin-1-amine

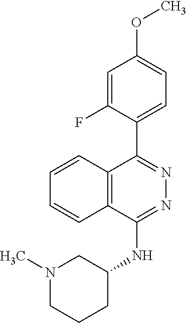

To a solution of (R)-4-chloro-N-(1-methylpiperidin-3-yl)phthalazin-1-amine (100 mg, 361 μmol), (2-fluoro-4-methoxyphenyl)boronic acid (79.83 mg, 469.71 μmol) and Cs₂CO₃ (235.45 mg, 722.64 μmol) in dioxane (2 mL) and H₂O (0.4 mL) was added Pd(dppf)Cl₂ (13.22 mg, 18.07 μmol, 0.05 eq). The mixture was stirred at 100° C. for 12 hours under N₂. LC-MS showed one main peak with desired m/z. The mixture was filtered and the filtrate was purified by preparative HPLC (Method B). The title compound was obtained as a yellow solid (52 mg, 34% yield, 96% purity). ¹H NMR (400 MHz, DMSO-d₆) δ ppm 1.41-1.69 (m, 2H), 1.73-1.83 (m, 1H), 1.94-2.08 (m, 3H), 2.27 (s, 3H), 2.77 (br d, J=11.1 Hz, 1H), 3.13 (br d, J=7.8 Hz, 1H), 3.87 (s, 3H), 4.44 (br s, 1H), 6.93-7.06 (m, 2H), 7.18 (br d, J=6.1 Hz, 1H), 7.40-7.52 (m, 2H), 7.78-7.93 (m, 2H), 8.19 (s, 1H), 8.42 (d, J=8.1 Hz, 1H); ESI-MS m/z [M+H]⁺ 367.

### Example 54: 4-(4-chlorophenyl)-N-((3R,5R)-1-cyclopropyl-5-fluoropiperidin-3-yl)phthalazin-1-amine

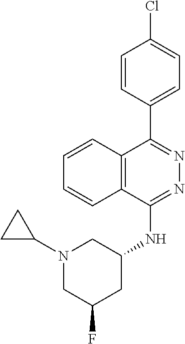 [SMILES: F[C@@H]1C[C@@H](Nc2nnc(-c3ccc(Cl)cc3)c3ccccc23)CN(C2CC2)C1]

In a 40 mL vial equipped with stir bar, nitrogen was bubbled through a solution of 4-chloro-N-((3R,5R)-1-cyclopropyl-5-fluoropiperidin-3-yl)phthalazin-1-amine (39.7 mg, 0.124 mmol) and 2-(4-chlorophenyl)-4,4,5,5-tetramethyl-1,3,2-dioxaborolane (36.9 mg, 0.155 mmol) in dioxane (1.24 mL) for 5 minutes. To the mixture was added aq K₂CO₃ (2 M, 186 μL, 0.371 mmol), followed by PdCl₂(dppf) (9.06 mg, 0.012 mmol). The reaction mixture was stirred under nitrogen atmosphere at 90° C. overnight using a heating block and then diluted with water and extracted with EtOAc (3×). The organic layers were combined, washed with brine, dried over Na₂SO₄, filtered and concentrated in vacuo. The residue was taken up in DMF (1 mL) and MeOH (1 mL) and was filtered through a hydrophilic PTFE 0.45 m syringe filter (VWR®). The filtrate was purified by preparative HPLC (Method A). The product-containing fractions were evaporated and dried in vacuo to give a TFA salt of the title compound as a yellow film (7.0 mg, 11%). ESI-MS m/z [M+H]⁺ 397.2.

### Example 55: N-((3R,5R)-1-cyclopropyl-5-fluoropiperidin-3-yl)-4-(4-methoxyphenyl)phthalazin-1-amine

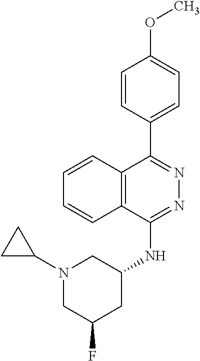 [SMILES: COc1ccc(-c2nnc(N[C@@H]3C[C@@H](F)CN(C4CC4)C3)c3ccccc23)cc1]

The title compound was prepared like Example 54, using 4-chloro-N-((3R,5R)-1-cyclopropyl-5-fluoropiperidin-3-yl)phthalazin-1-amine (81 mg, 0.252 mmol) and (4-methoxyphenyl)boronic acid (48.0 mg, 0.316 mmol), and was obtained as a white solid (2.8 mg, 2.8%). ¹H NMR (400 MHz, DMSO-d₆) δ ppm 0.26-0.54 (m, 4H), 1.65-1.95 (m, 2H), 2.28 (br t, J=10.2 Hz, 2H), 2.42 (br d, J=13.2 Hz, 1H), 3.01-3.18 (m, 1H), 3.28 (br d, J=4.3 Hz, 1H), 3.86 (s, 3H), 4.59-4.77 (m, 1H), 4.89-5.11 (m, 1H), 7.05-7.16 (m, 3H), 7.48-7.61 (m, 2H), 7.77-7.97 (m, 3H), 8.39 (d, J=7.8 Hz, 1H); ESI-MS m/z [M+H]⁺ 393.20.

### Example 56: (R)-4-(4-methoxyphenyl)-8-methyl-N-(1-methylpiperidin-3-yl)phthalazin-1-amine

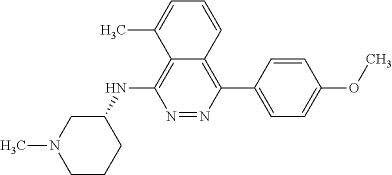

A mixture of (R)-4-chloro-8-methyl-N-(1-methylpiperidin-3-yl)phthalazin-1-amine and (R)-4-chloro-5-methyl-N-(1-methylpiperidin-3-yl)phthalazin-1-amine (50 mg), (4-methoxyphenyl)boronic acid (31.35 mg, 206.34 μmol), Pd(dppf)Cl₂·CH₂Cl₂ (28.08 mg, 34.39 μmol) and Cs₂CO₃ (112.05 mg, 343.90 μmol) in dioxane (2 mL) and H₂O (0.3 mL) was stirred at 100° C. for 12 hours under N₂. LC-MS showed one main peak with desired m/z. The mixture was diluted with 1 M HCl aq (2 mL), extracted with EtOAc (3 mL×2) and the aqueous layer was concentrated and purified by preparative HPLC (Method C). The title compound was obtained as a yellow solid (25 mg, 40%). ¹H NMR (400 MHz, DMSO-d₆) δ ppm 1.51-1.91 (m, 4H), 2.34 (s, 3H), 2.39-2.50 (m, 2H), 2.53-2.58 (m, 1H), 2.96 (s, 4H), 3.85 (s, 3H), 4.46 (br s, 1H), 6.23 (br s, 1H), 7.09 (d, J=8.6 Hz, 2H), 7.49 (d, J=8.6 Hz, 2H), 7.59-7.73 (m, 3H), 8.23 (s, 1H); ESI-MS m/z [M+H]⁺ 363.2.

### Example 57: (R)-1-(2-fluoro-4-methoxyphenyl)-N-(1-methylpiperidin-3-yl)pyrido[3,4-d]pyridazin-4-amine

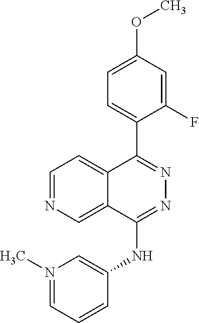

To a solution of (R)-1-chloro-N-(1-methylpiperidin-3-yl)pyrido[3,4-d]pyridazin-4-amine (75 mg, 270.03 μmol), (2-fluoro-4-methoxyphenyl)boronic acid (59.66 mg, 351.03 μmol) and Cs₂CO₃ (175.96 mg, 540.05 μmol) in dioxane (2 mL) and H₂O (0.4 mL) was added Pd(dppf)Cl₂ (9.88 mg, 13.50 μmol). The mixture was stirred at 100° C. for 12 hours under N₂. LC-MS showed one main peak with desired m/z. The mixture was filtered and the filtrate was purified by preparative HPLC (Method B) to give a formic acid salt of the title compound as a yellow solid (21.5 mg, 18.6% yield, 96.7% purity). ¹H NMR (400 MHz, DMSO-d₆) δ ppm 1.41-1.53 (m, 1H), 1.55-1.68 (m, 1H), 1.77 (br d, J=13.0 Hz, 1H), 1.90-2.05 (m, 3H), 2.23 (s, 3H), 2.74 (br d, J=10.9 Hz, 1H), 3.11 (br d, J=8.1 Hz, 1H), 3.87 (s, 3H), 4.47 (br d, J=5.9 Hz, 1H), 6.99 (dd, J=8.5, 2.4 Hz, 1H), 7.04 (dd, J=12.1, 2.4 Hz, 1H), 7.33 (dd, J=5.5, 3.1 Hz, 1H), 7.50 (t, J=8.6 Hz, 1H), 7.68 (d, J=7.4 Hz, 1H), 8.19 (s, 1H), 8.89 (d, J=5.6 Hz, 1H), 9.81 (s, 1H); ESI-MS m/z [M+H]⁺ 368.

### Example 58: (R)—N-(1-(2-fluoroethyl)piperidin-3-yl)-4-(4-methoxyphenyl)phthalazin-1-amine

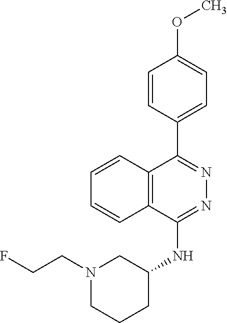 [SMILES: COc1ccc(-c2nnc(N[C@@H]3CCCN(CCF)C3)c3ccccc23)cc1]

The title compound was prepared like Example 57, using (R)-4-chloro-N-(1-(2-fluoroethyl)piperidin-3-yl)phthalazin-1-amine (150 mg, 485.78 μmol) and (4-methoxyphenyl)boronic acid (147.63 mg, 971.56 μmol), and was obtained as a gray solid (24.1 mg, 13.4%). ¹H NMR (400 MHz, DMSO-d₆) δ ppm 1.39-1.68 (m, 2H), 1.69-1.82 (m, 1H), 1.93-2.14 (m, 3H), 2.64 (t, J=4.88 Hz, 1H), 2.71 (t, J=4.94 Hz, 1H), 2.86 (br d, J=11.01 Hz, 1H), 3.16-3.25 (m, 1H), 3.85 (s, 3H), 4.35-4.47 (m, 1H), 4.49 (t, J=4.88 Hz, 1H), 4.61 (t, J=4.94 Hz, 1H), 7.05 (d, J=7.75 Hz, 1H), 7.10 (d, J=8.76 Hz, 2H), 7.54 (d, J=8.63 Hz, 2H), 7.77-7.92 (m, 3H), 8.40 (d, J=8.00 Hz, 1H); ESI-MS m/z [M+H]⁺ 381.

### Example 59: (R)-5-chloro-2-(4-((1-(2-fluoroethyl)piperidin-3-yl)amino)phthalazin-1-yl)phenol

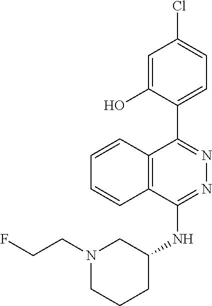 [SMILES: Oc1cc(Cl)ccc1-c1nnc(N[C@@H]2CCCN(CCF)C2)c2ccccc12]

The title compound was prepared like Example 57, using (R)-4-chloro-N-(1-(2-fluoroethyl)piperidin-3-yl)phthalazin-1-amine (200 mg, 647.71 μmol) and (4-chloro-2-hydroxyphenyl)boronic acid (133.98 mg, 777.25 μmol), and was obtained as a yellow solid (37.3 mg, 20.4%). ¹H NMR (400 MHz, DMSO-d₆) δ ppm 1.41-1.67 (m, 2H), 1.70-1.80 (m, 1H), 1.94-2.13 (m, 3H), 2.61-2.67 (m, 1H), 2.71 (t, J=4.89 Hz, 1H), 2.86 (br d, J=11.04 Hz, 1H), 3.21 (br d, J=7.03 Hz, 1H), 4.35-4.47 (m, 1H), 4.49 (t, J=5.02 Hz, 1H), 4.61 (t, J=4.89 Hz, 1H), 6.95-7.05 (m, 2H), 7.09 (d, J=7.78 Hz, 1H), 7.30 (d, J=8.28 Hz, 1H), 7.46 (d, J=7.78 Hz, 1H), 7.73-7.80 (m, 1H), 7.81-7.89 (m, 1H), 8.37 (d, J=8.28 Hz, 1H), 10.17 (br s, 1H); ¹⁹F NMR (376 MHz, DMSO-d₆) δ ppm −216.54 (br s, 1 F); ESI-MS m/z [M+H]⁺ 401.2.

### Example 60: (R)-2-(4-((1-(2-fluoroethyl)piperidin-3-yl)amino)phthalazin-1-yl)phenol

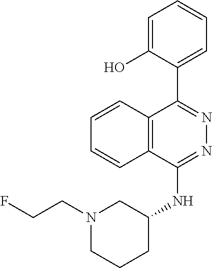 [SMILES: Oc1ccccc1-c1nnc(N[C@@H]2CCCN(CCF)C2)c2ccccc12]

The title compound was prepared like Example 57, using (R)-4-chloro-N-(1-(2-fluoroethyl)piperidin-3-yl)phthalazin-1-amine (200 mg, 647.71 μmol) and (2-hydroxyphenyl)boronic acid (178.68 mg, 1.30 mmol), and was obtained as a yellow solid (31.5 mg, 15.5%). ¹H NMR (400 MHz, DMSO-d₆) δ ppm 1.42-1.68 (m, 2H), 1.71-1.80 (m, 1H), 1.94-2.13 (m, 3H), 2.65 (t, J=4.89 Hz, 1H), 2.72 (t, J=4.89 Hz, 1H), 2.86 (br d, J=10.79 Hz, 1H), 3.17-3.26 (m, 1H), 4.35-4.47 (m, 1H), 4.50 (t, J=4.89 Hz, 1H), 4.62 (t, J=4.89 Hz, 1H), 6.88-7.01 (m, 2H), 7.05 (d, J=7.78 Hz, 1H), 7.24-7.36 (m, 2H), 7.48 (d, J=8.03 Hz, 1H), 7.72-7.80 (m, 1H), 7.81-7.87 (m, 1H), 8.36 (d, J=8.28 Hz, 1H), 9.65 (br s, 1H); ¹⁹F NMR (376 MHz, DMSO-d₆) δ ppm −216.55 (br s, 1 F); ESI-MS m/z [M+H]⁺ 367.2.

### Example 61: (R)-4-(2-fluoro-4-methoxyphenyl)-N-(1-(2-fluoroethyl)piperidin-3-yl)phthalazin-1-amine

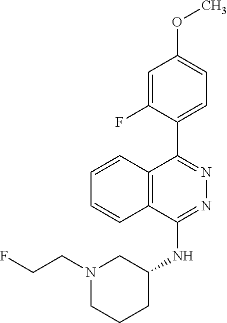 [SMILES: COc1ccc(-c2nnc(N[C@@H]3CCCN(CCF)C3)c3ccccc23)c(F)c1]

A formic acid salt of the title compound was prepared like Example 57, using (R)-4-chloro-N-(1-(2-fluoroethyl)piperidin-3-yl)phthalazin-1-amine (150 mg, 485.78 μmol) and (2-fluoro-4-methoxyphenyl)boronic acid (165.11 mg, 971.56 μmol), and was obtained as a yellow solid (56.1 mg, 26.5%). ¹H NMR (400 MHz, DMSO-d₆) δ ppm 1.41-1.67 (m, 2H), 1.76 (br d, J=13.01 Hz, 1H), 1.94-2.14 (m, 3H), 2.65 (br t, J=4.88 Hz, 1H), 2.72 (br t, J=4.75 Hz, 1H), 2.87 (br d, J=10.76 Hz, 1H), 3.21 (br d, J=7.88 Hz, 1H), 3.87 (s, 3H), 4.43 (br s, 1H), 4.50 (br t, J=4.75 Hz, 1H), 4.62 (br t, J=4.75 Hz, 1H), 6.94-7.05 (m, 2H), 7.15 (br s, 1H), 7.40-7.55 (m, 2H), 7.76-7.95 (m, 2H), 8.18 (s, 1H), 8.41 (br d, J=8.13 Hz, 1H); ESI-MS m/z [M+H]⁺ 399.2.

### Example 62: 4-(4-chlorophenyl)-N-(1-methylpiperidin-3-yl)pyrido[3,4-d]pyridazin-1-amine

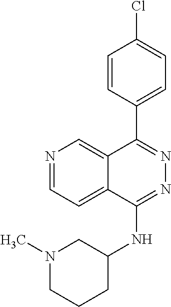 [SMILES: CN1CCCC(Nc2nnc(-c3ccc(Cl)cc3)c3cnccc23)C1]

### Example 63: 1-(4-chlorophenyl)-N-(1-methylpiperidin-3-yl)pyrido[3,4-d]pyridazin-4-amine

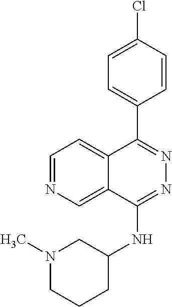 [SMILES: CN1CCCC(Nc2nnc(-c3ccc(Cl)cc3)c3ccncc23)C1]

The title compounds were prepared like Example 57, using a mixture of 1-chloro-N-(1-methylpiperidin-3-yl)pyrido[3,4-d]pyridazin-4-amine and 4-chloro-N-(1-methylpiperidin-3-yl)pyrido[3,4-d]pyridazin-1-amine (300 mg, 1.08 mmol), and (4-chlorophenyl)boronic acid (337.80 mg, 2.16 mmol). The title compound of Example 62 was obtained as a white solid (18.7 mg, 4.81%). ¹H NMR (400 MHz, DMSO-d₆) δ ppm 1.35-1.51 (m, 1H), 1.54-1.69 (m, 1H), 1.72-1.84 (m, 1H), 1.85-2.07 (m, 3H), 2.15-2.27 (m, 3H), 2.70-2.81 (m, 1H), 3.03-3.14 (m, 1H), 4.32-4.59 (m, 1H), 7.60-7.72 (m, 6H), 8.89-8.96 (m, 1H), 9.74-9.88 (m, 1H); ESI-MS m/z [M+H]⁺ 354.3. The title compound of Example 63 was obtained as a white solid (23 mg, 5.9%). ¹H NMR (400 MHz, DMSO-d₆) δ ppm 1.38-1.52 (m, 1H), 1.54-1.69 (m, 1H), 1.73-1.81 (m, 1H), 1.89-2.05 (m, 3H), 2.19-2.27 (m, 3H), 2.70-2.79 (m, 1H), 3.04-3.14 (m, 1H), 4.36-4.51 (m, 1H), 7.44-7.50 (m, 1H), 7.61-7.69 (m, 2H), 7.71-7.81 (m, 2H), 8.13-8.23 (m, 1H), 8.32-8.40 (m, 1H), 8.97-9.05 (m, 1H), 9.19 (s, 1H); ESI-MS m/z [M+H]⁺ 354.3.

### Example 64: (R)-1-(4-chlorophenyl)-N-(1-methylpiperidin-3-yl)pyrido[3,4-d]pyridazin-4-amine

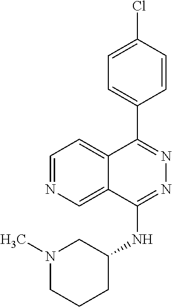 [SMILES: CN1CCC[C@@H](Nc2nnc(-c3ccc(Cl)cc3)c3ccncc23)C1]

and

### Example 65: (S)-1-(4-chlorophenyl)-N-(1-methylpiperidin-3-yl)pyrido[3,4-d]pyridazin-4-amine

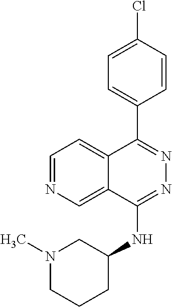 [SMILES: CN1CCC[C@H](Nc2nnc(-c3ccc(Cl)cc3)c3ccncc23)C1]

Racemic 1-(4-chlorophenyl)-N-(1-methylpiperidin-3-yl)pyrido[3,4-d]pyridazin-4-amine (22 mg) was resolved by chiral SFC (DAICEL CHIRALPAK® AD-10 μm, 30 mm×250 mm column) using a mobile phase of CO₂ and 40% IPA (with 0.1% NH₃H₂O). Example 64 was designated the R-enantiomer and was obtained as a white solid (8.1 mg, 10.6% yield). ¹H NMR (400 MHz, DMSO-d₆) δ ppm 1.48-1.59 (m, 1H), 1.68 (q, J 11.97 Hz, 1H), 1.83 (br d, J=13.26 Hz, 1H), 1.96-2.12 (m, 3H), 2.28 (s, 3H), 2.77 (br d, J=12.51 Hz, 1H), 3.13 (br d, J=10.26 Hz, 1H), 4.52 (br s, 1H), 7.66-7.72 (m, 3H), 7.72-7.78 (m, 3H), 8.98 (d, J=5.63 Hz, 1H), 9.87 (s, 1H); ESI-MS m/z [M+H]⁺ 354.3. Example 65 was designated the S-enantiomer and was obtained as a white solid (3.1 mg, 4.0%). ¹H NMR (400 MHz, DMSO-d₆) δ ppm 1.47-1.59 (m, 1H), 1.62-1.75 (m, 1H), 1.84 (br d, J=12.88 Hz, 1H), 1.99-2.12 (m, 3H), 2.29 (s, 3H), 2.81 (br s, 1H), 3.16 (br d, J=7.63 Hz, 1H), 4.53 (br d, J=6.88 Hz, 1H), 7.66-7.72 (m, 3H), 7.72-7.78 (m, 3H), 8.25 (br s, 1H), 8.97 (d, J=5.63 Hz, 1H), 9.88 (s, 1H); ESI-MS m/z [M+H]⁺ 354.3.

### Example 66: (R)-1-(4-chlorophenyl)-N-(1-methylpiperidin-3-yl)pyrido[3,4-d]pyridazin-4-amine

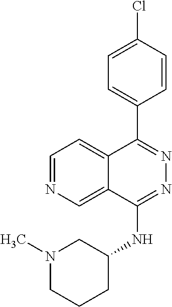 [SMILES: CN1CCC[C@@H](Nc2nnc(-c3ccc(Cl)cc3)c3ccncc23)C1]

and

### Example 67: (R)-4-(4-chlorophenyl)-N-(1-methylpiperidin-3-yl)pyrido[3,4-d]pyridazin-1-amine

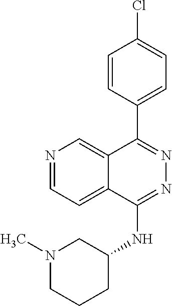 [SMILES: CN1CCC[C@@H](Nc2nnc(-c3ccc(Cl)cc3)c3cnccc23)C1]

The title compounds were prepared like Example 57, using a mixture of (R)-4-chloro-N-(1-methylpiperidin-3-yl)pyrido[3,4-d]pyridazin-1-amine and (R)-1-chloro-N-(1-methylpiperidin-3-yl)pyrido[3,4-d]pyridazin-4-amine (100.00 mg, 360.03 μmol), and (4-chlorophenyl)boronic acid (112.60 mg, 720.07 μmol). The title compound of Example 66 was obtained as a yellow solid (12.1 mg, 9.5%). ¹H NMR (400 MHz, DMSO-d₆) δ ppm 1.44-1.58 (m, 1H), 1.61-1.72 (m, 1H), 1.75-1.90 (m, 1H), 1.95-2.21 (m, 3H), 2.28-2.37 (m, 3H), 2.78-2.97 (m, 1H), 3.21 (br s, 1H), 4.45-4.63 (m, 1H), 7.58-7.80 (m, 6H), 8.87-9.01 (m, 1H), 9.84 (s, 1H); ESI-MS m/z [M+H]⁺ 354.3. The title compound of Example 67 was obtained as a yellow solid (5.2 mg, 4.1%). ¹H NMR (400 MHz, DMSO-d₆) δ ppm 1.64-1.82 (m, 2H), 1.84-1.98 (m, 2H), 2.00-2.16 (m, 2H), 2.47-2.67 (m, 5H), 4.51 (br s, 1H), 7.47-7.64 (m, 4H), 8.11-8.19 (m, 1H), 8.85-8.93 (m, 1H), 9.05-9.16 (m, 1H); ESI-MS m/z [M+H]⁺ 354.3.

### Example 68: 1-(4-chloro-2-fluorophenyl)-N-(1-methylpiperidin-3-yl)pyrido[3,4-d]pyridazin-4-amine

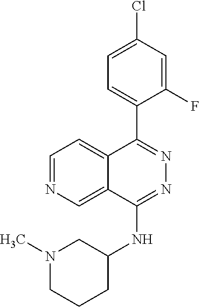 [SMILES: CN1CCCC(Nc2nnc(-c3ccc(Cl)cc3F)c3ccncc23)C1]

and

### Example 69: 4-(4-chloro-2-fluorophenyl)-N-(1-methylpiperidin-3-yl)pyrido[3,4-d]pyridazin-1-amine

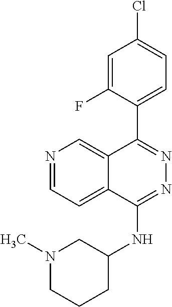 [SMILES: CN1CCCC(Nc2nnc(-c3ccc(Cl)cc3F)c3cnccc23)C1]

The title compounds were prepared like Example 57, using a mixture of 1-chloro-N-(1-methylpiperidin-3-yl)pyrido[3,4-d]pyridazin-4-amine and 4-chloro-N-(1-methylpiperidin-3-yl)pyrido[3,4-d]pyridazin-1-amine (120 mg, 0.43 μmol), and (4-chloro-2-fluorophenyl)boronic acid (113.00 mg, 648.06 μmol). The title compound of Example 68 was obtained as a white solid (50 mg, 62%). ¹H NMR (400 MHz, DMSO-d₆) δ ppm 1.42-1.54 (m, 1H), 1.57-1.70 (m, 1H), 1.74-1.83 (m, 1H), 1.92-2.07 (m, 3H), 2.25 (s, 3H), 2.75 (br d, J=11.38 Hz, 1H), 3.12 (br d, J=9.38 Hz, 1H), 4.43-4.56 (m, 1H), 7.36 (dd, J=5.44, 2.69 Hz, 1H), 7.52 (dd, J=8.25, 1.88 Hz, 1H), 7.61-7.71 (m, 2H), 7.79 (d, J=7.50 Hz, 1H), 8.90 (d, J=5.63 Hz, 1H), 9.84 (s, 1H); ESI-MS m/z [M+H]⁺ 372.1. The title compound of Example 69 was obtained as a white solid (11.4 mg, 12.5%). ¹H NMR (400 MHz, DMSO-d₆) δ ppm 1.48 (br s, 1H), 1.63 (br s, 1H), 1.79 (br s, 1H), 2.00 (br s, 3H), 2.28 (br s, 3H), 2.79 (br s, 1H), 3.13 (br s, 1H), 4.46 (br s, 1H), 7.54 (br s, 1H), 7.60 (br s, 1H), 7.68 (br s, 2H), 8.29-8.43 (m, 1H), 8.37 (br s, 1H), 8.91 (br s, 1H), 9.01 (br s, 1H); ESI-MS m/z [M+H]+ 372.1.

### Example 70: (S)-1-(4-chloro-2-fluorophenyl)-N-(1-methylpiperidin-3-yl)pyrido[3,4-d]pyridazin-4-amine

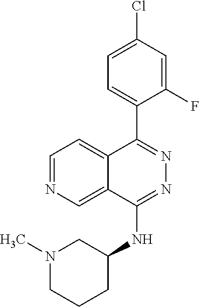 [SMILES: CN1CCC[C@H](Nc2nnc(-c3ccc(Cl)cc3F)c3ccncc23)C1]

and

### Example 71: (R)-1-(4-chloro-2-fluorophenyl)-N-(1-methylpiperidin-3-yl)pyrido[3,4-d]pyridazin-4-amine

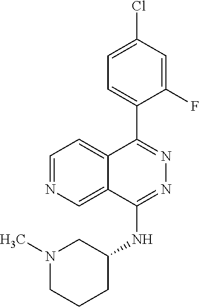 [SMILES: CN1CCC[C@@H](Nc2nnc(-c3ccc(Cl)cc3F)c3ccncc23)C1]

Racemic 1-(4-chloro-2-fluorophenyl)-N-(1-methylpiperidin-3-yl)pyrido[3,4-d]pyridazin-4-amine (20 mg) was separated by chiral SFC (DAICEL CHIRALCEL® OJ-10 μm, 30 mm×250 mm column) using a mobile phase of CO₂ and 30% EtOH (with 0.1% NH₃H₂O). Example 70 was designated the S-enantiomer and was obtained as a white solid (4.4 mg, 8.7%). ¹H NMR (400 MHz, DMSO-d₆) δ ppm 1.46-1.54 (m, 1H), 1.60-1.70 (m, 1H), 1.74-1.85 (m, 1H), 1.88-2.07 (m, 3H), 2.20-2.31 (m, 3H), 2.72-2.81 (m, 1H), 3.09-3.17 (m, 1H), 4.46-4.54 (m, 1H), 7.30-7.39 (m, 1H), 7.48-7.56 (m, 1H), 7.62-7.74 (m, 2H), 7.78-7.85 (m, 1H), 8.86-8.96 (m, 1H), 9.79-9.89 (m, 1H); ESI-MS m/z [M+H]⁺ 372.1. Example 71 was designated the R-enantiomer and was obtained as a white solid (9.8 mg, 19%). ¹H NMR (400 MHz, DMSO-d₆) δ ppm 1.41-1.54 (m, 1H), 1.55-1.67 (m, 1H), 1.71-1.83 (m, 1H), 1.87-2.05 (m, 3H), 2.23 (s, 3H), 2.70-2.78 (m, 1H), 3.07-3.13 (m, 1H), 4.43-4.53 (m, 1H), 7.33-7.38 (m, 1H), 7.46-7.57 (m, 1H), 7.61-7.71 (m, 2H), 7.76-7.83 (m, 1H), 8.85-8.94 (m, 1H), 9.82-9.87 (m, 1H); ESI-MS m/z [M+H]⁺ 372.1.

### Example 72: 1-(4-methoxyphenyl)-N-(1-methylpiperidin-3-yl)pyrido[3,4-d]pyridazin-4-amine

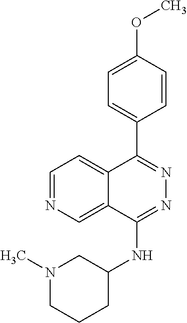

and

### Example 73: 4-(4-methoxyphenyl)-N-(1-methylpiperidin-3-yl)pyrido[3,4-d]pyridazin-1-amine

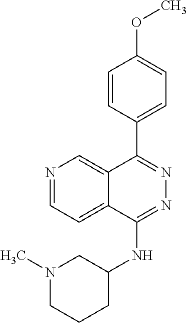

The title compounds were prepared like Example 57, using a mixture of 1-chloro-N-(1-methylpiperidin-3-yl)pyrido[3,4-d]pyridazin-4-amine and 4-chloro-N-(1-methylpiperidin-3-yl)pyrido[3,4-d]pyridazin-1-amine (200 mg, 0.72 mmol), and (4-methoxyphenyl)boronic acid (218.84 mg, 1.44 mmol). The title compound of Example 73 was obtained as a white solid (6.4 mg, 12.5%). ¹H NMR (400 MHz, DMSO-d₆) δ ppm 1.37-1.52 (m, 1H), 1.53-1.68 (m, 1H), 1.71-1.81 (m, 1H), 1.84-1.96 (m, 2H), 1.98-2.06 (m, 1H), 2.17-2.26 (m, 3H), 2.65-2.76 (m, 1H), 3.03-3.17 (m, 1H), 3.81-3.91 (m, 3H), 4.31-4.59 (m, 1H), 7.10-7.15 (m, 2H), 7.53-7.62 (m, 3H), 7.63-7.67 (m, 1H), 8.84-8.97 (m, 1H), 9.74-9.86 (m, 1H); ESI-MS m/z [M+H]⁺ 350.2. The title compound of Example 72 was obtained as a white solid (53.8 mg, 21.2%). ¹H NMR (400 MHz, DMSO-d₆) δ ppm 1.35-1.51 (m, 1H), 1.53-1.66 (m, 1H), 1.67-1.79 (m, 1H), 1.83-2.06 (m, 3H), 2.11-2.30 (m, 3H), 2.63-2.81 (m, 1H), 2.97-3.16 (m, 1H), 3.78-3.95 (m, 3H), 4.26-4.56 (m, 1H), 6.99-7.22 (m, 2H), 7.26-7.43 (m, 1H), 7.53-7.80 (m, 2H), 8.22-8.38 (m, 1H), 8.85-9.05 (m, 1H), 9.08-9.30 (m, 1H); ESI-MS m/z [M+H]⁺ 350.2.

### Example 74: (R)-5-chloro-2-(4-((1-methylpiperidin-3-yl)amino)pyrido[3,4-d]pyridazin-1-yl)phenol

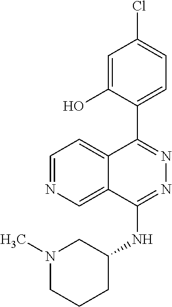 [SMILES: CN1CCC[C@@H](Nc2nnc(-c3ccc(Cl)cc3O)c3ccncc23)C1]

and

### Example 75: (R)-5-chloro-2-(1-((1-methylpiperidin-3-yl)amino)pyrido[3,4-d]pyridazin-4-yl)phenol

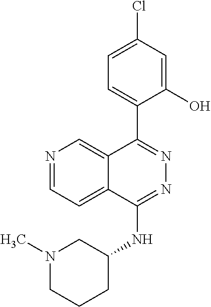 [SMILES: CN1CCC[C@@H](Nc2nnc(-c3ccc(Cl)cc3O)c3cnccc23)C1]

The title compounds were prepared like Example 57, using a mixture of (R)-1-chloro-N-(1-methylpiperidin-3-yl)pyrido[3,4-d]pyridazin-4-amine and (R)-4-chloro-N-(1-methylpiperidin-3-yl)pyrido[3,4-d]pyridazin-1-amine (300 mg, 1.08 mmol), and (4-chloro-2-hydroxyphenyl)boronic acid (465.45 mg, 2.70 mmol). The title compound of Example 74 was obtained as a yellow solid (64 mg, 16%). ¹H NMR (400 MHz, DMSO-d₆) δ ppm 1.43-1.56 (m, 1H), 1.59-1.70 (m, 1H), 1.77-1.82 (m, 1H), 1.98-2.11 (m, 3H), 2.26-2.31 (m, 3H), 2.75-2.83 (m, 1H), 3.12-3.20 (m, 1H), 4.41-4.57 (m, 1H), 7.01-7.07 (m, 2H), 7.27-7.32 (m, 1H), 7.32-7.37 (m, 1H), 7.58-7.67 (m, 1H), 8.15-8.22 (m, 1H), 8.82-8.89 (m, 1H), 9.75-9.81 (m, 1H); ESI-MS m/z [M+H]⁺ 370.4. The title compound of Example 75 was obtained as a yellow solid (24.5 mg, 5.94% yield, 96.8% purity). ¹H NMR (400 MHz, DMSO-d₆) δ ppm 1.43-1.57 (m, 1H), 1.58-1.71 (m, 1H), 1.75-1.85 (m, 1H), 1.96-2.04 (m, 1H), 2.05-2.15 (m, 2H), 2.27-2.34 (m, 3H), 2.76-2.84 (m, 1H), 3.11-3.20 (m, 1H), 4.39-4.51 (m, 1H), 7.03-7.08 (m, 2H), 7.37-7.42 (m, 1H), 7.42-7.49 (m, 1H), 8.18-8.23 (m, 1H), 8.29-8.34 (m, 1H), 8.83-8.86 (m, 1H), 8.91-8.96 (m, 1H); ESI-MS m/z [M+H]+ 370.3.

### Example 76: 1-(4-chlorophenyl)-5-methyl-N-(1-methylpiperidin-3-yl)pyrido[3,4-d]pyridazin-4-amine

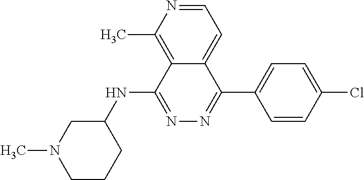 [SMILES: Cc1nccc2c(-c3ccc(Cl)cc3)nnc(NC3CCCN(C)C3)c12]

and

### Example 77: 4-(4-chlorophenyl)-5-methyl-N-(1-methylpiperidin-3-yl)pyrido[3,4-d]pyridazin-1-amine

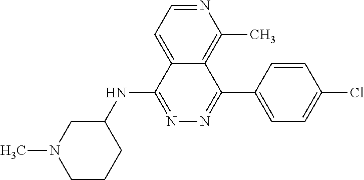 [SMILES: Cc1nccc2c(NC3CCCN(C)C3)nnc(-c3ccc(Cl)cc3)c12]

The title compounds were prepared like Example 57, using a mixture of 1-chloro-5-methyl-N-(1-methylpiperidin-3-yl)pyrido[3,4-d]pyridazin-4-amine and 4-chloro-5-methyl-N-(1-methylpiperidin-3-yl)pyrido[3,4-d]pyridazin-1-amine (80 mg), and (4-chlorophenyl)boronic acid (55.55 mg, 355.23 μmol). The title compound of Example 77 was obtained as a white solid (4.1 mg, 4.1%). ¹H NMR (400 MHz, CD₃OD) δ ppm 1.71-2.01 (m, 2H), 2.06-2.25 (m, 2H), 2.30 (s, 3H), 2.82 (s, 3H), 2.96 (br s, 2H), 3.33-3.38 (m, 1H), 3.71 (br dd, J=4.3, 1.8 Hz, 1H), 4.64 (br s, 1H), 7.48 (br d, J=8.0 Hz, 2H), 7.58 (br d, J=8.0 Hz, 2H), 8.08 (br d, J=5.3 Hz, 1H), 8.51 (br s, 1H), 8.82 (br d, J=5.5 Hz, 1H); ESI-MS m/z [M+H]+ 368.2. The title compound of Example 76 was obtained as a white solid (7 mg, 7%). ¹H NMR (400 MHz, CD₃OD) δ ppm 1.83-2.06 (m, 2H), 2.12 (br s, 1H), 2.25 (br s, 1H), 2.83 (s, 3H), 3.02 (br d, J=10.0 Hz, 2H), 3.23 (s, 4H), 3.71 (br s, 1H), 4.71 (br s, 1H), 7.57 (br d, J=5.6 Hz, 1H), 7.63 (s, 4H), 8.49 (br s, 1H), 8.73 (d, J=5.6 Hz, 1H); ESI-MS m/z M+H]+368.2.

### Example 78: (R)-5-methoxy-2-(5-methyl-4-((1-methylpiperidin-3-yl)amino)pyrido[3,4-d]pyridazin-1-yl)phenol

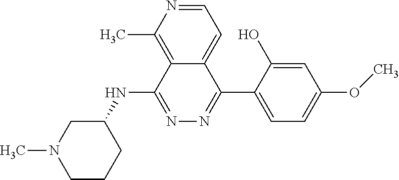

and

### Example 79: (R)-5-methoxy-2-(5-methyl-1-((1-methylpiperidin-3-yl)amino)pyrido[3,4-d]pyridazin-4-yl)phenol

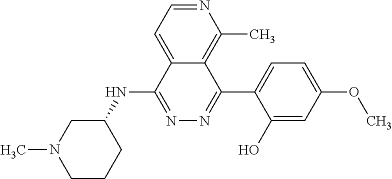

The title compounds were prepared like Example 57, using a mixture of (R)-1-chloro-5-methyl-N-(1-methylpiperidin-3-yl)pyrido[3,4-d]pyridazin-4-amine and (R)-4-chloro-5-methyl-N-(1-methylpiperidin-3-yl)pyrido[3,4-d]pyridazin-1-amine (45 mg, 154 μmol), and 5-methoxy-2-(4,4,5,5-tetramethyl-1,3,2-dioxaborolan-2-yl)phenol (57.86 mg, 231.34 μmol). The title compound of Example 78 was obtained as a yellow solid (5.6 mg, 9.5% yield, 99% purity). ¹H NMR (400 MHz, DMSO-d₆) δ ppm 1.42-1.52 (m, 1H), 1.61 (br d, J=12.01 Hz, 1H), 1.76 (br d, J=10.88 Hz, 1H), 1.97 (br t, J=10.13 Hz, 3H), 2.24 (s, 3H), 2.31 (s, 3H), 2.74 (br d, J=10.51 Hz, 1H), 3.09 (br d, J=6.25 Hz, 1H), 3.78 (s, 3H), 4.37 (br d, J=3.63 Hz, 1H), 6.49 (s, 1H), 6.55 (br d, J=8.38 Hz, 1H), 7.13-7.23 (m, 2H), 8.11 (d, J=5.63 Hz, 1H), 8.21 (s, 1H), 8.74 (d, J=5.63 Hz, 1H), 9.37-9.76 (m, 1H); ESI-MS m/z [M+H]+ 380.4. The title compound of Example 79 was obtained as a yellow solid (9.6 mg, 16% yield, 97% purity). ¹H NMR (400 MHz, DMSO-d₆) δ ppm 1.53-1.65 (m, 1H), 1.66-1.90 (m, 3H), 2.26 (s, 3H), 2.37-2.45 (m, 2H), 2.79 (br d, J=7.38 Hz, 1H), 3.10 (s, 3H), 3.19-3.24 (m, 1H), 3.77-3.84 (m, 3H), 4.38-4.56 (m, 1H), 6.35-6.46 (m, 1H), 6.53-6.59 (m, 2H), 7.09-7.16 (m, 1H), 7.18-7.24 (m, 1H), 8.11-8.26 (m, 1H), 8.52-8.69 (m, 1H), 9.43-9.98 (m, 1H); ESI-MS m/z [M+H]+ 380.4.

### Example 80: 1-(4-chlorophenyl)-7-methyl-N-(1-methylpiperidin-3-yl)pyrido[3,4-d]pyridazin-4-amine

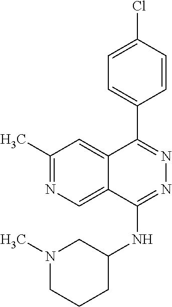 [SMILES: Cc1cc2c(-c3ccc(Cl)cc3)nnc(NC3CCCN(C)C3)c2cn1]

and

### Example 81: 4-(4-chlorophenyl)-7-methyl-N-(1-methylpiperidin-3-yl)pyrido[3,4-d]pyridazin-1-amine

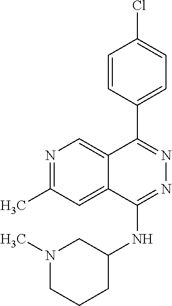 [SMILES: Cc1cc2c(NC3CCCN(C)C3)nnc(-c3ccc(Cl)cc3)c2cn1]

The title compounds were prepared like Example 57, using a mixture of 1-chloro-7-methyl-N-(1-methylpiperidin-3-yl)pyrido[3,4-d]pyridazin-4-amine and 4-chloro-7-methyl-N-(1-methylpiperidin-3-yl)pyrido[3,4-d]pyridazin-1-amine (100 mg), and (4-chlorophenyl)boronic acid (80.39 mg, 514.09 μmol). The title compound of Example 80 was obtained as a yellow solid (24.2 mg, 16.6%). ¹H NMR (400 MHz, CD₃OD) δ ppm 1.77-2.01 (m, 2H), 2.08-2.34 (m, 2H), 2.72 (s, 3H), 2.81 (br d, J=2.0 Hz, 3H), 2.95 (br d, J=3.4 Hz, 2H), 3.30 (br d, J=1.5 Hz, 1H), 3.60-3.81 (m, 1H), 4.70 (br s, 1H), 7.58 (s, 1H), 7.60-7.69 (m, 4H), 8.50 (br s, 1H), 9.63 (s, 1H); ESI-MS m/z [M+H]⁺ 368.3. The title compound of Example 81 was obtained as a yellow solid (9.9 mg, 6.8%). ¹H NMR (400 MHz, CD₃OD) δ ppm 1.72-2.03 (m, 2H), 2.08-2.35 (m, 2H), 2.79 (s, 3H), 2.83 (s, 3H), 3.01 (br s, 2H), 3.32-3.39 (m, 1H), 3.70 (br d, J=9.4 Hz, 1H), 4.60-4.71 (m, 1H), 7.51-7.75 (m, 4H), 8.12 (s, 1H), 8.48 (br s, 1H), 9.09 (s, 1H); ESI-MS m/z [M+H]⁺ 368.3.

### Example 82: 4-(4-chlorophenyl)-7-methoxy-N-(1-methylpiperidin-3-yl)pyrido[3,4-d]pyridazin-1-amine

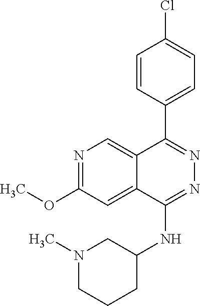 [SMILES: COc1cc2c(NC3CCCN(C)C3)nnc(-c3ccc(Cl)cc3)c2cn1]

The title compound was prepared like Example 57, using 4-chloro-7-methoxy-N-(1-methylpiperidin-3-yl)pyrido[3,4-d]pyridazin-1-amine (11 mg, 35.7 μmol) and (4-chlorophenyl)boronic acid (6.71 mg, 42.89 μmol), and was obtained as a yellow solid (3.9 mg, 28% yield, 99% purity). ¹H NMR (400 MHz, DMSO-d₆) δ ppm 1.34-1.51 (m, 1H), 1.53-1.69 (m, 1H), 1.71-1.81 (m, 1H), 1.87-2.06 (m, 3H), 2.18-2.27 (m, 3H), 2.73-2.77 (m, 1H), 3.10 (br d, J=9.01 Hz, 1H), 3.99-4.06 (m, 3H), 4.33-4.43 (m, 1H), 7.23-7.32 (m, 1H), 7.59-7.65 (m, 2H), 7.68-7.77 (m, 1H), 8.14-8.21 (m, 1H), 8.88 (s, 1H); ESI-MS m/z [M+H]+ 384.3.

### Example 83: 1-(4-chlorophenyl)-7-methoxy-N-(1-methylpiperidin-3-yl)pyrido[3,4-d]pyridazin-4-amine

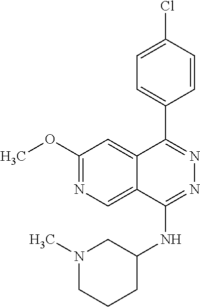 [SMILES: COc1cc2c(-c3ccc(Cl)cc3)nnc(NC3CCCN(C)C3)c2cn1]

The title compound was prepared like Example 57, using 1-chloro-7-methoxy-N-(1-methylpiperidin-3-yl)pyrido[3,4-d]pyridazin-4-amine (33 mg, 107.22 μmol) and (4-chlorophenyl)boronic acid (20.12 mg, 128.66 μmol), and was obtained as a yellow solid (16 mg, 38% yield, 98% purity). ¹H NMR (400 MHz, DMSO-d₆) δ ppm 1.44-1.56 (m, 1H), 1.59-1.71 (m, 1H), 1.78-1.87 (m, 1H), 1.98-2.24 (m, 3H), 2.31-2.37 (m, 3H), 2.81-2.90 (m, 1H), 3.16-3.23 (m, 1H), 3.95-4.02 (m, 3H), 4.49 (br s, 1H), 6.81-6.90 (m, 1H), 7.59-7.64 (m, 2H), 7.66-7.70 (m, 2H), 8.09-8.21 (m, 1H), 9.48-9.58 (m, 1H); ESI-MS m/z [M+H]+ 384.3.

### Example 84: (R)—N-(1-(2-fluoroethyl)piperidin-3-yl)-1-(4-methoxyphenyl)pyrido[3,4-d]pyridazin-4-amine

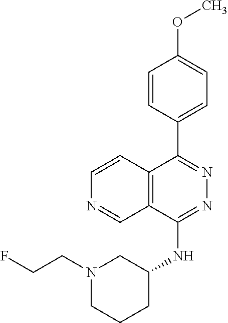 [SMILES: COc1ccc(-c2nnc(N[C@@H]3CCCN(CCF)C3)c3cnccc23)cc1]

The title compound was prepared like Example 57, using (R)-1-chloro-N-(1-(2-fluoroethyl)piperidin-3-yl)pyrido[3,4-d]pyridazin-4-amine (200 mg, 645.64 μmol) and (4-methoxyphenyl)boronic acid (196.22 mg, 1.29 mmol), and was obtained as a yellow solid (27.3 mg, 13.4%). ¹H NMR (400 MHz, DMSO-d₆) δ ppm 1.41-1.69 (m, 2H), 1.71-1.84 (m, 1H), 1.96-2.16 (m, 3H), 2.66 (t, J=4.88 Hz, 1H), 2.73 (t, J=4.88 Hz, 1H), 2.88 (br d, J=10.76 Hz, 1H), 3.23 (br d, J=7.00 Hz, 1H), 3.85 (s, 3H), 4.39-4.53 (m, 2H), 4.62 (t, J=4.88 Hz, 1H), 7.12 (d, J=8.63 Hz, 2H), 7.49-7.61 (m, 3H), 7.64 (d, J=5.63 Hz, 1H), 8.15 (s, 1H), 8.90 (d, J=5.63 Hz, 1H), 9.79 (s, 1H); ESI-MS m/z [M+H]+ 382.

### Example 85: (R)-5-chloro-2-(4-((1-(2-fluoroethyl)piperidin-3-yl)amino)pyrido[3,4-d]pyridazin-1-yl)phenol

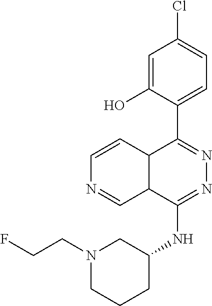 [SMILES: Oc1cc(Cl)ccc1C1=NN=C(N[C@@H]2CCCN(CCF)C2)C2C=NC=CC12]

A formic acid salt of the title compound was prepared like Example 57, using (R)-1-chloro-N-(1-(2-fluoroethyl)piperidin-3-yl)pyrido[3,4-d]pyridazin-4-amine (300 mg, 968.46 μmol) and (4-chloro-2-hydroxyphenyl)boronic acid (200.33 mg, 1.16 mmol), and was obtained as a yellow solid (27.3 mg, 9.97%). ¹H NMR (400 MHz, DMSO-d₆) δ ppm 1.42-1.68 (m, 2H), 1.72-1.83 (m, 1H), 1.97-2.14 (m, 3H), 2.65 (t, J=4.89 Hz, 1H), 2.73 (t, J=4.89 Hz, 1H), 2.88 (br d, J=10.79 Hz, 1H), 3.23 (br d, J=10.79 Hz, 1H), 4.39-4.53 (m, 2H), 4.62 (t, J=4.89 Hz, 1H), 6.98-7.08 (m, 2H), 7.28 (d, J=5.77 Hz, 1H), 7.34 (d, J=8.78 Hz, 1H), 7.61 (br d, J=7.53 Hz, 1H), 8.18 (s, 1H), 8.84 (d, J=5.52 Hz, 1H), 9.76 (s, 1H), 10.30 (br s, 1H); ¹⁹F NMR (376 MHz, DMSO-d₆) δ ppm −216.55 (br s, 1 F); ESI-MS m/z [M+H]⁺ 402.2.

### Example 86: (R)-2-(4-((1-(2-fluoroethyl)piperidin-3-yl)amino)pyrido[3,4-d]pyridazin-1-yl)phenol

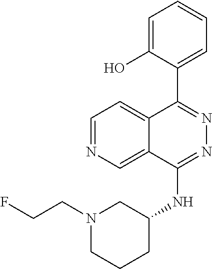 [SMILES: Oc1ccccc1-c1nnc(N[C@@H]2CCCN(CCF)C2)c2cnccc12]

The title compound was prepared like Example 57, using (R)-1-chloro-N-(1-(2-fluoroethyl)piperidin-3-yl)pyrido[3,4-d]pyridazin-4-amine (200 mg, 645.64 μmol) and (2-hydroxyphenyl)boronic acid (178.11 mg, 1.29 mmol), and was obtained as a yellow solid (27.4 mg, 20.8%). ¹H NMR (400 MHz, DMSO-d₆) δ ppm 1.42-1.69 (m, 2H), 1.73-1.83 (m, 1H), 1.99-2.15 (m, 3H), 2.66 (t, J=4.94 Hz, 1H), 2.73 (t, J=4.88 Hz, 1H), 2.88 (br d, J=11.26 Hz, 1H), 3.24 (br d, J=7.00 Hz, 1H), 4.39-4.55 (m, 2H), 4.62 (t, J=4.94 Hz, 1H), 6.93-7.06 (m, 2H), 7.23-7.40 (m, 3H), 7.56 (d, J=7.63 Hz, 1H), 8.84 (d, J=5.63 Hz, 1H), 9.69 (s, 1H), 9.76 (s, 1H); ¹⁹F NMR (376 MHz, DMSO-d₆) δ ppm −216.57 (s, 1 F); ESI-MS m/z [M+H]⁺ 368.2.

### Example 87: (R)-1-(2-fluoro-4-methoxyphenyl)-N-(1-(2-fluoroethyl)piperidin-3-yl)pyrido[3,4-d]pyridazin-4-amine

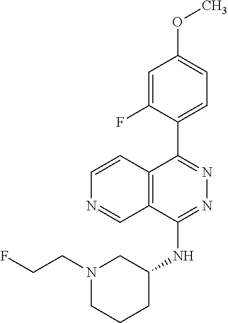 [SMILES: COc1ccc(-c2nnc(N[C@@H]3CCCN(CCF)C3)c3cnccc23)c(F)c1]

The title compound was prepared like Example 57, using (R)-1-chloro-N-(1-(2-fluoroethyl)piperidin-3-yl)pyrido[3,4-d]pyridazin-4-amine (200 mg, 645.64 μmol) and (2-fluoro-4-methoxyphenyl)boronic acid (219.45 mg, 1.29 mmol), and was obtained as a yellow solid (28.8 mg, 36%). ¹H NMR (400 MHz, DMSO-d₆) δ ppm 1.42-1.67 (m, 2H), 1.72-1.82 (m, 1H), 1.97-2.14 (m, 3H), 2.65 (t, J=4.88 Hz, 1H), 2.72 (t, J=4.88 Hz, 1H), 2.88 (br d, J=11.01 Hz, 1H), 3.23 (br dd, J=10.32, 3.31 Hz, 1H), 3.87 (s, 3H), 4.38-4.55 (m, 2H), 4.62 (t, J=4.88 Hz, 1H), 6.92-7.09 (m, 2H), 7.33 (dd, J=5.50, 3.00 Hz, 1H), 7.50 (t, J=8.57 Hz, 1H), 7.70 (d, J=7.63 Hz, 1H), 8.24 (s, 1H), 8.89 (d, J=5.63 Hz, 1H), 9.80 (s, 1H); ESI-MS m/z [M+H]⁺ 400.2.

### Example 88: 1-(4-chlorophenyl)-N-(1-cyclopropylpiperidin-3-yl)pyrido[3,4-d]pyridazin-4-amine

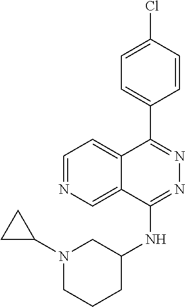 [SMILES: Clc1ccc(-c2nnc(NC3CCCN(C4CC4)C3)c3cnccc23)cc1]

The title compound was prepared like Example 57, using 1-chloro-N-(1-cyclopropylpiperidin-3-yl)pyrido[3,4-d]pyridazin-4-amine (100 mg, 329.18 μmol) and (4-chlorophenyl)boronic acid (102.95 mg, 658.35 μmol), and was obtained as a yellow solid (30 mg, 22% yield, 96% purity). ¹H NMR (400 MHz, DMSO-d₆) δ ppm 0.29-0.38 (m, 2H), 0.38-0.48 (m, 2H), 1.42-1.59 (m, 2H), 1.62-1.71 (m, 1H), 1.71-1.80 (m, 1H), 1.96-2.09 (m, 1H), 2.14-2.27 (m, 2H), 2.91 (br d, J=11.01 Hz, 1H), 3.29 (br dd, J=10.32, 3.31 Hz, 1H), 4.38 (br d, J=6.25 Hz, 1H), 7.56-7.76 (m, 6H), 8.90 (d, J=5.63 Hz, 1H), 9.80 (s, 1H); ESI-MS m/z [M+H]⁺ 380.1.

### Example 89: 4-(4-chlorophenyl)-N-(1-cyclopropylpiperidin-3-yl)pyrido[3,4-d]pyridazin-1-amine

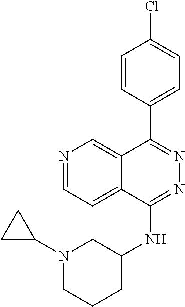 [SMILES: Clc1ccc(-c2nnc(NC3CCCN(C4CC4)C3)c3ccncc23)cc1]

The title compound was prepared like Example 57, using 4-chloro-N-(1-cyclopropylpiperidin-3-yl)pyrido[3,4-d]pyridazin-1-amine (96 mg, 316.01 μmol) and (4-chlorophenyl)boronic acid (98.83 mg, 632.02 μmol), and was obtained as a yellow solid (30 mg, 25% yield, 99% purity). ¹H NMR (400 MHz, DMSO-d₆) δ ppm 0.31-0.38 (m, 2H), 0.39-0.48 (m, 2H), 1.41-1.58 (m, 2H), 1.63-1.71 (m, 1H), 1.71-1.79 (m, 1H), 2.02 (br d, J=9.01 Hz, 1H), 2.19 (br t, J=10.26 Hz, 2H), 2.91 (br d, J=11.01 Hz, 1H), 3.23-3.31 (m, 1H), 4.33 (br s, 1H), 7.45 (br d, J=7.50 Hz, 1H), 7.64 (d, J=8.50 Hz, 2H), 7.71-7.76 (m, 2H), 8.33 (d, J=5.63 Hz, 1H), 9.00 (d, J=5.63 Hz, 1H), 9.15 (s, 1H); ESI-MS m/z [M+H]+ 380.1.

### Example 90: (R)-2-(4-((1-methylpiperidin-3-yl)amino)pyrido[3,4-d]pyridazin-1-yl)-5-(trifluoromethyl)phenol

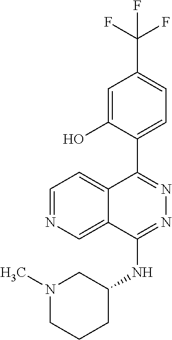 [SMILES: CN1CCC[C@@H](Nc2nnc(-c3ccc(C(F)(F)F)cc3O)c3ccncc23)C1]

To a 5 mL vial equipped with a stir bar were added (2-hydroxy-4-(trifluoromethyl)phenyl) boronic acid (33.4 mg, 0.162 mmol), (R)-1-chloro-N-(1-methylpiperidin-3-yl)pyrido[3,4-d]pyridazin-4-amine (30 mg, 0.108 mmol), XPhos-Pd-G2 (8.50 mg, 10.80 μmol), K₂CO₃ (29.9 mg, 0.216 mmol), dioxane (1.5 mL) and water (0.5 mL). The mixture was heated in a microwave reactor (Biotage® Initiator) at 110° C. for 30 minutes and then diluted with DMF (0.5 mL) and filtered through a hydrophilic PTFE 0.45 m filter (Millipore® Millex-LCR). The filtrate was purified by preparative HPLC (Method A) to give the title compound (22 mg, 41%). ¹H NMR (400 MHz, CD₃OD) δ 1.82-2.11 (m, 2H), 2.13-2.28 (m, 1H), 2.28-2.43 (m, 1H), 2.89-3.14 (m, 5H), 3.44-3.66 (m, 1H), 3.87-4.07 (m, 1H), 4.66-4.68 (m, 1H), 7.32 (s, 1H), 7.39 (d, J=8.10 Hz, 1H), 7.56-7.72 (m, 2H), 9.05 (d, J=5.52 Hz, 1H), 9.86 (s, 1H); ESI-MS m/z [M+H]⁺ 404.2.

### Example 91: (R)-1-(2-(difluoromethyl)phenyl)-N-(1-methylpiperidin-3-yl)pyrido[3,4-d]pyridazin-4-amine

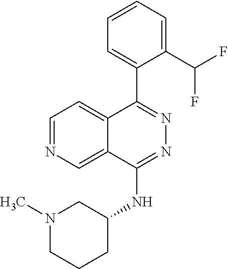 [SMILES: CN1CCC[C@@H](Nc2nnc(-c3ccccc3C(F)F)c3ccncc23)C1]

The title compound (10 mg, 20%) was prepared like Example 90, using (2-(difluoromethyl)phenyl)boronic acid (27.9 mg, 0.162 mmol) and (R)-1-chloro-N-(1-methylpiperidin-3-yl)pyrido[3,4-d]pyridazin-4-amine (30 mg, 0.108 mmol). ¹H NMR (400 MHz, CD₃OD) δ 1.86 (m, 1H), 1.93-2.11 (m, 1H), 2.12-2.29 (m, 1H), 2.29-2.45 (m, 1H), 2.85-3.07 (m, 5H), 3.51-3.69 (m, 1H), 3.92-4.12 (m, 1H), 4.55-4.79 (m, 1H), 6.76 (t, J=54.90 Hz, 1H), 7.43 (d, J=5.77 Hz, 1H), 7.49-7.58 (m, 1H), 7.72-7.81 (m, 2H), 7.85-7.94 (m, 1H), 8.98 (d, J=5.52 Hz, 1H), 9.81 (s, 1H); ESI-MS m/z [M+H]⁺ 370.2.

### Example 92: (R)-1-(4-(difluoromethoxy)-2-fluorophenyl)-N-(1-methylpiperidin-3-yl)pyrido[3,4-d]pyridazin-4-amine

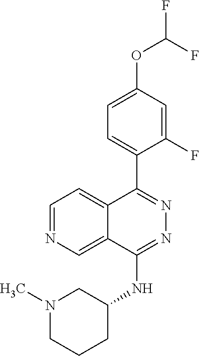 [SMILES: CN1CCC[C@@H](Nc2nnc(-c3ccc(OC(F)F)cc3F)c3ccncc23)C1]

To a 5 mL vial equipped with a stir bar were added (R)-1-chloro-N-(1-methylpiperidin-3-yl)pyrido[3,4-d]pyridazin-4-amine (30 mg, 0.108 mmol), 2-(4-(difluoromethoxy)-2-fluorophenyl)-4,4,5,5-tetramethyl-1,3,2-dioxaborolane (47 mg, 0.162 mmol), XPhos-Pd-G2 (8.5 mg, 0.0108 mmol), K₂CO₃ (30 mg, 0.216 mmol), 1,4-dioxane (1.5 mL) and water (0.5 mL). The mixture was heated in a microwave reactor (Biotage® Initiator) at 110° C. for 30 minutes and then diluted with DMF (0.3 mL) and filtered through a hydrophilic PTFE 0.45 m filter (Millipore® Millex-LCR), rinsing with MeOH. The filtrate was purified by preparative HPLC (Phenomenex Gemini® C18-5 μm, 30 mm×150 mm column) using a gradient of 10-100% ACN (0.035% TFA) in water (0.05% TFA) (slow ramp from 10-60% ACN). The product-containing fractions were evaporated to give the title compound (27 mg, 48%). ¹H NMR (400 MHz, CD₃OD) δ ppm 1.87 (br d, J 11.04 Hz, 1H), 1.93-2.11 (m, 1H), 2.19 (br d, J=14.31 Hz, 1H), 2.27-2.45 (m, 1H), 2.83-3.13 (m, 5H), 3.60 (br d, J=11.29 Hz, 1H), 3.98 (br d, J=10.54 Hz, 1H), 4.70 (br d, J=11.80 Hz, 1H), 6.88 (s, 1H), 7.06-7.31 (m, 3H), 7.54-7.73 (m, 2H), 9.04 (d, J=5.77 Hz, 1H), 9.84 (s, 1H); ESI-MS m/z [M+H]+ 404.1.

### Example 93: (R)-5-fluoro-2-(4-((1-methylpiperidin-3-yl)amino)pyrido[3,4-d]pyridazin-1-yl)phenol

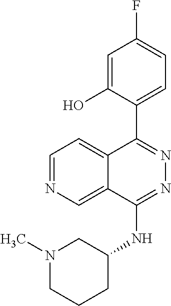 [SMILES: CN1CCC[C@@H](Nc2nnc(-c3ccc(F)cc3O)c3ccncc23)C1]

The title compound (14 mg, 28%) was prepared like Example 92, using (R)-1-chloro-N-(1-methylpiperidin-3-yl)pyrido[3,4-d]pyridazin-4-amine (30 mg, 0.108 mmol) and (4-fluoro-2-hydroxyphenyl)boronic acid (25 mg, 0.162 mmol). ¹H NMR (400 MHz, CD₃OD) δ ppm 1.73-1.93 (m, 1H), 2.00 (br d, J=13.55 Hz, 1H), 2.09-2.27 (m, 1H), 2.35 (br d, J=11.29 Hz, 1H), 2.81-3.10 (m, 5H), 3.60 (br d, J=11.29 Hz, 1H), 3.95 (br d, J=9.79 Hz, 1H), 4.54-4.74 (m, 1H), 6.73-6.91 (m, 2H), 7.48 (dd, J=8.53, 6.53 Hz, 1H), 7.76 (d, J=5.52 Hz, 1H), 9.11 (d, J=5.77 Hz, 1H), 9.90 (br s, 1H); ESI-MS m/z [M+H]⁺ 354.1.

### Example 94: cis-5-chloro-2-(1-((3-hydroxy-3-methylcyclobutyl)amino)pyrido[3,4-d]pyridazin-4-yl)phenol

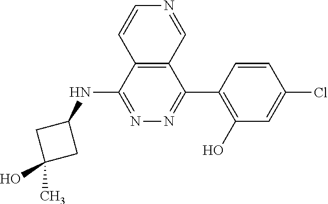 [SMILES: C[C@]1(O)C[C@@H](Nc2nnc(-c3ccc(Cl)cc3O)c3cnccc23)C1]

and

### Example 95: cis-5-chloro-2-(4-((-3-hydroxy-3-methylcyclobutyl)amino)pyrido[3,4-d]pyridazin-1-yl)phenol

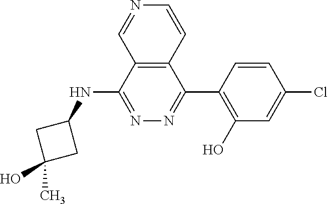 [SMILES: C[C@]1(O)C[C@@H](Nc2nnc(-c3ccc(Cl)cc3O)c3ccncc23)C1]

A mixture of cis-3-((1-chloropyrido[3,4-d]pyridazin-4-yl)amino)-1-methylcyclobutan-1-ol and cis-3-((4-chloropyrido[3,4-d]pyridazin-1-yl)amino)-1-methylcyclobutan-1-ol (37 mg, 0.139 mmol), (4-chloro-2-hydroxyphenyl)boronic acid (60 mg, 0.348 mmol), Pd(dppf)Cl₂·CH₂Cl₂ (11 mg, 0.0139 mmol) and K₂CO₃ (115 mg, 0.834 mmol) in 1,4-dioxane (2.0594 mL) and water (0.5148 mL) was stirred in an oil bath at 100° C. for 1 hour. The reaction mixture was cooled to room temperature, concentrated via rotary evaporation, dissolved in DMSO (1 mL), filtered through a 0.45 μm PTFE Membrane filter (VWR®), rinsed with DMSO (0.5 mL) and purified via reversed-phase chromatography (ISCO® AccqPrep C18 column) using a gradient of 10-100% ACN with H₂O (10 mM NH₄HCO₃). The appropriate fractions were combined and concentrated via rotary evaporation at 45° C. The resulting mixture was dried in vacuo to provide a crude mixture of products (50.3 mg) as a yellow solid. The crude material was dissolved in MeOH (2 mL) and purified via preparative SFC (Waters®) using a gradient of 20-40% MeOH (with 0.1% NH₃H₂O) and CO₂. The early fractions were combined, concentrated via rotary evaporation and dried in vacuo to give the title compound of Example 94 as a yellow solid (6.6 mg, 27%). ¹H NMR (400 MHz, DMSO-d₆) δ ppm 1.34 (s, 3H), 2.18 (td, J=8.91, 2.26 Hz, 2H), 2.47 (br d, J=2.76 Hz, 2H), 4.16-4.30 (m, 1H), 5.03 (s, 1H), 7.01-7.11 (m, 2H), 7.35-7.43 (m, 1H), 7.88 (br d, J=4.77 Hz, 1H), 8.31 (br d, J=5.52 Hz, 1H), 8.76-9.01 (m, 2H), 10.30 (s, 1H); ESI-MS m/z [M+H]⁺ 357.0. The later fractions were combined, concentrated via rotary evaporation and dried in vacuo to give the title compound of Example 95 as a yellow solid (18 mg, 71%). ¹H NMR (400 MHz, DMSO-d₆) δ ppm 1.28 (s, 3H), 2.13 (td, J=8.91, 2.26 Hz, 2H), 2.44-2.51 (m, 2H), 4.12-4.29 (m, 1H), 4.98 (s, 1H), 6.90-7.01 (m, 2H), 7.22 (d, J=5.52 Hz, 1H), 7.24-7.31 (m, 1H), 8.00 (br d, J=5.77 Hz, 1H), 8.77 (br d, J=5.52 Hz, 1H), 9.72 (s, 1H), 10.16 (s, 1H); ESI-MS m/z [M+H]⁺ 357.0.

### Example 96: 4-((1-(4-chloro-2-hydroxyphenyl)pyrido[3,4-d]pyridazin-4-yl)amino)bicyclo[2.2.1]heptan-1-ol

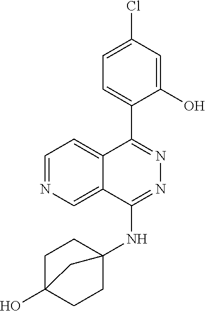 [SMILES: Oc1cc(Cl)ccc1-c1nnc(NC23CCC(O)(CC2)C3)c2cnccc12]

The title compound was prepared like Example 94, using 4-((1-chloropyrido[3,4-d]pyridazin-4-yl)amino)bicyclo[2.2.1]heptan-1-ol (18 mg, 0.0609 mmol) and (4-chloro-2-hydroxyphenyl)boronic acid (26 mg, 0.152 mmol), and was obtained as a yellow solid (14 mg, 60%). ¹H NMR (400 MHz, DMSO-d₆) δ ppm 1.59-1.68 (m, 2H), 1.72-1.83 (m, 2H), 2.05 (s, 2H), 2.10-2.25 (m, 4H), 4.95 (s, 1H), 6.98-7.07 (m, 2H), 7.27 (d, J=5.77 Hz, 1H), 7.33-7.39 (m, 1H), 7.75 (s, 1H), 8.83 (br d, J=5.52 Hz, 1H), 9.79 (br s, 1H), 10.22 (s, 1H); ESI-MS m/z [M+H]⁺ 383.1.

### Example 97: 4-(4-chlorophenyl)-N-(1-methylpiperidin-3-yl)-5,6,7,8-tetrahydropyrido[3,4-d]pyridazin-1-amine

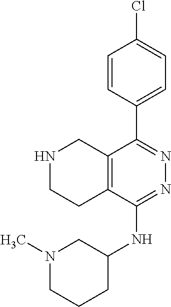 [SMILES: CN1CCCC(Nc2nnc(-c3ccc(Cl)cc3)c3c2CCNC3)C1]

To a mixture of 4-(4-chlorophenyl)-N-(1-methylpiperidin-3-yl)pyrido[3,4-d]pyridazin-1-amine (100 mg, 0.283) in HOAc (2 mL) and THE (2 mL) at 0° C. was slowly added NaBH₄ (0.095 g, 2.51 mmol). The mixture was stirred at 5° C. for 0.5 hours and then filtered, and the filtrate was purified by preparative HPLC (Method B). The title compound was obtained as a yellow solid (60 mg, 69.8%). ESI-MS m/z [M+H]⁺ 358.1.

### Example 98: 1-(4-chlorophenyl)-N-(1-methylpiperidin-3-yl)-5,6,7,8-tetrahydropyrido[3,4-d]pyridazin-4-amine

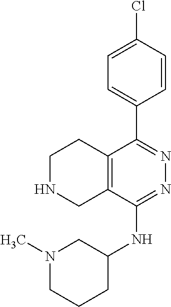 [SMILES: CN1CCCC(Nc2nnc(-c3ccc(Cl)cc3)c3c2CNCC3)C1]

A mixture of 1-(4-chlorophenyl)-N-(1-methylpiperidin-3-yl)pyrido[3,4-d]pyridazin-4-amine (400 mg, 1.12 mmol, 99% purity) and NaBH₄ (261.23 mg, 6.90 mmol) in HOAc (7 mL) was stirred at 25° C. for 2 hours. LC-MS showed the starting material was completely consumed. The mixture was dissolved in H₂O (15 mL), adjusted pH to 7 with NaHCO₃ and extracted with DCM (20 mL×3). The organic layers were combined and evaporated to dryness. The residue was purified by column chromatography (SiO₂), using a gradient of 0 to 10% DCM in MeOH. The title compound was obtained as a white solid (190 mg, 43.6%). ESI-MS m/z [M+H]+ 358.2.

### Example 99: 4-(4-chlorophenyl)-6-methyl-N-(1-methylpiperidin-3-yl)-5,6,7,8-tetrahydropyrido[3,4-d]pyridazin-1-amine

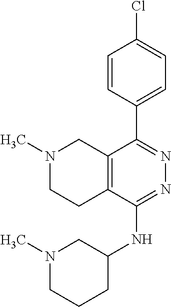 [SMILES: CN1CCc2c(NC3CCCN(C)C3)nnc(-c3ccc(Cl)cc3)c2C1]

To a mixture of 4-(4-chlorophenyl)-N-(1-methylpiperidin-3-yl)-5,6,7,8-tetrahydropyrido[3,4-d]pyridazin-1-amine (25 mg, 69.86 μmol) in DCM (1 mL) were added NaBH₃CN (6.58 mg, 104.78 μmol) and formaldehyde (56.69 mg, 698.56 μmol, 52.01 μL, 37% purity). The mixture was stirred at 20° C. for 2 hours and then filtered, and the filtrate was purified by preparative HPLC (Method B). The title compound was obtained as a yellow solid (7.1 mg, 70%). ¹H NMR (400 MHz, DMSO-d₆) δ ppm 1.35-1.49 (m, 2H), 1.55 (br dd, J=10.07, 3.31 Hz, 1H), 1.70 (br dd, J=9.19, 4.06 Hz, 1H), 1.81-2.05 (m, 4H), 2.19 (s, 3H), 2.28 (s, 3H), 2.60 (br d, J=11.88 Hz, 1H), 2.67 (br d, J=5.13 Hz, 2H), 2.90 (br d, J=9.63 Hz, 1H), 3.23-3.26 (m, 2H), 4.22-4.32 (m, 1H), 5.72 (br d, J=7.88 Hz, 1H), 7.47-7.55 (m, 4H); ESI-MS m/z [M+H]⁺ 372.4.

### Example 100: 4-(4-chlorophenyl)-6-ethyl-N-(1-methylpiperidin-3-yl)-5,6,7,8-tetrahydropyrido[3,4-d]pyridazin-1-amine

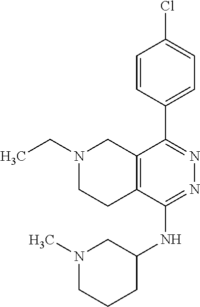 [SMILES: CCN1CCc2c(NC3CCCN(C)C3)nnc(-c3ccc(Cl)cc3)c2C1]

The title compound was prepared like Example 99, using 4-(4-chlorophenyl)-N-(1-methylpiperidin-3-yl)pyrido[3,4-d]pyridazin-1-amine (250 mg, 706.52 μmol) and acetaldehyde (305.2 mg, 6.91 mmol, 391.2 μL), and was obtained as a yellow oil (32 mg, 12%). ¹H NMR (400 MHz, DMSO-d₆) δ ppm 0.97 (t, J 7.13 Hz, 3H), 1.35-1.47 (m, 1H), 1.49-1.61 (m, 1H), 1.64-1.74 (m, 1H), 1.85 (br dd, J=11.82, 3.31 Hz, 3H), 2.15-2.21 (m, 3H), 2.42-2.50 (m, 4H), 2.55-2.63 (m, 1H), 2.68-2.74 (m, 2H), 2.86-2.94 (m, 1 H), 3.25-3.32 (m, 2H), 4.18-4.37 (m, 1H), 5.59-5.84 (m, 1H), 7.38-7.60 (m, 4H); ESI-MS m/z [M+H]+ 386.2.

### Example 101: 1-(4-chlorophenyl)-6-methyl-N-(1-methylpiperidin-3-yl)-5,6,7,8-tetrahydropyrido[3,4-d]pyridazin-4-amine

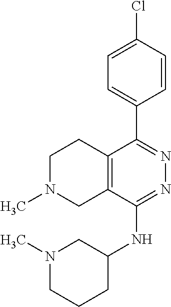 [SMILES: CN1CCc2c(-c3ccc(Cl)cc3)nnc(NC3CCCN(C)C3)c2C1]

The title compound was prepared like Example 99, using 1-(4-chlorophenyl)-N-(1-methyl-3-piperidyl)-5,6,7,8-tetrahydropyrido[3,4-d]pyridazin-4-amine (50 mg, 139.71 μmol), and was obtained as a yellow solid (23 mg, 39%). ¹H NMR (400 MHz, DMSO-d₆) δ ppm 1.34-1.49 (m, 1H), 1.51-1.63 (m, 1H), 1.68-1.77 (m, 1H), 1.83-1.92 (m, 1H), 1.94-2.06 (m, 2H), 2.19-2.26 (m, 3H), 2.40 (s, 3H), 2.58-2.63 (m, 2H), 2.65-2.74 (m, 1H), 2.95-3.03 (m, 1H), 3.24-3.32 (m, 2H), 3.55-3.65 (m, 1H), 4.24-4.33 (m, 1H), 5.67-5.79 (m, 1H), 7.41-7.61 (m, 4H), 8.13-8.29 (m, 1H); ESI-MS m/z [M+H]+ 372.4.

### Example 102: 1-(4-chlorophenyl)-6-ethyl-N-(1-methylpiperidin-3-yl)-5,6,7,8-tetrahydropyrido[3,4-d]pyridazin-4-amine

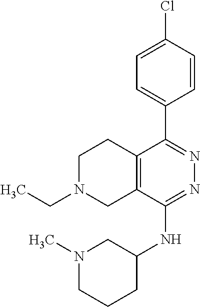 [SMILES: CCN1CCc2c(-c3ccc(Cl)cc3)nnc(NC3CCCN(C)C3)c2C1]

The title compound was prepared like Example 99, using 1-(4-chlorophenyl)-N-(1-methyl-3-piperidyl)-5,6,7,8-tetrahydropyrido[3,4-d]pyridazin-4-amine (90 mg, 251.48 μmol) and acetaldehyde (110.78 mg, 2.51 mmol, 141.13 μL), and was obtained as a yellow solid (40 mg, 41% yield, 99% purity). ¹H NMR (400 MHz, DMSO-d₆) δ ppm 1.06-1.21 (m, 3H), 1.34-1.52 (m, 1H), 1.55-1.69 (m, 1H), 1.72-1.83 (m, 1H), 1.86-1.96 (m, 1H), 2.05-2.19 (m, 2H), 2.27-2.39 (m, 3H), 2.53-2.64 (m, 6H), 2.81 (br d, J=11.01 Hz, 1H), 3.04-3.15 (m, 1H), 3.24-3.38 (m, 2H), 4.29-4.42 (m, 1H), 5.78-5.90 (m, 1H), 7.44-7.62 (m, 4H), 8.12-8.30 (m, 1H); ESI-MS m/z [M+H]⁺ 386.4.

### Example 103: 1-(4-chlorophenyl)-6-cyclopropyl-N-(1-methylpiperidin-3-yl)-5,6,7,8-tetrahydropyrido[3,4-d]pyridazin-4-amine

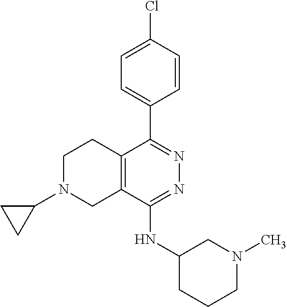 [SMILES: CN1CCCC(Nc2nnc(-c3ccc(Cl)cc3)c3c2CN(C2CC2)CC3)C1]

To a solution of 1-(4-chlorophenyl)-N-(1-methyl-3-piperidyl)-5,6,7,8-tetrahydropyrido[3,4-d]pyridazin-4-amine (90 mg, 251.48 μmol), (1-ethoxycyclopropoxy)trimethylsilane (219.18 mg, 1.26 mmol, 252.80 μL) and NaBH₃CN (23.71 mg, 377.22 μmol) in MeOH (4 mL) was added HOAc (30.20 mg, 502.96 μmol, 28.77 μL). The mixture was stirred at 25° C. for 12 hours and then filtered, and the residue was purified by preparative HPLC (Method B). The title compound was obtained as a white solid (20.3 mg, 20% yield, 98.6% purity). ¹H NMR (400 MHz, DMSO-d₆) δ ppm 0.38-0.46 (m, 2H), 0.49-0.59 (m, 2H), 1.36-1.48 (m, 1H), 1.50-1.62 (m, 1H), 1.68-1.77 (m, 1H), 1.84-1.97 (m, 4H), 2.22 (s, 3H), 2.55 (br s, 2H), 2.66-2.69 (m, 1H), 2.71-2.77 (m, 2H), 2.94-2.99 (m, 1H), 3.47-3.48 (m, 1H), 3.49 (br s, 2H), 4.25-4.38 (m, 1H), 5.74-5.87 (m, 1H), 7.43-7.62 (m, 4H), 8.17-8.25 (m, 1H); ESI-MS m/z [M+H]⁺ 398.4.

### Example 104: 1-(4-(4-chlorophenyl)-1-((1-methylpiperidin-3-yl)amino)-7,8-dihydropyrido[3,4-d]pyridazin-6(5H)-yl)ethan-1-one

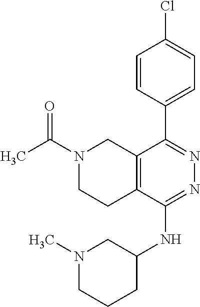 [SMILES: CC(=O)N1CCc2c(NC3CCCN(C)C3)nnc(-c3ccc(Cl)cc3)c2C1]

To a mixture of 4-(4-chlorophenyl)-N-(1-methyl-3-piperidyl)-5,6,7,8-tetrahydropyrido[3,4-d]pyridazin-1-amine (20 mg, 55.88 μmol) in DCM (1 mL) were added Ac₂O (7.13 mg, 69.86 μmol, 6.54 μL) and Et₃N (14.14 mg, 139.71 μmol, 19.45 μL). The mixture was stirred at 20° C. for 2 hours and then filtered. The filtrate was purified by preparative HPLC (Method B) to give the title compound (17.6 mg, 76.8%). ¹H NMR (400 MHz, DMSO-d₆) δ ppm 1.43 (br d, J=11.38 Hz, 1H), 1.50-1.61 (m, 1H), 1.69 (br s, 1H), 1.80-1.99 (m, 4H), 2.06 (s, 3H), 2.19 (s, 3H), 2.60 (br t, J=5.57 Hz, 2H), 2.90 (br s, 1H), 3.70-3.80 (m, 2H), 4.28 (br s, 1H), 4.41 (br s, 2H), 5.83 (br d, J=7.63 Hz, 1H), 7.50-7.59 (m, 4H); ESI-MS m/z [M+H]⁺ 400.4.

### Example 105: 1-(1-(4-chlorophenyl)-4-((1-methylpiperidin-3-yl)amino)-7,8-dihydropyrido[3,4-d]pyridazin-6(5H)-yl)ethan-1-one

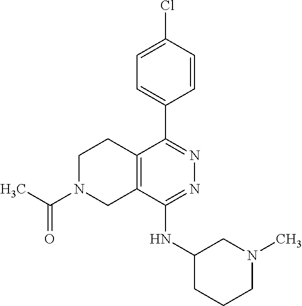 [SMILES: CC(=O)N1CCc2c(-c3ccc(Cl)cc3)nnc(NC3CCCN(C)C3)c2C1]

The title compound was prepared like Example 104, using 1-(4-chlorophenyl)-N-(1-methyl-3-piperidyl)-5,6,7,8-tetrahydropyrido[3,4-d]pyridazin-4-amine (70 mg, 195.60 μmol), and was obtained as a white solid (40 mg, 45%). ¹H NMR (400 MHz, DMSO-d₆) δ ppm 1.42-1.81 (m, 3H), 1.85-2.05 (m, 3H), 2.09-2.18 (m, 3H), 2.23-2.27 (m, 3H), 2.54-2.59 (m, 1H), 2.64-2.78 (m, 3H), 2.99 (br d, J=8.63 Hz, 1H), 3.57-3.59 (m, 2H), 4.33-4.43 (m, 3H), 5.96-6.00 (m, 1H), 7.51-7.58 (m, 4H), 8.13-8.22 (m, 1H); ESI-MS m/z [M+H]+ 400.3.

### Example 106: 4-(4-chlorophenyl)-N-(1-methylpiperidin-3-yl)-6-(methylsulfonyl)-5,6,7,8-tetrahydropyrido[3,4-d]pyridazin-1-amine

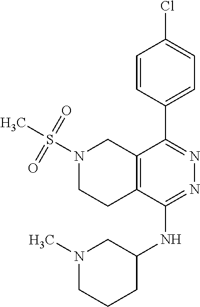 [SMILES: CN1CCCC(Nc2nnc(-c3ccc(Cl)cc3)c3c2CCN(S(C)(=O)=O)C3)C1]

To a solution of 4-(4-chlorophenyl)-N-(1-methyl-3-piperidyl)-5,6,7,8-tetrahydropyrido[3,4-d]pyridazin-1-amine (25 mg, 69.86 μmol) in DCM (1 mL) were added methanesulfonyl chloride (0.040 g, 349.19 μmol, 27.03 μL) dropwise at 0° C. followed by DIPEA (13.54 mg, 104.78 μmol, 18.25 μL). The mixture was stirred at 20° C. for 1 hour and then filtered, and the filtrate was purified by preparative HPLC (Method B). The title compound was obtained as a white solid (2.1 mg, 47%). ¹H NMR (400 MHz, DMSO-d₆) δ ppm 1.39-1.48 (m, 1H), 1.56 (br d, J=10.88 Hz, 1H), 1.68-1.75 (m, 1H), 1.83-2.03 (m, 4H), 2.20 (s, 3H), 2.63 (br t, J=5.75 Hz, 2H), 2.92 (br d, J=10.26 Hz, 1H), 2.96 (s, 3H), 3.51 (br t, J=5.82 Hz, 2H), 4.15 (s, 2H), 4.24-4.34 (m, 1H), 5.88 (d, J=8.00 Hz, 1H), 7.54 (d, J=2.00 Hz, 4H); ESI-MS m/z [M+H]⁺ 436.3.

### Example 107: 1-(4-chlorophenyl)-N-(1-methylpiperidin-3-yl)-6-(methylsulfonyl)-5,6,7,8-tetrahydropyrido[3,4-d]pyridazin-4-amine

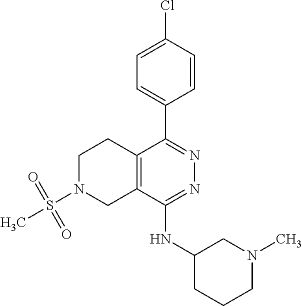

The title compound was prepared like Example 106, using 1-(4-chlorophenyl)-N-(1-methyl-3-piperidyl)-5,6,7,8-tetrahydropyrido[3,4-d]pyridazin-4-amine (40.00 mg, 111.77 μmol), and was obtained as a white solid (6.7 mg, 24%). ¹H NMR (400 MHz, DMSO-d₆) δ ppm 1.17-1.49 (m, 2H), 1.51-1.61 (m, 1H), 1.67-1.77 (m, 1H), 1.90 (br t, J=8.32 Hz, 4H), 2.19 (s, 3H), 2.66 (br d, J=10.63 Hz, 1H), 2.73 (br s, 2H), 2.97 (br d, J=10.38 Hz, 1H), 3.01 (s, 3H), 4.14 (s, 2H), 4.25-4.37 (m, 1H), 5.99 (br d, J=7.63 Hz, 1H), 7.52 (m, J=8.38 Hz, 2H), 7.58 (m, J=8.38 Hz, 2H); ESI-MS m/z [M+H]⁺ 436.2.

### Example 108: 4-(4-chlorophenyl)-N-(5,5-difluoro-1-methylpiperidin-3-yl)phthalazin-1-amine

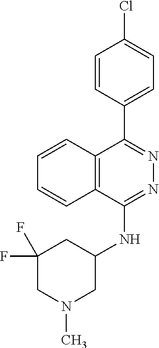 [SMILES: CN1CC(Nc2nnc(-c3ccc(Cl)cc3)c3ccccc23)CC(F)(F)C1]

A mixture of 5,5-difluoro-1-methylpiperidin-3-amine (150 mg, 998.87 μmol), 1-chloro-4-(4-chlorophenyl)phthalazine (91.61 mg, 332.96 μmol) and DIPEA (1.85 g, 14.35 mmol, 2.50 mL) in NMP (2 mL) was stirred at 120° C. for 12 hours. The reaction mixture was diluted with MeOH and purified by preparative HPLC (Method B). The title compound was obtained as a yellow solid (7.5 mg, 5.5%). ¹H NMR (400 MHz, CD₃Cl) 6 ppm 1.92-2.15 (m, 1H), 2.30-2.48 (m, 5H), 2.63 (br s, 1H), 3.09 (br d, J=12.6 Hz, 2H), 5.05 (br s, 1H), 6.05 (br s, 1H), 7.51 (d, J=8.4 Hz, 2H), 7.66 (d, J=8.3 Hz, 2H), 7.74-7.86 (m, 2H), 7.87-7.97 (m, 2H); ESI-MS m/z [M+H]⁺ 389.1.

### Example 109: 3-((4-(4-chlorophenyl)phthalazin-1-yl)amino)-1-methylpyrrolidin-2-one

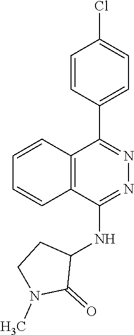 [SMILES: CN1CCC(Nc2nnc(-c3ccc(Cl)cc3)c3ccccc23)C1=O]

The title compound was prepared like Example 108, using 1-chloro-4-(4-chlorophenyl)phthalazine (50 mg, 0.182 mmol) and 3-amino-1-methylpyrrolidin-2-one (22.3 mg, 0.2 mmol), and was obtained as a clear oil (9.3 mg, 11%)¹H NMR (400 MHz, CD₃OD) δ ppm 2.34-2.47 (m, 1H), 2.69-2.81 (m, 1H), 2.99 (s, 3H), 3.62 (s, 2H), 5.00-5.09 (m, 1H), 7.72 (d, J=7.15 Hz, 4H), 8.12 (d, J=0.88 Hz, 1H), 8.19 (s, 2H), 8.63-8.68 (m, 1H); ESI-MS m/z [M+H]+ 353.1.

### Example 110: 1-(3-((4-(4-chlorophenyl)phthalazin-1-yl)amino)piperidin-1-yl)ethan-1-one

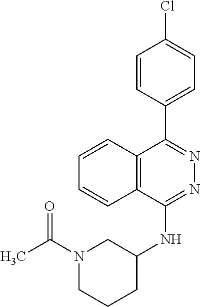 [SMILES: CC(=O)N1CCCC(Nc2nnc(-c3ccc(Cl)cc3)c3ccccc23)C1]

The title compound was prepared like Example 108, using 1-chloro-4-(4-chlorophenyl)phthalazine (50 mg, 0.182 mmol) and 3-amino-1-methylpyrrolidin-2-one (22.3 mg, 0.2 mmol), and was obtained as a colorless film (70.6 mg, 78%). ¹H NMR (400 MHz, CD₃OD) δ ppm 1.79 (br dd, J=11.48, 2.95 Hz, 1H), 1.90-2.24 (m, 1H), 2.30-2.38 (m, 1H), 3.29 (br d, J=12.92 Hz, 1H), 3.52 (br s, 1H), 3.65 (s, 4H), 4.09-4.24 (m, 1H), 7.64-7.74 (m, 4H), 8.04-8.11 (m, 1H), 8.13-8.20 (m, 2H), 8.69 (br d, J=6.15 Hz, 1H); ESI-MS m/z [M+H]+ 381.2.

### Example 111: 3-((4-(4-chlorophenyl)phthalazin-1-yl)amino)-1-methylpiperidin-2-one

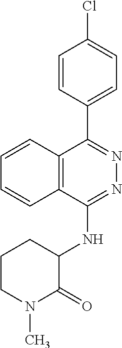 [SMILES: CN1CCCC(Nc2nnc(-c3ccc(Cl)cc3)c3ccccc23)C1=O]

The title compound was prepared like Example 108, using 1-chloro-4-(4-methoxyphenyl)phthalazine (0.05 g, 0.182 mmol) and 3-amino-1-methylpiperidin-2-one hydrochloride (32.9 mg, 0.200 mmol), and was obtained as a colorless film (70.6 mg, 78%). H NMR (400 MHz, CD₃OD) δ ppm 2.14-2.23 (m, 2H), 2.75 (s, 3H), 2.99-3.06 (m, 3H), 3.11-3.16 (m, 2H), 7.69 (s, 2H), 7.75-7.79 (m, 2H), 7.83-7.89 (m, 1H), 7.98-8.03 (m, 1H), 8.08 (s, 1H), 8.45-8.50 (m, 1H); ESI-MS m/z [M+H]+ 367.15.

### Example 112: 4-(4-chlorophenyl)-N-methyl-N-(1-methylpiperidin-3-yl)phthalazin-1-amine

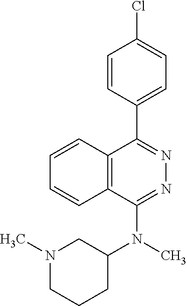

A solution of 1-chloro-4-(4-chlorophenyl)phthalazine (91 mg, 0.330 mmol), N,1-dimethylpiperidin-3-amine (38 mg, 0.300 mmol), DMSO (1.5 mL) and aq K₂CO₃ (124 mg, 0.900 mmol) was stirred at 100° C. overnight and then diluted with water and saturated aq NaHCO₃. The organic phase was extracted EtOAc (3×) and washed with brine. The residue was purified by flash column chromatography (ISCO®, 60 g half-amine column) using a gradient of 0-100% EtOAc in heptanes. The product-containing fractions, and another fraction with a peak having the same mass, were collected and concentrated under reduced pressure. The residue was taken up in MeOH and filtered through a hydrophilic PTFE 0.45 m syringe filter (VWR®), rinsing with MeOH. The filtrate was purified by preparative HPLC (Shimadzu, Phenomenex Gemini NX-C18, 5 μm, ID 30 mm×100 mm) using a gradient of 10-60% ACN (0.035% TFA) in water (0.05% TFA). The first peak to elute was collected and lyophilized to give the title compound as a light-yellow film (0.8 mg, 0.7%). ¹H NMR (400 MHz, CD₃CN) 6 ppm 2.07-2.30 (m, 4H), 2.89 (s, 3H), 2.90-2.98 (m, 1H), 3.28 (m, 1H) 3.42 (s, 3H), 3.52 (br d, J=12.0 Hz, 1H) 3.87 (br d, J=11.3 Hz, 1H) 4.69 (tt, J=11.5, 3.6 Hz, 1H), 7.69 (s, 4H), 8.02-8.17 (m, 3H), 8.42-8.54 (m, 1H), 11.30-11.67 (m, 1H); ESI-MS m/z [M+H]f 367.20.

### Example 113: 4-(4-chlorophenyl)-N-(1-methylpiperidin-3-yl)phthalazin-1-amine

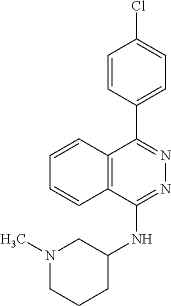 [SMILES: CN1CCCC(Nc2nnc(-c3ccc(Cl)cc3)c3ccccc23)C1]

A TFA salt of the title compound was prepared like Example 112, using 1-chloro-4-(4-chlorophenyl)phthalazine (60 mg, 0.218 mmol) and 1-methylpiperidin-3-amine dihydrochloride (53 mg, 0.284 mmol), and was obtained as a light-brown solid (42 mg, 55%). ¹H NMR (400 MHz, CD₃OD) δ ppm 1.76-1.92 (m, 1H), 1.92-2.08 (m, 1H), 2.12-2.28 (m, 1H), 2.28-2.43 (m, 1H), 2.84-3.01 (m, 3H), 3.01-3.10 (m, 1H), 3.52-3.67 (m, 1H), 3.83-3.98 (m, 1H), 4.55-4.72 (m, 1H), 7.63-7.82 (m, 3H), 7.98-8.19 (m, 2H), 8.21-8.32 (m, 1H), 8.62 (br d, J=7.52 Hz, 1H), 8.69-8.93 (m, 1H); ESI-MS m/z [M+H]⁺ 353.1.

### Example 114: cis-3-((4-(4-chlorophenyl)phthalazin-1-yl)amino)-1-(trifluoromethyl)cyclohexanol (racemic)

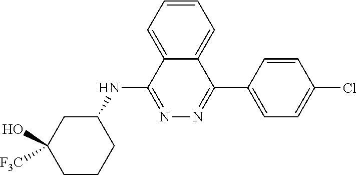 [SMILES: O[C@]1(C(F)(F)F)CCC[C@@H](Nc2nnc(-c3ccc(Cl)cc3)c3ccccc23)C1]

The title compound was prepared like Example 112, using cis-3-amino-1-(trifluoromethyl)cyclohexanol (17 mg, 0.0909 mmol) and 1-chloro-4-(4-chlorophenyl)phthalazine (25 mg, 0.0909 mmol), and was obtained as a solid (12 mg, 25%). ¹H NMR (400 MHz, DMSO-d₆) δ ppm 1.40-1.65 (m, 2H), 1.73-2.04 (m, 4H), 2.10-2.26 (m, 2H), 4.36-4.57 (m, 1H), 6.10 (br s, 1H), 7.69-7.83 (m, 4H), 7.90-8.02 (m, 1H), 8.09-8.32 (m, 2H), 8.73-8.92 (m, 1H), 9.17-9.56 (m, 1H); ESI-MS m/z [M+H]⁺ 422.3.

### Example 115: (1R,3S)-3-((4-(4-chlorophenyl)phthalazin-1-yl)amino)-1-(trifluoromethyl)cyclohexan-1-ol

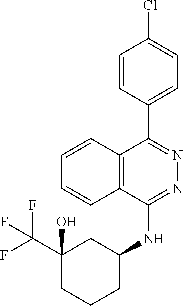 [SMILES: O[C@]1(C(F)(F)F)CCC[C@H](Nc2nnc(-c3ccc(Cl)cc3)c3ccccc23)C1]

The title compound was prepared like Example 112, using (1R,3S)-3-amino-1-(trifluoromethyl)cyclohexanol (17 mg, 0.0909 mmol) and 1-chloro-4-(4-chlorophenyl)phthalazine (25 mg, 0.0909 mmol), and was obtained as a solid (12 mg, 25%). ¹H NMR (400 MHz, DMSO-d₆) δ ppm 1.54-1.70 (m, 1H), 1.73-2.02 (m, 5H), 2.04-2.20 (m, 2H), 4.50 (br s, 1H), 6.55-6.78 (m, 1H), 7.63-7.79 (m, 4H), 7.89-8.00 (m, 1H), 8.07-8.28 (m, 2H), 8.39-8.56 (m, 1H), 8.78-9.20 (m, 1H); ESI-MS m/z [M+H]⁺ 422.3.

### Example 116: (1R,3R)-3-((4-(4-chlorophenyl)phthalazin-1-yl)amino)-1-(trifluoromethyl)cyclohexan-1-ol

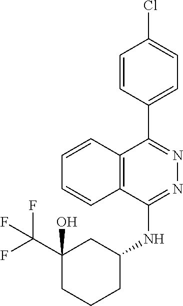 [SMILES: O[C@]1(C(F)(F)F)CCC[C@@H](Nc2nnc(-c3ccc(Cl)cc3)c3ccccc23)C1]

The title compound was prepared like Example 112, using (1R,3R)-3-amino-1-(trifluoromethyl)cyclohexanol (17 mg, 0.0909 mmol) and 1-chloro-4-(4-chlorophenyl)phthalazine (25 mg, 0.0909 mmol), and was obtained as a solid (9.0 mg, 19%). ¹H NMR (400 MHz, DMSO-d₆) δ ppm 1.35-1.61 (m, 2H), 1.65-2.01 (m, 4H), 2.15 (br d, J=9.54 Hz, 2H), 4.30-4.54 (m, 1H), 5.97-6.22 (m, 1H), 7.66-7.81 (m, 4H), 7.85-7.98 (m, 1H), 8.07-8.28 (m, 2H), 8.69-8.88 (m, 1H), 9.14-9.50 (m, 1H); ESI-MS m/z [M+H]+ 422.2.

### Example 117: trans-3-((4-(4-chlorophenyl)phthalazin-1-yl)amino)-1-(trifluoromethyl)cyclobutan-1-ol

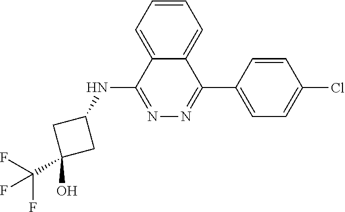 [SMILES: O[C@]1(C(F)(F)F)C[C@@H](Nc2nnc(-c3ccc(Cl)cc3)c3ccccc23)C1]

The title compound was prepared like Example 112, using trans-3-amino-1-(trifluoromethyl)cyclobutan-1-ol hydrochloride (17 mg, 0.0909 mmol) and 1-chloro-4-(4-chlorophenyl)phthalazine (25 mg, 0.0909 mmol), and was obtained as a solid (9.2 mg, 20%). ¹H NMR (400 MHz, DMSO-d₆) δ ppm 2.53-2.75 (m, 4H), 4.60-4.81 (m, 1H), 6.53-6.77 (m, 1H), 7.56-7.71 (m, 4H), 7.85 (d, J=7.91 Hz, 1H), 7.98-8.21 (m, 2H), 8.71 (br d, J=8.03 Hz, 1H), 9.34-9.71 (m, 1H); ESI-MS m/z [M+H]+ 394.2.

### Example 118: cis-3-((4-(4-chlorophenyl)phthalazin-1-yl)amino)-1-methylcyclobutan-1-ol

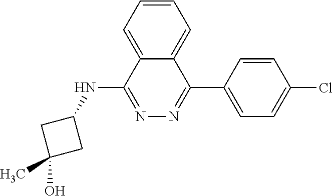 [SMILES: C[C@]1(O)C[C@@H](Nc2nnc(-c3ccc(Cl)cc3)c3ccccc23)C1]

The title compound was prepared like Example 112, using cis-3-amino-1-methylcyclobutan-1-ol hydrochloride (13 mg, 0.0909 mmol) and 1-chloro-4-(4-chlorophenyl)phthalazine (25 mg, 0.0909 mmol), and was obtained as a solid (13 mg, 32%). ¹H NMR (400 MHz, DMSO-d₆) δ ppm 1.30-1.39 (m, 3H), 2.26-2.40 (m, 2H), 2.58-2.71 (m, 2H), 4.03-4.22 (m, 1H), 5.11-5.46 (m, 1H), 7.66-7.79 (m, 5H), 7.83-7.99 (m, 1H), 8.04-8.30 (m, 2H), 8.86 (d, J=7.91 Hz, 1H), 9.77-10.22 (m, 1H), 14.00-14.47 (m, 1H); ESI-MS m/z [M+H]⁺ 340.2.

### Example 119: trans-3-((4-(4-chlorophenyl)phthalazin-1-yl)amino)-1-methylcyclobutan-1-ol

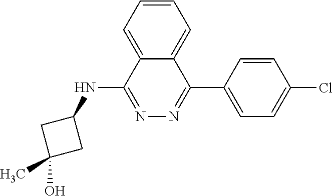 [SMILES: C[C@]1(O)C[C@H](Nc2nnc(-c3ccc(Cl)cc3)c3ccccc23)C1]

The title compound was prepared like Example 112, using trans-3-amino-1-methylcyclobutan-1-ol hydrochloride (13 mg, 0.0909 mmol) and 1-chloro-4-(4-chlorophenyl)phthalazine (0.0909 mmol), and was obtained as a solid (15 mg, 36%). ¹H NMR (400 MHz, DMSO-d₆) δ ppm 1.24-1.33 (m, 3H), 2.21-2.34 (m, 2H), 2.48-2.60 (m, 2H), 4.39-4.55 (m, 1H), 4.87-5.19 (m, 1H), 7.58-7.70 (m, 4H), 7.77-7.92 (m, 1H), 7.99-8.20 (m, 2H), 8.63-8.99 (m, 1H), 9.35-10.10 (m, 1H); ESI-MS m/z [M+H]⁺ 340.2.

### Example 120: cis-3-((4-(4-chlorophenyl)phthalazin-1-yl)amino)-1-(trifluoromethyl)cyclobutan-1-ol

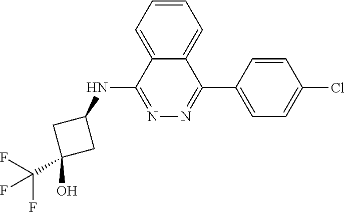 [SMILES: O[C@]1(C(F)(F)F)C[C@H](Nc2nnc(-c3ccc(Cl)cc3)c3ccccc23)C1]

The title compound was prepared like Example 112, using cis-3-amino-1-(trifluoromethyl)cyclobutan-1-ol hydrochloride (17 mg, 0.0909 mmol) and 1-chloro-4-(4-chlorophenyl)phthalazine (25 mg, 0.0909 mmol), and was obtained as a solid (7.0 mg, 15%). ¹H NMR (400 MHz, DMSO-d₆) δ ppm 2.54-2.75 (m, 2H), 2.97-3.16 (m, 2H), 4.19-4.43 (m, 1H), 6.76-7.06 (m, 1H), 7.64-7.80 (m, 4H), 7.88-7.99 (m, 1H), 8.07-8.26 (m, 2H), 8.73-8.87 (m, 1H), 9.45-9.87 (m, 1H); ESI-MS m/z [M+H]⁺ 394.1.

### Example 121: trans-4-((4-(4-chlorophenyl)phthalazin-1-yl)amino)-1-methylcyclohexan-1-ol

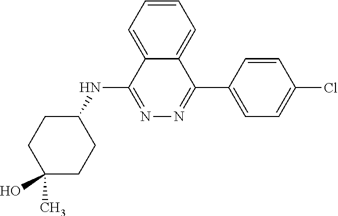 [SMILES: C[C@]1(O)CC[C@H](Nc2nnc(-c3ccc(Cl)cc3)c3ccccc23)CC1]

The title compound was prepared like Example 112, using trans-4-amino-1-methyl-cyclohexanol (12 mg, 0.0909 mmol) and 1-chloro-4-(4-chlorophenyl)phthalazine (25 mg, 0.0909 mmol), and was obtained as a solid (9.2 mg, 21%). ¹H NMR (400 MHz, DMSO-d₆) δ ppm 1.15-1.31 (m, 3H), 1.48-1.79 (m, 6H), 1.88-2.13 (m, 2H), 3.94-4.14 (m, 1H), 4.31-4.72 (m, 1H), 7.65-7.79 (m, 4H), 7.87-7.98 (m, 1H), 8.04-8.27 (m, 2H), 8.81 (br d, J=8.03 Hz, 1H), 9.31 (br s, 1H), 14.17-14.73 (m, 1H); ESI-MS m/z [M+H]⁺ 368.2.

### Example 122: cis-3-((4-(4-chlorophenyl)phthalazin-1-yl)amino)cyclobutan-1-ol

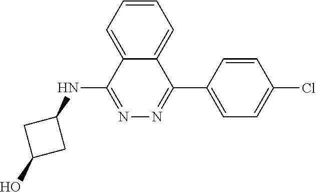 [SMILES: O[C@H]1C[C@@H](Nc2nnc(-c3ccc(Cl)cc3)c3ccccc23)C1]

The title compound was prepared like Example 112, using cis-3-aminocyclobutan-1-ol hydrochloride (11 mg, 0.0909 mmol) and 1-chloro-4-(4-chlorophenyl)phthalazine (25 mg, 0.0909 mmol), and was obtained as a solid (11 mg, 28%). ¹H NMR (400 MHz, DMSO-d₆) δ ppm 2.10-2.27 (m, 2H), 2.82-2.99 (m, 2H), 3.88-4.10 (m, 2H), 7.63-7.77 (m, 5H), 7.86-7.97 (m, 1H), 8.08-8.27 (m, 2H), 8.79-8.96 (m, 1H), 9.67-9.92 (m, 1H); ESI-MS m/z [M+H]+ 326.1.

### Example 123: 4-(4-methoxyphenyl)-N-(1-methylpiperidin-3-yl)phthalazin-1-amine

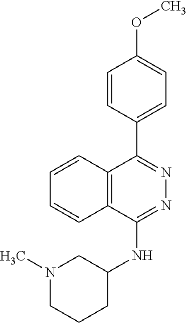

The title compound was prepared like Example 112, using 1-chloro-4-(4-methoxyphenyl)phthalazine (30 mg, 0.111 mmol) and 1-methylpiperidin-3-amine dihydrochloride (27 mg, 0.144 mmol) (17 mg, 44%). ¹H NMR (400 MHz, CD₃OD) δ ppm 1.76-1.92 (m, 1H), 1.92-2.08 (m, 1H), 2.12-2.28 (m, 1H), 2.28-2.43 (m, 1H), 2.84-3.01 (m, 3H), 3.01-3.10 (m, 1H), 3.52-3.67 (m, 1H), 3.83-3.98 (m, 1H), 4.55-4.72 (m, 1H), 7.53-7.83 (m, 4H), 7.98-8.19 (m, 2H), 8.21-8.32 (m, 1H), 8.62 (br d, J=7.52 Hz, 1H), 8.69-8.93 (m, 1H); ESI-MS m/z [M+H]⁺ 349.1.

### Example 124: cis-3-((4-(4-methoxyphenyl)phthalazin-1-yl)amino)-1-methylcyclobutan-1-ol

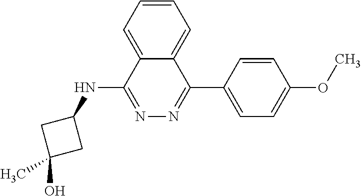

The title compound was prepared like Example 112, using cis-3-amino-1-methylcyclobutan-1-ol hydrochloride (14.23 mg, 0.103 mmol) and 1-chloro-4-(4-methoxyphenyl)phthalazine (28 mg, 0.103 mmol), and was obtained as a solid (10 mg, 22%). ¹H NMR (400 MHz, DMSO-d₆) δ ppm 1.22-1.33 (m, 3H), 2.21-2.33 (m, 2H), 2.49-2.60 (m, 2H), 3.73-3.86 (m, 3H), 3.93-4.16 (m, 1H), 7.03-7.20 (m, 2H), 7.48-7.64 (m, 2H), 7.84-7.97 (m, 1H), 8.01-8.23 (m, 2H), 8.70-8.89 (m, 1H), 9.56-9.95 (m, 1H), 13.88-14.44 (m, 1H). ESI-MS m/z [M+H]⁺ 336.2.

### Example 125: N-((1-ethylpyrrolidin-2-yl)methyl)-4-(4-methoxyphenyl)phthalazin-1-amine

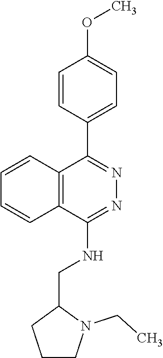 [SMILES: CCN1CCCC1CNc1nnc(-c2ccc(OC)cc2)c2ccccc12]

The title compound (29 mg, 81%) was prepared like Example 112, using 1-chloro-4-(4-methoxyphenyl)phthalazine (27 mg, 0.10 mmol) and (1-ethylpyrrolidin-2-yl)methanamine (38 mg, 0.30 mmol). ¹H NMR (400 MHz, CD₃OD) δ ppm 1.38-1.47 (m, 3H), 2.05-2.22 (m, 3H), 2.32-2.45 (m, 1H), 3.17-3.28 (m, 2H), 3.61-3.72 (m, 1H), 3.73-3.84 (m, 1H), 3.89-3.98 (m, 4H), 4.01-4.13 (m, 2H), 7.23-7.29 (m, 2H), 7.68-7.75 (m, 2H), 8.13-8.19 (m, 1H), 8.21-8.30 (m, 2H), 8.49-8.56 (m, 1H); ESI-MS m/z [M+H]363.1.

### Example 126: 4-(4-methoxyphenyl)-N-(1-methylpyrrolidin-3-yl)phthalazin-1-amine

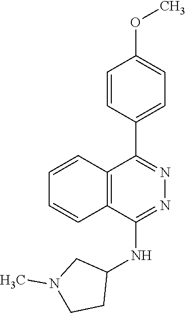

The title compound (15 mg, 45%) was prepared like Example 112, using 1-chloro-4-(4-methoxyphenyl)phthalazine (27 mg, 0.10 mmol) and 1-methylpyrrolidin-3-amine (30 mg, 0.30 mmol). ¹H NMR (400 MHz, CD₃OD) δ ppm 2.35-2.58 (m, 1H), 2.65-2.87 (m, 2H), 3.04 (s, 3H), 3.45-3.67 (m, 1H), 3.84-4.10 (m, 5H), 4.90-5.03 (m, 1H), 7.26 (d, J=7.79 Hz, 2H), 7.66-7.78 (m, 2H), 8.09-8.21 (m, 1H), 8.21-8.32 (m, 2H), 8.66 (d, J=8.39 Hz, 1H); ESI-MS m/z [M+H]+ 335.1.

### Example 127: 4-((4-(4-chlorophenyl)phthalazin-1-yl)amino)bicyclo[2.2.1]heptan-1-ol

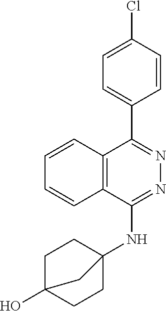 [SMILES: OC12CCC(Nc3nnc(-c4ccc(Cl)cc4)c4ccccc34)(CC1)C2]

The title compound was prepared like Example 112, using 4-aminobicyclo[2.2.1]heptan-1-ol hydrochloride (149 mg, 0.909 mmol) and 1-chloro-4-(4-chlorophenyl)phthalazine (250 mg, 0.909 mmol), and was obtained as a solid (125 mg, 37.6%). ¹H NMR (400 MHz, DMSO-d₆) δ ppm 1.57-1.70 (m, 2H), 1.71-1.85 (m, 2H), 1.95-2.27 (m, 6H), 4.91-5.05 (m, 1H), 7.22-7.37 (m, 1H), 7.55-7.69 (m, 4H), 7.73-7.79 (m, 1H), 7.80-7.96 (m, 2H), 8.40-8.56 (m, 1H); ESI-MS m/z [M+H]⁺ 366.1.

### Example 128: trans-4-((4-(4-methoxyphenyl)phthalazin-1-yl)amino)-1-(trifluoromethyl)cyclohexan-1-ol

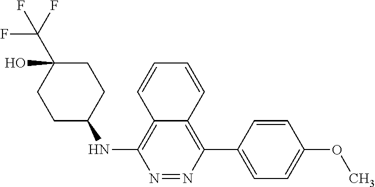 [SMILES: COc1ccc(-c2nnc(N[C@H]3CC[C@](O)(C(F)(F)F)CC3)c3ccccc23)cc1]

The title compound (12 mg, 24%) was prepared like Example 112, using trans-4-amino-1-(trifluoromethyl)cyclohexanol (16.92 mg, 0.092 mmol) and 1-chloro-4-(4-methoxyphenyl)phthalazine (25 mg, 0.092 mmol). ¹H NMR (400 MHz, DMSO-d₆) δ ppm 1.38-1.74 (m, 10H), 1.80-2.07 (m, 6H), 3.76-3.87 (m, 3H), 4.09-4.25 (m, 2H), 5.61-5.73 (m, 1H), 5.83-5.89 (m, 1H), 5.90-5.98 (m, 1H), 7.08-7.22 (m, 2H), 7.53-7.64 (m, 2H), 7.88-7.98 (m, 1H), 8.03-8.22 (m, 2H), 8.76-8.91 (m, 1H); ESI-MS m/z [M+H]⁺ 418.2.

### Example 129: 4-(4-methoxyphenyl)-N-(2-(pyrrolidin-1-yl)propyl)phthalazin-1-amine

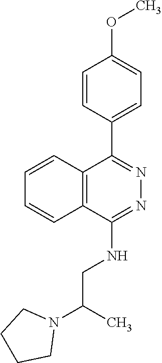 [SMILES: COc1ccc(-c2nnc(NCC(C)N3CCCC3)c3ccccc23)cc1]

The title compound was prepared like Example 112, using 1-chloro-4-(4-methoxyphenyl)phthalazine and 2-pyrrolidin-1-ylpropan-1-amine. ESI-MS m/z [M+H]⁺ 363.1.

### Example 130: 4-(4-methoxyphenyl)-N-((1-methylpiperidin-2-yl)methyl)phthalazin-1-amine

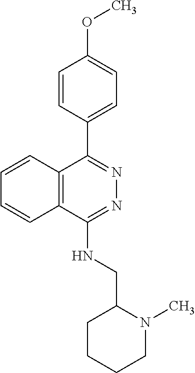 [SMILES: COc1ccc(-c2nnc(NCC3CCCCN3C)c3ccccc23)cc1]

The title compound was prepared like Example 112, using 1-chloro-4-(4-methoxyphenyl)phthalazine and (1-methylpiperidin-2-yl)methanamine. ESI-MS m/z [M+H]363.2.

### Example 131: 4-(4-methoxyphenyl)-N-((1-(pyrrolidin-1-yl)cyclopropyl)methyl)phthalazin-1-amine

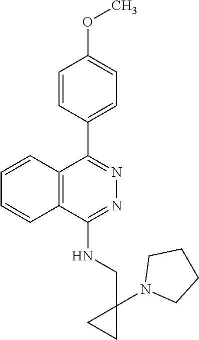 [SMILES: COc1ccc(-c2nnc(NCC3(N4CCCC4)CC3)c3ccccc23)cc1]

The title compound was prepared like Example 112, using 1-chloro-4-(4-methoxyphenyl)phthalazine and (1-(pyrrolidin-1-yl)cyclopropyl)methanamine. ESI-MS m/z [M+H]+ 375.1.

### Example 132: N-(1-(azetidin-1-ylmethyl)cyclopropyl)-4-(4-methoxyphenyl)phthalazin-1-amine

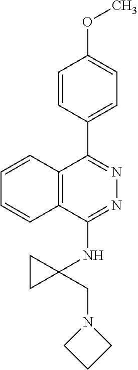 [SMILES: COc1ccc(-c2nnc(NC3(CN4CCC4)CC3)c3ccccc23)cc1]

The title compound was prepared like Example 112, using 1-chloro-4-(4-methoxyphenyl)phthalazine and 1-(azetidin-1-ylmethyl)cyclopropan-1-amine. ESI-MS m/z [M+H]+ 361.1.

### Example 133: 6-((4-(4-chlorophenyl)phthalazin-1-yl)amino)spiro[3.3]heptan-2-ol

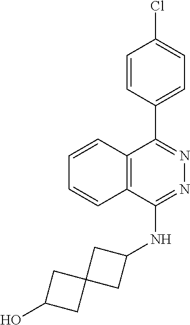 [SMILES: OC1CC2(C1)CC(Nc1nnc(-c3ccc(Cl)cc3)c3ccccc13)C2]

A TFA salt of the title compound was prepared like Example 112, using 6-aminospiro[3.3]heptan-2-ol hydrochloride (14.87 mg, 0.091 mmol) and 1-chloro-4-(4-chlorophenyl)phthalazine (25 mg, 0.091 mmol), and was obtained as a mixture of diastereomers (24.6 mg, 56.4%). ¹H NMR (400 MHz, DMSO-d₆) δ ppm 1.77-1.92 (m, 2H), 2.11-2.30 (m, 3H), 2.32-2.40 (m, 1H), 2.50-2.63 (m, 1H), 3.89-4.03 (m, 1H), 4.24-4.43 (m, 1H), 4.80-5.07 (m, 1H), 7.55-7.70 (m, 4H), 7.79-7.90 (m, 1H), 7.98-8.17 (m, 2H), 8.65-8.82 (m, 1H), 9.60-9.86 (m, 1H); ESI-MS m/z [M+H]⁺ 366.2.

### Example 134: 4-(4-chlorophenyl)-N-(cis-3-methoxycyclobutyl)phthalazin-1-amine

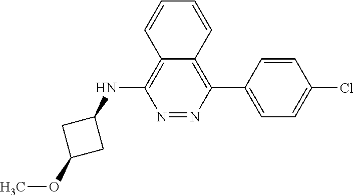 [SMILES: CO[C@H]1C[C@@H](Nc2nnc(-c3ccc(Cl)cc3)c3ccccc23)C1]

The title compound (21 mg, 51%) was prepared like Example 112, using cis-3-methoxycyclobutan-1-amine hydrochloride (12.50 mg, 0.091 mmol) and 1-chloro-4-(4-chlorophenyl)phthalazine (25 mg, 0.091 mmol). ¹H NMR (400 MHz, DMSO-d₆) δ ppm 2.15-2.28 (m, 2H), 2.87-3.01 (m, 2H), 3.21 (s, 3H), 3.68-3.81 (m, 1H), 4.07-4.21 (m, 1H), 7.64-7.78 (m, 4H), 7.84-7.99 (m, 1H), 8.06-8.25 (m, 2H), 8.72-8.90 (m, 1H), 9.56-9.95 (m, 1H); ESI-MS m/z [M+H]⁺ 340.1.

### Example 135: 4-(4-chlorophenyl)-N-(3-methoxy-2,2,3-trimethylcyclobutyl)phthalazin-1-amine

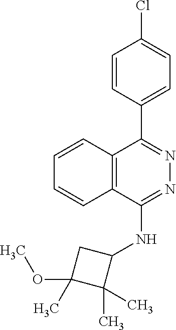 [SMILES: COC1(C)CC(Nc2nnc(-c3ccc(Cl)cc3)c3ccccc23)C1(C)C]

The title compound was prepared like Example 112, using 3-methoxy-2,2,3-trimethylcyclobutan-1-amine (13.01 mg, 0.091 mmol) and 1-chloro-4-(4-chlorophenyl)phthalazine (25 mg, 0.091 mmol), and was obtained as a solid (1.2 mg, 2.7%). ESI-MS m/z [M+H]+ 382.4.

### Example 136: 4-(4-chlorophenyl)-N-(1-methyl-2-oxabicyclo[2.2.1]heptan-4-yl)phthalazin-1-amine

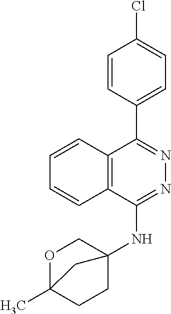 [SMILES: CC12CCC(Nc3nnc(-c4ccc(Cl)cc4)c4ccccc34)(CO1)C2]

The title compound was prepared like Example 112, using 1-methyl-2-oxabicyclo[2.2.1]heptan-4-amine hydrochloride (14.87 mg, 0.091 mmol) and 1-chloro-4-(4-chlorophenyl)phthalazine (25 mg, 0.091 mmol), and was obtained as a solid (2 mg, 5%). ESI-MS m/z [M+H]+ 366.3.

### Example 137: 3-((4-(4-methoxyphenyl)phthalazin-1-yl)amino)-1-methylcyclohexan-1-ol

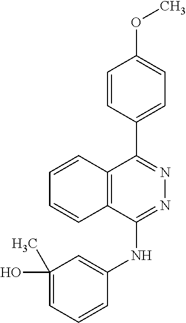

The title compound was prepared like Example 112, using 1-chloro-4-(4-methoxyphenyl)phthalazine (100 mg, 0.369 mmol) and 3-amino-1-methylcyclohexan-1-ol hydrochloride (1.00 eq, 61 mg, 0.369 mmol), and was obtained as an off-white solid (36 mg, 27%). ¹H NMR (400 MHz, CD₃OD) δ ppm 1.30-1.32 (m, 3H), 1.40-1.56 (m, 2H), 1.59-1.66 (m, 1H), 1.70-1.77 (m, 2H), 1.88-2.00 (m, 1H), 2.10-2.21 (m, 2H), 3.89-3.92 (m, 3H), 4.14-4.38 (m, 1H), 7.13-7.19 (m, 2H), 7.59-7.65 (m, 2H), 8.08-8.16 (m, 3H) 8.63-8.70 (m, 1H); ESI-MS m/z [M+H]+ 364.5.

### Example 138: 3-((4-(4-chlorophenyl)phthalazin-1-yl)amino)-1-methylcyclohexan-1-ol

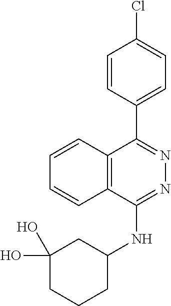 [SMILES: OC1(O)CCCC(Nc2nnc(-c3ccc(Cl)cc3)c3ccccc23)C1]

The title compound was prepared like Example 112, using 1-chloro-4-(4-chlorophenyl)phthalazine (100 mg, 0.363 mmol) and 3-amino-1-methylcyclohexan-1-ol hydrochloride (60 mg, 0.363 mmol), and was obtained as an off-white solid (37 mg, 28%). ¹H NMR (400 MHz, DMSO-d₆) δ ppm 1.14-1.18 (m, 3H), 1.18-1.29 (m, 2H), 1.42-1.50 (m, 1H), 1.50-1.61 (m, 2H), 1.70-1.83 (m, 1H), 1.91-1.97 (m, 1H), 2.06-2.13 (m, 1H), 4.18-4.20 (m, 1H), 4.59-4.70 (m, 1H), 7.08-7.15 (m, 1H), 7.58-7.66 (m, 4H), 7.73-7.78 (m, 1H), 7.80-7.91 (m, 2H), 8.38-8.45 (m, 1H); ESI-MS m/z [M+H]⁺ 368.2.

### Example 139: (R)-4-(4-chlorophenyl)-N-(1-methylpiperidin-3-yl)phthalazin-1-amine

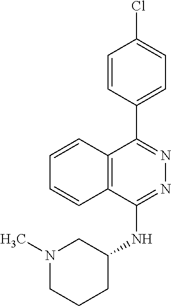 [SMILES: CN1CCC[C@@H](Nc2nnc(-c3ccc(Cl)cc3)c3ccccc23)C1]

The title compound was prepared like Example 112, using 1-chloro-4-(4-chlorophenyl)phthalazine (0.138 g, 0.500 mmol) and (R)-1-methylpiperidin-3-amine dihydrochloride (0.094 g, 0.5 mmol), and was obtained as an off-white solid (59.4 mg, 33.7%). ¹H NMR (400 MHz, CD₃OD) δ ppm 1.75-1.93 (m, 1H), 1.94-2.03 (m, 1H), 2.10-2.23 (m, 1H), 2.25-2.35 (m, 1H), 2.84-2.94 (m, 3H), 2.95-3.16 (m, 2H), 3.50-3.62 (m, 1H), 3.81-3.97 (m, 1H), 4.52-4.68 (m, 1H), 7.66-7.72 (m, 4H), 8.06-8.15 (m, 2H), 8.17-8.25 (m, 1H), 8.63 (br d, J=8.03 Hz, 1H); ESI-MS m/z (M+H)+354.1.

### Example 140: 4-(4-chlorophenyl)-N-(1-(2-fluoroethyl)piperidin-3-yl)phthalazin-1-amine

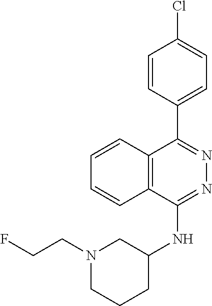 [SMILES: FCCN1CCCC(Nc2nnc(-c3ccc(Cl)cc3)c3ccccc23)C1]

The title compound was prepared like Example 112, using 1-chloro-4-(4-chlorophenyl)phthalazine (28 mg, 0.100 mmol) and 1-(2-fluoroethyl)piperidin-3-amine (15 mg, 0.100 mmol), and was obtained as an off-white solid (13 mg, 34%). ¹H NMR (400 MHz, CD₃OD) δ ppm 1.86-2.13 (m, 2H), 2.13-2.24 (m, 1H), 2.27 (br s, 1H), 3.05-3.26 (m, 2H), 3.43-3.69 (m, 3H), 3.82-4.08 (m, 1H), 4.61-4.68 (m, 1H), 4.78-4.82 (m, 1H), 4.90-4.94 (m, 1H), 7.66-7.73 (m, 4H), 8.06-8.15 (m, 2H), 8.22 (td, J=7.62, 1.44 Hz, 1H), 8.66 (br d, J=8.16 Hz, 1H); ESI-MS m/z [M+H]385.3.

### Example 141: N-(1-(2-fluoroethyl)piperidin-3-yl)-4-(4-methoxyphenyl)phthalazin-1-amine

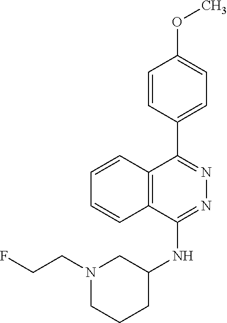 [SMILES: COc1ccc(-c2nnc(NC3CCCN(CCF)C3)c3ccccc23)cc1]

The title compound was prepared like Example 112, using 1-chloro-4-(4-methoxyphenyl)phthalazine (129.59 mg, 0.479 mmol) and 1-(2-fluoroethyl)piperidin-3-amine (70 mg, 0.479 mmol) as an off-white solid (13 mg, 7.1%). ¹H NMR (400 MHz, CD₃OD) δ ppm 1.91-2.09 (m, 2H), 2.17-2.35 (m, 2H), 2.64-2.64 (m, 2H), 2.88-3.21 (m, 2H), 3.56-3.60 (m, 1H), 3.63-3.67 (m, 1H), 3.93 (s, 3H), 4.62-4.69 (m, 1H), 4.80-4.83 (m, 1H), 4.91-4.95 (m, 1H), 7.22-7.28 (m, 2H), 7.67-7.73 (m, 2H), 8.13-8.29 (m, 3H), 8.53-8.68 (m, 1H); ESI-MS m/z [M+H]⁺ 381.3.

### Example 142: 4-(4-chlorophenyl)-N-(1-(2,2-difluoroethyl)piperidin-3-yl)phthalazin-1-amine

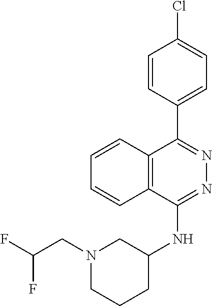 [SMILES: FC(F)CN1CCCC(Nc2nnc(-c3ccc(Cl)cc3)c3ccccc23)C1]

The title compound was prepared like Example 112, using 1-chloro-4-(4-chlorophenyl)phthalazine (0.275 g, 1 mmol) and 1-(2,2-difluoroethyl)piperidin-3-amine dihydrochloride (0.237 g, 1.00 mmol), and was obtained as an off-white solid (35.9 mg, 8.9%). ¹H NMR (400 MHz, CD₃OD) δ ppm 1.56-1.83 (m, 3H), 1.94-2.06 (m, 1H), 2.40 (q, J=9.66 Hz, 2H), 2.73-2.85 (m, 3H), 3.18-3.26 (m, 1H), 4.45-4.54 (m, 1H), 5.84-6.17 (m, 1H), 7.49-7.61 (m, 4H), 7.75-7.88 (m, 3H), 8.24 (d, J=8.16 Hz, 1H); ESI-MS m/z [M+H]⁺ 403.3.

### Example 143: 4-(4-chlorophenyl)-N-(1-(2-fluoroethyl)pyrrolidin-3-yl)phthalazin-1-amine

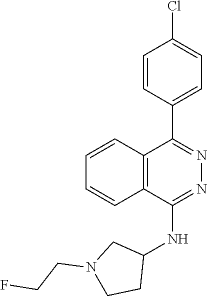 [SMILES: FCCN1CCC(Nc2nnc(-c3ccc(Cl)cc3)c3ccccc23)C1]

The title compound was prepared like Example 112, using 1-(2-fluoroethyl)pyrrolidin-3-amine (26 mg, 0.200 mmol) and 1-chloro-4-(4-chlorophenyl)phthalazine (55 mg, 0.20 mmol), and was obtained as an off-white solid (17 mg, 23%). ¹H NMR (400 MHz, CD₃OD) δ ppm 1.30-1.40 (m, 3H), 1.79-2.08 (m, 2H), 2.08-2.24 (m, 1H), 2.32 (br d, J 12.30 Hz, 1H), 2.85-3.09 (m, 2H), 3.62 (br d, J 12.05 Hz, 1H), 4.52-4.74 (m, 1H), 7.20-7.27 (m, 2H), 7.69 (d, J=8.66 Hz, 2H), 8.11-8.29 (m, 3H), 8.60 (d, J=8.28 Hz, 1H); ESI-MS m/z [M+H]⁺ 371.2.

### Example 144: 4-(4-chlorophenyl)-N-(1-cyclopropylpiperidin-3-yl)phthalazin-1-amine

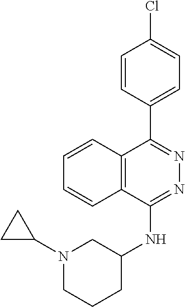 [SMILES: Clc1ccc(-c2nnc(NC3CCCN(C4CC4)C3)c3ccccc23)cc1]

The title compound was prepared like Example 112, using 1-cyclopropylpiperidin-3-amine (28 mg, 0.20 mmol) and 1-chloro-4-(4-chlorophenyl)phthalazine (55 mg, 0.20 mmol) as an off-white solid (17 mg, 22%). ¹H NMR (400 MHz, CD₃OD) δ ppm 0.97-1.04 (m, 2H), 1.04-1.13 (m, 2H), 1.91-2.04 (m, 2H), 2.17-2.26 (m, 1H), 2.26-2.36 (m, 1H), 2.91-2.97 (m, 1H), 3.25 (br s, 2H), 3.59-3.77 (m, 1H), 3.92-4.09 (m, 1H), 4.55-4.64 (m, 1H), 7.70-7.76 (m, 4H), 8.10-8.19 (m, 2H), 8.22-8.28 (m, 1H), 8.67 (d, J=8.16 Hz, 1H); ESI-MS m/z [M+H]⁺ 379.2.

### Example 145: 4-(4-chlorophenyl)-N-(1-cyclobutylpiperidin-3-yl)phthalazin-1-amine

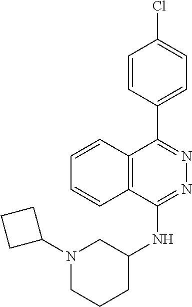 [SMILES: Clc1ccc(-c2nnc(NC3CCCN(C4CCC4)C3)c3ccccc23)cc1]

The title compound was prepared like Example 112, using 1-chloro-4-(4-chlorophenyl)phthalazine (55 mg, 0.20 mmol) and 1-cyclobutylpiperidin-3-amine (31 mg, 0.20 mmol) as an off-white solid (23 mg, 30%). ¹H NMR (400 MHz, CD₃OD) δ ppm 1.76-1.99 (m, 4H), 2.11-2.19 (m, 1H), 2.25-2.36 (m, 5H), 2.70-2.97 (m, 2H), 3.41-3.63 (m, 1H), 3.63-3.77 (m, 1H), 3.82 (br d, J=10.42 Hz, 1H), 4.47-4.69 (m, 1H), 7.64-7.72 (m, 4H), 8.04-8.22 (m, 3H), 8.63 (br d, J=8.16 Hz, 1H); ESI-MS m/z [M+H]⁺ 393.2.

### Example 146: 4-(4-chlorophenyl)-N-(1-cyclopentylpiperidin-3-yl)phthalazin-1-amine

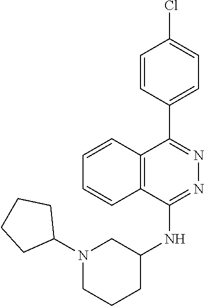 [SMILES: Clc1ccc(-c2nnc(NC3CCCN(C4CCCC4)C3)c3ccccc23)cc1]

The title compound was prepared like Example 112, using 1-chloro-4-(4-chlorophenyl)phthalazine (28 mg, 0.10 mmol) and 1-cyclopentylpiperidin-3-amine (17 mg, 0.10 mmol) obtained as an off-white solid (15 mg, 37%). ¹H NMR (400 MHz, CD₃OD) δ ppm 1.59-1.69 (m, 2H), 1.72-1.82 (m, 4H), 1.83-2.02 (m, 2H), 2.11-2.22 (m, 3H), 2.23-2.35 (m, 1H), 2.98 (br t, J=11.86 Hz, 2H), 3.56-3.72 (m, 2H), 3.97 (br d, J=11.17 Hz, 1H), 4.56 (br t, J=11.36 Hz, 1H), 7.65-7.73 (m, 4H), 8.06-8.24 (m, 3H), 8.64 (br d, J=8.16 Hz, 1H); ESI-MS m/z [M+H]⁺ 407.3.

### Example 147: 4-(4-chlorophenyl)-N-(1-(2,2,2-trifluoroethyl)piperidin-3-yl)phthalazin-1-amine

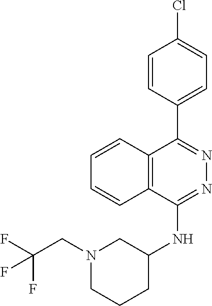 [SMILES: FC(F)(F)CN1CCCC(Nc2nnc(-c3ccc(Cl)cc3)c3ccccc23)C1]

The title compound was prepared like Example 112, using 1-chloro-4-(4-chlorophenyl)phthalazine (28 mg, 0.10 mmol) and 1-(2,2,2-trifluoroethyl)piperidin-3-amine (18 mg, 0.10 mmol), and was obtained as an off-white solid (10 mg, 24%). ¹H NMR (400 MHz, CD₃OD) δ ppm 1.60-1.72 (m, 1H), 1.72-1.89 (m, 2H), 2.08-2.16 (m, 1H), 2.53-2.63 (m, 1H), 2.70 (t, J=10.10 Hz, 1H), 2.93-3.00 (m, 1H), 3.19-3.26 (m, 2H), 3.28-3.31 (m, 1H), 4.16-4.24 (m, 1H), 7.60-7.68 (m, 4H), 7.99-8.04 (m, 1H), 8.08-8.16 (m, 2H), 8.62-8.67 (m, 1H); ESI-MS m/z [M+H]⁺ 421.3.

### Example 148: 4-(4-chlorophenyl)-N-(1-(methylsulfonyl)piperidin-3-yl)phthalazin-1-amine

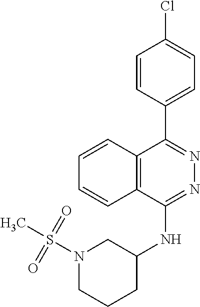 [SMILES: CS(=O)(=O)N1CCCC(Nc2nnc(-c3ccc(Cl)cc3)c3ccccc23)C1]

The title compound was prepared like Example 112, using 1-chloro-4-(4-chlorophenyl)phthalazine (28 mg, 0.10 mmol) and 1-(methylsulfonyl)piperidin-3-amine hydrochloride (21 mg, 0.100 mmol), and was obtained as an off-white solid (5.0 mg, 12%). ¹H NMR (400 MHz, CD₃OD) δ ppm 1.75-1.88 (m, 2H), 1.91-2.06 (m, 1H), 2.19-2.30 (m, 1H), 2.89-2.90 (m, 3H), 2.91-3.00 (m, 2H), 3.67 (br d, J=12.05 Hz, 1H), 4.01 (dd, J=11.61, 4.08 Hz, 1H), 4.21-4.32 (m, 1H), 7.64-7.72 (m, 4H), 8.04-8.21 (m, 3H), 8.66-8.70 (m, 1H); ESI-MS m/z [M+H]⁺ 417.2.

### Example 149: Ni-(4-(4-chlorophenyl)phthalazin-1-yl)-N³,N³-dimethylcyclobutane-1,3-diamine

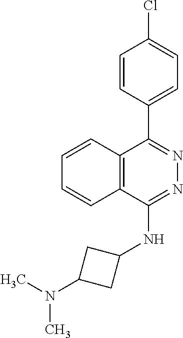 [SMILES: CN(C)C1CC(Nc2nnc(-c3ccc(Cl)cc3)c3ccccc23)C1]

The title compound was prepared like Example 112, using 1-chloro-4-(4-chlorophenyl)phthalazine (28 mg, 0.10 mmol) and N^{l},N^{l}-dimethylcyclobutane-1,3-diamine (11 mg, 0.10 mmol), and was obtained as an off-white solid (4.8 mg, 14%). ¹H NMR (400 MHz, CD₃OD) δ ppm 2.55-2.74 (m, 1H), 2.79-2.84 (m, 1H), 2.84-2.87 (m, 6H), 2.89-3.00 (m, 1H), 3.02-3.15 (m, 1H), 3.62-4.11 (m, 1H), 4.31-4.59 (m, 1H), 7.63-7.70 (m, 4H), 8.03-8.21 (m, 3H), 8.70-8.77 (m, 1H); ESI-MS m/z [M+H]⁺ 353.2.

### Example 150: 7-(4-methoxyphenyl)-1-methyl-N-(1-methylpiperidin-3-yl)-1H-pyrazolo[3,4-d]pyridazin-4-amine

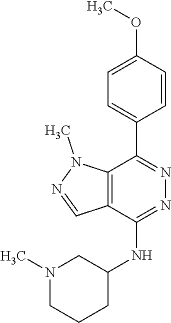 [SMILES: COc1ccc(-c2nnc(NC3CCCN(C)C3)c3cnn(C)c23)cc1]

The title compound was prepared like Example 112, using 4-chloro-7-(4-methoxyphenyl)-1-methyl-1H-pyrazolo[3,4-d]pyridazine (0.087 g, 0.316 mmol) and 1-methylpiperidin-3-amine (0.036 g, 0.316 mmol), and was obtained as a clear oil (0.8 mg, 0.71%). ¹H NMR (400 MHz, CD₃OD) δ ppm 1.27-1.38 (m, 1H), 1.47-1.59 (m, 1H), 1.68-1.91 (m, 2H), 2.01-2.12 (m, 1H), 2.15-2.28 (m, 2H), 2.33 (s, 3H), 2.67-2.76 (m, 1H), 3.09-3.17 (m, 1H), 3.73 (s, 3H), 3.88 (s, 3H), 4.42-4.51 (m, 1H), 7.08-7.14 (m, 2H), 7.46-7.53 (m, 2H), 8.26-8.29 (m, 1H); ESI-MS m/z [M+H]⁺ 353.4.

### Example 151: 4-(4-chlorophenyl)-N-(3-methyl-3-azabicyclo[3.3.1]nonan-9-yl)phthalazin-1-amine

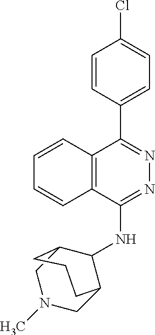 [SMILES: CN1CC2CCCC(C1)C2Nc1nnc(-c2ccc(Cl)cc2)c2ccccc12]

A mixture of 1-chloro-4-(4-chlorophenyl)phthalazine (30 mg, 0.109 mmol, 1 eq), 3-methyl-3-azabicyclo[3.3.1]nonan-9-amine (50.5 mg, 0.327 mmol, 3 eq) and K₂CO₃ (30.1 mg, 0.218 mmol, 2 eq) in NMP (0.5 mL) was stirred at 150° C. for 2 hours. DMSO (1.0 mL) was added and the resulting solution was purified by preparative HPLC (Phenomenex Gemini® C18-5 μm, 30 mm×150 mm column) using a gradient of 20-100% ACN (A: water, B: 20% water in ACN, both modified with 10 mM NH₄HCO₃). The title compound was obtained as a light-yellow solid (1.2 mg, 2.8%). ESI-MS m/z [M+H]⁺ 393.

### Example 152: 4-(4-chlorophenyl)-N-(3-methyl-3-azabicyclo[3.1.0]hexan-1-yl)phthalazin-1-amine

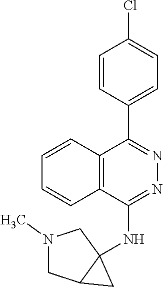 [SMILES: CN1CC2CC2(Nc2nnc(-c3ccc(Cl)cc3)c3ccccc23)C1]

The title compound was prepared like Example 151, using 1-chloro-4-(4-chlorophenyl)phthalazine and 3-methyl-3-azabicyclo[3.1.0]hexan-1-amine, and was obtained as an orange solid (5 mg, 13%). ESI-MS m/z [M+H]⁺ found, 351.1.

### Example 153: 4-(4-chlorophenyl)-N-phenylphthalazin-1-amine

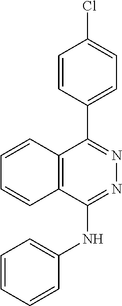 [SMILES: Clc1ccc(-c2nnc(Nc3ccccc3)c3ccccc23)cc1]

The title compound was prepared like Example 151, using 1-chloro-4-(4-chlorophenyl)phthalazine and aniline, and was obtained as an orange solid (1.1 mg, 3.04%). ESI-MS m/z [M+H]+ 332.1.

### Example 154: 4-(4-chlorophenyl)-N-(1-(pyridin-2-yl)piperidin-3-yl)phthalazin-1-amine

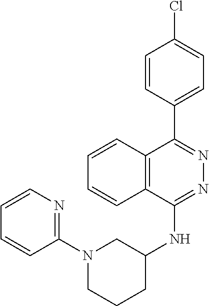 [SMILES: Clc1ccc(-c2nnc(NC3CCCN(c4ccccn4)C3)c3ccccc23)cc1]

The title compound was prepared like Example 151, using 1-chloro-4-(4-chlorophenyl)phthalazine and 1-(pyridin-2-yl)piperidin-3-amine, and was obtained as a white solid (0.9 mg, 1.9%). ESI-MS m/z [M+H]+ 416.15.

### Example 155: N-(1-benzylpiperidin-3-yl)-4-(4-chlorophenyl)phthalazin-1-amine

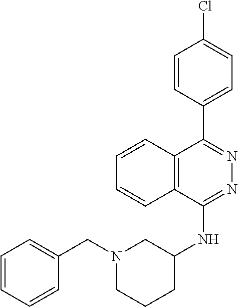 [SMILES: Clc1ccc(-c2nnc(NC3CCCN(Cc4ccccc4)C3)c3ccccc23)cc1]

The title compound was prepared like Example 151, using 1-chloro-4-(4-chlorophenyl)phthalazine and 1-benzylpiperidin-3-amine, and was obtained as an orange solid (3 mg, 6.4%). ESI-MS m/z [M+H]⁺ 429.2.

### Example 156: 2-((4-(4-chlorophenyl)phthalazin-1-yl)amino)phenol

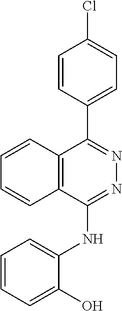 [SMILES: Oc1ccccc1Nc1nnc(-c2ccc(Cl)cc2)c2ccccc12]

The title compound was prepared like Example 151, using 1-chloro-4-(4-chlorophenyl)phthalazine and 2-aminophenol, and was obtained as a green solid (27.5 mg, 72.5%). ¹H NMR (400 MHz, CDCl₃) δ ppm 7.07-7.15 (m, 1H), 7.20-7.32 (m, 2H), 7.66-7.75 (m, 2H), 7.75-7.84 (m, 2H), 7.90 (br d, J=7.53 Hz, 1H), 7.99-8.07 (m, 1H), 8.07-8.20 (m, 2H), 8.47 (br d, J=8.53 Hz, 1H); ESI-MS m/z [M+H]f 348.1.

### Example 157: 4-(4-chlorophenyl)-N-(1,4,4-trimethylpyrrolidin-3-yl)phthalazin-1-amine

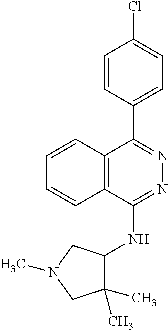 [SMILES: CN1CC(Nc2nnc(-c3ccc(Cl)cc3)c3ccccc23)C(C)(C)C1]

The title compound was prepared like Example 151, using 1-chloro-4-(4-chlorophenyl)phthalazine and 1,4,4-trimethylpyrrolidin-3-amine dihydrochloride, and was obtained as an orange solid (1.5 mg, 3.7%). ESI-MS m/z [M+H]⁺ 367.2.

### Example 158: N-(1-benzylpyrrolidin-3-yl)-4-(4-chlorophenyl)phthalazin-1-amine

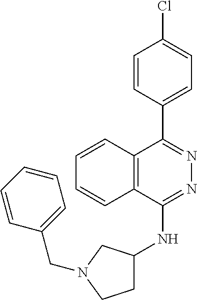 [SMILES: Clc1ccc(-c2nnc(NC3CCN(Cc4ccccc4)C3)c3ccccc23)cc1]

The title compound was prepared like Example 151, using 1-chloro-4-(4-chlorophenyl)phthalazine and 1-benzylpyrrolidin-3-amine, and was obtained as a brown solid (22.7 mg, 50.2%). ¹H NMR (400 MHz, CD₃OD) δ ppm 2.53 (dt, J=13.05, 6.53 Hz, 1H), 2.64-2.94 (m, 1H), 3.37-4.03 (m, 4H), 4.43-4.63 (m, 2H), 4.98-5.05 (m, 1H), 7.44-7.54 (m, 3H), 7.59 (d, J=5.59 Hz, 2H), 7.70-7.78 (m, 4H), 8.08-8.20 (m, 2H), 8.20-8.27 (m, 1H), 8.72 (br d, J=8.03 Hz, 1H); ESI-MS m/z [M+H]⁺ 415.2.

### Example 159: 4-(4-chlorophenyl)-N-(1-ethyl-4-methylpyrrolidin-3-yl)phthalazin-1-amine

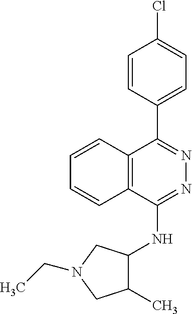 [SMILES: CCN1CC(C)C(Nc2nnc(-c3ccc(Cl)cc3)c3ccccc23)C1]

The title compound was prepared like Example 151, using 1-chloro-4-(4-chlorophenyl)phthalazine and 1-ethyl-4-methylpyrrolidin-3-amine, and was obtained as a red solid (1.6 mg, 3.99%). ESI-MS m/z [M+H]⁺ 367.1.

### Example 160: 1-(3-((4-(4-chlorophenyl)phthalazin-1-yl)amino)pyrrolidin-1-yl)ethan-1-one

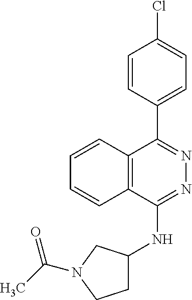 [SMILES: CC(=O)N1CCC(Nc2nnc(-c3ccc(Cl)cc3)c3ccccc23)C1]

The title compound was prepared like Example 151, using 1-chloro-4-(4-chlorophenyl)phthalazine and 1-(3-aminopyrrolidin-1-yl)ethan-1-one, and was obtained as a brown solid (10.1 mg, 25.2%). ¹H NMR (400 MHz, CD₃OD) δ ppm 1.96-2.17 (m, 3H), 2.26-2.62 (m, 2H), 3.64-3.90 (m, 3H), 3.93-4.14 (m, 1H), 4.64-4.83 (m, 1H), 7.65-7.77 (m, 4H), 8.06-8.14 (m, 1H), 8.14-8.25 (m, 2H), 8.79 (dd, J=7.84, 0.82 Hz, 1H); ESI-MS m/z [M+H]+ 367.1.

### Example 161: 4-(4-chlorophenyl)-N-(1-(cyclopropylmethyl)pyrrolidin-3-yl)phthalazin-1-amine

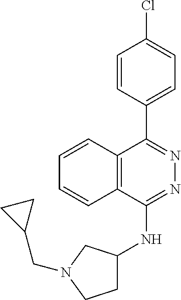 [SMILES: Clc1ccc(-c2nnc(NC3CCN(CC4CC4)C3)c3ccccc23)cc1]

The title compound was prepared like Example 151, using 1-chloro-4-(4-chlorophenyl)phthalazine and 1-(cyclopropylmethyl)pyrrolidin-3-amine, and was obtained as a brown solid (6.0 mg, 14.5%). ESI-MS m/z [M+H]+ 379.2.

### Example 162: 4-(4-chlorophenyl)-N-(1-ethylpiperidin-3-yl)phthalazin-1-amine

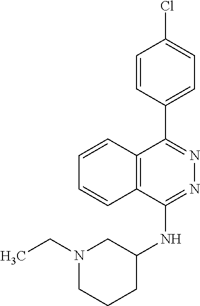 [SMILES: CCN1CCCC(Nc2nnc(-c3ccc(Cl)cc3)c3ccccc23)C1]

The title compound was prepared like Example 151, using 1-chloro-4-(4-chlorophenyl)phthalazine and 1-ethylpiperidin-3-amine, and was obtained as a white solid (2.3 mg, 5.7%). ESI-MS m/z [M+H]⁺ 367.2.

### Example 163: N-(1-benzylpiperidin-4-yl)-4-(4-chlorophenyl)phthalazin-1-amine

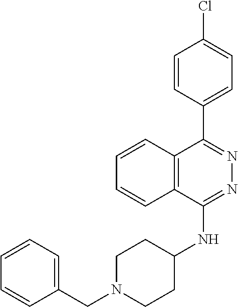 [SMILES: Clc1ccc(-c2nnc(NC3CCN(Cc4ccccc4)CC3)c3ccccc23)cc1]

The title compound was prepared like Example 151, using 1-chloro-4-(4-chlorophenyl)phthalazine and 1-benzylpiperidin-4-amine, and was obtained as an orange solid (19.7 mg, 42.1%). ¹H NMR (400 MHz, CD₃OD) δ ppm 2.10-2.25 (m, 2H), 2.43-2.54 (m, 2H), 3.20-3.31 (m, 2H), 3.71 (br d, J=12.67 Hz, 2H), 4.31-4.41 (m, 1H), 4.43 (s, 2H), 7.53-7.60 (m, 5H), 7.69-7.75 (m, 4H), 8.08-8.14 (m, 1H), 8.14-8.24 (m, 2H), 8.71 (br d, J=7.28 Hz, 1H); ESI-MS m/z [M+H]⁺ 429.2.

### Example 164: N-(4-(4-chlorophenyl)phthalazin-1-yl)quinuclidin-3-amine

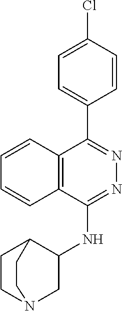 [SMILES: Clc1ccc(-c2nnc(NC3CN4CCC3CC4)c3ccccc23)cc1]

The title compound was prepared like Example 151, using 1-chloro-4-(4-chlorophenyl)phthalazine and quinuclidin-3-amine dihydrochloride, and was obtained as a yellow solid (7.0 mg, 17.6%). ESI-MS m/z [M+H]⁺ 365.1.

### Example 165: 4-(4-chlorophenyl)-N-((3S,4R)-4-isopropyl-1-methylpyrrolidin-3-yl)phthalazin-1-amine

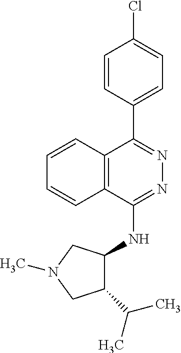 [SMILES: CC(C)[C@@H]1CN(C)C[C@H]1Nc1nnc(-c2ccc(Cl)cc2)c2ccccc12]

The title compound was prepared like Example 151, using 1-chloro-4-(4-chlorophenyl)phthalazine and (3S,4R)-4-isopropyl-1-methylpyrrolidin-3-amine hydrochloride, and was obtained as a yellow solid (1.2 mg, 2.8%). ESI-MS m/z [M+H]⁺ 381.2.

### Example 166: N-(1-azabicyclo[2.2.1]heptan-3-yl)-4-(4-chlorophenyl)phthalazin-1-amine

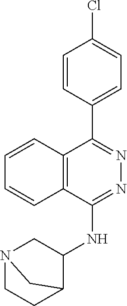 [SMILES: Clc1ccc(-c2nnc(NC3CN4CCC3C4)c3ccccc23)cc1]

The title compound was prepared like Example 151, using 1-chloro-4-(4-chlorophenyl)phthalazine and 1-azabicyclo[2.2.1]heptan-3-amine dihydrochloride, and was obtained as a yellow solid (1.0 mg, 2.6%). ESI-MS m/z [M+H]⁺ 351.15.

### Example 167: N-(1-azabicyclo[3.2.1]octan-4-yl)-4-(4-chlorophenyl)phthalazin-1-amine

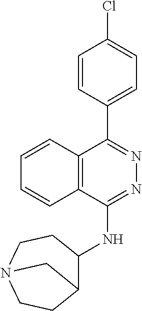 [SMILES: Clc1ccc(-c2nnc(NC3CCN4CCC3C4)c3ccccc23)cc1]

The title compound was prepared like Example 151, using 1-chloro-4-(4-chlorophenyl)phthalazine and 1-azabicyclo[3.2.1]octan-4-amine dihydrochloride, and was obtained as a light-yellow solid (1.1 mg, 2.8%). ESI-MS m/z [M+H]⁺ 365.15.

### Example 168: (R)-1-(4-methoxyphenyl)-N-(1-(1-methylcyclopropyl)piperidin-3-yl)pyrido[3,4-d]pyridazin-4-amine

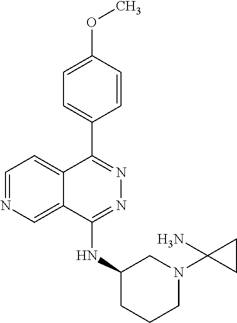 [SMILES: COc1ccc(-c2nnc(N[C@@H]3CCCN(C4(N)CC4)C3)c3cnccc23)cc1]

A mixture of (R)-1-(1-methylcyclopropyl)piperidin-3-amine hydrochloride (120 mg, 629.22 μmol), 4-chloro-1-(4-methoxyphenyl)pyrido[3,4-d]pyridazine (170.96 mg, 629.22 μmol) and DIPEA (325.29 mg, 2.52 mmol, 438.39 μL) in DMSO (2 mL) was stirred at 100° C. for 12 hours. LC-MS showed one main peak with desired m/z. The mixture was filtered and the filtrate was purified by preparative HPLC (Method C). The title compound was obtained as a white solid (40 mg, 15%, 93% purity). ¹H NMR (400 MHz, DMSO-d₆) δ ppm 0.22-0.39 (m, 2H), 0.51 (d, J=2.6 Hz, 2H), 1.05 (s, 3H), 1.37-1.57 (m, 2H), 1.70-1.82 (m, 1H), 1.96-2.09 (m, 1H), 2.35-2.44 (m, 2H), 2.76 (br d, J=10.4 Hz, 1H), 3.16 (br d, J=6.9 Hz, 1H), 3.86 (s, 3H), 4.33 (br s, 1H), 7.13 (d, J=8.6 Hz, 2H), 7.53 (d, J=7.6 Hz, 1H), 7.59 (d, J=8.8 Hz, 2H), 7.64 (d, J=5.6 Hz, 1H), 8.90 (d, J=5.6 Hz, 1H), 9.78 (s, 1H); ESI-MS m/z [M+H]+ 390.2.

### Example 169: (R)—N-(1-cyclopropylpiperidin-3-yl)-1-(4-methoxyphenyl)pyrido[3,4-d]pyridazin-4-amine

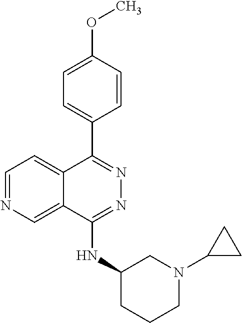 [SMILES: COc1ccc(-c2nnc(N[C@@H]3CCCN(C4CC4)C3)c3cnccc23)cc1]

The title compound was prepared like Example 168, using (R)-1-cyclopropylpiperidin-3-amine hydrochloride (130.06 mg, 736.10 μmol) and 4-chloro-1-(4-methoxyphenyl)pyrido[3,4-d]pyridazine (100 mg, 368.05 μmol), and was obtained as a yellow solid (30 mg, 21% yield, 97% purity). ¹H NMR (400 MHz, DMSO-d₆) δ ppm 0.28-0.49 (m, 4H), 1.40-1.59 (m, 2H), 1.62-1.82 (m, 2H), 1.96-2.09 (m, 1H), 2.13-2.26 (m, 2H), 2.90 (br d, J=11.04 Hz, 1H), 3.28 (br dd, J=10.29, 3.76 Hz, 1H), 3.84 (s, 3H), 4.25-4.45 (m, 1H), 7.02-7.25 (m, 2H), 7.50-7.61 (m, 3H), 7.63 (d, J=5.52 Hz, 1H), 8.16 (s, 1H), 8.89 (d, J=5.77 Hz, 1H), 9.78 (s, 1H); ESI-MS m/z [M+H]⁺ 376.2.

### Example 170: 4-(4-chlorophenyl)-N-(1-(2,2-difluoroethyl)pyrrolidin-3-yl)phthalazin-1-amine

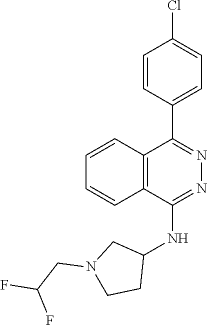 [SMILES: FC(F)CN1CCC(Nc2nnc(-c3ccc(Cl)cc3)c3ccccc23)C1]

A mixture of 1-(2,2-difluoroethyl)pyrrolidin-3-amine (30 mg, 0.20 mmol), 1-chloro-4-(4-chlorophenyl)phthalazine (55 mg, 0.20 mmol) and K₂CO₃ (84 mg, 0.60 mmol) in DMSO (3.33 mL) was heated at 100° C. overnight. The mixture was filtered and purified by preparative HPLC (Method A). The title compound was obtained as an off-white solid (16 mg, 21%). ¹H NMR (400 MHz, CD₃OD) δ ppm 2.43-2.52 (m, 1H), 2.65-2.80 (m, 1H), 3.63-3.67 (m, 1H), 3.69-3.73 (m, 1H), 3.81-4.00 (m, 2H), 4.73-4.77 (m, 1H), 4.87-4.89 (m, 2H), 4.91-4.97 (m, 1H), 7.66-7.73 (m, 4H), 8.06-8.23 (m, 3H), 8.67 (d, J=8.03 Hz, 1H); ESI-MS m/z [M+H]⁺ 389.2.

### Example 171: N-(1-ethylpiperidin-3-yl)-4-(4-methoxyphenyl)phthalazin-1-amine

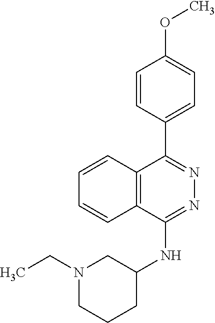 [SMILES: CCN1CCCC(Nc2nnc(-c3ccc(OC)cc3)c3ccccc23)C1]

The title compound was prepared like Example 170, using 1-ethylpiperidin-3-amine (26 mg, 0.20 mmol) and 1-chloro-4-(4-methoxyphenyl)phthalazine (54 mg, 0.20 mmol) as an off-white solid (8.9 mg, 12%). ¹H NMR (400 MHz, CD₃OD) δ ppm 1.31-1.39 (m, 3H), 1.76-2.02 (m, 2H), 2.12-2.21 (m, 1H), 2.26-2.37 (m, 1H), 2.85-3.13 (m, 2H), 3.22-3.26 (m, 2H), 3.57-3.67 (m, 1H), 3.89-3.92 (m, 3H), 3.92-3.99 (m, 1H), 4.52-4.70 (m, 1H), 7.20-7.27 (m, 2H), 7.69 (d, J=8.66 Hz, 2H), 8.11-8.29 (m, 3H), 8.60 (d, J=8.28 Hz, 1H); ESI-MS m/z [M+H]⁺ 363.3.

### Example 172: 2-(4-((trans-5-fluoro-1-methylpiperidin-3-yl)amino)pyrido[3,4-d]pyridazin-1-yl)phenol

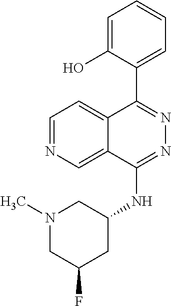 [SMILES: CN1C[C@H](F)C[C@@H](Nc2nnc(-c3ccccc3O)c3ccncc23)C1]

To a 5 mL vial equipped with a stir bar were added 1-chloro-N-(trans-5-fluoro-1-methylpiperidin-3-yl)pyrido[3,4-d]pyridazin-4-amine (250 mg, 0.845 mmol), (2-hydroxyphenyl)boronic acid (210 mg, 1.52 mmol), XPhos-Pd-G2 (67 mg, 0.0845 mmol) and K₂CO₃ (234 mg, 1.69 mmol) in 1,4-dioxane (11.74 mL) and water (3.9132 mL). The reaction mixture was heated in a microwave reactor (Biotage® Initiator) at 100° C. for 60 minutes and the extracted with EtOAc (50 mL×2) and brine (50 mL). The organic phases were separated, dried over MgSO₂, concentrated with silica gel and purified on an amine column, using a gradient of 0-30% MeOH in DCM. The resulting crude product (300 mg) was further purified via SFC to give the title compound (53 mg, 18%). ¹H NMR (400 MHz, DMSO-d₆) δ ppm 1.72-1.90 (m, 1H), 1.99-2.08 (m, 1H), 2.15 (br d, J=12.30 Hz, 1H), 2.25 (s, 4H), 2.28-2.32 (m, 1H), 2.88-3.01 (m, 1H), 3.06-3.16 (m, 1H), 3.28-3.30 (m, 1H), 4.71-4.84 (m. 1H), 4.96 (br s, 1H), 5.08 (br s, 1H), 6.95-7.03 (m, 2H), 7.28-7.38 (m, 3H), 7.59 (d, J=7.78 Hz, 1H), 8.85 (d, J=5.52 Hz, 1H), 9.68 (s, 1H), 9.75 (s, 1H); ESI-MS m/z [M+H]⁺ 354.1.

### Example 173: 2-(4-((trans-5-fluoro-1-methylpiperidin-3-yl)amino)pyrido[3,4-d]pyridazin-1-yl)-5-methylphenol

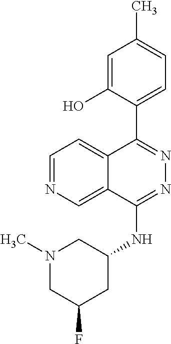 [SMILES: Cc1ccc(-c2nnc(N[C@@H]3C[C@@H](F)CN(C)C3)c3cnccc23)c(O)c1]

To a 5 mL vial equipped with a stir bar were added 1-chloro-N-(trans-5-fluoro-1-methylpiperidin-3-yl)pyrido[3,4-d]pyridazin-4-amine (30 mg, 0.101 mmol), (2-hydroxy-4-methylphenyl)boronic acid (28 mg, 0.183 mmol), XPhos-Pd-G2 (8.0 mg, 0.0101 mmol) and K₂CO₃ (28 mg, 0.203 mmol) in 1,4-dioxane (1.4088 mL) and water (0.4696 mL). The reaction mixture was heated in a microwave reactor (Biotage® Initiator) at 120° C. for 30 minutes and then diluted with DMF (0.3 mL) and purified using HPLC Method C to give the title compound (11 mg, 30%). 1H NMR (400 MHz, CD₃OD) δ ppm 1.80-1.93 (m, 1H), 1.93-2.01 (m, 1H), 2.18-2.30 (m, 1H), 2.36 (d, J=4.27 Hz, 8H), 2.88-3.06 (m, 1H), 3.25 (br dd, J=10.79, 3.01 Hz, 1H), 4.90-4.99 (m, 2H), 4.99-5.08 (m, 1H), 6.75-6.89 (m, 2H), 7.25 (d, J=7.53 Hz, 1H), 7.53 (dd, J=5.65, 0.88 Hz, 1H), 8.81 (d, J=5.77 Hz, 1H), 9.63 (d, J=0.75 Hz, 1H); ESI-MS m/z [M+H]⁺ 368.1.

### Example 174: N-((3R,5R)-5-fluoro-1-methylpiperidin-3-yl)-1-(4-methoxyphenyl)pyrido[3,4-d]pyridazin-4-amine

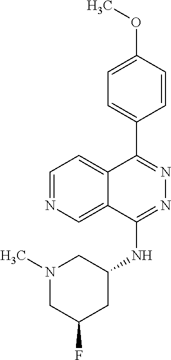 [SMILES: COc1ccc(-c2nnc(N[C@@H]3C[C@@H](F)CN(C)C3)c3cnccc23)cc1]

A mixture of tert-butyl (3R,5R)-3-fluoro-5-((1-(4-methoxyphenyl)pyrido[3,4-d]pyridazin-4-yl)amino)piperidine-1-carboxylate (55 mg, 121.28 μmol), formaldehyde (7.28 mg, 242.55 μmol, 6.68 μL) in formic acid (1 mL) was degassed and purged with N₂ (3×) and stirred at 80° C. for 1 hour under N₂ atmosphere. The reaction mixture was concentrated under vacuum and the residue was purified by preparative HPLC (Method B). The title compound was obtained as a white solid (19.5 mg, 43.6% yield, 99.7% purity). ¹H NMR (400 MHz, DMSO-d₆) δ ppm 1.68-1.92 (m, 1H), 1.99-2.10 (m, 1H), 2.15 (s, 1H), 2.24-2.27 (m, 3H), 2.27 (br s, 1H), 2.91-3.02 (m, 1H), 3.07-3.16 (m, 1H), 3.84-3.87 (m, 3H), 4.72-4.86 (m, 1H), 4.93-5.14 (m, 1H), 7.09-7.16 (m, 2H), 7.56-7.69 (m, 4H), 8.16 (s, 1H), 8.88-8.93 (m, 1H), 9.77-9.82 (m, 1H); ESI-MS m/z [M+H]⁺ 368.4.

### Example 175: 4-(4-chlorophenyl)-N-((3R,5R)-5-fluoro-1-methylpiperidin-3-yl)pyrido[3,4-d]pyridazin-1-amine

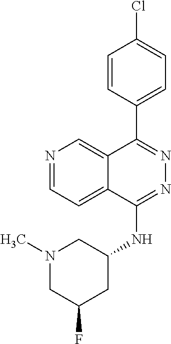 [SMILES: CN1C[C@H](F)C[C@@H](Nc2nnc(-c3ccc(Cl)cc3)c3cnccc23)C1]

The title compound was prepared like Example 174, using tert-butyl (3R,5R)-3-((4-(4-chlorophenyl)pyrido[3,4-d]pyridazin-1-yl)amino)-5-fluoropiperidine-1-carboxylate (15.00 mg, 32.76 μmol), and was obtained as a white solid (2.5 mg, 21% yield, 99% purity). ¹H NMR (400 MHz, DMSO-d₆) δ ppm 1.70-1.90 (m, 2H), 1.99 (br s, 1H), 2.11-2.18 (m, 1H), 2.23-2.25 (m, 3H), 2.94 (br s, 1H), 3.10 (br s, 1H), 4.71-4.78 (m, 1H), 5.08 (br s, 1 H), 7.50-7.55 (m, 1H), 7.62-7.68 (m, 2H), 7.72-7.78 (m, 2H), 8.19-8.28 (m, 1H), 8.32 (br d, J=5.50 Hz, 1H), 8.99-9.07 (m, 1H), 9.16-9.19 (m, 1H); ESI-MS m/z [M+H]⁺ 372.3.

### Example 176: 4-(4-chloro-2-fluorophenyl)-N-((3R,5R)-5-fluoro-1-methylpiperidin-3-yl)phthalazin-1-amine

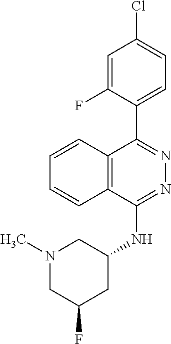 [SMILES: CN1C[C@H](F)C[C@@H](Nc2nnc(-c3ccc(Cl)cc3F)c3ccccc23)C1]

The title compound was prepared like Example 174, using tert-butyl (3R,5R)-3-((4-(4-chloro-2-fluorophenyl)phthalazin-1-yl)amino)-5-fluoropiperidine-1-carboxylate (50.00 mg, 105.28 μmol), and was obtained as a white solid (7.5 mg, 19%). ¹H NMR (400 MHz, DMSO-d₆) δ ppm 1.72-1.94 (m, 1H), 2.02 (br t, J=10.07 Hz, 1H), 2.15 (br d, J=12.51 Hz, 0.5H), 2.20-2.29 (m, 4.5H), 2.94 (br t, J=11.51 Hz, 1H), 3.07-3.13 (m, 1H), 4.70-4.87 (m, 1H), 4.92-5.12 (m, 1H), 7.30 (br d, J=7.75 Hz, 1H), 7.50 (br d, J=8.13 Hz, 2H), 7.57-7.68 (m, 2H), 7.82-7.88 (m, 1H), 7.89-7.96 (m, 1H), 8.19 (s, 1H), 8.43 (d, J=8.13 Hz, 1H); ESI-MS m/z [M+H]⁺ 389.2.

### Example 177: N-((3R,5R)-5-fluoro-1-methylpiperidin-3-yl)-4-(4-methoxyphenyl)phthalazin-1-amine

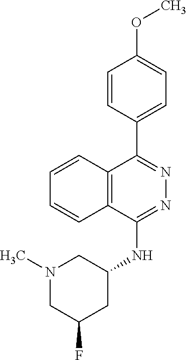

The title compound was prepared like Example 174, using of tert-butyl (3R,5R)-3-fluoro-5-((4-(4-methoxyphenyl)phthalazin-1-yl)amino)piperidine-1-carboxylate (28 mg, 0.062 mmol) and aq formaldehyde (37 wt %, 0.1 mL, 1.34 mmol), and was obtained as an ivory solid (8.4 mg, 37%). ¹H NMR (400 MHz, DMSO-d₆) δ ppm 1.19-1.50 (m, 1H), 1.69-1.93 (m, 1H), 1.99 (t, J=9.9 Hz, 1H), 2.08-2.21 (m, 1H), 2.09-2.28 (m, 3H), 2.87-3.02 (m, 1H), 3.10 (br dd, J=10.3, 3.3 Hz, 1H), 3.86 (s, 3H), 4.67-4.82 (m, 1H), 4.91-5.14 (m, 1H), 7.05-7.16 (m, 3H), 7.51-7.59 (m, 2H), 7.78-7.95 (m, 3H), 8.40 (d, J=8.0 Hz, 1H); ESI-MS m/z [M+H]⁺ 367.20.

### Example 178: N-((3R,5R)-5-fluoro-1-methylpiperidin-3-yl)-4-(2-fluoro-4-methoxyphenyl)phthalazin-1-amine

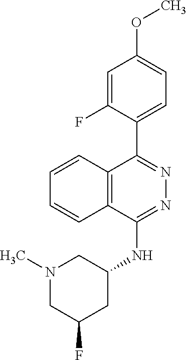

The title compound was prepared like Example 174, using of tert-butyl (3R,5R)-3-fluoro-5-((4-(2-fluoro-4-methoxyphenyl)phthalazin-1-yl)amino)piperidine-1-carboxylate (178.7 mg, 0.380 mmol) and aq formaldehyde (37 wt %, 0.400 mL, 5.37 mmol), and was obtained as a white solid (43.3 mg, 30%). ¹H NMR (400 MHz, DMSO-d₆) δ ppm 1.68-1.93 (m, 1H), 2.00 (t, J=10.0 Hz, 1H), 2.06-2.32 (m, 5H), 2.95 (br t, J=11.6 Hz, 1H), 3.10 (br dd, J=10.1, 3.2 Hz, 1H), 3.34 (s, 3H), 4.66-4.84 (m, 1H), 4.91-5.12 (m, 1H), 6.94-7.07 (m, 2H), 7.22 (d, J=7.8 Hz, 1H), 7.41-7.55 (m, 2H), 7.78-7.95 (m, 2H), 8.41 (d, J=8.2 Hz, 1H); ESI-MS m/z [M+H]⁺ 385.20.

### Example 179: N-((3R,5R)-5-fluoro-1-methylpiperidin-3-yl)-4-(4-methoxyphenyl)pyrido[3,4-d]pyridazin-1-amine

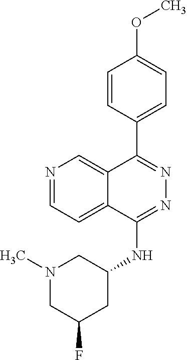

A formic acid salt of the title compound was prepared like Example 174, using tert-butyl (3R,5R)-3-fluoro-5-((4-(4-methoxyphenyl)pyrido[3,4-d]pyridazin-1-yl)amino)piperidine-1-carboxylate (15 mg, 33.08 μmol), and was obtained as a white solid (6.6 mg, 48% yield, 98% purity). ¹H NMR (400 MHz, DMSO-d₆) δ ppm 1.65-1.93 (m, 1H), 1.96-2.11 (m, 1H), 2.11-2.19 (m, 1H), 2.21-2.28 (m, 4H), 2.87-2.99 (m, 1H), 3.06-3.13 (m, 1H), 3.85-3.89 (m, 3H), 4.64-4.84 (m, 1H), 4.92-5.12 (m, 1H), 7.10-7.20 (m, 2H), 7.37-7.49 (m, 1H), 7.63-7.68 (m, 2H), 8.27-8.32 (m, 1H), 8.34 (s, 1H), 8.97-9.03 (m, 1H), 9.18 (s, 1H); ESI-MS m/z [M+H]⁺ 368.4.

### Example 180: N-((3R,5R)-5-fluoro-1-methylpiperidin-3-yl)-1-(2-fluoro-4-methoxyphenyl)pyrido[3,4-d]pyridazin-4-amine

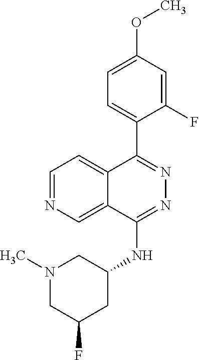 [SMILES: COc1ccc(-c2nnc(N[C@@H]3C[C@@H](F)CN(C)C3)c3cnccc23)c(F)c1]

The title compound was prepared like Example 174, using tert-butyl (3R,5R)-3-fluoro-5-((1-(2-fluoro-4-methoxyphenyl)pyrido[3,4-d]pyridazin-4-yl)amino)piperidine-1-carboxylate (140.00 mg, 296.93 μmol), and was obtained as a yellow solid (55 mg, 47% yield, 98% purity). ¹H NMR (400 MHz, DMSO-d₆) δ ppm 1.71-1.94 (m, 1H), 2.00-2.10 (m, 1H), 2.15-2.21 (m, 1H), 2.22-2.33 (m, 4H), 2.88-3.01 (m, 1H), 3.04-3.18 (m, 1H), 4.70-4.86 (m, 1H), 4.93-5.15 (m, 1H), 6.94-7.08 (m, 2H), 7.31-7.38 (m, 1H), 7.45-7.58 (m, 1H), 7.66-7.79 (m, 1H), 8.08-8.20 (m, 1H), 8.83-8.98 (m, 1H), 9.73-9.90 (m, 1H); ESI-MS m/z [M+H]⁺ 386.4.

### Example 181: N-((3R,5R)-5-fluoro-1-methylpiperidin-3-yl)-1-(4-fluoro-2-methoxyphenyl)pyrido[3,4-d]pyridazin-4-amine

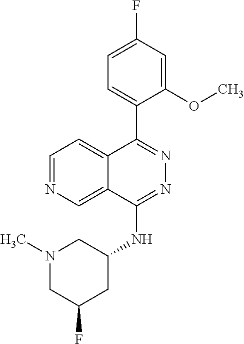

The title compound was prepared like Example 174, using tert-butyl (3R,5R)-3-fluoro-5-((1-(4-fluoro-2-methoxyphenyl)pyrido[3,4-d]pyridazin-4-yl)amino)piperidine-1-carboxylate (90.00 mg, 190.88 μmol), and was obtained as a yellow solid (50 mg, 67%). ¹H NMR (400 MHz, DMSO-d₆) δ ppm 1.70-1.93 (m, 1H), 1.96-2.09 (m, 1H), 2.12-2.35 (m, 5H), 2.85-3.14 (m, 3H), 3.67-3.78 (m, 3H), 4.69-4.82 (m, 1H), 4.92-5.13 (m, 1H), 6.89-7.03 (m, 1H), 7.08-7.24 (m, 2H), 7.30-7.50 (m, 1H), 7.59-7.75 (m, 1H), 8.03-8.29 (m, 1H), 8.78-8.94 (m, 1H), 9.67-9.86 (m, 1H); ESI-MS m/z [M+H]⁺ 386.4.

### Example 182: N-((3R,5R)-5-fluoro-1-methylpiperidin-3-yl)-4-(4-fluoro-2-methoxyphenyl)pyrido[3,4-d]pyridazin-1-amine

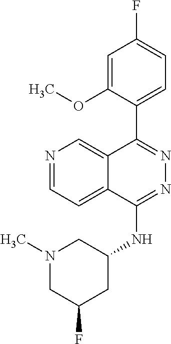 [SMILES: COc1cc(F)ccc1-c1nnc(N[C@@H]2C[C@@H](F)CN(C)C2)c2ccncc12]

The title compound was prepared like Example 174, using tert-butyl (3R,5R)-3-fluoro-5-((4-(4-fluoro-2-methoxyphenyl)pyrido[3,4-d]pyridazin-1-yl)amino)piperidine-1-carboxylate (50.00 mg, 106.04 μmol), and was obtained as a yellow solid (10 mg, 24% yield, 99% purity). ¹H NMR (400 MHz, DMSO-d₆) δ ppm 1.66-1.91 (m, 1H), 1.94-2.08 (m, 1 H), 2.10-2.38 (m, 5H), 2.82-3.17 (m, 3H), 4.72 (br s, 1H), 4.92-5.12 (m, 1H), 6.93-7.04 (m, 1H), 7.08-7.19 (m, 1H), 7.37-7.53 (m, 2H), 8.19 (s, 1H), 8.17-8.20 (m, 1H), 8.24-8.34 (m, 1H), 8.67-8.78 (m, 1H), 8.90-9.01 (m, 1H); ESI-MS m/z [M+H]⁺ 386.4.

### Example 183: 4-(4-chloro-2-fluorophenyl)-N-((3R,5R)-5-fluoro-1-methylpiperidin-3-yl)pyrido[3,4-d]pyridazin-1-amine

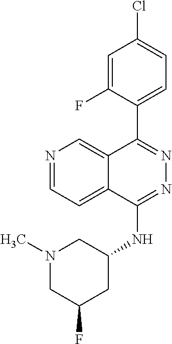 [SMILES: CN1C[C@H](F)C[C@@H](Nc2nnc(-c3ccc(Cl)cc3F)c3cnccc23)C1]

### Example 184: 1-(4-chloro-2-fluorophenyl)-N-((3R,5R)-5-fluoro-1-methylpiperidin-3-yl)pyrido[3,4-d]pyridazin-4-amine

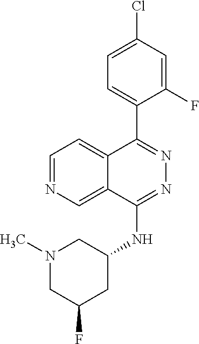 [SMILES: CN1C[C@H](F)C[C@@H](Nc2nnc(-c3ccc(Cl)cc3F)c3ccncc23)C1]

The title compounds were prepared like Example 174, using a mixture of tert-butyl (3R,5R)-3-((1-(4-chloro-2-fluorophenyl)pyrido[3,4-d]pyridazin-4-yl)amino)-5-fluoropiperidine-1-carboxylate and tert-butyl (3R,5R)-3-((4-(4-chloro-2-fluorophenyl)pyrido[3,4-d]pyridazin-1-yl)amino)-5-fluoropiperidine-1-carboxylate (100.00 mg, 210.12 μmol). The title compound of Example 183 was obtained as a yellow solid (8 mg, 9.6%). ¹H NMR (400 MHz, DMSO-d₆) δ ppm 1.69-1.92 (m, 1H), 1.96-2.07 (m, 1H), 2.11-2.18 (m, 1H), 2.20-2.30 (m, 4H), 2.87-3.01 (m, 1H), 3.04-3.15 (m, 1H), 4.62-4.86 (m, 1H), 4.90-5.13 (m, 1H), 7.24-7.78 (m, 4H), 8.16-8.23 (m, 1H), 8.29-8.41 (m, 1H), 8.92 (d, J=3.00 Hz, 1H), 8.97-9.09 (m, 1H); ESI-MS m/z [M+H]⁺ 390.3. The title compound of Example 184 was obtained as a white solid (25 mg, 30%). ¹H NMR (400 MHz, DMSO-d₆) δ ppm 1.68-1.92 (m, 1H), 1.97-2.09 (m, 1H), 2.11-2.28 (m, 5H), 2.89-3.01 (m, 1H), 3.06-3.15 (m, 1H), 4.80 (br s, 1H), 4.92-5.14 (m, 1H), 7.32-7.43 (m, 1H), 7.52 (dd, J=8.25, 2.00 Hz, 1H), 7.59-7.72 (m, 2H), 7.78-7.88 (m, 1H), 8.86-8.96 (m, 1H), 9.77-9.87 (m, 1H); ESI-MS m/z [M+H]⁺ 390.3.

### Example 185: 4-(4-chlorophenyl)-N-((3R,5S)-5-fluoro-1-methylpiperidin-3-yl)phthalazin-1-amine

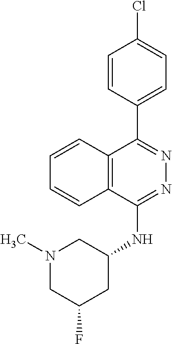 [SMILES: CN1C[C@@H](F)C[C@@H](Nc2nnc(-c3ccc(Cl)cc3)c3ccccc23)C1]

The title compound was prepared like Example 174, using tert-butyl (3R,5S)-3-((4-(4-chlorophenyl)phthalazin-1-yl)amino)-5-fluoropiperidine-1-carboxylate (100 mg, 218.85 μmol), and was obtained as a white solid (20.7 mg, 25.4% yield, 99.5% purity). ¹H NMR (400 MHz, DMSO-d₆) δ ppm 1.71 (quin, J=11.3 Hz, 1H), 1.93 (t, J=10.2 Hz, 1H), 2.03 (td, J=9.8, 6.0 Hz, 1H), 2.29 (s, 3H), 2.44 (br d, J=2.1 Hz, 1H), 3.08 (br d, J=5.9 Hz, 2H), 4.51 (br d, J=5.6 Hz, 1H), 4.65-4.89 (m, 1H), 7.26 (d, J=7.8 Hz, 1H), 7.58-7.68 (m, 4H), 7.75-7.81 (m, 1H), 7.86 (t, J=7.2 Hz, 1H), 7.90-7.96 (m, 1H), 8.38 (d, J=8.1 Hz, 1H); ESI-MS m/z [M+H]⁺ 371.1.

### Example 186: 2-(4-(((3R,5R)-5-fluoro-1-methylpiperidin-3-yl)amino)-5-methylpyrido[3,4-d]pyridazin-1-yl)-5-methylphenol

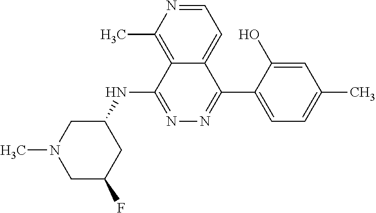

The title compound was prepared like Example 174 and was obtained as a solid. ¹H NMR (400 MHz, DMSO-d₆) δ ppm 1.96-2.16 (m, 2H), 2.31 (d, J=15.2 Hz, 7H), 2.54 (br d, J=3.9 Hz, 1H), 2.67 (br s, 1H), 2.81-2.96 (m, 1H), 3.08 (s, 3H), 4.71 (br s, 1H), 4.84-5.09 (m, 1H), 6.29 (d, J=7.3 Hz, 1H), 6.65-6.85 (m, 2H), 7.01-7.27 (m, 2H), 8.62 (d, J=5.5 Hz, 1H), 9.54 (s, 1H); ESI-MS m/z [M+H]⁺ 382.2.

### Example 187: 4-(4-chlorophenyl)-N-((3R,5R)-5-fluoropiperidin-3-yl)pyrido[3,4-d]pyridazin-1-amine

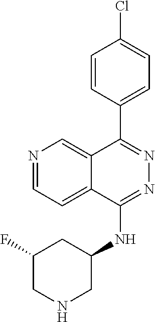 [SMILES: F[C@H]1CNC[C@H](Nc2nnc(-c3ccc(Cl)cc3)c3cnccc23)C1]

### Example 188: 1-(4-chlorophenyl)-N-((3R,5R)-5-fluoropiperidin-3-yl)pyrido[3,4-d]pyridazin-4-amine

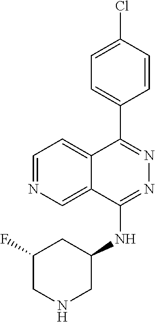 [SMILES: F[C@H]1CNC[C@H](Nc2nnc(-c3ccc(Cl)cc3)c3ccncc23)C1]

A solution of tert-butyl (3R,5R)-3-((1-(4-chlorophenyl)pyrido[3,4-d]pyridazin-4-yl)amino)-5-fluoropiperidine-1-carboxylate and tert-butyl (3R,5R)-3-((4-(4-chlorophenyl)pyrido[3,4-d]pyridazin-1-yl)amino)-5-fluoropiperidine-1-carboxylate (172.6 mg total, 0.377 mmol) in dioxane (1.88 mL) was treated with HCl (4 M in dioxane, 942 μL, 3.77 mmol). The reaction mixture was stirred at room temperature for 1 hour and then concentrated in vacuo. The residue was taken up in MeOH and filtered through a hydrophilic PTFE 0.45 m syringe filter (VWR®). The filtrate was purified by preparative HPLC (Method A). The first peak to elute was repurified separately by preparative HPLC (Method C) to give the title compound of Example 187, which was obtained as a white solid (7.7 mg, 5.7%). ¹H NMR (400 MHz, DMSO-d₆) δ ppm 1.84-2.06 (m, 1H), 2.24-2.40 (m, 1H), 2.57 (br d, J=10.0 Hz, 1H), 2.66-2.82 (m, 1H), 2.94-3.06 (m, 1H), 3.20 (br dd, J=12.4, 2.8 Hz, 1H), 4.49-4.70 (m, 1H), 4.76-5.01 (m, 1H), 7.46 (d, J=7.6 Hz, 1H), 7.61-7.69 (m, 2 H), 7.71-7.78 (m, 2H), 8.32 (d, J=5.6 Hz, 1H), 9.02 (d, J=5.8 Hz, 1H), 9.16 (s, 1H); ESI-MS m/z [M+H]+ 358.10. The second peak to elute was repurified separately by preparative HPLC (Method C) to give the title compound of Example 188, which was obtained as a pale yellow solid (22.7 mg, 17%). ¹H NMR (400 MHz, DMSO-d₆) δ ppm 1.86-2.07 (m, 1H), 2.25-2.39 (m, 1H), 2.54-2.63 (m, 1H), 2.67-2.85 (m, 1H), 2.93-3.06 (m, 1H), 3.21 (br dd, J=12.6, 2.8 Hz, 1H), 4.58-4.74 (m, 1H), 4.78-5.03 (m, 1H), 7.60-7.72 (m, 6H), 8.92 (d, J=5.6 Hz, 1H), 9.81 (s, 1H). ESI-MS m/z [M+H]+ 358.15.

### Example 189: 1-(4-chlorophenyl)-N-((3R,5R)-5-fluoro-1-methylpiperidin-3-yl)pyrido[3,4-d]pyridazin-4-amine

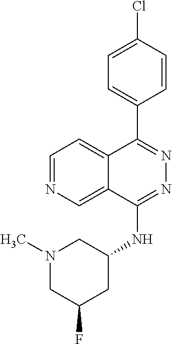 [SMILES: CN1C[C@H](F)C[C@@H](Nc2nnc(-c3ccc(Cl)cc3)c3ccncc23)C1]

A solution of 1-(4-chlorophenyl)-N-((3R,5R)-5-fluoropiperidin-3-yl)pyrido[3,4-d]pyridazin-4-amine (14 mg, 0.039 mmol) in MeOH (0.783 mL) was treated with paraformaldehyde (5.87 mg, 0.196 mmol) and sodium triacetoxyborohydride (41.5 mg, 0.196 mmol). The reaction mixture was stirred for 2 days. Additional paraformaldehyde (5.87 mg, 0.196 mmol) was added and the reaction mixture was stirred for 5 minutes. Then sodium borohydride (7.40 mg, 0.196 mmol) was added (vigorous gas evolution was observed). After 10 minutes, the reaction mixture was quenched with several drops of water. The reaction mixture was filtered through a hydrophilic PTFE 0.45 m syringe filter (VWR®), rinsing with MeOH. The filtrate was purified by preparative HPLC (Method C). The fractions containing the product were evaporated and repurified by preparative HPLC (Method A). The fraction containing clean product was evaporated and lyophilized to give a TFA salt of the title compound as a yellow solid (6.2 mg, 33%). ¹H NMR (400 MHz, CD₃OD) δ ppm 2.06-2.34 (m, 1H), 2.62-2.76 (m, 1H), 3.03 (s, 4H), 3.38-3.54 (m, 1H), 3.90 (br t, J=11.9 Hz, 1H), 4.07 (br d, J=11.5 Hz, 1H), 5.02-5.16 (m, 1H), 5.28-5.46 (m, 1H), 7.62-7.76 (m, 4H), 7.84 (dd, J=5.7, 0.8 Hz, 1H), 9.01 (d, J=5.6 Hz, 1H), 9.75 (s, 1H); ESI-MS m/z [M+H]+ 372.10.

### Example 190: 4-(4-chlorophenyl)-N-((3R,5R)-5-fluoropiperidin-3-yl)phthalazin-1-amine

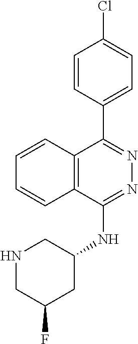 [SMILES: F[C@H]1CNC[C@H](Nc2nnc(-c3ccc(Cl)cc3)c3ccccc23)C1]

The title compound was prepared like Preparation 41, using tert-butyl (3R,5R)-3-((4-(4-chlorophenyl)phthalazin-1-yl)amino)-5-fluoropiperidine-1-carboxylate (75.6 mg, 0.165 mmol), and was obtained as a white solid (38.4 mg, 65%). ¹H NMR (400 MHz, DMSO-d₆) δ ppm 1.76-1.99 (m, 1H), 2.16-2.29 (m, 1H), 2.49 (br d, J=10.7 Hz, 1H), 2.58-2.77 (m, 1H), 2.84-2.99 (m, 1H), 3.12 (br dd, J=12.1, 3.2 Hz, 1H), 4.45-4.62 (m, 1H), 4.69-4.94 (m, 1H), 7.10 (d, J=7.6 Hz, 1H), 7.50-7.61 (m, 4H), 7.68-7.73 (m, 1H), 7.79 (td, J=7.6, 1.1 Hz, 1H), 7.82-7.89 (m, 1H), 8.35 (d, J=8.2 Hz, 1H); ESI-MS m/z [M+H]+ 357.10.

### Example 191: 4-(4-chlorophenyl)-N-(5,5-difluoropiperidin-3-yl)phthalazin-1-amine

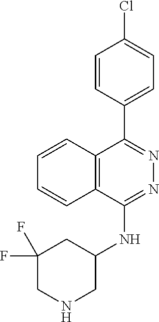 [SMILES: FC1(F)CNCC(Nc2nnc(-c3ccc(Cl)cc3)c3ccccc23)C1]

A TFA salt of the title compound was prepared like Preparation 41, using tert-butyl 5-((4-(4-chlorophenyl)phthalazin-1-yl)amino)-3,3-difluoropiperidine-1-carboxylate (237 mg, 0.500 mmol), and was obtained as a yellow oil (6.1 mg, 2.5%). ¹H NMR (400 MHz, CD₃OD) δ ppm 2.44-2.69 (m, 1H), 2.86-2.99 (m, 1H), 3.29-3.37 (m, 1H), 3.57-3.75 (m, 1H), 3.78-3.95 (m, 2H), 4.86-4.91 (m, 1H), 7.79 (d, J=1.9 Hz, 4H), 8.14-8.23 (m, 2H), 8.33 (ddd, J=8.3, 6.3, 2.2 Hz, 1H), 8.65 (d, J=8.3 Hz, 1H); ESI-MS m/z [M+H]+ 375.10.

### Example 192: 4-(4-chlorophenyl)-N-((3R,5R)-5-fluoro-1-methylpiperidin-3-yl)phthalazin-1-amine

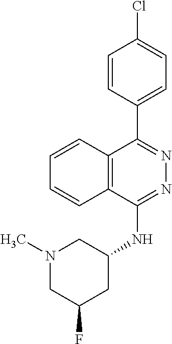 [SMILES: CN1C[C@H](F)C[C@@H](Nc2nnc(-c3ccc(Cl)cc3)c3ccccc23)C1]

A solution of 4-(4-chlorophenyl)-N-((3R,5R)-5-fluoropiperidin-3-yl)phthalazin-1-amine TFA salt (64.4 mg, 0.137 mmol) in MeOH (1.37 mL) was treated with paraformaldehyde (20.5 mg, 0.684 mmol), followed by sodium borohydride (25.9 mg, 0.684 mmol). The reaction mixture was stirred at room temperature for 45 minutes. Additional paraformaldehyde (20.53 mg, 0.684 mmol) was added and the mixture was stirred for 5 minutes. Next, additional sodium borohydride (25.9 mg, 0.684 mmol) was added in small portions and the reaction mixture was stirred for another 1 hour. The reaction mixture was quenched with water (1 mL) and filtered through a hydrophilic PTFE 0.45 m syringe filter (VWR®), rinsing with MeOH. The filtrate was purified by preparative HPLC (Method C). The fractions containing the product were evaporated and lyophilized to give the title compound as an ivory solid (16 mg, 32%). ¹H NMR (400 MHz, DMSO-d₆) δ ppm 1.62-1.84 (m, 1H), 1.86-2.23 (m, 5H), 2.79-2.96 (m, 1H), 2.97-3.13 (m, 1H), 4.58-4.81 (m, 1H), 4.84-5.08 (m, 1H), 7.16 (br d, J=5.4 Hz, 1H), 7.51-7.62 (m, 4H), 7.67-7.74 (m, 1H), 7.76-7.90 (m, 2H), 8.36 (d, J=8.2 Hz, 1H); ESI-MS m/z [M+H]+ 371.10.

### Example 193: N-((3R,5R)-1-ethyl-5-fluoropiperidin-3-yl)-4-(4-methoxyphenyl)phthalazin-1-amine

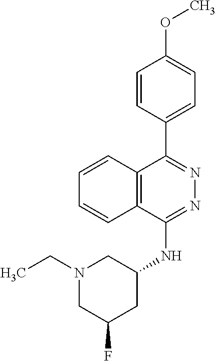 [SMILES: CCN1C[C@H](F)C[C@@H](Nc2nnc(-c3ccc(OC)cc3)c3ccccc23)C1]

A solution of N-((3R,5R)-5-fluoropiperidin-3-yl)-4-(4-methoxyphenyl)phthalazin-1-amine (38 mg, 0.108 mmol) and acetaldehyde (5 M in THF, 108 μL, 0.539 mmol) in MeOH (0.5 mL) was stirred at room temperature for 5 minutes and then treated with sodium cyanoborohydride (13.6 mg, 0.216 mmol). The reaction mixture was stirred at room temperature overnight and subsequently quenched with a few drops of water, diluted with additional MeOH (1 mL) and filtered through a hydrophilic PTFE 0.45 μm syringe filter (VWR®). The filtrate was purified by preparative HPLC (Method C). The product-containing fraction containing was evaporated and repurified by preparative HPLC (Method C). The fractions containing the product were evaporated and lyophilized to give the title compound as a white solid in (1.6 mg, 3.9% yield, 93% purity). ¹H NMR (400 MHz, DMSO-d₆) δ ppm 0.95 (t, J=7.2 Hz, 3H), 1.02-1.03 (m, 1H), 1.64-1.88 (m, 1H), 1.94 (t, J=10.0 Hz, 1H), 2.04-2.22 (m, 2H), 2.37 (q, J=7.2 Hz, 2H), 2.91-3.02 (m, 1H), 3.11 (br dd, J=10.2, 3.5 Hz, 1H), 3.78 (s, 3H), 4.57-4.75 (m, 1H), 4.82-5.07 (m, 1H), 7.04 (d, J=8.7 Hz, 3H), 7.44-7.53 (m, 2H), 7.71-7.87 (m, 3H), 8.32 (d, J=8.2 Hz, 1H); ESI-MS m/z [M+H]⁺ 381.20.

### Example 194: N-((3R,5R)-1-(cyclopropylmethyl)-5-fluoropiperidin-3-yl)-4-(4-methoxyphenyl)phthalazin-1-amine

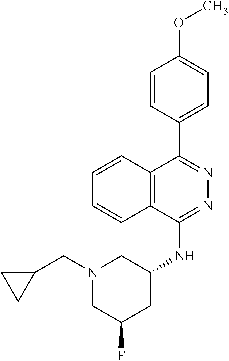 [SMILES: COc1ccc(-c2nnc(N[C@@H]3C[C@@H](F)CN(CC4CC4)C3)c3ccccc23)cc1]

The title compound was prepared like Example 193, using N-((3R,5R)-5-fluoropiperidin-3-yl)-4-(4-methoxyphenyl)phthalazin-1-amine (38 mg, 0.108 mmol) and cyclopropanecarbaldehyde (37.8 mg, 0.539 mmol), and was obtained as an ivory solid (23.5 mg, 54%). ¹H NMR (400 MHz, DMSO-d₆) δ ppm 0.01 (br d, J=4.9 Hz, 2H), 0.24-0.49 (m, 2H), 0.67-0.88 (m, 1H), 1.62-1.84 (m, 1H), 2.03 (br t, J=10.0 Hz, 1H), 2.11-2.32 (m, 4H), 3.06 (br s, 1H), 3.17 (br d, J=7.6 Hz, 1H), 3.77 (s, 3H), 4.68 (br d, J=4.6 Hz, 1H), 4.82-5.06 (m, 1H), 6.97-7.08 (m, 3H), 7.42-7.51 (m, 2H), 7.70-7.85 (m, 3H), 8.31 (d, J=8.0 Hz, 1H); ESI-MS m/z [M+H]⁺ 407.20.

### Example 195: N-((3R,5R)-1-ethyl-5-fluoropiperidin-3-yl)-1-(4-methoxyphenyl)pyrido[3,4-d]pyridazin-4-amine

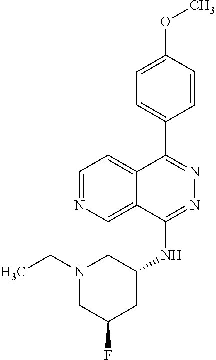 [SMILES: CCN1C[C@H](F)C[C@@H](Nc2nnc(-c3ccc(OC)cc3)c3ccncc23)C1]

To a solution of N-((3R,5R)-5-fluoropiperidin-3-yl)-1-(4-methoxyphenyl)pyrido[3,4-d]pyridazin-4-amine (40 mg, 113.19 μmol) in DCM (2 mL) were added NaBH₃CN (10.67 mg, 169.78 μmol) and acetaldehyde (49.86 mg, 1.13 mmol, 63.52 μL). The mixture was stirred at 25° C. for 2 hours. LC-MS showed the starting material was completely consumed. The reaction mixture was concentrated and purified by preparative HPLC (Method C). The title compound was obtained as a yellow solid (25 mg, 51% yield, 88% purity). ¹H NMR (400 MHz, DMSO-d₆) δ ppm 0.94-1.03 (m, 3H), 1.71-1.93 (m, 1H), 2.01-2.14 (m, 1H), 2.15-2.36 (m, 2H), 2.39-2.44 (m, 2H), 2.92-3.04 (m, 1H), 3.07-3.17 (m, 1H), 3.78-3.84 (m, 3H), 4.60-4.79 (m, 1H), 4.88-5.10 (m, 1H), 7.00-7.18 (m, 2H), 7.56 (br d, J=8.00 Hz, 2H), 7.60-7.72 (m, 1H) 8.79-8.96 (m, 1H) 9.60-9.77 (m, 1H); ESI-MS m/z [M+H]⁺ 382.2.

### Example 196: N-((3R,5R)-5-fluoro-1-isopropylpiperidin-3-yl)-4-(4-methoxyphenyl)phthalazin-1-amine

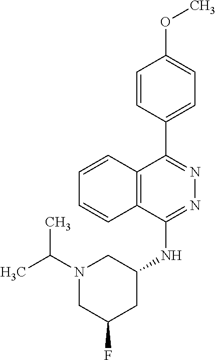 [SMILES: COc1ccc(-c2nnc(N[C@@H]3C[C@@H](F)CN(C(C)C)C3)c3ccccc23)cc1]

A solution of N-((3R,5R)-5-fluoropiperidin-3-yl)-4-(4-methoxyphenyl)phthalazin-1-amine (38 mg, 0.108 mmol) and 2-bromopropane (30.4 μL, 0.323 mmol) in DMF (0.5 mL) was treated with K₂CO₃ (29.8 mg, 0.216 mmol). The reaction mixture was stirred at 50° C. overnight and then quenched with a few drops of water, diluted with additional MeOH (1 mL) and filtered through a hydrophilic PTFE 0.45 m syringe filter (VWR®). The filtrate was purified by preparative HPLC (Method C). The product-containing fractions were evaporated and repurified by preparative HPLC (Method C). The fractions containing the product were evaporated and lyophilized to give the title compound as an ivory solid (6.3 mg, 15%). ¹H NMR (400 MHz, DMSO-d₆) δ ppm 0.99 (br d, J 4.5 Hz, 6H), 1.71-1.97 (m, 1H), 2.14-2.30 (m, 2H), 2.36-2.46 (m, 1H), 2.74-2.88 (m, 1H), 2.94 (br t, J=11.5 Hz, 1H), 3.11 (br d, J=7.4 Hz, 1H), 3.86 (s, 3H), 4.59-4.82 (m, 1H), 4.89-5.12 (m, 1H), 7.04-7.16 (m, 3H), 7.51-7.60 (m, 2H), 7.78-7.95 (m, 3H), 8.39 (d, J=8.0 Hz, 1H); ESI-MS m/z [M+H]⁺ 395.20.

### Example 197: 1-(4-chlorophenyl)-N-(1-(3-methoxycyclobutyl)piperidin-3-yl)pyrido[3,4-d]pyridazin-4-amine

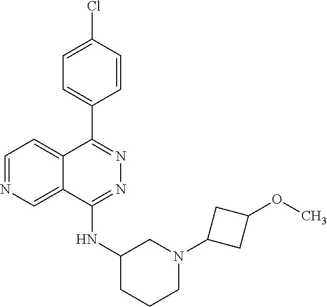 [SMILES: COC1CC(N2CCCC(Nc3nnc(-c4ccc(Cl)cc4)c4ccncc34)C2)C1]

To a vial charged with 3-methoxycyclobutan-1-one (0.050 g, 0.500 mmol), 1-(4-chlorophenyl)-N-(piperidin-3-yl)pyrido[3,4-d]pyridazin-4-amine (0.170 g, 0.5 mmol), acetic acid (0.029 mL, 0.500 mmol) and THE (1.000 mL) was added NaBH(OAc)₃ (0.148 g, 0.700 mmol). The resulting yellow suspension was stirred overnight under N₂ at room temperature. DMF (1 mL) was added and the mixture was filtered and purified by preparative HPLC to give the title compound (1 mg, 0.47%). ¹H NMR (400 MHz, CD₃OD) δ ppm 1.77-1.86 (m, 4H), 2.05-2.16 (m, 4H), 2.47-2.50 (m, 2H), 3.22-3.22 (m, 3H), 3.34-3.41 (m, 1H), 3.63-3.67 (m, 1H), 4.59-4.64 (m, 1H), 7.58-7.64 (m, 4H), 7.71-7.74 (m, 1H), 8.86-8.90 (m, 1H), 9.68-9.73 (m, 1H); ESI-MS m/z [M+H]⁺ 424.4.

### Example 198: 3-((5-(4-methoxyphenyl)pyrido[2,3-d]pyridazin-8-yl)amino)phenol

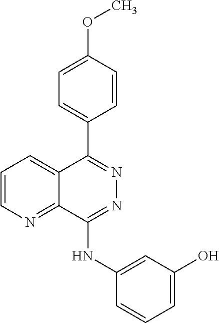 [SMILES: COc1ccc(-c2nnc(Nc3cccc(O)c3)c3ncccc23)cc1]

A mixture of 8-chloro-5-(4-methoxyphenyl)pyrido[2,3-d]pyridazine (33.00 mg, 121.46 μmol), 3-aminophenol (13.25 mg, 121.46 μmol), TFA (27.70 mg, 242.91 μmol, 17.99 μL) and tert-butyl alcohol (1.5 mL) was stirred at 30° C. for 12 hours. LC-MS showed one main peak with desired m/z. The mixture was filtered and the filtrate was concentrated and purified by preparative HPLC (Method C). The title compound was obtained as a yellow solid (5.0 mg, 11% yield, 96% purity). ¹H NMR (400 MHz, DMSO-d₆) δ ppm 3.87 (br s, 3H), 6.48 (br d, J=6.50 Hz, 1H), 7.04-7.28 (m, 3H), 7.44 (br d, J=6.38 Hz, 1H), 7.67 (br d, J=7.00 Hz, 2H), 7.91 (br s, 1H), 7.99 (br s, 1H), 8.37 (br d, J=7.25 Hz, 1H), 9.26 (br s, 1H), 9.42 (br d, J=6.25 Hz, 2H); ESI-MS m/z [M+H]⁺ 345.3.

### Example 199: 3-((4-(4-methoxyphenyl)-5,6,7,8-tetrahydrophthalazin-1-yl)amino)phenol

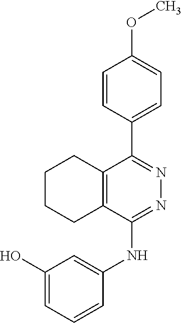 [SMILES: COc1ccc(-c2nnc(Nc3cccc(O)c3)c3c2CCCC3)cc1]

A mixture of 1-chloro-4-(4-methoxyphenyl)-5,6,7,8-tetrahydrophthalazine (100 mg, 363.97 μmol), 3-aminophenol (47.66 mg, 436.77 μmol), BrettPhos (19.54 mg, 36.40 μmol), BrettPhos-Pd-G3 (32.99 mg, 36.40 μmol) and Cs₂CO₃ (237.18 mg, 727.95 μmol) in DMF (5 mL) was degassed and purged with N₂ (3×). The mixture was stirred at 90° C. for 12 hours under N₂ atmosphere and then concentrated under vacuum and purified by preparative HPLC (Method B). The title compound was obtained as a white solid (60 mg, 47% yield, 99% purity). ¹H NMR (400 MHz, DMSO-d₆) δ ppm 1.60-1.69 (m, 2H), 1.79-1.89 (m, 2H), 2.55 (br t, J=5.88 Hz, 2H), 2.60-2.68 (m, 2H), 3.82 (s, 3H) 6.27-6.47 (m, 1H) 6.98-7.14 (m, 4H) 7.37 (t, J=2.00 Hz, 1H) 7.42-7.49 (m, 2H) 7.78 (s, 1H) 8.27 (s, 1H); ESI-MS m/z [M+H]+ 348.1.

### Example 200: 5-chloro-2-(1-(((3R,5R)-5-fluoro-1-methylpiperidin-3-yl)amino)-7,8-dihydro-5H-pyrano[3,4-d]pyridazin-4-yl)phenol

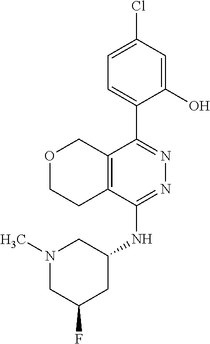 [SMILES: CN1C[C@H](F)C[C@@H](Nc2nnc(-c3ccc(Cl)cc3O)c3c2CCOC3)C1]

A mixture of 4-chloro-N-((3R,5R)-5-fluoro-1-methylpiperidin-3-yl)-7,8-dihydro-5H-pyrano[3,4-d]pyridazin-1-amine (70 mg, 232.74 μmol), (4-chloro-2-hydroxyphenyl)boronic acid (60.18 mg, 349.11 μmol), Pd(dppf)Cl₂·CH₂Cl₂ (38.01 mg, 46.55 μmol) and Cs₂CO₃ (151.67 mg, 465.48 μmol) in dioxane (1.5 mL) and H₂O (0.3 mL) was stirred at 100° C. for 1 hour under N₂. LC-MS showed one main peak with desired m/z. The reaction mixture was acidified with 1 M HCl aq (2 mL) and extracted with EtOAc (3 mL). The organic layer was discarded and the aqueous layer was filtered. The filtrate was purified by preparative HPLC (Boston Prime C18-5 μm, 30 mm×150 mm column) using a gradient of 0 to 28% ACN in water (with FA) to give a formic acid salt of the title compound as a white solid (31.7 mg, 31% yield, 95% purity). ¹H NMR (400 MHz, DMSO-d₆) δ ppm 1.65-1.85 (m, 1H), 1.95 (br t, J=9.9 Hz, 1H), 2.07-2.26 (m, 5H), 2.42-2.47 (m, 2H), 2.83-2.98 (m, 2H), 3.91 (br t, J=5.4 Hz, 2H), 4.31 (s, 2H), 4.56 (br s, 1H), 4.83-5.07 (m, 1H), 5.88 (br d, J=8.0 Hz, 1H), 6.90-7.01 (m, 2H), 7.16 (d, J=8.0 Hz, 1H), 8.17 (s, 1H), 9.90-10.85 (m, 1H); ESI-MS m/z [M+H]⁺ 393.1.

### Example 201: 2-(1-(((3R,5R)-5-fluoro-1-methylpiperidin-3-yl)amino)-7,8-dihydro-5H-pyrano[3,4-d]pyridazin-4-yl)-5-methylphenol

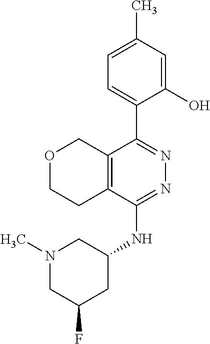 [SMILES: Cc1ccc(-c2nnc(N[C@@H]3C[C@@H](F)CN(C)C3)c3c2COCC3)c(O)c1]

A mixture of 4-chloro-N-((3R,5R)-5-fluoro-1-methylpiperidin-3-yl)-7,8-dihydro-5H-pyrano[3,4-d]pyridazin-1-amine (60 mg, 199.50 μmol), (2-hydroxy-4-methylphenyl)boronic acid (45.47 mg, 299.24 μmol), Pd(dppf)Cl₂·CH₂Cl₂ (32.58 mg, 39.90 μmol) and Cs₂CO₃ (130.00 mg, 398.99 μmol) in dioxane (1 mL) and H₂O (0.3 mL) was stirred at 100° C. for 1 hour under N₂. LC-MS showed one main peak with desired m/z. The reaction mixture was acidified with 1 M HCl aq (2 mL) and extracted with EtOAc (3 mL). The organic layer was discarded. The aqueous layer was filtered and the filtrate was purified by preparative HPLC (Boston Prime C18-5 μm, 30 mm×150 mm column) using a gradient of 0 to 25% ACN in water (with FA) to give a formic acid salt of the title compound as a white solid (23.5 mg, 28.1% yield, 99% purity). ¹H NMR (400 MHz, DMSO-d₆) δ ppm 1.67-1.87 (m, 1H), 1.98 (br t, J=10.2 Hz, 1H), 2.03-2.19 (m, 2H), 2.24 (s, 3H), 2.27 (s, 3H), 2.46 (br t, J=5.1 Hz, 2H), 2.89 (br t, J=11.0 Hz, 1H), 2.99 (br d, J=7.3 Hz, 1H), 3.91 (t, J=5.6 Hz, 2H), 4.33 (s, 2H), 4.57 (br d, J=6.3 Hz, 1H), 4.87-5.07 (m, 1H), 5.83 (d, J=8.0 Hz, 1H), 6.61-6.78 (m, 2H), 7.01 (d, J=7.5 Hz, 1H), 8.14 (s, 1H); ESI-MS m/z [M+H]⁺ 373.2.

### Example 202: 2-(1-(((3R,5R)-5-fluoro-1-methylpiperidin-3-yl)amino)-7,8-dihydro-5H-pyrano[3,4-d]pyridazin-4-yl)phenol

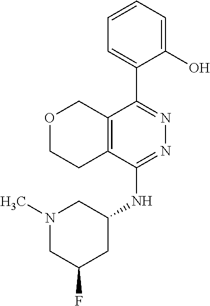 [SMILES: CN1C[C@H](F)C[C@@H](Nc2nnc(-c3ccccc3O)c3c2CCOC3)C1]

A mixture of 4-chloro-N-((3R,5R)-5-fluoro-1-methylpiperidin-3-yl)-7,8-dihydro-5H-pyrano[3,4-d]pyridazin-1-amine (70 mg, 232.74 μmol), (2-hydroxyphenyl)boronic acid (48.15 mg, 349.12 μmol), Pd(dppf)Cl₂·CH₂Cl₂ (38.01 mg, 46.55 μmol) and Cs₂CO₃ (151.67 mg, 465.49 μmol) in dioxane (1.5 mL) and H₂O (0.3 mL) was stirred at 100° C. for 1 hour under N₂. LC-MS showed one main peak with desired m/z. The reaction mixture was acidified with 1 M HCl aq (2 mL) and extracted with EtOAc (3 mL). The organic layer was discarded. The aqueous layer was filtered and the filtrate was purified by preparative HPLC (Boston Prime C18-5 μm, 30 mm×150 mm column) using a gradient of 0 to 20% ACN in water (with NH₃H₂O) to give a formic acid salt of the title compound as a white solid (31.2 mg, 33.1% yield, 95% purity). ¹H NMR (400 MHz, DMSO-d₆) δ ppm 1.66-1.87 (m, 1H), 1.96 (br t, J=9.9 Hz, 1H), 2.23 (s, 4H), 2.47 (br d, J=6.3 Hz, 2H), 2.88 (br t, J=11.2 Hz, 1H), 2.99 (br d, J=9.8 Hz, 1H), 3.92 (br t, J=5.5 Hz, 2H), 4.33 (s, 2H), 4.51-4.68 (m, 1H), 4.84-5.11 (m, 1H), 5.85 (br d, J=8.0 Hz, 1H), 6.83-6.98 (m, 2H), 7.13 (dd, J=7.5, 1.5 Hz, 1H), 7.21-7.32 (m, 1H), 8.18 (s, 1H), 9.18-10.48 (m, 1H); ESI-MS m/z [M+H]⁺ 359.1.

### Example 203: 5-fluoro-2-(1-(((3R,5R)-5-fluoro-1-methylpiperidin-3-yl)amino)-7,8-dihydro-5H-pyrano[3,4-d]pyridazin-4-yl)phenol

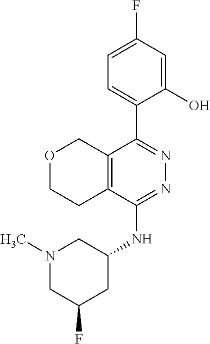 [SMILES: CN1C[C@H](F)C[C@@H](Nc2nnc(-c3ccc(F)cc3O)c3c2CCOC3)C1]

A mixture of 4-chloro-N-((3R,5R)-5-fluoro-1-methylpiperidin-3-yl)-7,8-dihydro-5H-pyrano[3,4-d]pyridazin-1-amine (60 mg, 199.50 μmol), (4-fluoro-2-hydroxyphenyl)boronic acid (46.66 mg, 299.25 μmol), Pd(dppf)Cl₂·CH₂Cl₂ (32.58 mg, 39.90 μmol) and Cs₂CO₃ (130 mg, 399 μmol) in dioxane (1 mL) and H₂O (0.3 mL) was stirred at 100° C. for 1 hour under N₂. LC-MS showed one main peak with desired m/z. The reaction mixture was acidified with 1 M HCl aq (2 mL) and extracted with EtOAc (3 mL). The organic layer was discarded. The aqueous layer was filtered and the filtrate was purified by preparative HPLC (Boston Prime C18-5 μm, 30 mm×150 mm column) using a gradient of 0 to 24% ACN in water (with FA) to give a formic acid salt of the title compound as a white solid (29.3 mg, 34.8% yield, 95% purity). ¹H NMR (400 MHz, DMSO-d₆) δ ppm 1.66-1.88 (m, 1H), 1.98 (br t, J=9.9 Hz, 1H), 2.06-2.29 (m, 5H), 2.43-2.49 (m, 2H), 2.90 (br t, J=10.9 Hz, 1H), 2.99 (br d, J=7.5 Hz, 1H), 3.92 (t, J=5.6 Hz, 2H), 4.32 (s, 2H), 4.51-4.65 (m, 1H), 4.87-5.06 (m, 1H), 5.87 (d, J=8.0 Hz, 1H), 6.65-6.80 (m, 2H), 7.10-7.21 (m, 1H), 8.15 (s, 1H); ESI-MS m/z [M+H]⁺ 377.2.

### Example 204: 1-(2-(difluoromethyl)-4-methylphenyl)-N-((3R,5R)-5-fluoro-1-methylpiperidin-3-yl)-7,8-dihydro-5H-pyrano[3,4-d]pyridazin-4-amine

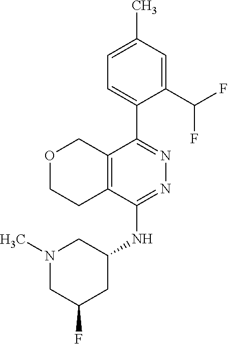 [SMILES: Cc1ccc(-c2nnc(N[C@@H]3C[C@@H](F)CN(C)C3)c3c2COCC3)c(C(F)F)c1]

To a mixture of 1-chloro-N-((3R,5R)-5-fluoro-1-methylpiperidin-3-yl)-7,8-dihydro-5H-pyrano[3,4-d]pyridazin-4-amine (40 mg, 133.00 μmol) and 2-(2-(difluoromethyl)-4-methylphenyl)-4,4,5,5-tetramethyl-1,3,2-dioxaborolane (53.49 mg, 199.50 μmol) in dioxane (1.5 mL) and H₂O (0.5 mL) were added Cs₂CO₃ (86.67 mg, 265.99 μmol) and Pd(dppf)Cl₂·CH₂Cl₂ (21.72 mg, 26.60 μmol). The reaction mixture was stirred at 100° C. for 1 hour and then acidified with 1 M HCl aq to pH 2 and extracted with EtOAc (3 mL). The aqueous phase was collected and purified by preparative HPLC (Boston Prime C18-5 μm, 30 mm×150 mm column) using a gradient of 13 to 33% ACN in water (with FA). A formic acid salt of the title compound was obtained as a yellow solid (26 mg, 43%). ¹H NMR (400 MHz, DMSO-d₆) δ ppm 1.60-1.80 (m, 1H), 1.90 (br t, J=10.1 Hz, 1H), 2.05-2.17 (m, 2H), 2.21 (s, 3H), 2.27 (br s, 2H), 2.43 (s, 3H), 2.84-3.03 (m, 2H), 3.76 (br t, J=5.4 Hz, 2H), 4.47 (s, 2H), 4.58 (br s, 1H), 4.87-5.04 (m, 1H), 5.79 (d, J=7.8 Hz, 1H), 6.58-6.88 (m, 1H), 7.30 (d, J=7.7 Hz, 1H), 7.42 (br d, J=7.7 Hz, 1H), 7.56 (s, 1H), 8.17 (s, 1H). ¹⁹F NMR (377 MHz, DMSO-d₆), δ ppm −180.74 (br s, 1 F), −109.11 (s, 1 F); ESI-MS m/z [M+H]+ 407.1.

### Example 205: N-((3R,5R)-5-fluoro-1-methylpiperidin-3-yl)-1-(2-fluoro-4-(trifluoromethoxy)phenyl)-7,8-dihydro-5H-pyrano[3,4-d]pyridazin-4-amine

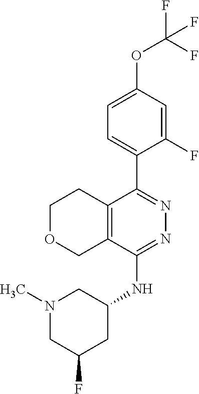 [SMILES: CN1C[C@H](F)C[C@@H](Nc2nnc(-c3ccc(OC(F)(F)F)cc3F)c3c2COCC3)C1]

To a mixture of 1-chloro-N-((3R,5R)-5-fluoro-1-methylpiperidin-3-yl)-7,8-dihydro-5H-pyrano[3,4-d]pyridazin-4-amine (40 mg, 133.00 μmol) and 2-(2-fluoro-4-(trifluoromethoxy)phenyl)-4,4,5,5-tetramethyl-1,3,2-dioxaborolane (61.06 mg, 199.50 μmol) in dioxane (1.5 mL) and H₂O (0.5 mL) were added Cs₂CO₃ (86.67 mg, 266.00 μmol) and Pd(dppf)Cl₂·CH₂Cl₂ (21.72 mg, 26.60 μmol). The mixture was stirred at 100° C.° C. for 1 hour. The reaction mixture was acidified with 1 M HCl aq to pH 2 and extracted with EtOAc (3 mL). The aqueous phase was collected and purified by preparative HPLC (Boston Prime C18-5 μm, 30 mm×150 mm column) using a gradient of 20 to 40% ACN in water (with FA). A formic acid salt of the title compound was obtained as a brown solid (15.7 mg, 24.1%). ¹H NMR (400 MHz, DMSO-d₆) δ ppm 1.61-1.81 (m, 1H), 1.92 (br t, J=10.0 Hz, 1H), 2.06-2.19 (m, 2H), 2.22 (s, 3H), 2.38 (br s, 2H), 2.84-3.03 (m, 2H), 3.78 (br t, J=5.3 Hz, 2H), 4.48 (s, 2H), 4.53-4.66 (m, 1H), 4.86-5.06 (m, 1H), 5.88 (br d, J=7.8 Hz, 1H), 7.37 (br d, J=8.6 Hz, 1H), 7.54 (br d, J=10.1 Hz, 1H), 7.61 (t, J=8.4 Hz, 1H), 8.14 (s, 1H). ¹⁹F NMR (377 MHz, DMSO-d₆), δ ppm −180.74 (br s, 1 F), −111.12 (s, 1 F), −56.94 (s, 1 F); ESI-MS m/z [M+H]+ 445.1.

### Example 206: 1-(2-(difluoromethyl)-4-methoxyphenyl)-N-((3R,5R)-5-fluoro-1-methylpiperidin-3-yl)-7,8-dihydro-5H-pyrano[3,4-d]pyridazin-4-amine

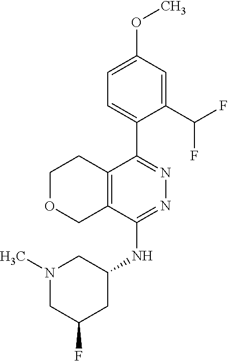 [SMILES: COc1ccc(-c2nnc(N[C@@H]3C[C@@H](F)CN(C)C3)c3c2CCOC3)c(C(F)F)c1]

To a mixture of 1-chloro-N-((3R,5R)-5-fluoro-1-methylpiperidin-3-yl)-7,8-dihydro-5H-pyrano[3,4-d]pyridazin-4-amine (40 mg, 133.00 μmol) and 2-(2-(difluoromethyl)-4-methoxyphenyl)-4,4,5,5-tetramethyl-1,3,2-dioxaborolane (56.68 mg, 199.50 μmol) in dioxane (1.5 mL) and H₂O (0.5 mL) were added Cs₂CO₃ (86.67 mg, 266.00 μmol) and Pd(dppf)Cl₂·CH₂Cl₂ (21.72 mg, 26.60 μmol, 0.2 eq). The reaction mixture was stirred at 100° C. for 1 hour and then adjusted to pH 2 with 1 M HCl aq and extracted with EtOAc (3 mL). The aqueous phase was collected and purified by preparative HPLC (Boston Prime C18-5 μm, 30 mm×150 mm column) using a gradient of 6 to 36% ACN in water (with FA) to give a formic acid salt of the title compound as a yellow solid. ¹H NMR (400 MHz, DMSO-d₆) δ ppm 1.59-1.80 (m, 1H), 1.90 (br t, J=10.1 Hz, 1H), 2.05-2.18 (m, 2H), 2.21 (s, 3H), 2.29 (br s, 2H), 2.83-3.02 (m, 2H), 3.76 (br t, J=5.4 Hz, 2H), 3.86 (s, 3H), 4.47 (s, 2H), 4.52-4.65 (m, 1H), 4.86-5.05 (m, 1H), 5.78 (d, J=7.8 Hz, 1H), 6.60-6.90 (m, 1H), 7.17 (dd, J=8.5, 2.4 Hz, 1H), 7.23 (d, J=2.4 Hz, 1H), 7.35 (d, J=8.4 Hz, 1H), 8.16 (s, 1H); ¹⁹F NMR (377 MHz, DMSO-d₆) δ ppm −180.74 (br s, 1 F), −110.02 (br s, 1 F); ESI-MS m/z [M+H]+ 423.1.

### Example 207: 4-(2-(difluoromethyl)-4-methylphenyl)-N-((3R,5R)-5-fluoro-1-methylpiperidin-3-yl)-7,8-dihydro-5H-pyrano[3,4-d]pyridazin-1-amine

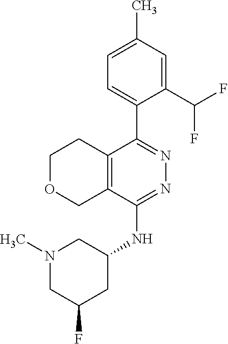 [SMILES: Cc1ccc(-c2nnc(N[C@@H]3C[C@@H](F)CN(C)C3)c3c2CCOC3)c(C(F)F)c1]

To a mixture of 4-chloro-N-((3R,5R)-5-fluoro-1-methylpiperidin-3-yl)-7,8-dihydro-5H-pyrano[3,4-d]pyridazin-1-amine (50 mg, 166.25 μmol) and 2-(2-(difluoromethyl)-4-methylphenyl)-4,4,5,5-tetramethyl-1,3,2-dioxaborolane (133.71 mg, 498.74 μmol) in dioxane (1.6 mL) and H₂O (0.4 mL) were added Cs₂CO₃ (162.50 mg, 498.74 μmol) and Pd(dppf)Cl₂·CH₂Cl₂ (27.15 mg, 33.25 μmol) in one portion under N₂. The reaction mixture was stirred at 100° C. for 8 hours and then diluted with 1 M HCl aq (2 mL) and extracted with EtOAc (3 mL×2). The aqueous phase was collected and purified by preparative HPLC (Boston Prime C18-5 μm, 30 mm×150 mm column) using a gradient of 2 to 32% ACN in water (with FA) to give a formic acid salt of the title compound as a brown solid (19.1 mg, 25.4% yield, 95.3% purity). ¹H NMR (400 MHz, DMSO-d₆) δ ppm 1.67-1.87 (m, 1H), 1.98 (br t, J=9.96 Hz, 1H), 2.14 (br d, J=11.49 Hz, 2H), 2.23 (s, 3H), 2.43 (s, 3H), 2.47 (br s, 2H), 2.90 (br t, J=11.31 Hz, 1H), 2.99 (br d, J=7.58 Hz, 1H), 3.92 (br t, J=5.44 Hz, 2H), 4.19 (br s, 2H), 4.60 (br d, J=5.62 Hz, 1H), 4.86-5.07 (m, 1H), 6.01 (br d, J=8.07 Hz, 1H), 6.58-6.93 (m, 1H), 7.26 (d, J=7.83 Hz, 1H), 7.41 (br d, J=7.82 Hz, 1H), 7.56 (s, 1H), 8.17 (s, 1H); ESI-MS m/z [M+H]+ 407.1.

### Example 208: 4-(2-(difluoromethyl)-4-methoxyphenyl)-N-((3R,5R)-5-fluoro-1-methylpiperidin-3-yl)-7,8-dihydro-5H-pyrano[3,4-d]pyridazin-1-amine

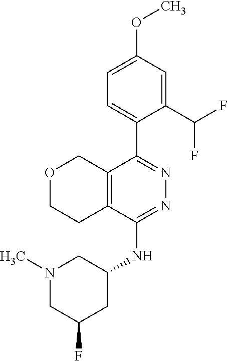 [SMILES: COc1ccc(-c2nnc(N[C@@H]3C[C@@H](F)CN(C)C3)c3c2COCC3)c(C(F)F)c1]

To a mixture of 4-chloro-N-((3R,5R)-5-fluoro-1-methylpiperidin-3-yl)-7,8-dihydro-5H-pyrano[3,4-d]pyridazin-1-amine (50 mg, 166.25 μmol) and 2-(2-(difluoromethyl)-4-methoxyphenyl)-4,4,5,5-tetramethyl-1,3,2-dioxaborolane (94.47 mg, 332.50 μmol) in dioxane (1.6 mL) and H₂O (0.4 mL) were added Cs₂CO₃ (108.33 mg, 332.50 μmol) and Pd(dppf)Cl₂·CH₂Cl₂ (13.58 mg, 16.62 μmol) in one portion under N₂. The reaction mixture was stirred at 100° C. for 8 hours and then diluted with 1 M HCl aq (2 mL) and extracted with EtOAc (3 mL×2). The aqueous phase was collected and purified by preparative HPLC (Kromasil Eternity XT-10 μm, 30 mm×150 mm column) using a gradient of 18 to 58% ACN in water (with NH₃H₂O) to give the title compound as a white solid (22.7 mg, 33.9% yield, 97% purity). ¹H NMR (400 MHz, DMSO-d₆) δ ppm 1.65-1.86 (m, 1H), 1.95 (br t, J=9.90 Hz, 1H), 2.08-2.19 (m, 2H), 2.21 (s, 3H), 2.42-2.47 (m, 2H), 2.88 (br t, J=11.07 Hz, 1H), 2.97 (br d, J=7.46 Hz, 1H), 3.86 (s, 3H), 3.92 (br t, J=5.62 Hz, 2H), 4.13-4.28 (m, 2H), 4.52-4.67 (m, 1H), 4.84-5.06 (m, 1H), 5.97 (d, J=8.07 Hz, 1H), 6.61-6.94 (m, 1H), 7.16 (dd, J=8.44, 2.45 Hz, 1H), 7.23 (d, J=2.45 Hz, 1H), 7.32 (d, J=8.44 Hz, 1H); ESI-MS m/z [M+H]+ 423.1.

### Example 209: N-((3R,5R)-5-fluoro-1-methylpiperidin-3-yl)-4-(2-fluoro-4-methoxyphenyl)-7,8-dihydro-5H-pyrano[3,4-d]pyridazin-1-amine

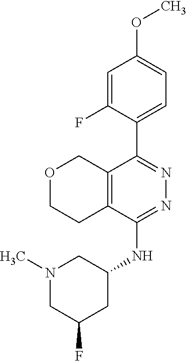

To a mixture of 4-chloro-N-((3R,5R)-5-fluoro-1-methylpiperidin-3-yl)-7,8-dihydro-5H-pyrano[3,4-d]pyridazin-1-amine (53.3 mg, 177.22 μmol) and (2-fluoro-4-methoxyphenyl)boronic acid (60.23 mg, 354.44 μmol) in dioxane (1.6 mL) and H₂O (0.4 mL) were added Cs₂CO₃ (115.48 mg, 354.44 μmol) and Pd(dppf)Cl₂·CH₂Cl₂ (14.47 mg, 17.72 μmol) in one portion under N₂. The reaction mixture was stirred at 100° C. for 1 hour and was then diluted with 1 M HCl aq (2 mL) and extracted with EtOAc (3 mL×2). The aqueous phase was collected and purified by preparative HPLC (Boston Prime C18-5 μm, 30 mm×150 mm column) using a gradient of 15 to 35% ACN in water (with FA) to give a formic acid salt of the title compound as a yellow solid (13 mg, 16.8% yield, 96.95% purity). ¹H NMR (400 MHz, DMSO-d₆) δ ppm 1.66-1.87 (m, 1H), 1.97 (br t, J=9.96 Hz, 1H), 2.05-2.17 (m, 2H), 2.23 (s, 3H), 2.47 (br s, 2H), 2.83-3.02 (m, 2H), 3.82 (s, 3H), 3.93 (br t, J=5.62 Hz, 2H), 4.27 (s, 2H), 4.52-4.65 (m, 1H), 4.86-5.06 (m, 1H), 5.95 (br d, J=7.95 Hz, 1H), 6.85-6.98 (m, 2H), 7.31 (t, J=8.68 Hz, 1H), 8.15 (s, 1H); ESI-MS m/z [M+H]⁺ 391.1.

### Example 210: N-((3R,5R)-5-fluoro-1-methylpiperidin-3-yl)-4-(4-methoxy-2-(trifluoromethyl)phenyl)-7,8-dihydro-5H-pyrano[3,4-d]pyridazin-1-amine

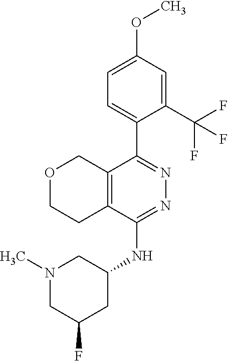 [SMILES: COc1ccc(-c2nnc(N[C@@H]3C[C@@H](F)CN(C)C3)c3c2COCC3)c(C(F)(F)F)c1]

To a mixture of 4-chloro-N-((3R,5R)-5-fluoro-1-methylpiperidin-3-yl)-7,8-dihydro-5H-pyrano[3,4-d]pyridazin-1-amine (53.3 mg, 177.22 μmol) and (4-methoxy-2-(trifluoromethyl)phenyl)boronic acid (77.96 mg, 354.44 μmol) in dioxane (1.6 mL) and H₂O (0.4 mL) were added Cs₂CO₃ (115.48 mg, 354.44 μmol) and Pd(dppf)Cl₂·CH₂Cl₂ (14.47 mg, 17.72 μmol) in one portion under N₂. The reaction mixture was stirred at 100° C. for 1 hour and was then diluted with 1 M HCl aq (2 mL) and extracted with EtOAc (3 mL×2). The aqueous phase was collected and purified by preparative HPLC (Boston Prime C18-5 μm, 30 mm×150 mm column) using a gradient of 20 to 40% ACN in water (with FA) to give a formic acid salt of the title compound as a brown solid (6.5 mg, 7.5% yield, 96.84% purity). ¹H NMR (400 MHz, DMSO-d₆) δ ppm 1.63-1.85 (m, 1H), 1.97 (br d, J=8.80 Hz, 1H), 2.06-2.18 (m, 2H), 2.21 (s, 3H), 2.45 (br s, 2H), 2.80-3.05 (m, 2H), 3.87 (s, 6H), 4.22 (br s, 1H), 4.48-4.62 (m, 1H), 4.82-5.05 (m, 1H), 7.25-7.39 (m, 3H), 8.20 (br s, 1H); ESI-MS m/z [M+H]+ 441.1.

### Example 211: 4-(4-chloro-2-fluorophenyl)-N-((3R,5R)-5-fluoro-1-methylpiperidin-3-yl)-7,8-dihydro-5H-pyrano[3,4-d]pyridazin-1-amine

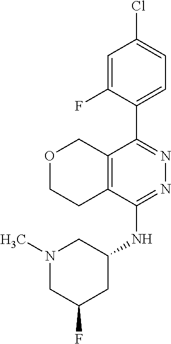 [SMILES: CN1C[C@H](F)C[C@@H](Nc2nnc(-c3ccc(Cl)cc3F)c3c2CCOC3)C1]

To a mixture of 4-chloro-N-((3R,5R)-5-fluoro-1-methylpiperidin-3-yl)-7,8-dihydro-5H-pyrano[3,4-d]pyridazin-1-amine (53.3 mg, 177.22 μmol) and (4-chloro-2-fluorophenyl)boronic acid (46.35 mg, 265.83 μmol) in dioxane (1.6 mL) and H₂O (0.4 mL) were added Cs₂CO₃ (115.48 mg, 354.44 μmol) and Pd(dppf)Cl₂·CH₂Cl₂ (14.47 mg, 17.72 μmol) in one portion under N₂. The reaction mixture was stirred at 100° C. for 1 hour and was then diluted with 1 M HCl aq (2 mL) and extracted with EtOAc (3 mL×2). The aqueous phase was collected and purified by preparative HPLC (Boston Prime C18-5 μm, 30 mm×150 mm column) using a gradient of 18 to 58% ACN in water (with FA) to give a formic acid salt of the title compound as a brown solid (12.4 mg, 15.9% yield, 100% purity). ¹H NMR (400 MHz, DMSO-d₆) δ ppm 1.70-1.91 (m, 1H), 2.02-2.20 (m, 3H), 2.27-2.35 (m, 4H), 2.90-3.09 (m, 2H), 3.94 (br t, J=5.62 Hz, 2H), 4.29 (s, 2H), 4.62 (br d, J=6.24 Hz, 1H), 4.89-5.12 (m, 1H), 6.10 (br d, J=7.82 Hz, 1H), 7.37-7.50 (m, 2H), 7.57 (dd, J=9.84, 1.77 Hz, 1H), 8.14 (s, 1H); ESI-MS m/z [M+H]⁺ 395.1.

### Example 212: 5-chloro-2-(4-(((3R,5R)-5-fluoro-1-methylpiperidin-3-yl)amino)-7,8-dihydro-5H-pyrano[3,4-d]pyridazin-1-yl)phenol

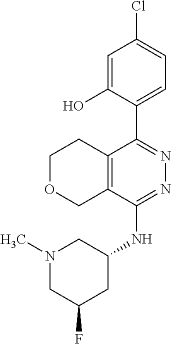 [SMILES: CN1C[C@H](F)C[C@@H](Nc2nnc(-c3ccc(Cl)cc3O)c3c2COCC3)C1]

To a mixture of 1-chloro-N-((3R,5R)-5-fluoro-1-methylpiperidin-3-yl)-7,8-dihydro-5H-pyrano[3,4-d]pyridazin-4-amine (80 mg, 265.99 μmol) and (4-chloro-2-hydroxyphenyl)boronic acid (50.44 mg, 292.59 μmol) in dioxane (1 mL) and H₂O (0.25 mL) were added Cs₂CO₃ (173.33 mg, 531.99 μmol) and Pd(dppf)Cl₂·CH₂Cl₂ (21.72 mg, 26.60 μmol) in one portion under N₂. The reaction mixture was stirred at 100° C. for 1 hour and was then diluted with 1 M HCl aq (2 mL) and extracted with EtOAc (3 mL×2). The aqueous phase was collected and purified by preparative HPLC (Boston Prime C18-5 μm, 30 mm×150 mm column) using a gradient of 0 to 28% ACN in water (with FA) to give a formic acid salt of the title compound as a brown solid (32.8 mg, yield 28.1%, purity 95.496%). ¹H NMR (400 MHz, DMSO-d₆) δ ppm 1.60-1.79 (m, 1H), 1.92 (br t, J=10.09 Hz, 1H), 2.07-2.19 (m, 2H), 2.22 (s, 3H), 2.42 (br t, J=5.14 Hz, 2H), 2.83-3.03 (m, 2H), 3.76 (br t, J=5.50 Hz, 2H), 4.46 (s, 2H), 4.50-4.62 (m, 1H), 4.86-5.06 (m, 1H), 5.70 (d, J=7.95 Hz, 1H), 6.91-6.97 (m, 2H), 7.19 (d, J=7.95 Hz, 1H), 8.16 (s, 1H); ESI-MS m/z [M+H]⁺ 393.1.

### Example 213: 2-(4-(((3R,5R)-5-fluoro-1-methylpiperidin-3-yl)amino)-7,8-dihydro-5H-pyrano[3,4-d]pyridazin-1-yl)phenol

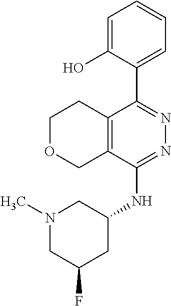 [SMILES: CN1C[C@H](F)C[C@@H](Nc2nnc(-c3ccccc3O)c3c2COCC3)C1]

To a mixture of 1-chloro-N-((3R,5R)-5-fluoro-1-methylpiperidin-3-yl)-7,8-dihydro-5H-pyrano[3,4-d]pyridazin-4-amine (60 mg, 199.50 μmol) and (2-hydroxyphenyl)boronic acid (55.03 mg, 398.99 μmol) in dioxane (1 mL) and H₂O (0.25 mL) were added Cs₂CO₃ (130 mg, 398.99 μmol) and Pd(dppf)Cl₂·CH₂Cl₂ (16.29 mg, 19.95 μmol) in one portion under N₂. The reaction mixture was stirred at 100° C. for 1 hour and was then diluted with 1 M HCl aq (2 mL) and extracted with EtOAc (3 mL×2). The aqueous phase was collected purified by preparative HPLC (Boston Prime C18-5 μm, 30 mm×150 mm column) using a gradient of 0 to 20% ACN in water (with FA) to give a formic acid salt of the title compound as a white solid (31 mg, yield 38%, purity 95.706%). ¹H NMR (400 MHz, DMSO-d₆) δ ppm 1.60-1.80 (m, 1H), 1.92 (br t, J=10.03 Hz, 1H), 2.07-2.19 (m, 2H), 2.22 (s, 3H), 2.44 (br t, J=5.14 Hz, 2H), 2.83-3.06 (m, 2H), 3.76 (brt, J=5.44 Hz, 2H), 4.46 (s, 2H), 4.51-4.65 (m, 1H), 4.84-5.07 (m, 1H), 5.65 (d, J=7.82 Hz, 1H), 6.85-6.94 (m, 2H), 7.17 (dd, J=7.46, 1.59 Hz, 1H), 7.20-7.28 (m, 1H), 8.15 (s, 1H); ESI-MS m/z [M+H]⁺ 359.2.

### Example 214: N-((3R,5R)-5-fluoro-1-methylpiperidin-3-yl)-4-(2-fluoro-4-(trifluoromethoxy)phenyl)-7,8-dihydro-5H-pyrano[3,4-d]pyridazin-1-amine

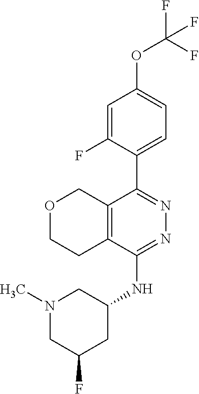 [SMILES: CN1C[C@H](F)C[C@@H](Nc2nnc(-c3ccc(OC(F)(F)F)cc3F)c3c2CCOC3)C1]

To a mixture of 4-chloro-N-((3R,5R)-5-fluoro-1-methylpiperidin-3-yl)-7,8-dihydro-5H-pyrano[3,4-d]pyridazin-1-amine (50 mg, 166.25 μmol), 2-(2-fluoro-4-(trifluoromethoxy)phenyl)-4,4,5,5-tetramethyl-1,3,2-dioxaborolane (76.32 mg, 249.38 μmol) and Cs₂CO₃ (216.67 mg, 665.00 μmol) in dioxane (3 mL) and H₂O (0.5 mL) was added Pd(dppf)Cl₂·CH₂Cl₂ (27.15 mg, 33.25 μmol). The reaction mixture was stirred at 100° C. for 1 hour under N₂ and was then diluted with 1 M HCl aq (2 mL) and extracted with EtOAc (3 mL×2). The aqueous phase was collected and purified by preparative HPLC (Phenomenex C18-5 μm, 30 mm×80 mm column) using a gradient of 20 to 40% ACN in water (with FA). A formic acid salt of the title compound was obtained as a brown solid (11.7 mg, 14.3% yield, 99.7% purity). ¹H NMR (400 MHz, CD₃OD) δ ppm 1.75-1.99 (m, 1H), 2.36 (br t, J=10.54 Hz, 2H), 2.50 (s, 3H), 2.54-2.79 (m, 3H), 3.18 (br s, 1H), 3.35 (br s, 1H), 4.03 (t, J=5.65 Hz, 2H), 4.40 (s, 2H), 4.74-4.85 (m, 1H), 4.96-5.12 (m, 1H), 7.29 (br d, J=8.78 Hz, 2H), 7.53 (t, J=8.28 Hz, 1H), 8.45 (br s, 1H); ESI-MS m/z [M+H]⁺ 444.2.

### Example 215: N-((3R,5R)-5-fluoro-1-methylpiperidin-3-yl)-1-(2-fluoro-4-methoxyphenyl)-7,8-dihydro-5H-pyrano[3,4-d]pyridazin-4-amine

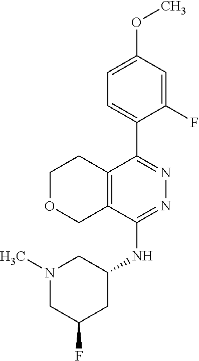 [SMILES: COc1ccc(-c2nnc(N[C@@H]3C[C@@H](F)CN(C)C3)c3c2CCOC3)c(F)c1]

A mixture of 1-chloro-N-((3R,5R)-5-fluoro-1-methylpiperidin-3-yl)-7,8-dihydro-5H-pyrano[3,4-d]pyridazin-4-amine (40 mg, 133.00 μmol), (2-fluoro-4-methoxyphenyl)boronic acid (33.90 mg, 199.50 μmol), Pd(dppf)Cl₂·CH₂Cl₂ (10.86 mg, 13.30 μmol) and Cs₂CO₃ (130 mg, 399.00 μmol) in dioxane (1 mL) and H₂O (0.25 mL) was degassed and purged with N₂ (3×). The reaction mixture was stirred at 100° C. for 1 hour under N₂ and then adjusted to pH 2 with 1 M HCl aq, diluted with EtOAc (40 mL) and extracted with EtOAc (20 mL×2). The aqueous phase was collected and purified by preparative HPLC (YMC-Triart Prep C18-7 μm, 40 mm×150 mm column) using a gradient of 9 to 49% ACN in water (with FA). A formic acid salt of the title compound was obtained as a white solid (7.3 mg, 12.6% yield, 98% purity). ¹H NMR (400 MHz, CD₃OD) δ ppm 1.81-2.02 (m, 1H), 2.53 (br s, 2H), 2.61-2.67 (m, 3H), 2.72-2.91 (m, 1H), 3.33-3.42 (m, 1H), 3.49-3.55 (m, 1H), 3.78-3.93 (m, 5H), 4.50-4.61 (m, 2H), 4.73-4.87 (m, 2H), 4.97-5.22 (m, 1H), 6.76-6.97 (m, 2H), 7.22-7.37 (m, 1H), 8.25-8.50 (m, 1H); ESI-MS m/z [M+H]+ 391.4.

### Example 216: N-((3R,5R)-5-fluoro-1-methylpiperidin-3-yl)-1-(4-methoxy-2-(trifluoromethyl)phenyl)-7,8-dihydro-5H-pyrano[3,4-d]pyridazin-4-amine

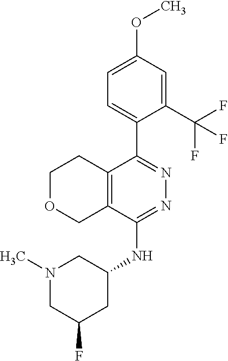 [SMILES: COc1ccc(-c2nnc(N[C@@H]3C[C@@H](F)CN(C)C3)c3c2CCOC3)c(C(F)(F)F)c1]

A mixture of 1-chloro-N-((3R,5R)-5-fluoro-1-methylpiperidin-3-yl)-7,8-dihydro-5H-pyrano[3,4-d]pyridazin-4-amine (40 mg, 133.00 μmol), (4-methoxy-2-(trifluoromethyl)phenyl)boronic acid (43.88 mg, 199.50 μmol), Pd(dppf)Cl₂·CH₂Cl₂ (10.86 mg, 13.30 μmol) and Cs₂CO₃ (130.00 mg, 399.00 μmol) in dioxane (1 mL) and H₂O (0.25 mL) was degassed and purged with N₂ (3×). The reaction mixture was stirred at 100° C. for 1 hour under N₂ atmosphere and was then adjusted to pH 2 with 1 M HCl aq, diluted with EtOAc (10 mL) and extracted with EtOAc (10 mL×2). The aqueous phase was collected and purified by preparative HPLC (YMC-Triart Prep C18-7 μm, 40 mm×150 mm column) using a gradient of 8 to 48% ACN in water (with FA). A formic acid salt of the title compound was obtained as a white solid (6.7 mg, 10.4% yield, 98% purity). ¹H NMR (400 MHz, CD₃OD) δ ppm 1.79-2.00 (m, 1H), 2.17 (br s, 4H), 2.53-2.62 (m, 3H), 2.64-2.85 (m, 1H), 3.23-3.29 (m, 1H), 3.38-3.51 (m, 1H), 3.76-3.96 (m, 5H), 4.56 (s, 1H), 4.53-4.54 (m, 1H), 4.55-4.56 (m, 1H), 4.71-4.84 (m, 2H), 4.96-5.20 (m, 1H), 7.20-7.41 (m, 3H), 8.23-8.58 (m, 1H); ESI-MS m/z [M+H]⁺ 441.2.

### Example 217: 1-(4-chloro-2-fluorophenyl)-N-((3R,5R)-5-fluoro-1-methylpiperidin-3-yl)-7,8-dihydro-5H-pyrano[3,4-d]pyridazin-4-amine

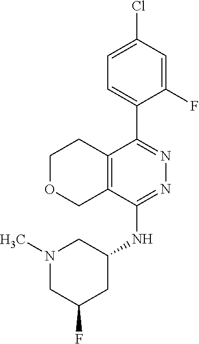 [SMILES: CN1C[C@H](F)C[C@@H](Nc2nnc(-c3ccc(Cl)cc3F)c3c2COCC3)C1]

A mixture of 1-chloro-N-((3R,5R)-5-fluoro-1-methylpiperidin-3-yl)-7,8-dihydro-5H-pyrano[3,4-d]pyridazin-4-amine (40 mg, 133.00 μmol), (4-chloro-2-fluorophenyl)boronic acid (34.79 mg, 199.50 μmol), Pd(dppf)Cl₂·CH₂Cl₂ (10.86 mg, 13.30 μmol) and Cs₂CO₃ (130 mg, 399 μmol) in dioxane (1 mL) and H₂O (0.25 mL) was degassed and purged with N₂ (3×). The reaction mixture was stirred at 100° C. for 1 hour under N₂ atmosphere and was then adjusted to pH 2 with 1 M HCl aq, diluted with EtOAc 10 mL and extracted with EtOAc (10 mL×2). The aqueous phase was collected and purified by preparative HPLC (YMC-Triart Prep C18-7 μm, 40 mm×150 mm column) using a gradient of 0 to 30% ACN in water (with FA). A formic acid salt of the title compound was obtained as a white solid (1.7 mg, 2.9% yield, 98% purity). ¹H NMR (400 MHz, CD₃OD) δ ppm 1.78-1.98 (m, 1H), 2.31-2.45 (m, 2H), 2.46-2.60 (m, 5H), 2.62-2.79 (m, 1H), 3.18-3.28 (m, 1H), 3.37-3.47 (m, 1H), 3.81-3.94 (m, 2H), 4.53-4.61 (m, 2H), 4.74-4.86 (m, 2H), 4.99-5.14 (m, 1H), 7.23-7.52 (m, 3H), 8.30-8.46 (m, 1H); ESI-MS m/z [M+H]⁺ 395.1.

### Example 218: (R)-5-methyl-2-(4-((1-methylpiperidin-3-yl)amino)-7,8-dihydro-5H-pyrano[3,4-d]pyridazin-1-yl)phenol

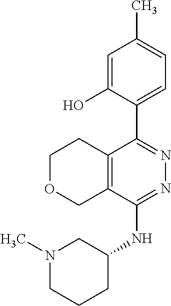 [SMILES: Cc1ccc(-c2nnc(N[C@@H]3CCCN(C)C3)c3c2CCOC3)c(O)c1]

To a mixture of (R)-1-chloro-N-(1-methylpiperidin-3-yl)-7,8-dihydro-5H-pyrano[3,4-d]pyridazin-4-amine (110 mg, 389.01 μmol), (2-hydroxy-4-methylphenyl)boronic acid (88.67 mg, 583.52 μmol) and Cs₂CO₃ (253.50 mg, 778.02 μmol) in dioxane (1 mL) and H₂O (0.25 mL) was added Pd(dppf)Cl₂·CH₂Cl₂ (63.54 mg, 77.80 μmol). The reaction mixture was stirred at 100° C. for 1 hour under N₂ and was then adjusted to pH 2 with 1 M HCl aq, diluted with EtOAc (10 mL) and extracted with EtOAc (10 mL×2). The aqueous phase was collected and purified by preparative HPLC (Boston Green ODS-5 μm, 30 mm×150 mm column) using a gradient of 0 to 29% ACN in water (with HCl). An HCl salt of the title compound was obtained as a white solid (70 mg, 46% yield, 99% purity). ¹H NMR (400 MHz, CD₃OD) δ ppm 1.65-2.02 (m, 2H), 2.07-2.33 (m, 2H), 2.38 (s, 3H), 2.66-2.80 (m, 2H), 2.83-3.17 (m, 4H), 3.48-3.60 (m, 1H), 3.67-3.85 (m, 1H), 3.87-4.02 (m, 2H), 4.41-4.59 (m, 1H), 4.75 (s, 1H), 4.92-5.10 (m, 1H), 6.82-6.96 (m, 2H), 7.19-7.30 (m, 1H); ESI-MS m/z [M+H]⁺ 355.21.

### Example 219: 5-fluoro-2-(4-(((3R,5R)-5-fluoro-1-methylpiperidin-3-yl)amino)-7,8-dihydro-5H-pyrano[3,4-d]pyridazin-1-yl)phenol

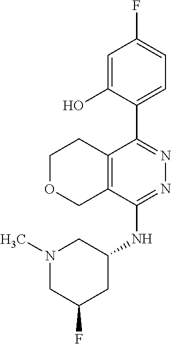 [SMILES: CN1C[C@H](F)C[C@@H](Nc2nnc(-c3ccc(F)cc3O)c3c2COCC3)C1]

To a mixture of 1-chloro-N-((3R,5R)-5-fluoro-1-methylpiperidin-3-yl)-7,8-dihydro-5H-pyrano[3,4-d]pyridazin-4-amine (60 mg, 199.50 μmol), (4-fluoro-2-hydroxyphenyl)boronic acid (46.66 mg, 299.25 μmol) and Cs₂CO₃ (130 mg, 399 μmol) in dioxane (1 mL) and H₂O (0.25 mL) was added Pd(dppf)Cl₂·CH₂Cl₂ (32.58 mg, 39.90 μmol). The reaction mixture was stirred at 100° C. for 1 hour under N₂ and was then adjusted to pH 2 with 1 M HCl aq, diluted with EtOAc (10 mL) and extracted with EtOAc (10 mL×2). The aqueous phase was collected and purified by preparative HPLC (Boston Green ODS-5 μm, 30 mm×150 mm column) using a gradient of 0 to 29% ACN in water (with HCl). An HCl salt of the title compound was obtained as a yellow solid (39 mg, 47% yield, 98% purity). ¹H NMR (400 MHz, CD₃OD) δ ppm 2.00-2.26 (m, 1H), 2.56 (br s, 1H), 2.74 (br s, 2H), 3.01 (s, 3H), 3.08-3.21 (m, 1H), 3.34-3.56 (m, 1H), 3.70-3.90 (m, 2H), 3.96 (t, J=5.38 Hz, 2H), 4.76-4.83 (m, 3H), 5.18-5.42 (m, 1H), 6.71-6.94 (m, 2H), 7.45 (dd, J=8.44, 6.44 Hz, 1H); ESI-MS m/z [M+H]⁺ 377.17.

### Example 220: 2-(4-(((3R,5R)-5-fluoro-1-methylpiperidin-3-yl)amino)-7,8-dihydro-5H-pyrano[3,4-d]pyridazin-1-yl)-5-methylphenol

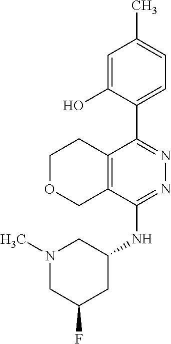 [SMILES: Cc1ccc(-c2nnc(N[C@@H]3C[C@@H](F)CN(C)C3)c3c2CCOC3)c(O)c1]

A mixture of 1-chloro-N-((3R,5R)-5-fluoro-1-methylpiperidin-3-yl)-7,8-dihydro-5H-pyrano[3,4-d]pyridazin-4-amine (60 mg, 199.50 μmol), (2-hydroxy-4-methylphenyl)boronic acid (45.47 mg, 299.24 μmol), Pd(dppf)Cl₂·CH₂Cl₂ (32.58 mg, 39.90 μmol) and Cs₂CO₃ (130.00 mg, 398.99 μmol) in dioxane (1 mL) and H₂O (0.25 mL) was stirred at 100° C. for 1 hour under N₂. The reaction mixture was then adjusted to pH 2 with 1 M HCl aq, diluted with EtOAc (10 mL) and extracted with EtOAc (10 mL×2). The aqueous phase was collected and purified by preparative HPLC (Boston Green ODS-5 μm, 30 mm×150 mm column) using a gradient of 0 to 29% ACN in water (with HCl). An HCl salt of the title compound was obtained as a yellow solid (35 mg, 43% yield, 99% purity). ¹H NMR (400 MHz, CD₃OD) δ ppm 1.99-2.24 (m, 1H), 2.38 (s, 3H), 2.56 (br d, J=12.55 Hz, 1H), 2.75 (br s, 2H), 3.01 (s, 3H), 3.06-3.17 (m, 1H), 3.34-3.53 (m, 1H), 3.75-3.89 (m, 2H), 3.95 (t, J=5.40 Hz, 2H), 4.72-4.84 (m, 3H), 5.20-5.45 (m, 1H), 6.85-6.95 (m, 2H), 7.26 (d, J=7.78 Hz, 1H); ESI-MS m/z [M+H]⁺ 373.20.

### Example 221: (R)-5-methoxy-2-(4-((1-methylpiperidin-3-yl)amino)-7,8-dihydro-5H-pyrano[3,4-d]pyridazin-1-yl)phenol

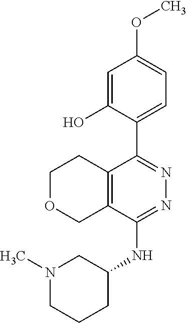

A mixture of (R)-1-chloro-N-(1-methylpiperidin-3-yl)-7,8-dihydro-5H-pyrano[3,4-d]pyridazin-4-amine (25 mg, 88.41 μmol), (2-hydroxy-4-methoxyphenyl)boronic acid (44.55 mg, 265.23 μmol), Pd(dppf)Cl₂ (6.47 mg, 8.84 μmol) and Cs₂CO₃ (86.42 mg, 265.23 μmol) in H₂O (0.25 mL) and dioxane (1 mL) was degassed and purged with N₂ (3×). The reaction mixture was stirred at 100° C. for 1 hour under N₂ atmosphere and was then filtered. The filtrate was purified by preparative HPLC (Phenomenex C18-3 μm, 30 mm×75 mm column) using a 10 to 40% gradient of ACN in water (with NH₃H₂O+NH₄HCO₃). The title compound was obtained as a white solid (5 mg, 15% yield, 96.564% purity). ¹H NMR (400 MHz, DMSO-d₆) δ ppm 1.37 (br d, J=8.0 Hz, 1H), 1.58 (br d, J=11.6 Hz, 1H), 1.72 (br s, 1H), 1.81-2.08 (m, 3H), 2.13-2.31 (m, 3H), 2.53 (d, J=1.9 Hz, 2H), 2.69-2.81 (m, 1H), 3.03 (br s, 1H), 3.71-3.80 (m, 5H), 4.17-4.32 (m, 1H), 4.47 (s, 2H), 5.60 (dd, J=2.7, 1.2 Hz, 1H), 6.37-6.54 (m, 2H), 7.06-7.15 (m, 1H), 10.10 (br s, 1H); ESI-MS m/z [M+H]⁺ 371.2.

### Example 222: (R)-1-(4-methoxyphenyl)-N-(1-methylpiperidin-3-yl)-7,8-dihydro-5H-pyrano[3,4-d]pyridazin-4-amine

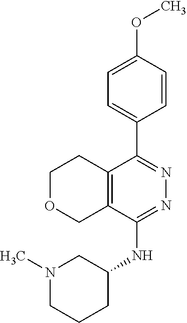

### Example 223: (R)-4-(4-methoxyphenyl)-N-(1-methylpiperidin-3-yl)-7,8-dihydro-5H-pyrano[3,4-d]pyridazin-1-amine

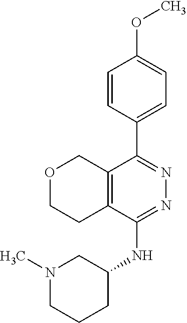

To a mixture of (R)-4-chloro-N-(1-methylpiperidin-3-yl)-7,8-dihydro-5H-pyrano[3,4-d]pyridazin-1-amine and (R)-1-chloro-N-(1-methylpiperidin-3-yl)-7,8-dihydro-5H-pyrano[3,4-d]pyridazin-4-amine (total mass 200 mg) in dioxane (5 mL) and H₂O (2 mL) was added (4-methoxyphenyl)boronic acid (322.43 mg, 2.12 mmol), Pd(dppf)Cl₂·CH₂Cl₂ (231.04 mg, 282.92 μmol) and Cs₂CO₃ (921.80 mg, 2.83 mmol). The reaction mixture was stirred at 100° C. for 12 hours under N₂ and was then diluted with 1 M HCl aq (8 mL) and extracted with EtOAc (10 mL). The organic layer was discarded and the aqueous layer was filtered. The filtrate was purified by preparative HPLC (Xtimate C18-10 μm, 40 mm×150 mm column) using a gradient of 25 to 55% ACN in water (with 0.05% NH₃H₂O) to give a crude product, which was further purified by chiral SFC (DAICEL CHIRALPAK® IG-10 μm, 30 mm×250 mm column) using a mobile phase of CO₂ and 55% MeOH (with 0.1% NH₃H₂O). The title compound of Example 222 was obtained as a white solid (48.4 mg, 97% purity). ¹H NMR (400 MHz, DMSO-d₆) δ ppm 1.40 (qd, J=11.6, 4.0 Hz, 1H), 1.53-1.67 (m, 1H), 1.70-1.82 (m, 1H), 1.82-2.01 (m, 3H), 2.24 (s, 3H), 2.64 (br t, J=5.1 Hz, 2H), 2.71 (br d, J=10.6 Hz, 1H), 3.02 (br d, J=8.0 Hz, 1H), 3.82 (t, J=5.3 Hz, 2H), 3.87 (s, 3H), 4.23-4.39 (m, 1H), 4.53 (s, 2H), 5.62 (d, J=7.6 Hz, 1H), 7.07 (d, J=8.8 Hz, 2H), 7.52 (d, J=8.8 Hz, 2H); ESI-MS m/z [M+H]⁺ 355.0. The title compound of Example 223 was obtained as a white solid (48.6 mg, 95% purity). ¹H NMR (400 MHz, DMSO-d₆) δ ppm 1.41 (br d, J=10.0 Hz, 1H), 1.49-1.63 (m, 1H), 1.66-1.75 (m, 1H), 1.80-2.05 (m, 3H), 2.20 (s, 3H), 2.43-2.47 (m, 2H), 2.63 (br d, J=8.6 Hz, 1H), 2.93 (br dd, J=7.6, 1.3 Hz, 1H), 3.80 (s, 3H), 3.95 (t, J=5.7 Hz, 2H), 4.15-4.34 (m, 1H), 4.47 (s, 2H), 5.73 (br d, J=7.6 Hz, 1H), 7.00 (d, J=8.9 Hz, 2H), 7.33-7.46 (m, 2H); ESI-MS m/z [M+H]⁺ 355.0.

### Example 224: (R)-5-chloro-2-(1-((1-methylpiperidin-3-yl)amino)-7,8-dihydro-5H-pyrano[3,4-d]pyridazin-4-yl)phenol

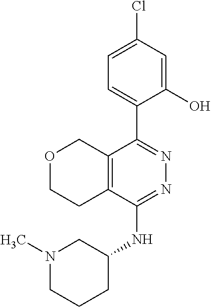 [SMILES: CN1CCC[C@@H](Nc2nnc(-c3ccc(Cl)cc3O)c3c2CCOC3)C1]

### Example 225: (R)-5-chloro-2-(4-((1-methylpiperidin-3-yl)amino)-7,8-dihydro-5H-pyrano[3,4-d]pyridazin-1-yl)phenol

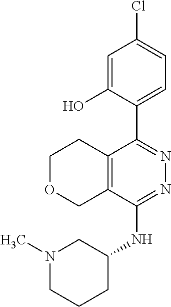 [SMILES: CN1CCC[C@@H](Nc2nnc(-c3ccc(Cl)cc3O)c3c2COCC3)C1]

A mixture of (R)-4-chloro-N-(1-methylpiperidin-3-yl)-7,8-dihydro-5H-pyrano[3,4-d]pyridazin-1-amine and (R)-1-chloro-N-(1-methylpiperidin-3-yl)-7,8-dihydro-5H-pyrano[3,4-d]pyridazin-4-amine (total mass 250 mg), (4-chloro-2-hydroxyphenyl)boronic acid (304.80 mg, 1.77 mmol), Pd(dppf)Cl₂ (64.69 mg, 88.41 μmol) and Cs₂CO₃ (864.19 mg, 2.65 mmol) in H₂O (0.25 mL) and dioxane (1 mL) was degassed and purged with N₂ (3×). The reaction mixture was stirred at 100° C. for 1 hour under N₂ atmosphere and was then adjusted to pH 2 with 1 M HCl aq, diluted with EtOAc (10 mL) and extracted with EtOAc (10 mL×2). The aqueous phase was collected and purified by preparative HPLC to give a mixture of the title compounds as a white solid (210 mg). The desired products were further separated by SFC (DAICEL CHIRALPAK® AD-10 μm, 30 mm×250 mm column) using a mobile phase of CO₂ and 40% MeOH (with 0.1% NH₃H₂O). The title compound of Example 224 was obtained as a white solid (70 mg, 98% purity). ¹H NMR (400 MHz, DMSO-d₆) δ ppm 1.36-1.48 (m, 1H), 1.50-1.62 (m, 1H), 1.70 (br s, 1H), 1.81-2.04 (m, 3H), 2.22 (s, 3H), 2.29-2.45 (m, 2H), 2.60-2.70 (m, 1H), 2.93 (br s, 1H), 3.91 (t, J=5.63 Hz, 2H), 4.31 (s, 3H), 5.80 (br d, J=7.75 Hz, 1H), 6.87-6.98 (m, 2H), 7.15 (d, J=7.88 Hz, 1H), 10.38 (br s, 1H); ESI-MS m/z [M+H]⁺ 375.1. The title compound of Example 225 was obtained as a white solid (80 mg 98% purity). ¹H NMR (400 MHz, DMSO-d₆) δ ppm 1.26-1.44 (m, 1H), 1.48-1.64 (m, 1H), 1.66-1.77 (m, 1H), 1.90 (br d, J=3.38 Hz, 3H), 2.24 (br s, 3H), 2.31-2.45 (m, 3H), 2.63-2.79 (m, 1H), 3.76 (t, J=5.50 Hz, 3H), 4.27 (br s, 1H), 4.47 (s, 2H), 5.64 (br d, J=7.38 Hz, 1H), 6.86-7.01 (m, 2H), 7.19 (d, J=8.13 Hz, 1H) 10.16-10.54 (m, 1H); ESI-MS m/z [M+H]⁺ 375.1.

### Example 226: (R)-2-(4-((1-methylpiperidin-3-yl)amino)-7,8-dihydro-5H-pyrano[3,4-d]pyridazin-1-yl)phenol

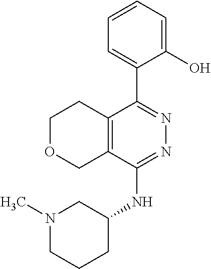 [SMILES: CN1CCC[C@@H](Nc2nnc(-c3ccccc3O)c3c2COCC3)C1]

and

### Example 227: (R)-2-(1-((1-methylpiperidin-3-yl)amino)-7,8-dihydro-5H-pyrano[3,4-d]pyridazin-4-yl)phenol

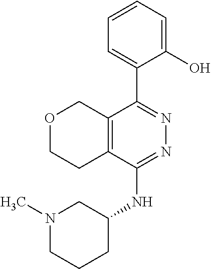 [SMILES: CN1CCC[C@@H](Nc2nnc(-c3ccccc3O)c3c2CCOC3)C1]

A mixture of (R)-4-chloro-N-(1-methylpiperidin-3-yl)-7,8-dihydro-5H-pyrano[3,4-d]pyridazin-1-amine and (R)-1-chloro-N-(1-methylpiperidin-3-yl)-7,8-dihydro-5H-pyrano[3,4-d]pyridazin-4-amine (total mass 100 mg), (2-hydroxyphenyl)boronic acid (97.56 mg, 707.29 μmol), Pd(dppf)Cl₂ (25.88 mg, 35.36 μmol) and Cs₂CO₃ (345.68 mg, 1.06 mmol) in H₂O (0.25 mL) and dioxane (1 mL) was degassed and purged with N₂ (3×). The reaction mixture was stirred at 100° C. for 2 hours under N₂ atmosphere and was then adjusted to pH 2 with 1 M HCl aq, diluted with EtOAc (10 mL) and extracted with EtOAc (10 mL×2). The aqueous phase was collected and purified by preparative HPLC (Xtimate C18-10 μm, 30 mm×100 mm column) using a gradient of 0 to 30% ACN in water (with FA) to give a mixture of the title compounds as a white solid (110 mg). The products were further separated by SFC (DAICEL CHIRALPAK® AD-10 μm, 30 mm×250 mm column) using a mobile phase of CO₂ and 40% EtOH (with 0.1% NH₃H₂O). The title compound of Example 226 was obtained as a white solid (52 mg, 98% purity). ¹H NMR (400 MHz, DMSO-d₆) δ ppm 1.30-1.47 (m, 1H), 1.53-1.65 (m, 1H), 1.72 (br s, 1H), 1.88 (br dd, J=8.13, 4.13 Hz, 1H), 2.00 (br s, 2H), 2.26 (s, 3H), 2.43 (br t, J=5.19 Hz, 2H), 2.73 (br d, J=10.88 Hz, 1H), 3.03 (br d, J=9.51 Hz, 1H), 3.75-3.87 (m, 2H), 4.12-4.33 (m, 1H), 4.47 (s, 2H), 5.62 (br d, J=7.50 Hz, 1H), 6.76-7.03 (m, 2H), 7.10-7.35 (m, 2H), 8.19 (s, 1H), 9.59-10.16 (m, 1H); ESI-MS m/z [M+H]+ 341.1. The title compound of Example 227 was obtained as a white solid (46 mg, 97.618% purity) following further purification of the crude regioisomer (48 mg) by preparative HPLC (Phenomenex C18-3 μm, 30 mm×75 mm column) using a gradient of 15 to 75% ACN in water (with NH₃H₂O+NH₄HCO₃). ¹H NMR (400 MHz, DMSO-d₆) δ ppm 1.35-1.50 (m, 1H), 1.52-1.65 (m, 1H), 1.73 (br d, J=3.13 Hz, 1H), 1.82-1.94 (m, 1H), 2.07 (br s, 1H), 2.27 (br s, 3H), 2.35-2.47 (m, 2H), 2.62-2.78 (m, 1H), 3.00 (br dd, J=5.00, 2.00 Hz, 1H), 3.91 (t, J=5.69 Hz, 2H), 4.20-4.41 (m, 3H), 5.79 (br d, J=7.38 Hz, 1H), 6.82-6.94 (m, 2H), 7.12 (dd, J=7.50, 1.63 Hz, 1H), 7.20-7.30 (m, 1H), 9.85 (s, 1H); ESI-MS m/z [M+H]+ 341.2.

### Example 228: (R)—N-(1-cyclopropylpiperidin-3-yl)-1-(4-methoxyphenyl)-7,8-dihydro-5H-pyrano[3,4-d]pyridazin-4-amine

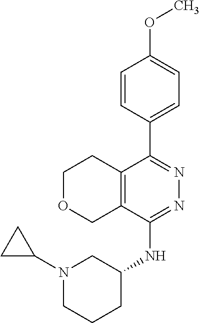 [SMILES: COc1ccc(-c2nnc(N[C@@H]3CCCN(C4CC4)C3)c3c2CCOC3)cc1]

A mixture of 4-chloro-1-(4-methoxyphenyl)-7,8-dihydro-5H-pyrano[3,4-d]pyridazine and 1-chloro-4-(4-methoxyphenyl)-7,8-dihydro-5H-pyrano[3,4-d]pyridazine (total mass 60 mg), (R)-1-cyclopropylpiperidin-3-amine (43.58 mg, 310.79 μmol), Pd(OAc)₂ (6.98 mg, 31.08 μmol), BINAP (19.35 mg, 31.08 μmol) and Cs₂CO₃ (101.26 mg, 310.79 μmol) in toluene (1 mL) was degassed and purged with N₂ (3×). The reaction mixture was stirred at 100° C. for 24 hours under N₂ atmosphere. LC-MS showed 8% conversion to a compound with desired m/z. The reaction mixture was concentrated in vacuo and purified by preparative HPLC (C18-6-5 μm, 30 mm×100 mm column) using a gradient of 0 to 30% ACN in water (with FA). A formic acid salt of the title compound was obtained as a white solid (4.7 mg). ¹H NMR (400 MHz, CD₃OD) δ ppm 0.60 (br d, J=3.63 Hz, 4H), 1.54-1.63 (m, 1H), 1.75 (br s, 1H), 1.78-1.89 (m, 1H), 1.93-2.11 (m, 2H), 2.53 (br s, 1H), 2.64 (s, 3H), 2.97-3.09 (m, 1H), 3.33-3.44 (m, 1H), 3.83-3.89 (m, 5H), 4.33-4.42 (m, 1H), 4.56 (s, 2H), 7.03 (d, J=8.76 Hz, 2H), 7.42 (d, J=8.75 Hz, 2H); ESI-MS m/z [M+H]⁺ 381.2.

### Example 229: 2-(4-(((1R,2R)-2-hydroxycyclohexyl)amino)-7,8-dihydro-5H-pyrano[3,4-d]pyridazin-1-yl)-5-methylphenol

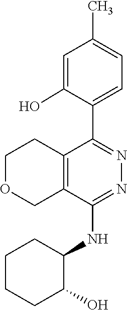 [SMILES: Cc1ccc(-c2nnc(N[C@@H]3CCCC[C@H]3O)c3c2CCOC3)c(O)c1]

The title compound was prepared like EXAMPLE 214. ¹H NMR (400 MHz, CD₃OD) δ ppm 1.31-1.42 (m, 4H), 1.68-1.83 (m, 2H), 2.01-2.20 (m, 2H), 2.30-2.36 (m, 3H), 2.57-2.62 (m, 2H), 3.50-3.61 (m, 1H), 3.81-3.91 (m, 2H), 4.00-4.09 (m, 1H), 4.55-4.63 (m, 3H), 6.70-6.82 (m, 2H), 7.05-7.15 (m, 1H); ESI-MS m/z [M+H]⁺ 356.4.

### Example 230: 2-(1-(((1R,2R)-2-hydroxycyclohexyl)amino)-7,8-dihydro-5H-pyrano[3,4-d]pyridazin-4-yl)-5-methylphenol

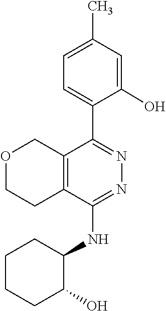 [SMILES: Cc1ccc(-c2nnc(N[C@@H]3CCCC[C@H]3O)c3c2COCC3)c(O)c1]

The title compound was prepared like EXAMPLE 214. ¹H NMR (400 MHz, CD₃OD) δ ppm 1.45 (br d, J=3.63 Hz, 5H), 1.70-1.85 (m, 2H), 2.00-2.11 (m, 1H), 2.14-2.26 (m, 1H), 2.32 (s, 3H), 2.51-2.63 (m, 2H), 3.52-3.62 (m, 1H), 4.01 (br d, J=2.38 Hz, 3H), 4.46 (s, 2H), 6.73 (s, 2H), 7.04 (s, 1H); ESI-MS m/z [M+H]⁺ 356.4.

### Example 231: 4-((1-(2-hydroxy-4-methylphenyl)-7,8-dihydro-5H-pyrano[3,4-d]pyridazin-4-yl)amino)bicyclo[2.2.1]heptan-1-ol

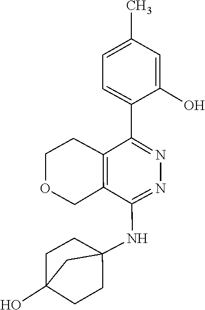 [SMILES: Cc1ccc(-c2nnc(NC34CCC(O)(CC3)C4)c3c2CCOC3)c(O)c1]

A formic acid salt of the title compound was prepared like EXAMPLE 214. ¹H NMR (400 MHz, CD₃OD) δ ppm 1.65-1.80 (m, 2H), 1.81-1.92 (m, 2H), 2.05-2.24 (m, 6H), 2.33 (s, 3H), 2.54 (br t, J=5.63 Hz, 2H), 4.00 (t, J=5.75 Hz, 2H), 4.45 (s, 2H), 6.72-6.83 (m, 2H), 7.07 (d, J=7.63 Hz, 1H), 8.27-8.45 (m, 1H); ESI-MS m/z [M+H]⁺ 368.4.

### Example 232: 4-((4-(2-hydroxy-4-methylphenyl)-7,8-dihydro-5H-pyrano[3,4-d]pyridazin-1-yl)amino)bicyclo[2.2.1]heptan-1-ol

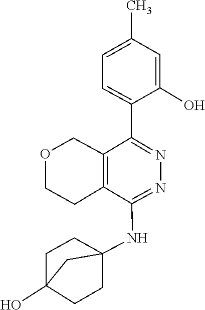 [SMILES: Cc1ccc(-c2nnc(NC34CCC(O)(CC3)C4)c3c2COCC3)c(O)c1]

A formic acid salt of the title compound was prepared like EXAMPLE 214. ¹H NMR (400 MHz, CD₃OD) δ ppm 1.65-1.97 (m, 4H), 2.01-2.22 (m, 6H), 2.33 (br s, 3H), 2.46-2.72 (m, 2H), 3.85 (br s, 2H), 4.56 (br s, 2H), 6.55-6.89 (m, 2H), 7.00-7.17 (m, 1H), 8.33 (br s, 1H); ESI-MS m/z [M+H]⁺ 368.4.

### Example 233: (R)-2-(4-((1-(2-methoxyethyl)piperidin-3-yl)amino)-7,8-dihydro-5H-pyrano[3,4-d]pyridazin-1-yl)-5-methylphenol

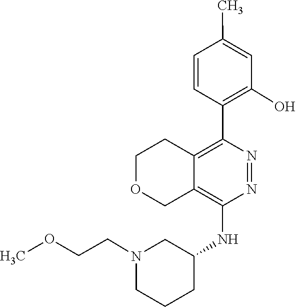 [SMILES: COCCN1CCC[C@@H](Nc2nnc(-c3ccc(C)cc3O)c3c2COCC3)C1]

A mixture of 4-chloro-1-(2-(methoxymethoxy)-4-methylphenyl)-7,8-dihydro-5H-pyrano[3,4-d]pyridazine (1.00 eq, 61 mg, 0.190 mmol), (R)-1-(2-methoxyethyl)piperidin-3-amine dihydrochloride (44 mg, 0.190 mmol), Pd₂(dba)₃ (0.100 eq, 17 mg), R-BINAP (24 mg, 0.0380 mmol) and Cs₂CO₃ (248 mg, 0.761 mmol) in toluene (6 mL) was purged with nitrogen for 5 minutes and heated at 120° C. overnight. The mixture was treated with water and extracted with EtOAc. The organic layers were washed with brine, dried over MgSO₄ and concentrated. The resulting residue was dissolved in dioxane (2 mL) and treated with HCl in dioxane (0.38 mL, 0.761 mmol). The mixture was stirred at room temperature for 3 hours and the product was purified by preparative HPLC (Phenomenex Gemini C18, 5 μm, 30 mm ID×150 mm column) eluting with a gradient of 10-100% ACN (containing 0.079% NH₄HCO₃) in water (containing 0.079% NH₄HCO₃), using a slow ramp from 10-60% ACN. Fractions containing product were evaporated to give the title compound as a clear oil (0.8 mg, 0.71%). ¹H NMR (400 MHz, CD₃OD) δ ppm 1.51-1.72 (m, 2H), 1.73-1.96 (m, 2H), 2.32 (s, 5H), 2.54-2.70 (m, 5H), 2.96-3.05 (m, 1H), 3.34 (s, 3H), 3.52-3.58 (m, 2H), 3.81-3.88 (m, 2H), 4.37-4.45 (m, 1H), 4.54-4.59 (m, 2H), 6.72-6.79 (m, 2H), 7.06-7.11 (m, 1H); ESI-MS [M+H]⁺ 399.1.

### Example 234: 2-(4-(((3R,5R)-5-fluoro-1-methylpiperidin-3-yl)amino)-7,8-dihydro-5H-pyrano[3,4-d]pyridazin-1-yl)-5-(trifluoromethyl)phenol

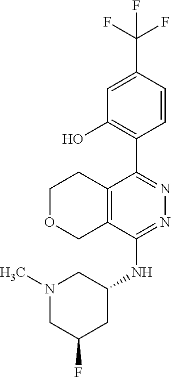 [SMILES: CN1C[C@H](F)C[C@@H](Nc2nnc(-c3ccc(C(F)(F)F)cc3O)c3c2COCC3)C1]

A mixture of 1-chloro-N-((3R,5R)-5-fluoro-1-methylpiperidin-3-yl)-7,8-dihydro-5H-pyrano[3,4-d]pyridazin-4-amine (50 mg, 166.25 μmol), (2-hydroxy-4-(trifluoromethyl)phenyl)boronic acid (51.35 mg, 249.37 μmol), Pd(dppf)Cl₂·CH₂Cl₂ (13.58 mg, 16.62 μmol) and Cs₂CO₃ (216.66 mg, 664.98 μmol) in dioxane (2 mL) and H₂O (0.5 mL) was degassed and purged with N₂ (3×) and then stirred at 100° C. for 2 hours under N₂ atmosphere. LC-MS showed about 56% of product with desired mass was obtained. The reaction mixture was evaporated and partitioned between DCM/H₂O (5 mL/mL). The organic layer was separated and the aqueous layer was extracted with DCM (4 mL×2). The organic layers were combined, dried, and concentrated to give a residue, which was purified by preparative HPLC (Method B) using a gradient of 0 to 30% ACN in water (0225% FA). A formic acid salt of the title compound was obtained as a white solid (37.9 mg, 52% yield, 98% purity). ¹H NMR (400 MHz, DMSO-d₆) δ ppm 1.56-1.82 (m, 1H), 1.86-1.98 (m, 1H), 2.03-2.28 (m, 5H), 2.36-2.46 (m, 2H), 2.84-2.95 (m, 1H), 2.96-3.05 (m, 1H), 3.78 (br t, J=5.44 Hz, 2H), 4.39-4.65 (m, 3H), 4.81-5.09 (m, 1H), 5.63-5.85 (m, 1H), 7.17-7.26 (m, 2H), 7.33-7.51 (m, 1H); ESI-MS [M+H]+ 427.2.

### Example 235: 2-(4-(((3R,5S)-5-fluoro-1-methylpiperidin-3-yl)amino)-7,8-dihydro-5H-pyrano[3,4-d]pyridazin-1-yl)-5-methylphenol

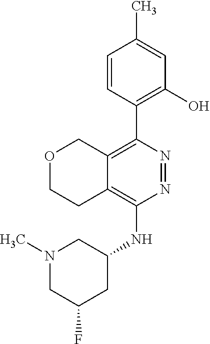 [SMILES: Cc1ccc(-c2nnc(N[C@@H]3C[C@H](F)CN(C)C3)c3c2COCC3)c(O)c1]

¹H NMR (400 MHz, CD₃OD) δ ppm 1.90-2.00 (m, 1H), 2.11-2.24 (m, 1H), 2.32 (s, 3H), 2.35 (s, 3H), 2.57-2.61 (m, 2H), 2.63-2.77 (m, 3H), 3.86 (t, J=5.65 Hz, 2H), 4.49-4.64 (m, 3H), 4.74-4.84 (m, 1H), 4.87-4.94 (m, 1H), 6.74 (s, 1H), 6.75-6.80 (m, 1H), 7.09 (d, J=7.53 Hz, 1H); ESI-MS [M+H]⁺ 373.1.

### Example 236: 2-(4-(((3R,5R)-5-fluoro-1-methylpiperidin-3-yl)amino)-7,8-dihydro-5H-pyrano[3,4-d]pyridazin-1-yl)-5-(trifluoromethoxy)phenol

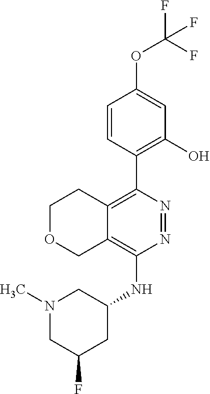 [SMILES: CN1C[C@H](F)C[C@@H](Nc2nnc(-c3ccc(OC(F)(F)F)cc3O)c3c2COCC3)C1]

A mixture of 1-chloro-N-((3R,5R)-5-fluoro-1-methylpiperidin-3-yl)-7,8-dihydro-5H-pyrano[3,4-d]pyridazin-4-amine (25 mg, 0.0831 mmol), (2-hydroxy-4-(trifluoromethoxy)phenyl)boronic acid (24 mg, 0.108 mmol), Pd(dppf)Cl₂·CH₂Cl₂ (6.8 mg, 0.00831 mmol) and Cs₂CO₃ (54 mg, 0.166 mmol) in 1,4-dioxane (0.4 mL) and water (0.1 mL) was stirred at 100° C. for 2 hours using a metal heating block. The reaction mixture was diluted with MeOH (1 mL), filtered through a 0.45 μm PTFE Membrane filter (VWR), rinsed with MeOH (1 mL), and purified by preparative HPLC (Method A). The product-containing fractions were combined, concentrated via rotary evaporation at 45° C. and dried in vacuo to give a TFA salt of the title compound as a white solid (19.0 mg, crude). The crude product was dissolved in MeOH (1 mL) and passed through a 100 mg Agilent StratoSpheres SPE cartridge (PL-HCO₃ MP SPE), rinsing with MeOH (1 mL×3), to remove TFA. The filtrate was concentrated via rotary evaporation and dried in vacuo to give the title compound as a light-yellow solid (8.1 mg, 22%). ¹H NMR (400 MHz, DMSO-d₆) δ ppm 1.65-1.76 (m, 1H), 1.90 (br t, J=10.04 Hz, 1H), 2.07-2.13 (m, 1H), 2.14-2.19 (m, 1H), 2.21 (s, 3H), 2.42 (br t, J=5.40 Hz, 2H), 2.83-2.93 (m, 1H), 2.98 (br dd, J=10.29, 3.76 Hz, 1H), 3.77 (t, J=5.52 Hz, 2H), 4.46 (s, 2H), 4.53-4.64 (m, 1H), 4.90-5.03 (m, 1H), 5.69 (d, J=8.03 Hz, 1H), 6.85-6.92 (m, 2H), 7.26-7.33 (m, 1H), 10.44 (br s, 1H); ESI-MS [M+H]⁺ 443.1.

### Example 237: 2-(4-(((3R,5R)-5-fluoro-1-methylpiperidin-3-yl)amino)-7,8-dihydro-5H-pyrano[3,4-d]pyridazin-1-yl)-5-methoxyphenol

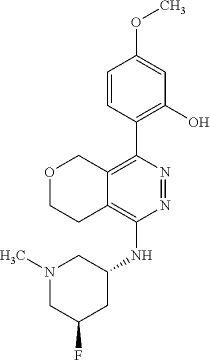 [SMILES: COc1ccc(-c2nnc(N[C@@H]3C[C@@H](F)CN(C)C3)c3c2COCC3)c(O)c1]

A mixture of 1-chloro-N-((3R,5R)-5-fluoro-1-methylpiperidin-3-yl)-7,8-dihydro-5H-pyrano[3,4-d]pyridazin-4-amine (30 mg, 0.0997 mmol), (2-hydroxy-4-methoxyphenyl)boronic acid (22 mg, 0.130 mmol), XPhos Palladacycle G4 (8.6 mg, 0.00997 mmol) and K₃PO₄ (42 mg, 0.199 mmol) in THE (2.3 mL) and water (0.6 mL) was stirred at 70° C. for 1 hour using a metal heating block. The reaction mixture was diluted with in MeOH (1 mL), filtered through a 0.45 μm PTFE Membrane filter (VWR), rinsed with MeOH (1 mL), and purified via preparative HPLC (Method A). The product-containing fractions were combined, concentrated by rotary evaporation at 45° C. and dried in vacuo to give a TFA salt of the title compound as a brown film (54.5 mg, crude). The crude product was dissolved in MeOH (1 mL) and passed through a 100 mg Agilent StratoSpheres SPE cartridge (PL-HCO₃ MP SPE), rinsing with MeOH (1 mL×3), to remove TFA. The filtrate was concentrated, filtered through a 0.45 μm PTFE Membrane filter (VWR), rinsed with MeOH (1 mL) and purified by preparative HPLC (Shimadzu) using a gradient 40 to 100% ACN (4:1 ACN/H₂O with 10 mM NH₄HCO₃) in H₂O (10 mM NH₄HCO₃). The pure fractions were combined, concentrated by rotary evaporation at 45° C. and dried in vacuo to give the title compound as a light-yellow solid (10.1 mg, 26.1%). ¹H NMR (400 MHz, DMSO-d₆) δ ppm 1.64-1.74 (m, 1H), 1.89 (br t, J=10.04 Hz, 1H), 2.09-2.18 (m, 2H), 2.21 (s, 3H), 2.45-2.48 (m, 2H), 2.85-2.91 (m, 1H), 2.98 (br dd, J=10.16, 2.89 Hz, 1H), 3.67-3.83 (m, 5H), 4.46 (s, 2H), 4.51-4.58 (m, 1H), 4.88-5.01 (m, 1H), 5.61 (br d, J=7.78 Hz, 1H), 6.48 (dq, J=4.58, 2.41 Hz, 2H), 7.12 (d, J=9.03 Hz, 1H), 10.06 (s, 1H); ESI-MS [M+H]⁺ 389.2.

### Example 238: 5-cyclopropyl-2-(4-(((3R,5R)-5-fluoro-1-methylpiperidin-3-yl)amino)-7,8-dihydro-5H-pyrano[3,4-d]pyridazin-1-yl)phenol

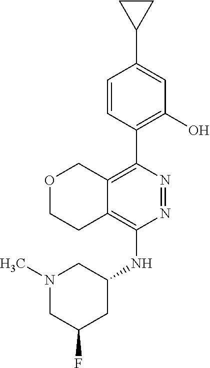 [SMILES: CN1C[C@H](F)C[C@@H](Nc2nnc(-c3ccc(C4CC4)cc3O)c3c2CCOC3)C1]

### Step 1: 1-(4-cyclopropyl-2-(methoxymethoxy)phenyl)-N-((3R,5R)-5-fluoro-1-methylpiperidin-3-yl)-7,8-dihydro-5H-pyrano[3,4-d]pyridazin-4-amine

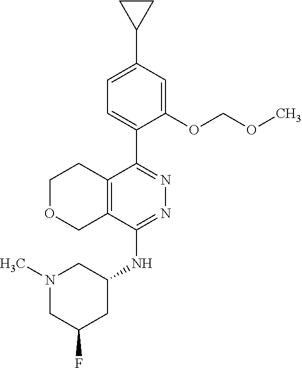

A mixture of 1-chloro-N-((3R,5R)-5-fluoro-1-methylpiperidin-3-yl)-7,8-dihydro-5H-pyrano[3,4-d]pyridazin-4-amine (50 mg, 166.25 mol), 2-(4-cyclopropyl-2-(methoxymethoxy)phenyl)-4,4,5,5-tetramethyl-1,3,2-dioxaborolane (75.86 mg, 249.37 mol), Cs₂CO₃ (216.67 mg, 664.98 mol), Pd(dppf)Cl₂·CH₂Cl₂ (13.58 mg, 16.62 mol) and dioxane (2 mL) in H₂O (0.5 mL) was degassed and purged with N₂ (3×) and then stirred at 100° C. for 2 hours under N₂ atmosphere. LC-MS showed 65% of product with desired mass was obtained. The reaction mixture was quenched with H₂O (3 mL) and extracted with EtOAc (2 mL×3). The organic layer was dried over Na₂SO₄, filtered and evaporated to dryness. The residue was purified by flash chromatography (ISCO® SepaFlash® 4 g silica gel column) using a gradient of 0 to 6% DCM in MeOH (20 mL/min). The title compound was obtained as a black solid (70 mg, 78% yield, 82% purity). ESI-MS m/z [M+H]⁺ 443.3.

### Step 2: 5-cyclopropyl-2-(4-(((3R,5R)-5-fluoro-1-methylpiperidin-3-yl)amino)-7,8-dihydro-5H-pyrano[3,4-d]pyridazin-1-yl)phenol

To a solution of 1-(4-cyclopropyl-2-(methoxymethoxy)phenyl)-N-((3R,5R)-5-fluoro-1-methylpiperidin-3-yl)-7,8-dihydro-5H-pyrano[3,4-d]pyridazin-4-amine (60 mg, 135.59 mol) in DCM (3 mL) was added TFA (0.6 mL). The reaction mixture was stirred at 25° C. for 12 hours. LC-MS showed 46% of product with desired mass was obtained. The mixture was filtered and evaporated to dryness. The residue was purified by preparative HPLC (Welch Xtimate C18 10 μm, 40 mm×150 mm) using a gradient of 0 to 24% ACN in water (0.225% FA) over 25 minutes to give the title compound as a yellow solid (16 mg, purity 90%). The product was further purified by SFC (DAICEL CHIRALPAK® AD-10 μm, 30 mm×250 mm column) using a mobile phase of CO₂ and 45% IPA (with 0.1% NH₃H₂O). The title compound was obtained as a white solid (8 mg, 14% yield, 95% purity). ¹H NMR (400 MHz, CD₃OD) δ ppm 0.66-0.73 (m, 2H), 0.95-1.03 (m, 2H), 1.72-1.94 (m, 2H), 2.09-2.21 (m, 1H), 2.25-2.45 (m, 5H), 2.56-2.65 (m, 2H), 2.90-3.02 (m, 1H), 3.11-3.18 (m, 1H), 3.86 (t, J=5.50 Hz, 2H), 4.51 (s, 2H), 4.68-4.79 (m, 1H), 4.99 (br d, J=2.13 Hz, 1H), 6.57-6.74 (m, 2H), 7.02-7.16 (m, 1H); ESI-MS m/z [M+H]⁺ 399.0.

### Example 239: 5-(difluoromethyl)-2-(4-(((3R,5R)-5-fluoro-1-methylpiperidin-3-yl)amino)-7,8-dihydro-5H-pyrano[3,4-d]pyridazin-1-yl)phenol

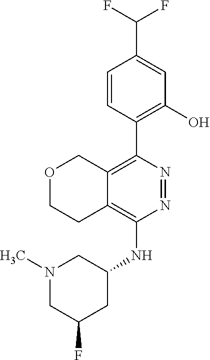 [SMILES: CN1C[C@H](F)C[C@@H](Nc2nnc(-c3ccc(C(F)F)cc3O)c3c2CCOC3)C1]

### Step 1: 1-(4-(difluoromethyl)-2-(methoxymethoxy)phenyl)-N-((3R,5R)-5-fluoro-1-methylpiperidin-3-yl)-7,8-dihydro-5H-pyrano[3,4-d]pyridazin-4-amine

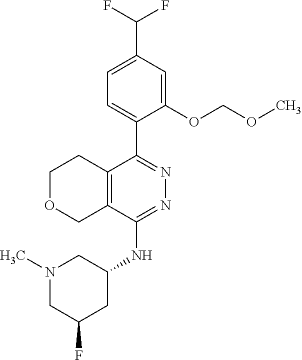

To a solution of 1-chloro-N-((3R,5R)-5-fluoro-1-methylpiperidin-3-yl)-7,8-dihydro-5H-pyrano[3,4-d]pyridazin-4-amine (50 mg, 166.25 mol) and 2-(4-(difluoromethyl)-2-(methoxymethoxy)phenyl)-4,4,5,5-tetramethyl-1,3,2-dioxaborolane (208.89 mg, 664.98 mol) in THE (2 mL) were added Xphos-Pd-G4 (14.30 mg, 16.62 mol) and K₃PO₄ (0.5 M, 2 mL). The mixture was stirred at 50° C. for 1 hour under N₂. LC-MS showed 80% of product with desired mass was obtained. The reaction mixture was quenched with H₂O (5 mL) and extracted with EtOAc (3 mL×3). The organic layer was dried over Na₂SO₄, filtered and evaporated to dryness. The residue was purified by flash chromatography (ISCO® SepaFlash® 4 g silica gel column) using a gradient of 0 to 10% DCM in MeOH (30 m/min). The title compound was obtained as a black solid (80 mg, 96% yield, 90% purity). ESI-MS m/z [M+H]⁺ 453.2.

### Step 2: 5-(difluoromethyl)-2-(4-(((3R,5R)-5-fluoro-1-methylpiperidin-3-yl)amino)-7,8-dihydro-5H-pyrano[3,4-d]pyridazin-1-yl)phenol

To a solution of 1-(4-(difluoromethyl)-2-(methoxymethoxy)phenyl)-N-((3R,5R)-5-fluoro-1-methylpiperidin-3-yl)-7,8-dihydro-5H-pyrano[3,4-d]pyridazin-4-amine (70 mg, 154.71 mol) in DCM (3 mL) was added TFA (0.6 mL). The reaction mixture was stirred at 25° C. for 12 hours. LC-MS showed 92% of product with desired mass was obtained. The mixture was filtered and evaporated to dryness. The residue was purified by preparative HPLC (Xtimate C18 10 μm, 40 mm×150 mm) using a gradient of 0 to 24% ACN in water (with TFA) over 36 minutes. A TFA salt of the title compound was obtained as a white solid (60 mg, 71% yield, 95% purity). ¹H NMR (400 MHz, CD₃OD) δ ppm 1.92-2.16 (m, 1H), 2.50-2.62 (m, 1H), 2.65-2.73 (m, 2H), 2.92-3.04 (m, 4H), 3.34-3.48 (m, 1H), 3.76-3.88 (m, 2H), 3.93 (t, J=5.50 Hz, 2H), 4.61-4.72 (m, 2H), 4.77-4.84 (m, 1H), 5.19-5.40 (m, 1H), 6.62-6.97 (m, 1H), 7.14-7.25 (m, 2H), 7.41-7.48 (m, 1H); ESI-MS m/z [M+H]+ 409.2.

Table 5 lists in vitro biological assay data (IL-10, TNF-α and MDCK-MDR1) for some of the compounds shown in the examples. These assays are described in the section entitled Biological Activity, above.

TABLE 5
Biological Assay Data

| Example | IL-1β IC₅₀ | TNF-α IC₅₀ | MDR1 P_{app A-B} | |
|---|---|---|---|---|
| No. | (nM) | (nM) | (nm/s) | MDR1 ER |
| 1 | 1883 | 7200 | | |
| 2 | 5248 | | | |
| 3 | 1122 | | | |
| 4 | 1413 | | | |
| 5 | 1445 | | | |
| 6 | 2884 | | | |
| 7 | 977 | | | |
| 8 | 891 | | | |
| 9 | 1660 | | | |
| 10 | 437 | | | |
| 11 | 933 | | | |
| 12 | 2570 | | | |
| 13 | 65 | | | |
| 14 | 2951 | | | |
| 15 | 115 | | | |
| 16 | 112 | >30000 | | |
| 17 | 4467 | | | |
| 18 | 1148 | | | |
| 19 | 1087 | 7760 | | |
| 20 | 626 | | | |
| 21 | 75 | >30000 | | |
| 22 | 897 | | | |
| 23 | 10000 | | | |
| 24 | 157 | | | |
| 25 | 199 | | | |
| 26 | 603 | | | |
| 27 | 38 | | | |
| 28 | 8 | >30000 | | |
| 29 | | | | |
| 30 | 97 | >30000 | | |
| 31 | 34 | | | |
| 32 | 36 | | | |
| 33 | 33 | | | |
| 34 | 28 | | | |
| 35 | 88 | >30000 | | |
| 36 | 10 | >30000 | | |
| 37 | 77 | | | |
| 38 | 1122 | | | |
| 39 | 1624 | >30000 | | |
| 40 | 3020 | >30000 | | |
| 41 | 394 | >30000 | | |
| 42 | 1150 | | | |
| 43 | 41 | >30000 | | |
| 44 | 97 | >30000 | | |
| 45 | 4117 | | | |
| 46 | 113 | >30000 | | |
| 47 | 692 | | | |
| 48 | 3090 | | | |
| 49 | 288 | | | |
| 50 | 1318 | | | |
| 51 | 158 | | | |
| 52 | 2399 | | | |
| 53 | 40 | | | |
| 54 | 1202 | | | |
| 55 | 575 | | | |
| 56 | 72 | >30000 | | |
| 57 | 21 | | | |
| 58 | 66 | >30000 | | |
| 59 | 2 | >30000 | | |
| 60 | 27 | 21400 | | |
| 61 | 134 | | | |
| 62 | 3548 | | | |
| 63 | 381 | | | |
| 64 | 58 | >30000 | | |
| 65 | 1218 | | | |
| 66 | | | | |
| 67 | 552 | >30000 | | |
| 68 | 46 | >30000 | | |
| 69 | 838 | | | |
| 70 | 1995 | | | |
| 71 | 46 | 9550 | | |
| 72 | 52 | >30000 | | |
| 73 | 270 | >30000 | | |
| 74 | 4 | >30000 | | |
| 75 | 3 | >30000 | | |
| 76 | 178 | >30000 | | |
| 77 | 1514 | | | |
| 78 | 183 | | | |
| 79 | 5 | 24300 | | |
| 80 | 36 | >30000 | | |
| 81 | 67 | >30000 | | |
| 82 | 95 | >10000 | | |
| 83 | 82 | >30000 | | |
| 84 | 79 | >30000 | | |
| 85 | 2 | >30000 | | |
| 86 | 21 | >30000 | | |
| 87 | 33 | >30000 | | |
| 88 | 8 | >30000 | | |
| 89 | 91 | >30000 | | |
| 90 | 3 | | | |
| 91 | 307 | | | |
| 92 | 60 | | | |
| 93 | 30 | >30000 | | |
| 94 | 102 | | | |
| 95 | 10 | >30000 | | |
| 96 | 5 | 26000 | | |
| 97 | | | | |
| 98 | | | | |
| 99 | 2042 | | | |
| 100 | 2692 | | | |
| 101 | 804 | | | |
| 102 | 692 | | | |
| 103 | 3631 | | | |
| 104 | 1660 | | | |
| 105 | 955 | | | |
| 106 | 3311 | | | |
| 107 | 1778 | | | |
| 108 | 3157 | | | |
| 109 | 4898 | | | |
| 110 | 4477 | | | |
| 111 | 3776 | >30000 | | |
| 112 | 4253 | | | |
| 113 | 47 | >30000 | | |
| 114 | 1230 | | | |
| 115 | 2188 | | | |
| 116 | 912 | | | |
| 117 | 624 | | | |
| 118 | 334 | >30000 | | |
| 119 | 2754 | | | |
| 120 | 933 | | | |
| 121 | 3981 | | | |
| 122 | 2296 | >30000 | | |
| 123 | 276 | >30000 | | |
| 124 | 1290 | >30000 | | |
| 125 | 2754 | | | |
| 126 | 2570 | | | |
| 127 | 228 | >30000 | | |
| 128 | 3020 | | | |
| 129 | 2818 | | | |
| 130 | 3890 | | | |
| 131 | 3631 | | | |
| 132 | 3890 | | | |
| 133 | 468 | | | |
| 134 | 2291 | | | |
| 135 | 4571 | | | |
| 136 | 3715 | | | |
| 137 | 3802 | | | |
| 138 | 1106 | | | |
| 139 | 31 | >30000 | | |
| 140 | 75 | >30000 | | |
| 141 | 269 | | | |
| 142 | 195 | | | |
| 143 | 1585 | | | |
| 144 | 43 | >30000 | | |
| 145 | 132 | | | |
| 146 | 56 | | | |
| 147 | 1479 | | | |
| 148 | 1047 | | | |
| 149 | 3311 | | | |
| 150 | 325 | | | |
| 151 | 3020 | | | |
| 152 | 490 | >30000 | | |
| 153 | 2257 | >30000 | | |
| 154 | 3388 | | | |
| 155 | 66 | 23400 | | |
| 156 | 3548 | | | |
| 157 | 4571 | | | |
| 158 | 2291 | | | |
| 159 | 3631 | | | |
| 160 | 4786 | | | |
| 161 | 977 | | | |
| 162 | 18 | >30000 | | |
| 163 | 3020 | | | |
| 164 | 372 | | | |
| 165 | 3467 | | | |
| 166 | 3311 | | | |
| 167 | 3388 | | | |
| 168 | 5 | >30000 | | |
| 169 | 7 | >30000 | | |
| 170 | 2138 | | | |
| 171 | 68 | >30000 | | |
| 172 | 20 | >30000 | | |
| 173 | 3 | >30000 | | |
| 174 | 159 | >30000 | | |
| 175 | 589 | | | |
| 176 | 37 | >30000 | | |
| 177 | 53 | >30000 | | |
| 178 | 81 | >30000 | | |
| 179 | 575 | | | |
| 180 | 136 | | | |
| 181 | 1000 | | | |
| 182 | 3467 | | | |
| 183 | 589 | | | |
| 184 | 152 | >30000 | | |
| 185 | 311 | >30000 | | |
| 186 | 5 | >30000 | | |
| 187 | 3090 | | | |
| 188 | 692 | | | |
| 189 | 110 | >30000 | | |
| 190 | 648 | 7586 | | |
| 191 | 1288 | | | |
| 192 | 60 | >30000 | | |
| 193 | 22 | >30000 | | |
| 194 | 35 | >30000 | | |
| 195 | 113 | >30000 | | |
| 196 | 12 | >30000 | | |
| 197 | 708 | | | |
| 198 | 4380 | | | |
| 199 | 2754 | | | |
| 200 | 3 | >30000 | | |
| 201 | 10 | >30000 | 17 | 17 |
| 202 | 210 | | | |
| 203 | 32 | >30000 | | |
| 204 | 856 | >30000 | | |
| 205 | 64 | >30000 | | |
| 206 | 425 | >30000 | | |
| 207 | 2685 | >30000 | | |
| 208 | 853 | >30000 | | |
| 209 | 1050 | >30000 | | |
| 210 | 757 | >30000 | | |
| 211 | 1146 | >30000 | | |
| 212 | 2 | >30000 | 21 | 9 |
| 213 | 28 | >30000 | 31 | 14 |
| 214 | 556 | >30000 | | |
| 215 | 160 | >30000 | | |
| 216 | 373 | >30000 | | |
| 217 | 876 | >30000 | | |
| 218 | 2 | >30000 | 12 | 26 |
| 219 | 8 | >30000 | 47 | 13 |
| 220 | 8 | >30000 | 15 | 9 |
| 221 | 3 | >30000 | 10 | 30 |
| 222 | 49 | >30000 | | |
| 223 | 457 | | | |
| 224 | 2 | >30000 | | |
| 225 | 2 | >30000 | 15 | 22 |
| 226 | 9 | >30000 | 13 | 21 |
| 227 | 21 | | | |
| 228 | 23 | | | |
| 229 | 36 | >10000 | 10 | 6 |
| 230 | 121 | >10000 | 13 | 7 |
| 231 | 195 | | | |
| 232 | 155 | >30000 | | |
| 233 | 155 | >30000 | 20 | 25 |
| 234 | 6 | >30000 | 15 | 12 |
| 235 | 20 | | 35 | 5 |
| 236 | 7 | >30000 | 22 | 10 |
| 237 | 11 | >30000 | 16 | 10 |
| 238 | 5 | >30000 | 6 | 41 |
| 239 | 11 | >30000 | 12 | 39 |

As used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. The above description is intended to be illustrative and not restrictive. Many embodiments will be apparent to those of skill in the art upon reading the above description. Therefore, the scope of the invention should be determined with reference to the appended claims and includes the full scope of equivalents to which such claims are entitled. The disclosures of all articles and references cited in the disclosure, including patents, patent applications and publications, are herein incorporated by reference in their entirety and for all purposes.

What is claimed is:

1. A compound of Formula 1, 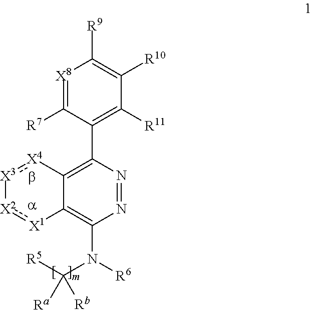
or a pharmaceutically acceptable salt thereof in which:
a is a single bond and p is a single bond; and
X¹ is CH₂;
X² is O and X³ is CH₂, or
X² is CH₂ and X³ is O; and
X⁴ is CH₂;
m is selected from 0, 1 and 2;
each R^{a} and R^{b} is independently selected from hydrogen and C₁₋₄ alkyl, or R^{a} and R^{b}, together with a carbon atom to which both R^{a} and R^{b} are attached, form a C₃₋₆ cycloalkylidene, provided if m is 2, then no more than one R^{a} and R^{b}, together with the carbon atom to which R^{a} and R^{b} are attached, form a C₃₋₆ cycloalkylidene;
R⁵ is selected from:
(a) C₃₋₈ cycloalkyl, which is substituted with 0 to 5 substituents independently selected from:
(i) halo, hydroxy, cyano and oxo;
(ii) amino, which is substituted with 0 to 2 substituents independently selected from C₁₋₄ alkyl; and
(iii) C₁₋₄ alkyl, C₁₋₄ alkylcarbonyl and C₁₋₄ alkoxy, each substituted with 0 to 3 substituents independently selected from halo;
provided:
if m is 0, X¹, X² and X⁴ are each CH₂, X³ is 0, X⁸ is CH, R⁶, R¹⁰ and R¹¹ are each hydrogen, R⁷ is hydroxy and R⁹ is CF₃ or OCF₃, then R⁵ is not 3-hydroxy-3-methylcyclobutyl;
if m is 0, X¹, X³ and X⁴ are each CH₂, X² is 0, X⁸ is CH, R⁶, R¹⁰ and R¹¹ are each hydrogen, R⁷ is hydroxy and R⁹ is CF₃ or OCF₃, then R⁵ is not 3-hydroxy-3-methylcyclobutyl;
if m is 0, X¹, X² and X⁴ are each CH₂, X³ is 0, X⁸ is CH, R⁶, R¹⁰ and R¹¹ are each hydrogen, R⁷ is hydroxy, and R⁹ is hydrogen, CF₃, OCF₃ or cyclobutyl, then R⁵ is not 2-hydroxycyclohexyl;
if m is 0, X¹, X³ and X⁴ are each CH₂, X² is 0, X⁸ is CH, R⁶, R¹⁰ and R¹¹ are each hydrogen, R⁷ is hydroxy, and R⁹ is hydrogen, chloro, CF₃, CHF₂, OCF₃, OCHF₂, OCH₃ or cyclobutyl, then R⁵ is not 2-hydroxycyclohexyl;
if m is 0, X¹, X² and X⁴ are each CH₂, X³ is 0, X⁸ is CH, R⁶ and R¹⁰ are each hydrogen, R⁷ is hydroxy, R⁹ is methyl, and R¹¹ is fluoro, then R⁵ is not 2-hydroxycyclohexyl;
if m is 0, X¹, X³ and X⁴ are each CH₂, X² is 0, X⁸ is CH, R⁶ and R¹⁰ are each hydrogen, R⁷ is hydroxy, R⁹ is methyl, and R¹¹ is fluoro, then R⁵ is not 2-hydroxycyclohexyl;
if m is 0, X¹, X³ and X⁴ are each CH₂, X² is 0, X⁸ is CH, R⁶ and R¹¹ are each hydrogen, R⁷ is hydroxy, R⁹ is CF₃, and R¹⁰ is fluoro, then R⁵ is not 2-hydroxycyclohexyl;
if m is 0, X¹, X² and X⁴ are each CH₂, X³ is 0, X⁸ is CH, R⁶, R¹⁰ and R¹¹ are each hydrogen, R⁷ is hydroxy, and R⁹ is CF₃, then R⁵ is not 2-cyanocyclohexyl or 2-aminocyclohexyl; and
if m is 0, X¹, X³ and X⁴ are each CH₂, X² is 0, X⁸ is CH, R⁶, R¹⁰ and R¹¹ are each hydrogen, R⁷ is hydroxy, and R⁹ is CF₃, then R⁵ is not 2-cyanocyclohexyl;
(b) C₃₋₈ heterocyclyl in which up to 3 carbon ring atoms are each independently substituted with 0 to 2 substituents independently selected from:
(i) halo, hydroxy, cyano and oxo;
(ii) amino, which is substituted with 0 to 2 substituents independently selected from C₁₋₄ alkyl; and
(iii) C₁₋₄ alkyl, C₁₋₄ alkylcarbonyl and C₁₋₄ alkoxy, each substituted with 0 to 3 substituents independently selected from halo;
and in which a nitrogen ring atom, if present, is unsubstituted or substituted with a substituent selected from:
(i) C₁₋₄ alkyl, C1.4 alkylcarbonyl and C₁₋₄ alkylsulfonyl, each substituted with 0 to 3 substituents independently selected from halo;
(ii) C₃₋₈ cycloalkyl-(CH₂)ₙ, which C₃₋₈ cycloalkyl moiety is substituted with 0 to 3 substituents independently selected from halo, C₁0.4 alkyl, C₁₋₄ alkylcarbonyl, C₁₋₄ alkoxy and oxo; and
(iii) phenyl-(CH₂)ₙ and pyridinyl-(CH₂)ₙ, which phenyl and pyridinyl moieties are substituted with 0 to 3 substituents independently selected from halo, hydroxy, cyano, C₁0.4 alkyl and C₁0.4 alkoxy;
wherein the C₃₋₈ heterocyclyl has only one ring heteroatom, the ring heteroatom being selected from nitrogen, oxygen, and sulfur; and
n is selected from 0 and 1;
provided:
if m is 0, X¹, X² and X⁴ are each CH₂, X³ is 0, X⁸ is CH, R⁶, R¹⁰ and R¹¹ are each hydrogen, R⁷ is hydroxy and R⁹ is CF₃, then R⁵ is not piperidin-3-yl or 1-methylpiperidin-3-yl;
if m is 0, X¹, X³ and X⁴ are each CH₂, X² is 0, X⁸ is CH, R⁶, R¹⁰ and R¹¹ are each hydrogen, R⁷ is hydroxy and R⁹ is CF₃, then R⁵ is not piperidin-3-yl or 1-methylpiperidin-3-yl;
if m is 0, X¹, X² and X⁴ are each CH₂, X³ is 0, X⁸ is CH, R⁶ and R¹⁰ are each hydrogen, R⁷ is hydroxy, R⁹ is CF₃, methyl or chloro, and R¹¹ is fluoro, then R⁵ is not 1-methylpiperidin-3-yl; and
if m is 0, X¹, X² and X⁴ are each CH₂, X³ is 0, X⁸ is CH, R⁶, R¹⁰ and R¹¹ are each hydrogen, R⁷ is hydroxy and R⁹ is methyl or chloro, then R⁵ is not 1-methylpiperidin-3-yl;
(c) phenyl, which is substituted with 0 to 3 substituents independently selected from halo, hydroxy, cyano, C₁₋₄ alkyl and C₁₋₄ alkoxy, provided at least one of the substituents is hydroxy;
R⁶ is selected from hydrogen and C₁₋₄ alkyl;
X⁸ is selected from N and CR⁸;
R⁷, R⁸ and R¹¹ are each independently selected from:
(i) hydrogen, halo, hydroxy and cyano;
(ii) C₁₋₄ alkyl and C₁₋₄ alkoxy, each substituted with 0 to 3 substituents independently selected from halo; and
(iii) C₃₋₈ cycloalkyl which is substituted with 0 to 3 substituents independently selected from halo, C₁₋₄ alkyl and C₁0.4 alkoxy; and
R⁹ and R¹⁰ are each independently selected from:
(i) hydrogen, halo, hydroxy and cyano;
(ii) C₁₋₄ alkyl and C₁₋₄ alkoxy, each substituted with 0 to 3 substituents independently selected from halo; and
(iii) C₃₋₈ cycloalkyl which is substituted with 0 to 3 substituents independently selected from halo, C₁₋₄ alkyl and C₁₋₄ alkoxy; or
R⁹ and R¹⁰ form an ethan-1,2-dioxy moiety bridging the carbon atoms to which they are attached.

2. The compound or pharmaceutically acceptable salt according to claim 1, wherein X² is O and X³ is CH₂.

3. The compound or pharmaceutically acceptable salt according to any one of claims 1 to 2, wherein m is 0.

4. The compound or pharmaceutically acceptable salt according to any one of claims 1 to 2, wherein m is 1 or 2.

5. The compound or pharmaceutically acceptable salt according to claim 4, wherein each R^{a} and R^{b} is independently selected from hydrogen and C₁₋₄ alkyl.

6. The compound or pharmaceutically acceptable salt according to any one of claims 1 to 5, wherein R⁵ is C₃₋₈ cycloalkyl, which is substituted with 0 to 5 substituents independently selected from:
(i) halo, hydroxy, cyano and oxo;
(ii) amino, which is substituted with 0 to 2 substituents independently selected from C₁₋₄ alkyl; and
(iii) C₁₋₄ alkyl, C₁₋₄ alkylcarbonyl and C₁₋₄ alkoxy, each substituted with 0 to 3 substituents independently selected from halo.

7. The compound or pharmaceutically acceptable salt according to any one of claims 1 to 5, wherein R⁵ is a cycloalkyl which is selected from cyclobutyl, cyclohexyl and bicyclo[2.2.1]heptan-1-yl, each substituted with 0 to 5 substituents independently selected from:
(i) halo, hydroxy, cyano and oxo;
(ii) amino, which is substituted with 0 to 2 substituents independently selected from C₁₋₄ alkyl; and
(iii) C₁₋₄ alkyl, C₁₋₄ alkylcarbonyl and C₁₋₄ alkoxy, each substituted with 0 to 3 substituents independently selected from halo.

8. The compound or pharmaceutically acceptable salt according to any one of claims 1 to 5, wherein R⁵ is C₃₋₈ heterocyclyl in which up to 3 carbon ring atoms are each independently substituted with 0 to 2 substituents independently selected from:
(i) halo, hydroxy, cyano and oxo;
(ii) amino, which is substituted with 0 to 2 substituents independently selected from C₁₋₄ alkyl; and
(iii) C₁₋₄ alkyl, C₁₋₄ alkylcarbonyl and C₁₋₄ alkoxy, each substituted with 0 to 3 substituents independently selected from halo;
and in which a nitrogen ring atom, if present, is unsubstituted or substituted with a substituent selected from:
(i) C₁₋₄ alkyl, C₁₋₄ alkylcarbonyl and C₁₋₄ alkylsulfonyl, each substituted with 0 to 3 substituents independently selected from halo;
(ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
(iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy.

9. The compound or pharmaceutically acceptable salt according to any one of claims 1 to 5, wherein R⁵ is C₃₋₈ heterocyclyl in which the ring heteroatom is nitrogen and up to 3 carbon ring atoms are each independently substituted with 0 to 2 substituents independently selected from:
(i) halo, hydroxy, cyano and oxo;
(ii) amino, which is substituted with 0 to 2 substituents independently selected from C₁₋₄ alkyl; and
(iii) C₁₋₄ alkyl, C₁₋₄ alkylcarbonyl and C₁₋₄ alkoxy, each substituted with 0 to 3 substituents independently selected from halo;
and in which the nitrogen ring atom is unsubstituted or substituted with a substituent selected from:
(i) C₁₋₄ alkyl, C₁₋₄ alkylcarbonyl and C₁₋₄ alkylsulfonyl, each substituted with 0 to 3 substituents independently selected from halo;
(ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
(iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy.

10. The compound or pharmaceutically acceptable salt according to any one of claims 1 to 5, wherein R⁵ is C₃₋₈ heterocyclyl which is selected from piperidin-3-yl in which up to 3 carbon ring atoms are each independently substituted with 0 to 2 substituents independently selected from:
(i) halo, hydroxy, cyano and oxo;
(ii) amino, which is substituted with 0 to 2 substituents independently selected from C₁₋₄ alkyl; and
(iii) C₁₋₄ alkyl, C₁₋₄ alkylcarbonyl and C₁₋₄ alkoxy, each substituted with 0 to 3 substituents independently selected from halo;
and in which the nitrogen ring atom of the piperidin-3-yl is unsubstituted or substituted with a substituent selected from:
(i) C₁₋₄ alkyl, C₁₋₄ alkylcarbonyl and C₁₋₄ alkylsulfonyl, each substituted with 0 to 3 substituents independently selected from halo;
(ii) C₃₋₈ cycloalkyl-(CH₂)ₙ, which C₃₋₈ cycloalkyl moiety is substituted with 0 to 3 substituents independently selected from halo, C₁₋₄ alkyl, C₁₋₄ alkylcarbonyl, C₁₋₄ alkoxy and oxo; and
(iii) phenyl-(CH₂)ₙ and pyridinyl-(CH₂)ₙ, which phenyl and pyridinyl moieties are substituted with 0 to 3 substituents independently selected from halo, hydroxy, cyano, C₁₋₄ alkyl and C₁₋₄ alkoxy.

11. The compound or pharmaceutically acceptable salt according to any one of claims 1 to 5, wherein R⁵ is C₃₋₈ heterocyclyl which is selected from piperidin-3-yl in which up to 3 carbon ring atoms are each independently substituted with 0 to 2 substituents independently selected from:
(i) halo and oxo;
(ii) amino, which is substituted with 0 to 2 substituents independently selected from methyl; and
(iii) methyl and methoxy, each substituted with 0 to 3 substituents independently selected from fluoro;
and in which the nitrogen ring atom of the piperidin-3-yl is unsubstituted or substituted with a substituent selected from:
(i) methyl, ethyl, isopropyl, methylcarbonyl, methylsulfonyl, each substituted with 0 to 3 substituents selected from fluoro;
(ii) C₃₋₅ cycloalkyl-(CH₂)ₙ, which C₃₋₅ cycloalkyl moiety is substituted with 0 to 3 substituents independently selected from methyl and methoxy; and
(iii) phenyl-(CH₂)ₙ and pyridinyl-(CH₂)ₙ, which phenyl and pyridinyl moieties are unsubstituted.

12. The compound or pharmaceutically acceptable salt according to any one of claims 1 to 5, wherein R⁵ is C₃₋₈ heterocyclyl which is selected from piperidin-3-yl in which up to 3 carbon ring atoms are each independently substituted with 0 to 2 substituents independently selected from halo, oxo, methyl, ethyl, propyl and isopropyl, and in which the nitrogen ring atom of the piperidin-3-yl is unsubstituted or substituted with a substituent selected from methyl, ethyl, isopropyl and C₃₋₅ cycloalkyl-(CH₂)ₙ.

13. The compound or pharmaceutically acceptable salt according to any one of claims 8 to 12, wherein R⁵ is C₃₋₈ heterocyclyl in which at least one of the carbon ring atoms of the R⁵ heterocyclyl is substituted.

14. The compound or pharmaceutically acceptable salt according to any one of claims 8 to 12, wherein R⁵ is C₃₋₈ heterocyclyl in which at least one of the carbon ring atoms of the R⁵ heterocyclyl is substituted with halo

15. The compound or pharmaceutically acceptable salt according to any one of claims 8 to 12, wherein R⁵ is C₃₋₈ heterocyclyl in which at least one of the carbon ring atoms of the R⁵ heterocyclyl is substituted with fluoro.

16. The compound or pharmaceutically acceptable salt according to any one of claims 8 to 15, wherein R⁵ is C₃₋₈ heterocyclyl in which a nitrogen ring atom, if present, is unsubstituted or substituted with a substituent selected from methyl, ethyl, isopropyl and C₃0.5 cycloalkyl-(CH₂)ₙ.

17. The compound or pharmaceutically acceptable salt according to any one of claims 8 to 15, wherein R⁵ is C₃₋₈ heterocyclyl in which a nitrogen ring atom, if present, is substituted with a substituent selected from methyl, ethyl, isopropyl and C₃₋₅ cycloalkyl-(CH₂)ₙ.

18. The compound or pharmaceutically acceptable salt according to any one of claims 8 to 17, wherein R⁵ is C₃₋₈ heterocyclyl and n is 0.

19. The compound or pharmaceutically acceptable salt according to any one of claims 1 to 5, wherein R⁵ is phenyl, which is substituted with 0 to 3 substituents independently selected from halo, hydroxy, cyano, C₁₋₄ alkyl and C₁₋₄ alkoxy, provided at least one of the substituents is hydroxy.

20. The compound or pharmaceutically acceptable salt according to any one of claims 1 to 19, wherein R⁶ is selected from hydrogen and methyl.

21. The compound or pharmaceutically acceptable salt according to any one of claims 1 to 19, wherein R⁶ is hydrogen.

22. The compound or pharmaceutically acceptable salt according to any one of claims 1 to 21, wherein X⁸ is CR⁸.

23. The compound or pharmaceutically acceptable salt according to any one of claims 1 to 22, wherein R⁸ and R¹¹ are both hydrogen, and R⁷ is selected from:
(i) hydrogen, halo and hydroxy; and
(ii) C₁₋₃ alkyl and C₁₋₃ alkoxy, each substituted with 0 to 3 substituents independently selected from halo.

24. The compound or pharmaceutically acceptable salt according to any one of claims 1 to 23, wherein R⁹ and R¹⁰ are each independently selected from:
(i) hydrogen, halo, hydroxy and cyano;
(ii) C₁₋₄ alkyl and C₁₋₃ alkoxy, each substituted with 0 to 3 fluoro; and
(iii) C₃₋₅ cycloalkyl which is substituted with 0 to 3 substituents independently selected from methyl and methoxy.

25. The compound according to claim 1, which is selected from the following compounds:
5-chloro-2-(1-(((3R,5R)-5-fluoro-1-methylpiperidin-3-yl)amino)-7,8-dihydro-5H-pyrano[3,4-d]pyridazin-4-yl)phenol;
2-(1-(((3R,5R)-5-fluoro-1-methylpiperidin-3-yl)amino)-7,8-dihydro-5H-pyrano[3,4-d]pyridazin-4-yl)-5-methylphenol;
2-(1-(((3R,5R)-5-fluoro-1-methylpiperidin-3-yl)amino)-7,8-dihydro-5H-pyrano[3,4-d]pyridazin-4-yl)phenol;
5-fluoro-2-(1-(((3R,5R)-5-fluoro-1-methylpiperidin-3-yl)amino)-7,8-dihydro-5H-pyrano[3,4-d]pyridazin-4-yl)phenol;
1-(2-(difluoromethyl)-4-methylphenyl)-N-((3R,5R)-5-fluoro-1-methylpiperidin-3-yl)-7,8-dihydro-5H-pyrano[3,4-d]pyridazin-4-amine;
N-((3R,5R)-5-fluoro-1-methylpiperidin-3-yl)-1-(2-fluoro-4-(trifluoromethoxy)phenyl)-7,8-dihydro-5H-pyrano[3,4-d]pyridazin-4-amine;
1-(2-(difluoromethyl)-4-methoxyphenyl)-N-((3R,5R)-5-fluoro-1-methylpiperidin-3-yl)-7,8-dihydro-5H-pyrano[3,4-d]pyridazin-4-amine;
4-(2-(difluoromethyl)-4-methylphenyl)-N-((3R,5R)-5-fluoro-1-methylpiperidin-3-yl)-7,8-dihydro-5H-pyrano[3,4-d]pyridazin-1-amine;
4-(2-(difluoromethyl)-4-methoxyphenyl)-N-((3R,5R)-5-fluoro-1-methylpiperidin-3-yl)-7,8-dihydro-5H-pyrano[3,4-d]pyridazin-1-amine;
N-((3R,5R)-5-fluoro-1-methylpiperidin-3-yl)-4-(2-fluoro-4-methoxyphenyl)-7,8-dihydro-5H-pyrano[3,4-d]pyridazin-1-amine;
N-((3R,5R)-5-fluoro-1-methylpiperidin-3-yl)-4-(4-methoxy-2-(trifluoromethyl)phenyl)-7,8-dihydro-5H-pyrano[3,4-d]pyridazin-1-amine;
4-(4-chloro-2-fluorophenyl)-N-((3R,5R)-5-fluoro-1-methylpiperidin-3-yl)-7,8-dihydro-5H-pyrano[3,4-d]pyridazin-1-amine;
5-chloro-2-(4-(((3R,5R)-5-fluoro-1-methylpiperidin-3-yl)amino)-7,8-dihydro-5H-pyrano[3,4-d]pyridazin-1-yl)phenol;
2-(4-(((3R,5R)-5-fluoro-1-methylpiperidin-3-yl)amino)-7,8-dihydro-5H-pyrano[3,4-d]pyridazin-1-yl)phenol;
N-((3R,5R)-5-fluoro-1-methylpiperidin-3-yl)-4-(2-fluoro-4-(trifluoromethoxy)phenyl)-7,8-dihydro-5H-pyrano[3,4-d]pyridazin-1-amine;
N-((3R,5R)-5-fluoro-1-methylpiperidin-3-yl)-1-(2-fluoro-4-methoxyphenyl)-7,8-dihydro-5H-pyrano[3,4-d]pyridazin-4-amine;
N-((3R,5R)-5-fluoro-1-methylpiperidin-3-yl)-1-(4-methoxy-2-(trifluoromethyl)phenyl)-7,8-dihydro-5H-pyrano[3,4-d]pyridazin-4-amine;
1-(4-chloro-2-fluorophenyl)-N-((3R,5R)-5-fluoro-1-methylpiperidin-3-yl)-7,8-dihydro-5H-pyrano[3,4-d]pyridazin-4-amine;
(R)-5-methyl-2-(4-((1-methylpiperidin-3-yl)amino)-7,8-dihydro-5H-pyrano[3,4-d]pyridazin-1-yl)phenol;
5-fluoro-2-(4-(((3R,5R)-5-fluoro-1-methylpiperidin-3-yl)amino)-7,8-dihydro-5H-pyrano[3,4-d]pyridazin-1-yl)phenol;
2-(4-(((3R,5R)-5-fluoro-1-methylpiperidin-3-yl)amino)-7,8-dihydro-5H-pyrano[3,4-d]pyridazin-1-yl)-5-methylphenol;
(R)-5-methoxy-2-(4-((1-methylpiperidin-3-yl)amino)-7,8-dihydro-5H-pyrano[3,4-d]pyridazin-1-yl)phenol;
(R)-1-(4-methoxyphenyl)-N-(1-methylpiperidin-3-yl)-7,8-dihydro-5H-pyrano[3,4-d]pyridazin-4-amine;
(R)-4-(4-methoxyphenyl)-N-(1-methylpiperidin-3-yl)-7,8-dihydro-5H-pyrano[3,4-d]pyridazin-1-amine;
(R)-5-chloro-2-(4-((1-methylpiperidin-3-yl)amino)-7,8-dihydro-5H-pyrano[3,4-d]pyridazin-1-yl)phenol;
(R)-2-(4-((1-methylpiperidin-3-yl)amino)-7,8-dihydro-5H-pyrano[3,4-d]pyridazin-1-yl)phenol;
(R)-2-(1-((1-methylpiperidin-3-yl)amino)-7,8-dihydro-5H-pyrano[3,4-d]pyridazin-4-yl)phenol;
(R)—N-(1-cyclopropylpiperidin-3-yl)-1-(4-methoxyphenyl)-7,8-dihydro-5H-pyrano[3,4-d]pyridazin-4-amine;
2-(4-(((1R,2R)-2-hydroxycyclohexyl)amino)-7,8-dihydro-5H-pyrano[3,4-d]pyridazin-1-yl)-5-methylphenol;
2-(1-(((1R,2R)-2-hydroxycyclohexyl)amino)-7,8-dihydro-5H-pyrano[3,4-d]pyridazin-4-yl)-5-methylphenol;
4-((1-(2-hydroxy-4-methylphenyl)-7,8-dihydro-5H-pyrano[3,4-d]pyridazin-4-yl)amino)bicyclo[2.2.1]heptan-1-ol;
4-((4-(2-hydroxy-4-methylphenyl)-7,8-dihydro-5H-pyrano[3,4-d]pyridazin-1-yl)amino)bicyclo[2.2.1]heptan-1-ol;
(R)-2-(4-((1-(2-methoxyethyl)piperidin-3-yl)amino)-7,8-dihydro-5H-pyrano[3,4-d]pyridazin-1-yl)-5-methylphenol;
2-(4-(((3R,5R)-5-fluoro-1-methylpiperidin-3-yl)amino)-7,8-dihydro-5H-pyrano[3,4-d]pyridazin-1-yl)-5-(trifluoromethyl)phenol;
2-(4-(((3R,5S)-5-fluoro-1-methylpiperidin-3-yl)amino)-7,8-dihydro-5H-pyrano[3,4-d]pyridazin-1-yl)-5-methylphenol;
2-(4-(((3R,5R)-5-fluoro-1-methylpiperidin-3-yl)amino)-7,8-dihydro-5H-pyrano[3,4-d]pyridazin-1-yl)-5-(trifluoromethoxy)phenol;
2-(4-(((3R,5R)-5-fluoro-1-methylpiperidin-3-yl)amino)-7,8-dihydro-5H-pyrano[3,4-d]pyridazin-1-yl)-5-methoxyphenol;
5-cyclopropyl-2-(4-(((3R,5R)-5-fluoro-1-methylpiperidin-3-yl)amino)-7,8-dihydro-5H-pyrano[3,4-d]pyridazin-1-yl)phenol;
5-(difluoromethyl)-2-(4-(((3R,5R)-5-fluoro-1-methylpiperidin-3-yl)amino)-7,8-dihydro-5H-pyrano[3,4-d]pyridazin-1-yl)phenol; and
a pharmaceutically acceptable salt of any one of the aforementioned compounds.

26. A compound or pharmaceutically acceptable salt as defined in any one of claims 1 to 25 for use as a medicament.

27. A pharmaceutical composition comprising:
a compound or pharmaceutically acceptable salt as defined in any one of claims 1 to 25; and
a pharmaceutically acceptable excipient.

28. A compound or pharmaceutically acceptable salt as defined in any one of claims 1 to 25 for use in treating a disease, disorder or condition associated with NLRP3.

29. A compound or pharmaceutically acceptable salt as defined in any one of claims 1 to 25 for use in treating a disease, disorder or condition associated with a heterozygous gain of function mutation in the NLRP3 gene.

30. A compound or pharmaceutically acceptable salt as defined in any one of claims 1 to 25 for use in treating a cryopyrin-associated periodic syndrome (CAPS).

31. A method of treating a disease, disorder or condition in a subject, the method comprising administering to the subject a compound or pharmaceutically acceptable salt as defined in any one of claims 1 to 25, wherein the disease, disorder or condition is associated with NLRP3.

32. A method of treating a disease, disorder or condition in a subject, the method comprising administering to the subject a compound or pharmaceutically acceptable salt as defined in any one of claims 1 to 25, wherein the disease, disorder or condition is associated with a heterozygous gain of function mutation in the NLRP3 gene.

33. A method of treating a disease, disorder or condition in a subject, the method comprising administering to the subject a compound or pharmaceutically acceptable salt as defined in any one of claims 1 to 25, wherein the disease, disorder or condition is cryopyrin-associated periodic syndrome (CAPS).

34. The method according to claim 33, wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).

35. A method of treating a neurodegenerative disease, disorder or condition in a subject, the method comprising administering to the subject a compound or pharmaceutically acceptable salt as defined in any one of claims 1 to 25.

36. A method of treating a disease, disorder or condition in a subject, the method comprising administering to the subject a compound or pharmaceutically acceptable salt as defined in any one of claims 1 to 25, wherein the disease, disorder or condition is selected from Parkinson's disease, Alzheimer's disease, Huntington's disease, amyotrophic lateral sclerosis and prion disease.

37. A combination comprising a compound or pharmaceutically acceptable salt as defined in any one of claims 1 to 25, and at least one additional pharmacologically active agent.

38. The combination according to claim 37, wherein the additional pharmacologically active agent is selected from beta-secretase inhibitors, gamma-secretase inhibitors, HMG-CoA reductase inhibitors, nonsteroidal anti-inflammatory drugs, vitamin E, anti-amyloid antibodies, antidepressants, antipsychotics, anxiolytics, and anticonvulsants.
